(19) United States
(12) Patent Application Publication
(10) Pub. No.: US 2025/0092092 A1
(43) Pub. Date: Mar. 20, 2025

(54) ANTIBODY/DRUG CONJUGATES AND METHODS OF USE

(71) Applicant: A28 THERAPEUTICS INC., NORWALK, CT (US)

(72) Inventors: Carola LEUSCHNER, Houston, TX (US); Hector ALILA, Katy, TX (US)

(21) Appl. No.: 18/629,543

(22) Filed: Apr. 8, 2024

Related U.S. Application Data
(63) Continuation of application No. 17/503,127, filed on Oct. 15, 2021, now abandoned.
Application No. 17/503,127 is a continuation of application No. 16/246,189, filed on Jan. 11, 2019, now abandoned.
Application No. 16/246,189 is a continuation of application No. 15/350,924, filed on Nov. 14, 2016, now Pat. No. 10,233,214.
Application No. 15/350,924 is a continuation of application No. 14/067,819, filed on Oct. 30, 2013, now Pat. No. 9,492,563.
(60) Provisional application No. 61/720,257, filed on Oct. 30, 2012.

(51) Int. Cl. C07K 7/08; A61K 38/00; A61K 47/68; C07K 16/28; C07K 16/30; C07K 16/32

(52) U.S. Cl. CPC C07K 7/08; A61K 47/6817; A61K 47/6849; C07K 16/2887; C07K 16/3015; C07K 16/3069; C07K 16/32; A61K 38/00; C07K 2317/524; C07K 2317/526; C07K 2317/622; C07K 2319/74

(57) ABSTRACT

The invention relates to conjugates that bind to targets, methods of using conjugates that bind to targets and methods of treating undesirable or aberrant cell proliferation or hyperproliferative disorders, such as tumors, cancers, neoplasia and malignancies that express a target.

### SEQUENCE LISTING

The instant application contains a Sequence Listing which has been submitted electronically in XML format and is hereby incorporated by reference in its entirety. Said XML copy, created on Dec. 10, 2024, is named 20241210_5334-0004CON3_TPO_Sequence Listing and is 243 KB in size.

### TECHNICAL FIELD

The invention relates to antibodies, and Heavy (H) chains and/or Light (L) chains of antibodies, and antibody fragments fused or conjugated to drugs, such as lytic peptide conjugates, methods of using conjugates, for example, in methods of treating undesirable or aberrant cell proliferation or hyperproliferative disorders, such as non-metastatic and metastatic neoplasias, cancers, tumors and malignancies that express targets that bind to such antibodies, and Heavy (H) chains and/or Light (L) chains of antibodies.

### INTRODUCTION

The need to develop new therapeutics for treatment of primary tumors and metastases is clearly evident when the five year survival rate of cancer patients is considered: Only 10-40% for patients with lung, colorectal, breast and prostate cancer survive if diagnosed with distant metastatic disease.

### SUMMARY

The invention is based, at least in part on lytic domains fused to an antibody, lytic domains fused or conjugated to Heavy (H) chains and/or Light (L) chains of antibodies, and lytic domains fused or conjugated to antibody fragments, that bind to a target (e.g., Her2/neu, Human Epidermal growth factor Receptor 2, also known as ErbB-2, CD20). Such fusions can also be referred to herein as antibody or polypeptide conjugates or fusion constructs. Contact of a cell with a lytic domain is believed to cause disruption of the cell membrane which results in cell death. The antibody, or Heavy (H) chain and/or Light (L) chain of an antibody that binds to the target allows the lytic domain to target expressing cells for destruction, including undesirable or aberrant proliferating cells or hyperproliferating cells, such as non-metastatic and metastatic neoplasias, cancers, tumors and malignancies, that express the target to which the antibody or Heavy (H) chain and/or Light (L) chain binds. A number of non-metastatic and metastatic neoplastic, cancer, tumor and malignant cells overexpress targets, such as receptors or ligands. For example, many non-metastatic and metastatic neoplasias, cancers, tumors and malignancies, express a receptor target (e.g., Her2/neu or CD20) that can be used as a target of the antibody or polypeptide conjugate or fusion construct.

Conjugates can be designed to bind to any cell or cell population that expresses a target of interest. An antibody, fragment thereof, or Heavy (H) chain and/or Light (L) chain of an antibody or fragment thereof, selected based upon the target to which it binds, can be linked to a lytic domain. The resulting antibody or polypeptide conjugate or fusion construct can in turn reduce or inhibit proliferation of cells that express or target, thereby reducing or inhibiting proliferation or growth of the target expressing cells.

Conjugates do not require cells to divide in order to kill the target cells. Accordingly, conjugates are useful against dividing and non-dividing cells.

In accordance with the invention, there are provided antibody and polypeptide conjugates that include a lytic domain. In one embodiment, an antibody conjugate includes or consists of an antibody (or fragment thereof) that binds to a target, linked to a lytic domain that includes or consists of an L- or D-amino acid sequence that includes a peptide sequence selected from for example, KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAKKFAKFAK (SEQ ID NOs.: 1-7), or an L- or D-amino acid sequence that includes a peptide selected from KFAKFAKKFAKFAKK, KFAKFAKKFAKFAKKF, KFAKFAKKFAKFAKKFA, KFAKFAKKFAKFAKKFAK, KFAKFAKKFAKFAKKFAKF and KFAKFAKKFAKFAKKFAKFA (SEQ ID NOs.: 1-6) having one or more of the K residues substituted with any of an F or L residue, one or more of the F residues substituted with any of a K, A or L residue, or one or more of the A residues substituted with any of a K, F or L residue. In another embodiment, a polypeptide conjugate includes or consists of a Heavy (H) chain and/or Light (L) chain of an antibody or fragment thereof that binds to a target, linked to a lytic domain that includes or consists of an L- or D-amino acid sequence selected from for example, KFAKFAKKFAKFAKK. KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAKKFAKFAK (SEQ ID NOs.: 1-7), or a sequence that includes a peptide selected from KFAKFAKKFAKFAKK, KFAKFAKKFAKFAKKF, KFAKFAKKFAKFAKKFA, KFAKFAKKFAKFAKKFAK, KFAKFAKKFAKFAKKFAKF and KFAKFAKKFAKFAKKFAKFA (SEQ ID NOs.: 1-6) having one or more of the K residues substituted with any of an F or L residue, one or more of the F residues substituted with any of a K, A or L residue, or one or more of the A residues substituted with any of a K, F or L residue.

In a more particular embodiment, an antibody or polypeptide conjugate includes an antibody (or fragment thereof) or Heavy (H) chain and/or Light (L) chain of an antibody (e.g., trastuzumab or pertuzumab) or fragment thereof (trastuzumab or pertuzumab fragment) that binds to Her2/neu, linked to a lytic domain that includes or consists of an L- or D-amino acid sequence selected from, KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAKKFAKFAK (SEQ ID NOs.: 1-7), or a sequence that includes a peptide selected from KFAKFAKKFAKFAKK, KFAKFAKKFAKFAKKF, KFAKFAKKFAKFAKKFA, KFAKFAKKFAKFAKKFAK, KFAKFAKKFAKFAKKFAKF and KFAKFAKKFAKFAKKFAKFA (SEQ ID NOs.: 1-6) having one or more of the K residues substituted with any of an F or L residue, one or more of the F residues substituted with any of a K, A or L residue, or one or more of the A residues substituted with any of a K, F or L residue.

In accordance with the invention, there are also provided isolated and purified conjugates that include or consist of an antibody (or fragment thereof) or a Heavy (H) chain and/or Light (L) chain of an antibody or fragment thereof that binds to a target, and a second domain. In various embodiments, a second domain includes or consists of a lytic domain: KFAKFAKKFAKFAKK, KFAKFAKKFAKFAKKF, KFAKFAKKFAKFAKKFA, KFAKFAKKFAKFAKKFAK, KFAKFAKKFAKFAKKFAKF or KFAKFAKKFAKFAKKFAKFA (SEQ ID NOs.: 1-7). In additional embodiments, a second domain includes or consists of: KFAKFAKKFAKFAKK, KFAKFAKKFAKFAKKF, KFAKFAKKFAKFAKKFA, KFAKFAKKFAKFAKKFAK, KFAKFAKKFAKFAKKFAKF or KFAKFAKKFAKFAKKFAKFA (SEQ ID NOs.: 1-6) having one or more of the K residues substituted with any of an F or L residue, one or more of the F residues substituted with any of a K, A or L residue, or one or more of the A residues substituted with any of a K, For L residue.

In accordance with the invention, there are further provided polypeptides that include one or more lytic domains. In various embodiments, a polypeptide includes or consists of a: 1) lytic domain linked to the amino (NH₂)-terminus of an antibody Heavy (H) chain; 2) a lytic domain linked to the amino (NH₂)-terminus of an antibody Light (L) chain; 3) a lytic domain linked to a carboxy (C)-terminus of the antibody Heavy (H) chain; or 4) a lytic domain linked to a carboxy (C)-terminus of the antibody Light (L) chain. In additional various embodiments, a polypeptide includes or consists of a: 1) lytic domain linked to an amino (NH₂)-terminus and a lytic domain linked to a carboxy (C)-terminus of an antibody Heavy (H) chain; or 2) a lytic domain linked to the amino (NH₂)-terminus and a lytic domain linked to a carboxy (C)-terminus of an antibody Light (L) chain.

Specific non-limiting examples of targets include amino acid sequences (e.g., polypeptides, peptides, proteins), polysaccharides, oligosaccharides, carbohydrates, and lipids. Specific non-limiting classes of targets include receptors and antigens.

Targets include receptors that bind to antigens, receptors or ligands, including hormones, growth factors, cluster of differentiation (collectively known as CD molecules or CD markers), hormone and growth factor analogues, and fragments of hormones, hormone analogs, growth factors, growth factor analogues, and fragments of growth factors and analogues. Particular non-limiting examples of receptor targets include Her2/neu (Human Epidermal growth factor Receptor 2, also known as ErbB-2), luteinizing hormone releasing hormone receptor (LHRH-R), epidermal growth factor (EGF) receptor, folate, and growth hormone (GH) receptor. Particular non-limiting examples of CD domains include CD19, CD20, CD22, CD23, CD27, CD28, CD30, CD31, CD33, CD34, CD40, CD52, CD56, CD70, CD123, CD138, or CD154, and others.

Antigen targets include viral, bacterial, fungal and parasite antigens. Antigen targets also include tumor associated antigens (TAAs).

An antibody includes 2 Heavy (H) chains and 2 Light (L) chains as well as antibody fragments. A polypeptide that includes or consists of a Heavy (H) chain and/or Light (L) chain of an antibody or fragment of a Heavy (H) chain or Light (L) chain can include a single H or L chain or a single H or L chain fragment, or a plurality (2, 3, 4 or more) of Heavy (H) chains and/or Light (L) chains, or a plurality of fragments of Heavy (H) chains and/or Light (L) chains. A polyeptide that includes a Heavy (H) chain and/or Light (L) chain of an antibody or fragment can but is not required to include 2 Heavy (H) chains and 2 Light (L) chains and therefore polypeptide conjugates as set forth herein can exclude native antibodies that comprise 2 Heavy (H) chains and 2 Light (L) chains.

An antibody or fragment thereof may be an oligomeric (higher order or valent) forms, such as a trimer, tetramer, pentamer, hexamer, heptamer, and so forth, with other antibodies, fragments thereof, Heavy (H) chain, Light (L) chain, or polyeptides sequence distinct from an antibody Heavy (H) or Light (L) chain. Antibodies include monoclonals and fragments of monoclonal antibodies. Antibodies include mammalian, human, humanized, primatized and chimeric sequences.

Amino acid sequences (e.g., polypeptides, peptides, proteins, antibodies, Heavy (H) chains, Light (L) chains, lytic domains, etc.) include or consist of natural (L-) or non-natural (e.g., D-) amino acids. In particular aspects, an amino acid sequence has about 2 to 10, 10 to 14, 15 to 20, (i.e., 15, 16, 17, 18, 19 or 20 amino acids), 10 to 20, 20 to 30, 30 to 40, 40 to 50, 60 to 70, 70 to 80, 80 to 90, 90 to 100, 100 to 125, 125 to 150, 150 to 175, 175 to 200, 200 to 250, 250 to 300, or more amino acid residues. Full-length antibody Heavy (H) chains, Light (L) chains, are typically 90 to 130 amino acids in length, but may be shorter, for example, comprise a variable Heavy (H) chain, or Light (L) chain sequence, comprising one, two, or three complemetarity determining regions (CDRs) with or without framework regions. Lytic domains are typically 10 to 14, 15 to 20, (i.e., 15, 16, 17, 18, 19 or 20 amino acids), 10 to 20, 20 to 30, 30 to 40, or 40 to 50, but may optionally be longer (50 or more) or shorter (less than 10). An amino acid sequence can include or consist of a linear or cyclic structure.

Conjugates that include lytic domains can have the lytic domain at any location of the antibody (or fragment thereof) or Heavy (H) chain or Light (L) chain of an antibody. Thus, a lytic domain can be positioned at any amino acid position (amino acid residue) of the antibody (or fragment thereof) or Heavy (H) chain or Light (L) chain of an antibody. In addition, a conjugate can include multiple lytic domains. Accordingly, one or more (e.g., two, three, four, five, six, seven, eight or more) lytic domains linked to the Heavy (H) chain or Light (L) chain) lytic domains can be included in a conjugate of the invention.

Lytic domains can also be positioned at the C-terminus, the NH₂-terminus, or both the C-terminus and the NH₂-terminus of a polyeptide sequence. In particular embodiments, a conjugate has a lytic domain positioned at either (or both) the NH₂-terminus or the C-terminus of the antibody (or fragment thereof) Heavy (H) chain or Light (L) chain, or Heavy (H) chain or Light (L) chain. In particular aspects, a lytic domain is linked to the amino (NH₂)-terminus of the Heavy (H) chain or linked to the carboxy (C)-terminus of the Heavy (H) chain; a lytic domain is linked to the amino (NH₂)-terminus of the Light (L) chain or linked to the carboxy (C)-terminus of the Light (L) chain. In a conjugate with a plurality of lytic domains, such domains can be linked to the Heavy (H) chain or Light (L) chain, to the amino (NH₂)-terminus of the Heavy (H) chain, to the carboxy (C)-terminus of the Heavy (H) chain, to the amino (NH₂)-terminus of the Light (L) chain, or to the carboxy (C)-terminus of the Light (L) chain. In more particular aspects, at least one of a plurality of lytic domains is linked to the amino (NH₂)-terminus of the Heavy (H) chain, and at least one is linked to the amino (NH₂)-terminus of the Light (L) chain; at least one of the lytic domains is linked to the amino (NH₂)-terminus of the Heavy (H) chain, at least one of the lytic domains is linked to the amino (NH₂)-terminus of the Light (L) chain, and at least one of the lytic domains is linked to the carboxy (C)-terminus of the Heavy (H) chain or is linked to the carboxy (C)-terminus of the Light (L) chain; and at least one of a plurality of lytic domains is linked to the amino (NH₂)-terminus of the Heavy (H) chain, at least one of the lytic domains is linked to the amino (NH₂)-terminus of the Light (L) chain, at least one of the lytic domains is linked to the carboxy (C)-terminus of the Heavy (H) chain and at least one of the lytic domains is linked to the carboxy (C)-terminus of the Light (L) chain.

In embodiments in which conjugates include a plurality of lytic domains, the lytic domains have an identical amino acid sequence, or have a different amino acid sequence. Accordingly a conjugate can include two, a third, fourth, fifth, sixth, seventh lytic domain, etc., any or all of which may be identical or different from each other.

In particular embodiments, a lytic domain is joined to a Heavy (H) chain or Light (L) chain immediately after the last amino acid at the amino (NH₂)-terminus or the carboxy (C)-terminus of the Heavy (H) chain or the Light (L) chain, for example, by a covalent (e.g., peptide or nonpeptide) bond thereby forming a continuous amino acid sequence between the lytic domain and the Heavy (H) chain or Light (L) chain. In additional embodiments, antibodies, Heavy (H) chains and Light (L) chains and lytic domains can be joined by a peptide or a non-peptide linker or spacer. In particular aspects, antibodies, Heavy (H) chains and Light (L) chains and lytic domains and lytic domains are joined by a peptide sequence having from about 1 to 25 amino acid residues, or are joined by a linear carbon chain, such as CN (where N=1-100 carbon atoms, e.g., C, CC, CCC, CCCC, CCCCC, CCCCCC, CCCCCCC, CCCCCCCC, etc.). In more particular aspects, antibodies, Heavy (H) chains and Light (L) chains and lytic domains and lytic domains are joined by a peptide sequence that includes or consist of one or more A, S or G amino acid residues (e.g., a peptide sequence including or consisting of GSGGS (SEQ ID No.: 8), ASAAS (SEQ ID NO.: 9), GS, AF, FK, VK, FFK, FA, GSGRSA (SEQ ID NO.: 10), RVRRSV (SEQ ID NO.: 11), SS, Cit-V (Cit=Citrulline (H₂NC(O)NH(CH₂)₃CH(NH₂)CO₂H); Val=Valine), F-Cit (F=Phenylalanine, Cit=Citrulline).

Conjugates further include or consist of additional (e.g., non-lytic) domains. Thus, in various aspects, a conjugate includes a second, third, fourth, fifth, sixth, seventh domain, etc., which may be distinct from one or more (or all) lytic domains included in the conjugate.

Conjugates include or consist of isolated and/or purified forms. Conjugates also include or consist of a formulation or a mixture. Such formulations and mixtures include compositions, such as a mixture of conjugate and a pharmaceutically acceptable carrier or excipient appropriate for use, administration to or in vivo contact with a subject, or a mixture of conjugate and an anti-cell proliferative or immune stimulating agent.

Conjugates include or consist of a unit dosage form, such as a dosage form for use or administration to a subject. In one embodiment, a conjugate is a unit dosage to administer or in an amount effective to or treat a subject having undesirable cell proliferation or a hyperproliferative disorder. In another embodiment, a conjugate is a unit dosage to administer or in an amount effective to treat a subject having a neoplasia, tumor or cancer. In an additional embodiment, a conjugate is a unit dosage to administer or in an amount effective to reduce fertility of a subject.

Conjugates can be included within kits, optionally with instructions for practicing a method or use of the invention. In one embodiment, a kit includes a conjugate and instructions for reducing or inhibiting proliferation of a cell, reducing or inhibiting proliferation of a hyperproliferating cell, reducing or inhibiting proliferation of a neoplastic, tumor or cancer cell, treating a subject having a hyperproliferative disorder, treating a subject having a neoplasia, tumor or cancer, or reducing fertility of an animal.

There are also provided nucleic acids that encode conjugates. In various embodiments, a nucleic acid sequence encodes a: 1) lytic domain linked to the amino (NH₂)-terminus of the antibody Heavy (H) chain; 2) a lytic domain linked to the amino (NH₂)-terminus of the antibody Light (L) chain; 3) a lytic domain linked to the carboxy (C)-terminus of the antibody Heavy (H) chain; or 4) a lytic domain linked to the carboxy (C)-terminus of the antibody Light (L) chain, of an antibody or polypeptide conjugate. In another embodiment, a nucleic acid sequence encodes a: 1) lytic domain linked to the amino (NH₂)-terminus and a lytic domain linked to the carboxy (C)-terminus of the antibody Heavy (H) chain; 2) a lytic domain linked to the amino (NH₂)-terminus and a lytic domain linked to the carboxy (C)-terminus of the antibody Light (L) chain, of the antibody or polypeptide conjugate.

Nucleic acids can be included in a vector, such as an expression vector that when expressed in a cell encodes a conjugate. Host cells can be transformed with a nucleic acid (e.g., encoding all or a portion of a conjugate, e.g., a Heavy (H) chain and/or Light (L) chain sequence linked to a lytic domain) in a vector, such that the cell expresses a conjugate encoded by the nucleic acid.

As disclosed herein, targets can be expressed in or on a cell. Cells that express a target to which a conjugate binds can be targeted for binding by the conjugates of the invention. Accordingly, such cells can be selectively targeted by selecting a conjugate that binds to a target expressed by the cells.

Non-limiting target expressing cells include, for example, hyperproliferative cells. Additional cells that express targets include, for example, breast, ovarian, uterine, cervical, stomach, lung, gastric, colon, bladder, glial, dermal (e.g., melanocytes), hematologic and endometrial cells.

Conjugates are useful for, among other things, reducing or inhibiting proliferation of a cell, reducing or inhibiting cell proliferation, reducing or inhibiting proliferation of a hyperproliferating cell, reducing or inhibiting proliferation of a neoplastic, tumor, cancer or malignant cell and treating undesirable or aberrant cell proliferation, such as hyperproliferating cells or hyperproliferative disorders. Non-limiting examples of hyperproliferative disorders include benign hyperplasia, non-metastatic and metastatic neoplasias, cancers tumors and malignancies.

In accordance with the invention, there are further provided methods of reducing or inhibiting proliferation of a cell; methods of reducing or inhibiting cell proliferation; methods of reducing or inhibiting proliferation of a hyperproliferating cell; and methods of reducing or inhibiting proliferation of a neoplastic, tumor, cancer or malignant cell. In various embodiments, a method includes contacting a cell with a conjugate in an amount sufficient to reduce or inhibit proliferation of the cell; contacting a cell with a conjugate in an amount sufficient to reduce or inhibit cell proliferation; contacting a cell with a conjugate in an amount sufficient to reduce or inhibit proliferation of the hyperproliferating cell; and contacting a cell with a conjugate in an amount sufficient to reduce or inhibit proliferation of the neoplastic, tumor, cancer or malignant cell.

In accordance with the invention, there are moreover provided methods of selectively reducing or inhibiting proliferation of a cell that expresses a target to which a conjugate binds; selectively reducing or inhibiting proliferation of a hyperproliferating cell that express a target to which a conjugated binds; and selectively reducing or inhibiting proliferation of a neoplastic, tumor, cancer or malignant cell that expresses a target to which a conjugated binds. In various embodiments, a method includes contacting a target expressing cell with a conjugate in an amount sufficient to reduce or inhibit proliferation of the cell; contacting a target expressing cell with the conjugate in an amount sufficient to reduce or inhibit proliferation of the hyperproliferating cell; and contacting a target expressing cell with a conjugate in an amount sufficient to reduce or inhibit proliferation of the neoplastic, tumor, cancer or malignant cell, wherein the conjugate binds to a target expressed by the cell.

Exemplary cells to be targeted in accordance with the invention uses and methods include cells that express any desired target. Such non-limiting cells therefore include, for example, breast, ovarian, uterine, cervical, stomach, lung, gastric, colon, bladder, glial, dermal (e.g., melanocytes), hematologic and endometrial cells. More particular non-limiting cells express a receptor, such as Her2/neu, an antigen (e.g., a tumor associated antigen) or a cluster of differentiation domain (CD).

Methods performed include, among others, administering to or contacting a subject in need of inhibiting, reducing or preventing proliferation, survival, differentiation, death, or activity of a cell, such as a hyperprolifertive cell or an undesirably proliferating cell. Exemplary subjects include a subject having or at risk of having undesirable or aberrant cell proliferation; a subject having or at risk of having a benign hyperplasia; or a non-metastatic or metastatic neoplasia, cancer, tumor or malignancy (e.g., a solid or liquid tumor, in any of breast, ovarian, uterine, cervical, stomach, lung, gastric, colon, bladder, glial, dermal (e.g., melanocytes), hematologic or endometrial cells).

In accordance with the invention, there are additionally provided uses and methods of treating a subject having a hyperproliferative disorder and uses and methods of treating a subject having a neoplasia, tumor, cancer or malignancy (metastatic, non-metastatic or benign). In various embodiments, a use or method includes, administering to a subject an amount of the conjugate sufficient to treat the hyperproliferative disorder; and administering to a subject an amount of the conjugate sufficient to reduce or inhibit proliferation of the neoplasia, tumor, cancer or malignancy.

Methods and uses include treating a subject having or at risk of having a metastasis. For example, an amount of a conjugate effective to reduce or inhibit spread or dissemination of a tumor, cancer or neoplasia to other sites, locations or regions within the subject. In various embodiments, a method or use reduces or inhibits metastasis of a primary tumor or cancer to one or more other sites, formation or establishment of a metastasis at one or more other sites, locations or regions thereby reducing or inhibiting tumor or cancer relapse or tumor or cancer progression. In further embodiments, a method or use reduces or inhibits growth, proliferation, mobility or invasiveness of tumor or cancer cells that potentially or do develop metastases (e.g., disseminated tumor cells); reduces or inhibits formation or establishment of metastases arising from a primary tumor or cancer to one or more other sites, locations or regions distinct from the primary tumor or cancer; reduces or inhibits growth or proliferation of a metastasis at one or more other sites, locations or regions distinct from the primary tumor or cancer after the metastasis has formed or has been established; or reduces or inhibits formation or establishment of additional metastasis after the metastasis has been formed or established. In yet another embodiment, a method or use reduces or inhibits relapse or progression of the neoplasia, tumor, cancer or malignancy.

In accordance with the invention, there are still further provided methods and uses of reducing or inhibiting metastasis of a neoplasia, tumor, cancer or malignancy to other sites, or formation or establishment of metastatic neoplasia, tumor, cancer or malignancy at other sites distal from a primary neoplasia, tumor, cancer or malignancy. In various embodiments, a method includes administering to a subject an amount of the conjugate sufficient to reduce or inhibit metastasis of the neoplasia, tumor, cancer or malignancy to other sites, or formation or establishment of metastatic neoplasia, tumor, cancer or malignancy at other sites distal from the primary neoplasia, tumor, cancer or malignancy.

Neoplasia, tumor, cancer and malignancy treatable in accordance with the invention therefore include metastatic, and non-metastatic or benign forms. Non-limiting examples include a solid cellular mass, hematopoietic cells, or a carcinoma, sarcoma (e.g. lymphosarcoma, liposarcoma, osteosarcoma, chondrosarcoma, leiomyosarcoma, rhabdomyosarcoma or fibrosarcoma), lymphoma, leukemia, adenoma, adenocarcinoma, melanoma, glioma, glioblastoma, meningioma, neuroblastoma, retinoblastoma, astrocytoma, oligodendrocytoma, mesothelioma, reticuloendothelial, lymphatic or haematopoietic (e.g., myeloma, lymphoma or leukemia) neoplasia, tumor, cancer or malignancy.

Neoplasia, tumor, cancer and malignancy treatable in accordance with the invention can be present in or affect a lung (small cell lung or non-small cell lung cancer), thyroid, head or neck, nasopharynx, throat, nose or sinuses, brain, spine, adrenal gland, pituitary gland, breast, ovarian, uterine, cervical, gastrointestinal (mouth, esophagus, stomach, duodenum, ileum, jejunum (small intestine), colon, rectum), lung, genito-urinary tract (uterus, ovary, cervix, endometrial, bladder, testicle, penis, prostate), glial, hematologic, endometrial, lymph, blood, muscle, dermal (e.g., melanocytes) or skin cell, kidney, pancreas, liver, bone, bone marrow, Wilm's tumors, biliary tract, B-ALL (B-cell lymphoblastic leukemia), stem cell, or hematologic neoplasia, tumor, cancer, or malignancy.

Methods and uses may be practiced alone, for example, in subjects that are not candidates for other therpaies (e.g., surgical resection, chemotherapy, immunotherapy, radiotherapy, thermal therapry, vaccination, etc.). Methods and uses may also be practiced with other treatments or therapies (e.g., surgical resection, radiotherapy, ionizing or chemical radiation therapy, chemotherapy, immunotherapy, local or regional thermal (hyperthermia) therapy, or vaccination). Such treatments or therapies can be administered prior to, substantially contemporaneously with (separately or in a mixture), or following administration of a conjugate. In one embodiment, a method or use includes administering an anti-cell proliferative, anti-neoplastic, anti-tumor, anti-cancer or immune-enhancing treatment or therapy. In further embodiments, a method or use includes administering an alkylating agent, anti-metabolite, plant extract, plant alkaloid, nitrosourea, hormone, nucleoside or nucleotide analog; cyclophosphamide, azathioprine, cyclosporin A, prednisolone, melphalan, chlorambucil, mechlorethamine, busulphan, methotrexate, 6-mercaptopurine, thioguanine, 5-fluorouracil, cytosine arabinoside, 5-azacytidine (5-AZC) and 5-azacytidine related compounds, bleomycin, actinomycin D, mithramycin, mitomycin C, carmustine, lomustine, semustine, streptozotocin, hydroxyurea, cisplatin, carboplatin, oxiplatin, mitotane, procarbazine, dacarbazine, a taxane (e.g., taxol or paclitaxel), vinblastine, vincristine, doxorubicin or dibromomannitol, topoisomerase inhibitors, (irinotecan, topotecan, etoposide, teniposide), gemcitabine, pemetrexed etc.

Cell or immunotherapies include lymphocytes, plasma cells, macrophages, dendritic cells, T-cells, NK cells or B-cells; an antibody, a cell growth factor, a cell survival factor, a cell differentiative factor, a cytokine or a chemokine. Additional agents that are applicable with conjugates in compostions, methods or uses of the invention include targeted drugs or biologicals, such as antibodies (monoclonal) or small molecules.

Methods of the invention include providing a subject with a benefit. In particular embodiments, a method of treatment results in partial or complete destruction of the neoplastic, tumor, cancer or malignant cell mass, volume, size or numbers of cells, stimulating, inducing or increasing neoplastic, tumor, cancer or malignant cell necrosis, lysis or apoptosis, reducing neoplasia, tumor, cancer or malignancy volume size, cell mass, inhibiting or preventing progression or an increase in neoplasia, tumor, cancer or malignancy volume, mass, size or cell numbers, or prolonging lifespan; results in reducing or decreasing severity, duration or frequency of an adverse symptom or complication associated with or caused by the neoplasia, tumor, cancer or malignancy; results in reducing or decreasing pain, discomfort, nausea, weakness or lethargy; or results in increased energy, appetite, improved mobility or psychological well being.

Subjects treatable in accordance with the methods include mammals. In particular embodiments, a subject is a human.

### DESCRIPTION OF DRAWINGS

FIGS. 1A and 1B show cytotoxicity of recombinantl scFv-C_{H}3 (naked antibody), scFv-C_{H}3-GS-Phor18 and scFv-C_{H}3-GS-(KLAKLAK)₂KLAK (SEQ ID NO.: 74) to Her2-neu receptor positive FIG. 1A) breast (SKBR-3); and FIG. 1B) ovarian (SKOV-3) cancer cell lines determined after 48 hours.

FIGS. 2A and 2B show median tumor volumes from mice treated with naked monoclonal antibody (MAb), FIG. 2A) MAb-Phor18 and FIG. 2B) recombinant scFv-C_{H}3 naked antibody and scFv-C_{H}3-Phor18 conjugates during the study period of 64 days in SKOV-3 xenografted mice in comparison with saline injected mice.

FIG. 3 shows mean tumor weights from mice treated with MAb (naked), MAb-Phor18 and recombinantly scFv-C_{H}3 naked antibody and scFv-C_{H}3-Phor18 conjugates on day 64 in SKOV-3 xenografted mice in comparison with saline injected mice.

FIG. 4 shows mean body weights from mice treated with MAb (naked), MAb-Phor18 and recombinantly scFv-C_{H}3 naked antibody and scFv-C_{H}3-Phor18 conjugates during the study period of 64 days in SKOV-3 xenografted mice in comparison with saline injected mice.

FIG. 5 shows the pCMVTnT expression vector (Promega) used for ADC heavy (H) and light (L) chain expression.

FIG. 6 shows an anti-Phor18 immunoblot of ADCs produced: H1L1 (IgG1), H3L1 (C_{H}3-Phor18-IgG1), H1L3 (C_{L}-Phor18-IgG1), H2L2 (Phor18-V_{L}-Phor18-V_{H}-IgG1) and H1L2 (Phor18-V_{L}-Phor18-IgG).

FIGS. 7A and 7B show a quality analysis of ADCs produced: anti Her2-antibody, H1L1 (naked), H1L3 (C_{L}-Phor18-IgG1) and H3L1 (C_{H}3-Phor18-IgG1) using FIG. 7A) and anti-Phor18 probe; and FIG. 7B) anti-kappa light (L) chain probe, of reduced proteins.

FIG. 8 shows anti IgG and anti-Phor18 Immunoblot of ADCs produced: H1L3 (C_{L}-Phor18-IgG1), H1L2 (Phor18-V_{L}-Phor18-IgG1 and H2L2 (Phor18-V_{L}-Phor18-V_{H}-IgG1).

FIG. 9 shows effect of whole antibody CD20 and antibody CD20-Phor18 conjugates-chemically linked-on Caspase 3/7 activities in Daudi cells.

FIGS. 10A and 10B show in vitro activity of recombinantly produced scFv-Phor18 compared to naked scFv in CD20 expressing Daudi cells. FIG. 10A) Cell membrane integrity was determined after 2 h, and FIG. 10B) cell viability was determined after 24 h of incubation with each naked and conjugated AB. Naked scFv did not disintegrate cell membranes or killed the target cells, whereas scFv-Phor18 conjugates showed membrane disintegration and cell killing.

FIG. 11 shows in vitro caspase 3/7 activation measured for naked scFv and scFv-Phor18 conjugates. scFv-Phor 18 increased caspase 3/7 activities in a concentration dependent manner. Staurosporine served as control for caspase activation. Naked scFv did not detectably activate caspases.

FIG. 12 illustrates expression vector for anti-CD20 ADC and naked antibody expression used for transfection in a yeast system.

FIG. 13A-13D show in vitro activities of scFvFc and Phor18-V_{L}-scFvFc-C_{H}3-Phor18 in CD20 positive Daudi cell lines after FIG. 13A) 4 and FIG. 13B) 24 hours, and in CD20 negative U937 cells after FIG. 13C) 4 and FIG. 13D) 24 hours.

FIG. 14 illustrates expression vector for anti-CD20 ADC and naked antibody expression.

### DETAILED DESCRIPTION

The invention is based at least in part on a conjugate that includes a portion that binds to a target joined or fused to a second lytic domain. In a typical configuration, a conjugate includes a first target binding domain (e.g., an antibody, or a Heavy (H) chain and/or Light (L) chain of an antibody) and a second domain that includes a lytic portion, which is/are directly or indirectly toxic to a cell, which can thereby reduce cell proliferation or survival, or stimulate, induce, increase or enhance cell death, killing or apoptosis.

In accordance with the invention, there are provided conjugates that include or consist of an antibody, or a Heavy (H) chain and/or Light (L) chain of an antibody, that bind to a target, and a second lytic or toxic domain. In one embodiment, a conjugate includes an antibody or fragment thereof and a lytic domain comprising or consisting of a 10-100 residue L- or D-amino acid sequence that includes a peptide sequence (selected from amino acids such as Lysine=K, Phenylalanine=F and Alanine=A), for example, KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAKKFAKFAK (SEQ ID NOs.: 1-7). In another embodiment, a conjugate includes a Heavy (H) chain and/or Light (L) chain of an antibody and a lytic domain comprising or consisting of a 10-100 residue L- or D-amino acid sequence selected from KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAKKFAKFAK (SEQ ID NOs.: 1-7).

As used herein, the term “conjugate” or “fusion construct” and grammatical variations thereof, means that the construct contains portions or sections that are derived from, obtained or isolated from, or are based upon or modeled after two different molecular entities that are distinct from each other and do not typically exist together in nature. That is, for example, a first portion of the conjugate includes or consists of an antibody or antibody fragment, or a Heavy (H) chain and/or Light (L) chain of an antibody, and a second portion of the conjugate includes or consists of a lytic portion or domain, each of the first and second portions/domains structurally distinct. A conjugate can also be referred to as a “fusion construct,” wherein the conjugate includes or consists of a of an antibody or antibody fragment, or a Heavy (H) chain and/or Light (L) chain of an antibody, that bind to a target, and a second lytic domain or portion.

First domains and or second (lytic) domains of conjugates include or consist of amino acid sequences (peptides, polypeptides, proteins, lectins), nucleic acids (DNA, RNA) and carbohydrates (saccharides, sialic acid, galactose, mannose, fucose, acetylneuraminic acid, etc.). The terms “amino acid sequence,” “protein,” “polypeptide” and “peptide” are used interchangeably herein to refer to two or more amino acids, or “residues,” covalently linked by an amide bond or equivalent. Amino acid sequences can be linked by non-natural and non-amide chemical bonds including, for example, those formed with glutaraldehyde, N-hydroxysuccinimide esters, bifunctional maleimides, or N,N′-dicyclohexylcarbodiimide (DCC). Non-amide bonds include, for example, ketomethylene, aminomethylene, olefin, ether, thioether and the like (see, e.g., Spatola in Chemistry and Biochemistry of Amino Acids, Peptides and Proteins, Vol. 7, pp 267-357 (1983), “Peptide and Backbone Modifications,” Marcel Decker, NY).

First and second (lytic) domains of a conjugate or fusion construct include L-amino acid sequences, D-amino acid sequences and amino acid sequences with mixtures of L-amino acids and D-amino acids. Conjugates of amino acid sequences of first and second domains can be a linear or a cyclic structure, and can be further conjugated to another distinct moiety (e.g., third, fourth, fifth, sixth, seventh, etc. domains), form intra or intermolecular disulfide bonds, and also form higher order multimers or oligomers (trimers, tetramers, pentamers, hexamers, heptamers, etc.) with other conjugates having the same or different antibody, Heavy (H) chain, Light (L) chain or lytic sequence, or with other entirely distinct molecules.

Exemplary lengths of conjugates are from about 10 to 15, 15 to 20, 20 to 25, 25 to 50, 50 to 100, 100 to 150, 150 to 200, or 200 to 300 or more amino acid residues in length. In particular embodiments, a first or second domain includes or consists of an amino acid sequence of about 1 to 10, 10 to 20, 15 to 20, 20 to 30, 30 to 40, 40 to 50, 60 to 70, 70 to 80, 80 to 90, 90 to 100 or more residues. In more particular embodiments, a lytic domain includes or consists of a 10, 11, 12, 13, 14, 15, 16, 17, 18, 19, 20, 21, 22, 23, 24, 25, 26, 27, 28, or more residue amino acid sequence, or a 10, 11, 12, 13, 15, 16, 17, 18, 19, 20, 22, 23, 24, 25, 26, 27, 28, or more residue amino acid sequence.

Conjugate that includes or consists of a first portion antibody or antibody fragment, or a Heavy (H) chain and/or Light (L) chain of an antibody, where the first portion binds to a target (e.g., receptor), and a second portion that includes or consists of a lytic domain, the lytic domain can form an amphipathic alpha-helix. An amphipathic alpha-helix contains mostly hydrophilic amino acids on one side of the alpha-helix and the other side contains mostly hydrophobic amino acids. Since the alpha helix makes a complete turn for every 3.6 residues, the amino acid sequence of an amphipathic alpha helix alternates between hydrophilic and hydrophobic residues every 3 to 4 residues. A PNNPNNP repeat pattern or motif is predicted to form an amphipathic alpha-helix where P represents a positively charged amino acid residue and N a neutral amino acid residue. A PNNPNNP repeat pattern provides a cationic binding site for the lytic peptide to interact with a negatively charged cell membrane and a hydrophobic site for membrane interaction/penetration. Conjugates therefore include that with lytic domains having one or more uninterrupted PNNPNNP repeat patterns or motifs, or one or more interrupted PNNPNNP repeat patterns or motifs, which can form an amphipathic alpha-helix. For example, a 15 or 18 residue amino acid sequence, such as KFAKFAKKFAKFAKK (SEQ ID NO.: 1) and KFAKFAKKFAKFAKKFAK (SEQ ID NO.: 4), has uninterrupted and interrupted PNNPNNP repeat motifs.

As disclosed herein, conjugates include antibodies and antibody fragments that bind to a target. An “antibody” refers to any monoclonal or polyclonal immunoglobulin molecule, such as IgM, IgG, IgA, IgE, IgD, and any subclass thereof. Exemplary subclasses for IgG are IgG₁, IgG₂, IgG₃ and IgG₄ including engineered antibody subclasses and recombinant antibodies with various glycosylation patterns.

Conjugates also include a Heavy (H) chain or a Light (L) chain of an antibody, or a fragment of a Heavy (H) chain or a Light (L) chain of an antibody, that bind to a target. Such conjugates can have a plurality of Heavy (H) chains and/or Light (L) chains of an antibody, or a fragment of Heavy (H) chains and/or Light (L) chains of an antibody, that bind to a target.

Antibody fragments, and fragments of Heavy (H) chains and/or Light (L) chains of an antibody, include the hypervariable (target binding) region, or any or all of the complementarity determining regions (CDRs) or framework regions (FRs) within a Heavy (H) chain and/or Light (L) chain of an antibody, sufficient to confer target binding. Specific non-limiting examples of antibody fragments include Fab, Fab′, F(ab′)₂, Fv, Fd, single-chain Fv (scFv), disulfide-linked Fvs (sdFv), V_{L}, V_{H}, Camel Ig, V-NAR, VHH, trispecific (Fab₃), bispecific (Fab₂), diabody ((V_{L}-V_{H})₂ or (V_{H}-V_{L})₂), triabody (trivalent), tetrabody (tetravalent), minibody ((scFv-C_{H}3)₂), bispecific single-chain Fv (Bis-scFv), IgGdeltaCH2, scFv-Fc, (scFv)₂-Fc, affibody (e.g., ZHer2-neu: 2, ZHer2-neu: 4 ZHer2-neu: 7 ZHer2-neu: 8), aptamer, avimer or nanobody.

Antibodies, Heavy (H) chains and Light (L) chains of an antibody, and fragments thereof, include those produced by or expressed on cells, such as B cells, or synthesized or engineered to be produced by other cells, e.g., CHO cells. Such antibodies, Heavy (H) chains and Light (L) chains of an antibody, and fragments thereof, include those with improved characteristics, such as increased serum stability and/or half life in vivo, PK, etc. (e.g., as described in Antibody Engineering Vol 1, Konterman R and Duebel S, eds., 2010, Springer, WO 2006/130834 and Horton et al., Cancer Res 68:8049 (2008)). Non-limiting mutations in the Fc include, for example, I253A, H310A, H435 R, H435Q, G236R, L328 R, S239D, 1332E. Non-limiting mutations in IgG1can be at residues 238, 252, 253, 254, 255, 256, 265, 272, 289, 288, 303, 305, 307, 309, 311, 312, 317, 340, 356, 360, 362, 376, 378, 380, 382, 386, 388, 400, 413, 415, 424, 433, 434, 435, 439 and/or 477 of the Fc region.

Antibodies, Heavy (H) chains and Light (L) chains of an antibody, and fragments thereof bind to a target. Specific non-limiting examples of targets include amino acid sequences (e.g., polypeptides, peptides, proteins), polysaccharides, oligosaccharides, carbohydrates, and lipids. Specific non-limiting classes of targets include receptors and antigens. Such targets can be expressed by or on a cell (e.g., on the cell membrane).

Targets include receptors that bind to hormones, growth factors, hormone and growth factor analogues, and fragments of hormones, hormone analogs, growth factors, growth factor analogues, and fragments of growth factors and analogues. Particular non-limiting examples of receptor targets include Her2/neu (Human Epidermal growth factor Receptor 2, also known as ErbB-2), luteinizing hormone releasing hormone receptor (LHRH-R), epidermal growth factor receptor (EGF-R), folate-, and growth hormone (GH) receptor. Further particular non-limiting examples of receptor targets include a tumor necrosis factor (TNF) family member receptor (e.g., TNF-alpha, TNF-beta (lymphtoxin, LT), TRAIL, Fas, LIGHT, or 4-1BB) or oncofetoprotein (5T4). Additional particular non-limiting examples of receptor targets include an immunoglobulin-like receptor (e.g., CD19, CD20, CD22, CD23, CD27, CD28, CD30, CD31, CD33, CD34, CD40, CD52, CD56, CD70, CD123, CD138, CD123, CD138, or CD154), or other receptors, (e.g., hormone receptor, a cytokine receptor, a growth factor receptor, or a chemokine receptor).

Antigen targets include viral, bacterial, fungal and parasite antigens. Antigen targets also include tumor associated antigens (TAAs). Particular non-limiting examples of TAA targets include carcinoembryonic antigen (CEA), alpha-fetoprotein (AFP), prostate specific antigen (PSA), prostate specific membrane antigen (PSMA), CA-125 (epithelial ovarian cancer), soluble Interleukin-2 (IL-2) receptor, RAGE-1, tyrosinase, MAGE-1, MAGE-2, NY-ESO-1, Melan-A/MART-1, glycoprotein (gp) 75, gp100, beta-catenin, PRAME, MUM-1, ZFP161, Ubiquilin-1, HOX-B6, YB-1, Osteonectin, ILF3, IGF-1, oncofetoprotein, luteinizing hormone releasing hormone receptor (LHRH-R), growth hormone receptor, phosphatidylserine, follicle stimulating hormone receptors, VGEF receptor, folate receptor, CD19, CD20, CD22, CD23, CD27, CD28, CD30, CD31, CD33, CD34, CD40, CD52, CD56, CD70, CD123, CD138, or CD154.

As disclosed herein, receptors, such as Her2/neu or CD20 are typically expressed by or present on (e.g., a membrane receptor) or within a cell. Receptors, such as Her2/neu, may associate with the cell membrane surface or traverse the cell membrane. CD20 is typically not internalized and is expressed on the surface of B cells and B-cell malignancies. Receptors therefore include full length intact native receptors containing an extracellular, transmembrane or cytoplasmic portion, as well as truncated forms or fragments thereof (e.g., an extracellular, transmembrane or cytoplasmic portion or subsequence of a receptor, such as Her2/neu alone, or in combination). For example, a soluble receptor such as Her2/neu typically lacks a transmembrane region and can optionally also lack all or a part of the native extracellular or cytoplasmic region (if present in native receptor, e.g., Her2/neu). Such truncated forms and fragments can retain at least partial binding to a conjugate.

Exemplary antibodies, Heavy (H) chains, Light (L) chains, and fragments thereof include those that bind to epitopes present on receptors. Exemplary Her2/neu epitopes to which antibodies, Heavy (H) chains or Light (L) chains of an antibody, or fragments thereof bind, include HER-2 (p5-13) A2, HER-2 (p8-16) A24, HER-2 (p48-56) A2, HER-2 (p63-71) A24, HER-2 (p106-114) A2, HER-2 (p369-377) A2, A3, A26, HER-2 (p435-443) A2, HER-2 (p654-662) A2, HER-2 (p665-673) A2, HER-2 (p689-697) A2, HER-2 (p754-762) A3, A11, A33, HER-2 (p773-782) A2, HER-2 (p780-788) A24, HER-2 (p785-794) A2, HER-2 (p789-797) A2, HER-2 (p799-807) A2, HER-2 (p952-961) A2 and HER-2 (p1023-1032) A2 (the amino acid position numbers of Her2/neu epitope is referred to by a “p” followed by arabic numbers).

Exemplary antibodies that bind to Her2/neu include humanized anti-ErbB2 antibodies huMAb4D1-1, huMAb4D5-1, huMAb4D5-2, huMAb4D5-3, huMAb4D5-4, huMAb4D5-5, huMAb4D5-6, huMAb4D5-7 and huMAb4D5-8 (HERCEPTIN™) as described in U.S. Pat. No. 5,821,337; humanized 520C9 (WO 93/21319) and humanized 2C4 (pertuzumab) as described in U.S. Pat. No. 7,097,840 and pertuzumab variants as described in US2009/0285837A1. Non-limiting representative antibodies, Heavy (H) chains, Light (L) chains, and fragments thereof that bind to Her2/neu are set forth in Table A.

```
TABLE A
(SEQ ID NOs.: 12-39)
huMAb4D5-1:
V_{L} (Light chain):
1 10 20 30 40
DIQMTQSPSSLSASVGDRVTITCRASQDVNTAVAWYQQKPG
              50 60 70 80
KAPKLLIYSASFLESGVPSRFSGSGSGTDFTLTISSLQPEDF
            90 100
ATYYCQQHYTTPPTFGQGTKVEIK
V_{H} (heavy chain):
1 10 20 30 40
EVQLVESGGGLVQPGGSLRLSCAASGFNIKDTYIHWVRQAP
              50 60 70 80
GKGLEWVARIYPTNGYTRYADSVKGRFTISRDDSKNTLYLQ
            90 100 110 120
MNSLRAEDTAVYYCARWGGDGFYAMDVWGQGTLVTVSS
huMAb4D5-2:
V_{L} (Light chain):
1 10 20 30 40
DIQMTQSPSSLSASVGDRVTITCRASQDVNTAVAWYQQKPG
              50 60 70 80
KAPKLLIYSASFLESGVPSRFSGSGSGTDFTLTISSLQPEDF
            90 100
ATYYCQQHYTTPPTFGQGTKVEIK
V_{H} (heavy chain):
1 10 20 30 40
EVQLVESGGGLVQPGGSLRLSCAASGFNIKDTYIHWVRQAP
              50 60 70 80
GKGLEWVARIYPTNGYTRYADSVKGRFTISADDSKNTLYLQ
            90 100 110 120
MNSLRAEDTAVYYCARWGGDGFYAMDVWGQGTLVTVSS
huMAb4D5-3:
V_{L} (Light chain):
1 10 20 30 40
DIQMTQSPSSLSASVGDRVTITCRASQDVNTAVAWYQQKPG
              50 60 70 80
KAPKLLIYSASFLESGVPSRFSGSGSGTDFTLTISSLQPEDF
            90 100
ATYYCQQHYTTPPTFGQGTKVEIK
V_{H} (heavy chain):
1 10 20 30 40
EVQLVESGGGLVQPGGSLRLSCAASGFNIKDTYIHWVRQAP
              50 60 70 80
GKGLEWVARIYPTNGYTRYADSVKGRFTISADTSKNTAYLQ
            90 100 110 120
MNSLRAEDTAVYYCSRWGGDGFYAMDVWGQGTLVTVSS
huMAb4D5-4:
V_{L} (Light chain):
1 10 20 30 40
DIQMTQSPSSLSASVGDRVTITCRASQDVNTAVAWYQQKPG
              50 60 70 80
KAPKLLIYSASFLESGVPSRFSGSRSGTDFTLTISSLQPEDF
            90 100
ATYYCQQHYTTPPTFGQGTKVEIK
V_{H} (heavy chain):
1 10 20 30 40
EVQLVESGGGLVQPGGSLRLSCAASGFNIKDTYIHWVRQAP
              50 60 70 80
GKGLEWVARIYPTNGYTRYADSVKGRFTISADTSKNTLYLQ
            90 100 110 120
MNSLRAEDTAVYYCSRWGGDGFYAMDVWGQGTLVTVSS
huMAb4D5-5:
V_{L} (Light chain):
1 10 20 30 40
DIQMTQSPSSLSASVGDRVTITCRASQDVNTAVAWYQQKPG
              50 60 70 80
KAPKLLIYSASFLESGVPSRFSGSRSGTDFTLTISSLQPEDF
            90 100
ATYYCQQHYTTPPTFGQGTKVEIK
V_{H} (heavy chain):
1 10 20 30 40
EVQLVESGGGLVQPGGSLRLSCAASGFNIKDTYIHWVRQAP
              50 60 70 80
GKGLEWVARIYPTNGYTRYADSVKGRFTISADTSKNTAYLQ
            90 100 110 120
MNSLRAEDTAVYYCSRWGGDGFYAMDVWGQGTLVTVSS
huMAb4D5-6:
V_{L} (Light chain):
1 10 20 30 40
DIQMTQSPSSLSASVGDRVTITCRASQDVNTAVAWYQQKPG
              50 60 70 80
KAPKLLIYSASFLYSGVPSRFSGSRSGTDFTLTISSLQPEDF
            90 100
ATYYCQQHYTTPPTFGQGTKVEIK
V_{H} (heavy chain):
1 10 20 30 40
EVQLVESGGGLVQPGGSLRLSCAASGFNIKDTYIHWVRQAP
              50 60 70 80
GKGLEWVARIYPTNGYTRYADSVKGRFTISADTSKNTAYLQ
            90 100 110 120
MNSLRAEDTAVYYCSRWGGDGFYAMDVWGQGTLVTVSS
huMAb4D5-7:
V_{L} (Light chain):
1 10 20 30 40
DIQMTQSPSSLSASVGDRVTITCRASQDVNTAVAWYQQKPG
              50 60 70 80
KAPKLLIYSASFLESGVPSRFSGSRSGTDFTLTISSLQPEDF
            90 100
ATYYCQQHYTTPPTFGQGTKVEIK
V_{H} (heavy chain):
1 10 20 30 40
EVQLVESGGGLVQPGGSLRLSCAASGFNIKDTYIHWVRQAP
              50 60 70 80
GKGLEWVARIYPTNGYTRYADSVKGRFTISADTSKNTAYLQ
            90 100 110 120
MNSLRAEDTAVYYCSRWGGDGFYAMDYWGQGTLVTVSS
huMAb4D5-8:
V_{L} (Light chain):
1 10 20 30 40
DIQMTQSPSSLSASVGDRVTITCRASQDVNTAVAWYQQKPG
              50 60 70 80
KAPKLLIYSASFLYSGVPSRFSGSRSGTDFTLTISSLQPEDF
            90 100
ATYYCQQHYTTPPTFGQGTKVEIK
V_{H} (heavy chain):
1 10 20 30 40
EVQLVESGGGLVQPGGSLRLSCAASGFNIKDTYIHWVRQAP
              50 60 70 80
GKGLEWVARIYPTNGYTRYADSVKGRFTISADTSKNTAYLQ
            90 100 110 120
MNSLRAEDTAVYYCSRWGGDGFYAMDYWGQGTLVTVSS
huMAb4D5:
V_{L} (Light chain):
1 10 20 30 40
DIQMTQSPSSLSASVGDRVTITCRASQDVNTAVAWYQQKPG
              50 60 70 80
KAPKLLIYSASFLYSGVPSRFSGSRSGTDFTLTISSLQPEDF
            90 100
ATYYCQQHYTTPPTFGQGTKVEIK
V_{H} (heavy chain):
1 10 20 30 40
EVQLVESGGGLVQPGGSLRLSCAASGFNIKDTYIHWVRQAP
              50 60 70 80
GKGLEWVARIYPTNGYTRYADSVKGRFTISADTSKNTAYLQ
            90 100 110 120
MNSLRAEDTAVYYCSRWGGDGFYAMDYWGQGTLVTVSS
Antibody sequence permutations(U.S. Pat. No. 7,435,797 SQ 1 and 2))
huMAb4D5:
V_{L} (Light chain):
1 10 20 30 40
DIQMTQSPSSLSASVGDRVTITCRASQDVNTAVAWYQQKPG
              50 60 70 80
KAPKLLIYSASFLYSGVPSRFSGSRSGTDFTLTISSLQPEDF
            90 100
ATYYCQQHYTTPPTFGQGTKVEIK
V_{H} (heavy chain):
1 10 20 30 40
EVQLVESGGGLVQPGGSLRLSCAASGFNIKDTYIHWVRQAP
              50 60 70 80
GKGLEWVARIYPTNGYTRYADSVKGRFTISADTSKNTAYLQ
            90 100 110 120
MNSLRAEDTAVYYCSRWGGDGFYAMDYWGQGTLVTVSS
huMAb4D5-8
(for VL: Q27, D28, N30, T31, A32, Y49, F53, Y55, R66 H91, Y92, T94; for VH: W95, D98, F100,
Y100, Y102):
V_{L} (Light chain): Claim 1 mutation
1 10 20 30 40
DIQMTQSPSSLSASVGDRVTITCRASQDVSSAVAWYQQKPG
              50 60 70 80
KAPKLLID/WSASFLYSGVPSRFSGSRSGTDFTLTISSLQPEDF
            90 100
ATYYCQQHYTTPPTFGQGTKVEIK
V_{H} (heavy chain):
1 10 20 30 40
EVQLVESGGGLVQPGGSLRLSCAASGFNIKDTYIHWVRQAP
              50 60 70 80
GKGLEWVARIYPINGYTRYADSVKGRFTISADTSKNTAYLQ
            90 100 110 120
MNSLRAEDTAVYYCSRWGGWGPKLAMDYWGQGTLVTVSS
Pertuzumab Sequences (US 2010/0015157A1)
V_{L} (Light chain):
1 10 20 30 40
DIQMTQSPSSLSASVGDRVTITCKASQDVSIGVAWYQQKPG
              50 60 70 80
KAPKLLIYSASYRYTGVPSRFSGSGSGTDFTLTISSLQPED
            90 100 110 120
FATYYCQQYYIYPYTFGQGTKVEIKRTVAAPSVFIFPPSDEQ
          130 140 150 160
LKSGTASVVCLLNNNFYPREAKVQWKVDNALQSGENSQES
            170 180 190 200
VTEQDSKDSTYSLSSTLTLSKADYEKHKVYACEVTHQGLSS
          210
PVTKSFNRGEC
V_{H} (heavy chain):
1 10 20 30 40
EVQLVESGGGLVQPGGSLRLSCAASGFTFTDYTMDWVRQA
              50 60 70 80
PGKGLEWVADVNPNSGGSIYNQRFKGRFTLSVDRSKNTLYL
            90 100 110 120
QMNSLRAEDTAVYYCARNLGPSFYFDYWGQGTLVTVSSAST
          130 140 150 160
KGPSVFPLAPSSKSTSGGTAALGCLVKDYFPEPVTVSWNSG
            170 180 190 200
ALTSGVHTFPAVLQSSGLYSLSSVVTVPSSSLGTQTYICNVN
      210 220 230 240
HKPSNTKVDKKVEPKSCDKTHTCPPCPAPELLGGPSVFLFP
      250 260 270 280
PKPKDTLMISRTPEVTCVVVDVSHEDPEVKFNWYVDGVEVH
    290 300 310 320
NAKTKPREEQYNSTYRVVSVLTVLHQDWLNGKEYKCKVSN
  330 340 350 360
KALPAPIEKTISKAKGQPREPQVYTLPPSREEMTKNQVSLTC
370 380 390 400 410
LVKGFYPSDIAVEWESNGQPENNYKTTPPVLDSDGSFFLYS
                  420 430 440
KLTVDKSRWQQGNVFSCSVMHEALHNHYTQKSLSLSPG
Pertuzumab variants (US2009/0285837 A1)
V_{L} (Light chain):
1 10 20 30 40
VHSDIQMTQSPSSLSASVGDRVTITCKASQDVSIGVAWYQQ
              50 60 70 80
KPGKAPKLLIYSASYRYTGVPSRFSGSGSGTDFTLTISSLQP
            90 100 110 120
EDFATYYCQQYYIYPYTFGQGTKVEIKRTVAAPSVFIFPPSD
          130 140 150 160
EQLKSGTASVVCLLNNFYPREAKVQWKVDNALQSGNSQES
          170 180 190 200
VTEQDSKDSTYSLSSTLTLSKADYEKHKVYACEVTHQGLSS
        210
PVTKSFNRGEC
V_{H} (Heavy chain):
1 10 20 30 40
EVQLVESGGGLVQPGGSLRLSCAASGFTFTDYTMDWVRQA
                  50 60 70 80
PGKGLEWVADVNPNSGGSIYNQRFKGRFTLSVDRSKNTLYL
                  90 100 110 120
QMNSLRAEDTAVYYCARNLGPSFYFDYWGQGTLVTVSSAS
              130 140 150 160
TKGPSVFPLAPSSKSTSGGTAALGCLVKDYFPEPVTVSWNS
                170 180 190 200
GALTSGVHTFPAVLQSSGLYSLSSVVTVPSSSLGTQTYICNV
            210 220 230 240
NHKPSNTKVDKKVEPKSCDKTHTCPPCPAPELLGGPSVFLV
          250 260 270 280
PPKPKDTLMISRTPEVTCVVVDVSHEDPEVKFNWYVDGVEV
          290 300 310 320
HNAKTKPREEQYNSTYRVVSVLTVLHQDWLNGKEYKCKVS
          330 340 350 360
NKALPAPIEKTISKAKGQPREPQVYTLPPSREEMTKNQVSLT
      370 380 390 400
CLVKGFYPSDIAVEWESNGQPENNYKTTPPVLDSDGSFFLY
    410 420 430 440
SKLTVDKSRWQQGNVFSCSVMHEALNHYTQKSLSLSPGK
Pertuzumab Sequences: humanized 2C4, 7C2, 7F3, 7D3, 3E8, 4D5, 2H11, 3H4 (U.S. Pat.
No. 7,097,840)
V_{L} (Light chain):
1 10 20 30 40
DIQMTQSPSSLSASVGDRVTITCKASQDVSIGVAWYQQKPG
              50 60 70 80
KAPKLLIYSASYRYTGVPSRFSGSGSGTDFTLTISSLQPED
            90 100 110 120
FATYYCQQYYIYPYTFGQGTKVEIKRTVAAPSVFIFPPSDEQ
        130 140 150 160
LKSGTASVVCLLNNNFYPREAKVQWKVDNALQSGENSQES
            170 180 190 200
VTEQDSKDSTYSLSSTLTLSKADYEKHKVYACEVTHQGLSS
          210
PVTKSFNRGEC
V_{H} (heavy chain):
1 10 20 30 40
EVQLVESGGGLVQPGGSLRLSCAASGFTFTDYTMDWVRQA
                50 60 70 80
PGKGLEWVADVNPNSGGSIYNQRFKGRFTLSVDRSKNTLYL
              90 100 110 120
QMNSLRAEDTAVYYCARNLGPSFYFDYWGQGTLVTVSSAST
              130 140 150 160
KGPSVFPLAPSSKSTSGGTAALGCLVKDYFPEPVTVSWNSG
            170 180 190 200
ALTSGVHTFPAVLQSSGLYSLSSVVTVPSSSLGTQTYICNVN
      210 220 230 240
HKPSNTKVDKKVEPKSCDKTHTCPPCPAPELLGGPSVFLFP
      250 260 270 280
PKPKDTLMISRTPEVTCVVVDVSHEDPEVKFNWYVDGVEVH
  290 300 310 320
NAKTKPREEQYNSTYRVVSVLTVLHQDWLNGKEYKCKVSN
  330 340 350 360
KALPAPIEKTISKAKGQPREPQVYTLPPSREEMTKNQVSLTC
370 380 390 400 410
LVKGFYPSDIAVEWESNGQPENNYKTTPPVLDSDGSFFLYS
                420 430 440
KLTVDKSRWQQGNVFSCSVMHEALHNHYTQKSLSLSPG
```

Additional anti-ErbB2 antibodies with various properties have been described in Tagliabue et al. Int. J. Cancer 47:933-937 (1991); Mckenzie et al. Oncogene 4:543 (1989); Maier et al. Cancer Res. 51:5361 (1991); Bacus et al. Molecular Carcinogenesis 3:350 (1990); Stancovski et al. PNAS (USA) 88:8691 (1991); Bacus et al. Cancer Research 52:2580 (1992); Xu et al. Int. J Cancer 53:401-408 (1993); WO 94/00136; Kasprzyk et al. Cancer Research 52:2771 (1992); Hancock et al. Cancer Res. 51:4575 (1991); Shawver et al. Cancer Res. 54:1367 (1994); Arteaga et al. Cancer Res. 54:3758 (1994); Harwerth et al. J Biol Chem. 267:15160 (1992); U.S. Pat. No. 5,783,186; and Klapper et al. Oncogene 14:2099 (1997). Conjugates based on such antibodies, Heavy (H) chains and/or Light (L) chains of an antibody, and fragments thereof, are included in the invention.

As disclosed herein, other scaffold like structures that bind to targets (e.g., receptors) can be included in an invention conjugate. Such structures include affibodies, aptamers, avimers and nanobodies. Conjugates that include such affibodies, aptamers, avimers and nanobodies are also included in the invention.

Exemplary Her2/neu binding affibodies, include ZHer2-neu: 2, ZHer2-neu: 4 ZHer2-neu: 7 ZHer2-neu: 8 and Fab63, which affibody sequences are in Table B.

TABLE B
(SEQ ID NOs.: 40-47)

| | |
|---|---|
| Z_{wt} | VDNKFNK EQQNAFYEILH LPNLNE EQRNAFIQSLKD DPSQ SANLLAEAKKLNDA QAPK |
| Z_{her2:4} | VDNKFNK ELRQAYWEIQA LPNLNW TQSRAFIRSLYD DPSQ SANLLAEAKKLNDA QAPK |
| Z_{her2:7} | VDNKFNK EPKTAYWEIVK LPNLNP EQRRAFIRSLYD DPSQ SANLLAEAKKLNDA QAPK |
| Z_{her2:24} | VDNKFNK EPREAYWEIQR LPNLNN KOKAAFIRSLYD DPSQ SANLLAEAKKLNDA QAPK |
| Z_{her2:79} | VDNKFNK EWMTAGKEIYR LPNLNG TQVRAFIQSLSD DPSQ SANLLAEAKKLNDA QAPK |
| Z_{her2:2} | VDNKFNK EWVQAGSEIYN LPNLNR AQMRAFIRSLSD DPSQ SANLLAEAKKLNDA QAPK |
| Z_{her2:8} | VDNKFNK EIKQAFHEIVR LPNLNA DQVRAFIYSLGD DPSQ SANLLAEAKKLNDA QAPK |
| Z_{her2:25} | VDNKFNK EMVDAGAEIWR LPNLNA KQM*AFIDSLGD DPSQ SANLLAEAKKLNDA QAPK |

CD20 targeting antibodies include Rituximab, Ofatumumab (Arzerra®), Tositumumab (GSK), Ibritumomab (IDEC) (reference Ivanov 2008), 2F2 (HuMax-CD20), 7D8, IgM2C6, IgG1 2C6, 11B8, B1, 2H7, LT20, 1F5 and AT80, as described in Teeling et al. (US 2004/0167319). Exemplary anti-CD20 V_{L} and V_{H} chains and whole antibodies are set forth below in Table C (Bold fonts indicate respective complimentary determining regions, CDRs, for each chain, CDR1, CDR2 and CDR3):

TABLE C
(SEQ ID NOs.: 48-69)
V_{L} (Light chain):
DIQMTQSPSSLSASVGDRVTITCRASSSVSYIHWYQQKPGKAPKLLIYATSNLASGVPSRFS
GSRSGTDFTLTISSLQPEDFATYYCQQWTSNPPTFGQGTKVEIK
V_{H} (heavy chain):
EVQLVESGGGLVQPGGSLRLSCAASGYTFTSYNMHWVRQAPGKGLEWVAAIYPGNGDTS
YNQKFKGRFTISADTSKNTAYLQMNSLRAEDTAVYYCSRSTYYGGDWYFDVWGQGTLV
TVSS
Rituxan® V_{L} and V_{H} chains:
V_{L} (Light chain):
QIVLSQSPAILSASPGEKVTMTCRASSSVSYIHWFQQKPGSSPKPWIYATSNLASGVPVRFS
GSGSGTSYSLTISRVEAEDAATYYCQQWTSNPPTFGGGTKLEIK
V_{H} (heavy chain):
QVQLQQPGAELVKPGASVKMSCKASGYTFTSYNMHWVKQTPGRGLEWIGAIYPGNGDTS
YNQKFKGKATLTADKSSSTAYMQLSSLTSEDSAVYYCARSTYYGGDWYFNVWGAGTTV
TVS
Ibritumomab (IDEC)
V_{L} (Light chain):
QIVLSQSPAILSASPGEKVTMTCRASSSVSYMHWYQQKPGSSPKPWIYAPSNLASGVPARF
SGSGSGTSYSLTISRVEAEDAATYYCQQWSFNPPTFGAGTKLELK
V_{H} (heavy chain):
QAYLQQSGAELVRPGASVKMSCKASGYTFTSYNMHWVKQTPRQGLEWIGAIYPGNGDTS
YNQKFKGKATLTVDKSSSTAYMQLSSLTSEDSAVYFCARVVYYSNSYWYFDVWGTGTTV
TVS
Tositumomab: >Mouse-Human chimeric Anti-CD20 (same V domains as ibritumomab- they differ
in constant regions only)
V_{L} (Light chain):
QIVLSQSPAILSASPGEKVTMTCRASSSVSYMHWYQQKPGSSPKPWIYAPSNLASGVPARF
SGSGSGTSYSLTISRVEAEDAATYYCQQWSFNPPTFGAGTKLELK
V_{H} (heavy chain):
QAYLQQSGAELVRPGASVKMSCKASGYTFTSYNMHWVKQTPRQGLEWIGAIYPGNGDTS
YNQKFKGKATLTVDKSSSTAYMQLSSLTSEDSAVYFCARVVYYSNSYWYFDVWGTGTTV
TVS
2C6 US7850962 B2 (Teeling et al)
V_{H} (heavy chain):
AVQLVESGGGLVQPGRSLRLSCAASGFTFGDYTMHWVRQAPGLGLEWVSGISWNSGSIGY
ADSVLGRFTISRDNALNSLYLQMNSLRAEDTALYYCTLDNQYGSGSTYGLGVWGQGTLVT
VSS
V_{L} (Light chain):
EIVLTQSPATLSLSPGERATLSCRASQSVSSYLAWYQQKPGQAPRLLIYDASNRATGIPARFS
GSGSGTDFTLTISSLEPEDFAVYYCQQRSNWPLTFGGGTKVEIL
2F2 US2004/0167319A1 (Teeling et al)
V_{H} (heavy chain):
MFLGLSWIFLLAILKGVQCEVQLVESGGGLVQPGRSLRLSCAASGFTFNDYAMHWVRQAP
GKGLEWVSTISWNSGSIGYADSVKGRFTISRDNAKKSLYLQMNSLRAEDTALYYCAKDIQ
YGNYYYGMDVWGQGTTVTVSS
V_{L} (Light chain):
MEAPAQLLFLLLLWLPDTTGEIVLTQSPATLSLSPGERATLSCRASQSVSSYLAWYQQKPG
QAPRLLIYDASNRATGIPARFSGSGSTDFTLTISSLEPEDFAVYYCQQRSNWPITFGQGTRLE
IK
11B8 US2004/0167319A1 (Teeling, et al.)
V_{H} (heavy chain):
MELGLSWVFLVAILKGVQCEVQLVQSGGGLVHPGGSLRLSCTGSGFTFSYHAMHWVRQA
P
GKGLEWVSIIGTGGVTYYADSVKGRFTISRDNVKNSLYLQMNSLRAEDMAVYYCARDYY
GAGSFYDGLYGMDVWGQGTTVTVSS
V_{L} (Light chain):
MEAPAQLLFLLLLWLPDTTGEIVLTQSPATLSLSPGERATLSCRASQSVSSYLAWYQQKPG
QAPRLLIYDASNRATGIPARFSGSGSGTDFTLTISSLEPEDFAVYYCQQRSDWPLTFGGGTK
VEIK
7D8 US2004/0167319A1 (Teeling et al)
V_{H} (heavy chain):
MELGLSWIFLLAILKGVQCEVQLVESGGGLVQPDRSLRLSCAASGFTFHDYAMHWVRQAP
GKGLEWVSTISWNSGTIGYADSVKGRFTISRDNAKNSLYLQMNSLRAEDTALYYCAKDIQ
YGNYYYGMDVWGQGTTVTVSS
V_{L} (Light chain):
MEAPAQLLFLLLLWLPDTTGEIVLTQSPATLSLSPGERATLSCRASQSVSSYLAWYQQKPGQ
APRLLIYDASNRATGIPARFSGSGSGTDFTLTISSLEPEDFAVYYCQQRSNWPITFGQGTRLEI
K
Whole antibody drug sequences:
Ibritumomab (IDEC)(reference Ivanov 2008)
Mouse Anti-CD20 Heavy chain 1:
QAYLQQSGAELVRPGASVKMSCKASGYTFTSYNMHWVKQTPRQGLEWIGAIYPGNGDTS
YNQKFKGKATLTVDKSSSTAYMQLSSLTSEDSAVYFCARVVYYSNSYWYFDVWGTGTTV
TVSAPSVYPLAPVCGDTTGSSVTLGCLVKGYFPEPVTLTWNSGSLSSGVHTFPAVLQSDLYT
LSSSVTVTSSTWPSQSITCNVAHPASSTKVDKKIEPRGPTIKPCPPCKCPAPNLLGGPSVFIFPP
KIKDVLMISLSPIVTCVVVDVSEDDPDVQISWFVNNVEVHTAQTQTHREDYNSTLRVVSAL
PIQHQDWMSGKEFKCKVNNKDLPAPIERTISKPKGSVRAPQVYVLPPPEEEMTKKQVTLTC
MVTDFMPEDIYVEWTNNGKTELNYKNTEPVLDSDGSYFMYSKLRVEKKNWVERNSYSCS
VVHEGLHNHHTTKSFSR
Mouse Anti-CD20 Light chain 1:
QIVLSQSPAILSASPGEKVTMTCRASSSVSYMHWYQQKPGSSPKPWIYAPSNLASGVPARFS
GSGSGTSYSLTISRVEAEDAATYYCQQWSFNPPTFGAGTKLELKRADAAPTVFIFPPSDEQL
KSGTASVVCLLNNFYPREAKVQWKVDNALQSGNSQESVTEQDSKDSTYSLSSTLTLSKAD
YEKHKVYACEVTHQGLSSPVTKSFN
Rituximab
Heavy chain chimeric:
QVQLQQPGAELVKPGASVKMSCKASGYTFTSYNMHWVKQTPGRGLEWIGAIYPGNGDTS
YNQKFKGKATLTADKSSSTAYMQLSSLTSEDSAVYYCARSTYYGGDWYFNVWGAGTTVT
VSAASTKGPSVFPLAPSSKSTSGGTAALGCLVKDYFPEPVTVSWNSGALTSGVHTFPAVLQS
SGLYSL
SSVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSV
MHEALHNHYTQKSLSLSPGK
Light chain chimeric:
QIVLSQSPAILSASPGEKVTMTCRASSSVSYIHWFQQKPGSSPKPWIYATSNLASGVPVRFSG
SGSGTSYSLTISRVEAEDAATYYCQQWTSNPPTFGGGTKLEIKRTVAAPSVFIFPPSDEQLKS
GTASVVCLLNNFYPREAKVQWKVDNALQSGNSQESVTEQDSKDSTYSLSSTLTLSKADYE
KHKVYACEVTHQGLSSPVTKSFNRGEC
Tositumomab
Mouse-Human chimeric Anti-CD20 Heavy Chain 1:
QAYLQQSGAELVRPGASVKMSCKASGYTFTSYNMHWVKQTPRQGLEWIGAIYPGNGDTS
YNQKFKGKATLTVDKSSSTAYMQLSSLTSEDSAVYFCARVVYYSNSYWYFDVWGTGTTV
TVSGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKAEPKSCDKTHTCPPCPAPELLGGPSVFLF
PPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSC
SVMHEALHNHYTQKSLSLSPGK
Mouse-Human chimeric Anti-CD20 Light Chain 1:
QIVLSQSPAILSASPGEKVTMTCRASSSVSYMHWYQQKPGSSPKPWIYAPSNLASGVPARFS
GSGSGTSYSLTISRVEAEDAATYYCQQWSFNPPTFGAGTKLELKRTVAAPSVFIFPPSDEQL
KSGTASVVCLLNNFYPREAKVQWKVDNALQSGNSQESVTEQDSKDSTYSLSSTLTLSKAD
YEKHKVYACEVTHQGLSSPVTKSFNR

Non-limiting exemplary anti-luteinizing hormone releasing hormone receptor (LHRH-R) Antibody Light (V_{L}) and Heavy (V_{H}) chain sequences are set for in Table D:

TABLE D
(SEQ ID NOs.: 70-73)

```
(A) Immunoglobulin light chains (κ) of GHR-106
                                  LEADER
ATGGATTGACAGGCCCAGGTTCTTATATTGCTGCTGCTATGGGTATCTGGTACCTGTGGG
  M D S Q A Q V L I L L L L W V S G T C G
                                                    FR1
GACATTGTGATGTCACAGTCTCCATCCTCCCTGGCTGTGTCAGCAGGAGAGAAGGTCACT
  D I V M S Q S P S S L A V S A G E K V T
                                                    CDR1
ATGAGCTGCAAATCCAGTCAGAGTCTGCTCAACAGTAGAACCCGAAAGAACTACTTGGCT
  M S C K S S Q S L L N S R T R K N Y L A
                                      FR2 CDR2
TGGTACCAGCAGAAACCAGGGCAGTCTCCTAAACTGCTGATCTACTGGGCATCCACTAGG
  W Y Q Q K P G Q S P K L L I Y W A S T R
                                                                  FR3
GAATCTGGGGTCCCTGATCGCTTCACAGGCAGTGGATCTGGGACAGATTTCACTCTCACC
  E S G V P D R F T G S G S G T D F T L T
                                                                                          CDR3
ATCAGCAGTGTGCAGGCTGAAGACCTGGCAGTTTATTACTGCAAGCAATCTTATAATCTT
  I S S V Q A E D L A V Y Y C K Q S Y N L
                              FR4
TACACGTTCGGAGGGGGGACCAAGCTGGAAATAAAA
  Y T F G G G T K L E I K
(B) Immunoglobulin heavy chains of GHR-106
                                                                FR1
CAGGTGCAGCTGAAGGAGTCAGGACCTGGCCTGGTGGCACCCTCACAGAGCCTGTCCATC
  Q V Q L K E S G P G L V A P S Q S L S I
                                                            CDR1
ACATGCACTGTCTCTGGGTTCTCATTATCCAGATATAGTGTACACTGGGTTCGCCAGCCT
  T C T V S G F S L S R Y S V H W V R Q P
                                                                                CDR2
CCAGGAAAGGGCCTGGAGTGGCTGGGAATGATATGGGGTGGTGGAAGCACAGACTATAAT
  P G K G L E W L G M I W G G G S T D Y N
                                                                          FR3
TCAGCTCTCAAATCCAGACTGAGCATCAGCAAGGACAACTCCAAGAGCCAAGTTTTCTTA
  S A L K S R L S I S K D N S K S Q V F L
AAAATGAACAGTCTGCAAACTGATGACACAGCCATGTACTACTGTGCCAGAGGCAATGAT
  K M N S L Q T D D T A M Y Y C A R G N D
            CDR3 FR4
GGTTACTACTCGTTTGCTTACTGGGGCCAAGGGACTCTGGTCACTGTCTCTTCA
  G Y Y S F A Y W G Q G T L V T V S S
```

The invention includes modifications or variations, such as amino acid modifications (e.g., conservative or non-conservative substitutions, additions or deletions) of a conjugate, in an antibody, Heavy (H) chain, Light (L) chain, or fragment thereof portion, a lytic domain, or any other domain of a conjugate. Thus, a conjugate that includes a modification of one or more residues of an antibody, an antibody Heavy (H) chain, an antibody Light (L) chain, a fragment thereof, or a lytic domain can incorporate any number of modifications or variations, as long as such modifications or variations do not destroy target binding and/or lytic activity. Thus, for example, a modified antibody, Heavy (H) chain, Light (L) chain, or fragment thereof, that binds to a target, such as a receptor (e.g., Her2/neu or CD20) can retain at least partial target (receptor) binding, or a modified lytic domain can retain at least partial lytic activity, such as cell killing or apoptosis.

A “conservative substitution” is a replacement of one amino acid by a biologically, chemically or structurally similar residue. Biologically similar means that the substitution is compatible with a biological activity, e.g., lytic activity. Structurally similar means that the amino acids have side chains with similar length, such as alanine, glycine and serine, or having similar size, or the structure of a first, second or additional domain is maintained, such as an amphipathic alpha helix. Chemical similarity means that the residues have the same charge or are both hydrophilic and hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, etc. Routine assays can be used to determine whether a conjugate variant has activity, e.g., target binding activity or lytic activity.

Modifications and variations therefore include of the various sequences set forth herein, such as of antibodies, Heavy (H) chains and Light (L) chains of an antibody, and fragments thereof, as well as lytic domains (or additional domains, if present). Non-limiting modifications of lytic domains are of KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAKKFAKFAK (SEQ ID NOs.: 1-7), or a sequence that includes or consists of such lytic domains.

In particular embodiments, a subsequence of an antibody, Heavy (H) chain, Light (L) chain, fragments thereof, or a lytic domain has at least 1 to 5, 5 to 10, 10 to 15, 15 to 20, 20 to 25, 25 to 30, 30 to 35, 35 to 40, 40 to 50, 50 to 60, 60 to 70, 70 to 80, 80 to 90, 90 to 100, or more amino acid residues identical to the reference sequence. In additional particular embodiments, a substitution or deletion of one or more amino acids (e.g., 1-3, 3-5, 5-10, 10-20, 20-30, or more) residues of an antibody, Heavy (H) chain, Light (L) chain, fragment thereof, and/or lytic domain can have a sequence with 50%, 60%, 70%, 75%, 80%, 85%, 90%, 95%, 96%, 97%, 98%, or more identity to a reference sequence (e.g., antibody, Heavy (H) chain, Light (L) chain, fragment thereof, or lytic domain, such as KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA or KFAKFAKKFAKFAKKFAKFAKKFAKFAK (SEQ ID NOs.: 1-7)).

In a particular embodiment, a conjugate includes an antibody, Heavy (H) chain, Light (L) chain, or fragment thereof, that binds to a target (e.g., receptor, such as Her2/neu or CD20) and a second lytic domain that includes or consists of an L- or D-amino acid sequence set forth as: KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA or KFAKFAKKFAKFAKKFAKFAKKFAKFAK (SEQ ID NOs.: 1-7) having one or more of the K residues substituted with an F or L residue, one or more of the F residues substituted with a K, A or L residue, or one or more of the A residues substituted with a K, F or L residue. In another particular embodiment, a conjugate includes an antibody, Heavy (H) chain, Light (L) chain, or fragment thereof, that binds to a target (e.g., receptor, such as Her2/neu or CD20) and a second domain consisting of an L- or D-amino acid sequence selected from KFAKFAKKFAKFAKK, KFAKFAKKFAKFAKKF, KFAKFAKKFAKFAKKFA, KFAKFAKKFAKFAKKFAK, KFAKFAKKFAKFAKKFAKF, KFAKFAKKFAKFAKKFAKFA and KFAKFAKKFAKFAKKFAKFAKKFAKFAK (SEQ ID NOs.: 1-7) having one or more of the K residues substituted with an F or L residue, one or more of the F residues substituted with a K, A or L residue, or one or more of the A residues substituted with a K, For L residue (e.g., 12, 13, 14, 15, 16, 17, 18, 19, 20, 21, 22, 23, 24, 25, 26, 27 or 28 residues in length).

The term “identity” and “homology” and grammatical variations thereof mean that two or more referenced entities are the same. Thus, where two amino acid sequences are identical, they have the same amino acid sequence. “Areas, regions or domains of identity” mean that a portion of two or more referenced entities are the same. Thus, where two amino acid sequences are identical or homologous over one or more sequence regions, they share identity in these regions. The term “complementary,” when used in reference to a nucleic acid sequence means the referenced regions are 100% complementary, i.e., exhibit 100% base pairing with no mismatches.

Due to variation in the amount of sequence conservation between structurally and functionally related proteins, the amount of sequence identity required to retain a function or activity (e.g., target binding or lytic) depends upon the protein, the region and the function or activity of that region. For example, for a lytic peptide sequence multiple PNNPNNP (SEQ ID NO.: 153) sequence repeat patterns or motifs can be present, but one or more interrupted or non-interrupted PNNPNNP (SEQ ID NO.: 153) sequence repeat patterns or motifs need not be present.

The extent of identity between two sequences can be ascertained using a computer program and mathematical algorithm known in the art. Such algorithms that calculate percent sequence identity (homology) generally account for sequence gaps and mismatches over the comparison region. For example, a BLAST (e.g., BLAST 2.0) search algorithm (see, e.g., Altschul et al., J. Mol. Biol. 215:403 (1990), publicly available through NCBI) has exemplary search parameters as follows: Mismatch-2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Conjugate amino acid residues can be joined by a covalent or a non-covalent bond. Non-limiting examples of covalent bonds are amide bonds, non-natural and non-amide chemical bonds, which include, for example, glutaraldehyde, N-hydroxysuccinimide esters, bifunctional maleimides, N,N′-dicyclohexylcarbodiimide (DCC) or N,N′-diisopropylcarbodiimide (DIC). Linking groups alternative to amide bonds include, for example, ketomethylene (e.g., —C(═O)—C_{H}2— for —C(═O)—NH—), aminomethylene (C_{H}2—NH), ethylene, olefin (CH═CH), ether (C_{H}2—O), thioether (C_{H}2—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide and Backbone Modifications,” Marcel Decker, NY).

Lytic domains (and additional domains, when present) may be positioned anywhere on the antibody, antibody fragment, Heavy (H) chain, Light (L) chain, or fragment of a Heavy (H) chain or a Light (L) chain of an antibody, provided that the lytic domain (or any additional domain) does not destroy binding to a target. Non-limiting positions include a lytic domain positioned at the amino-terminus, or at the carboxyl-terminus, of an antibody Heavy (H) chain or a Light (L) chain. Where additional domains are present (e.g., third, fourth, fifth, sixth, seventh, etc. domains), the additional domain(s) can also be positioned anywhere.

First portion antibodies, Heavy (H) chains, Light (L) chains, and fragments thereof, and second (lytic) domains (and additional domains, when present), can be fused or joined to each other by a covalent or a non-covalent bond. First portion antibodies, Heavy (H) chains, Light (L) chains, and fragments thereof, and second (lytic) domains (and additional domains, when present), can be immediately adjacent to each other or separated by an intervening region, such as a hinge, spacer or linker positioned between the two domains.

Examples of linkers or spacers include a non-peptide linker or spacer, such as a continuous carbon atom (C) chain (e.g., CCCCC). Multi-carbon chains include carboxylic acids (e.g., dicarboxylic acids) such as glutaric acid, succinic acid and adipic acid. A particular non-limiting example is a 6 carbon linker such as α-amino-caproic acid.

Additional examples of linkers or spacers include one or more amino acid residues, such as a peptide spacer or linker positioned between the antibody, Heavy (H) chain, Light (L) chainy, or fragment thereof, and the second (lytic) domains (or additional domains, when present). Peptide spacer or linker sequences can be any length, but typically range from about 1-10, 10-20, 20-30, 30-40, or 40-50 amino acid residues. In particular embodiments, a peptide spacer or linker positioned between two (or more) domains is from 1 to 5, 1 to 10, 1 to 20, 1 to 25 L- or D-amino acid residues, or 1 to 4, 1 to 6 or 1 to 8 L- or D-amino acid residues. Particular amino acid residues that are included in sequences positioned between the first and second domains include one or more of or A, S or G amino acid residues. Specific non-limiting examples of peptides positioned between the first and second domains include a sequence within or set forth as: GSGGS (SEQ ID NO.: 9), ASAAS (SEQ ID NO.: 8) or multiples of the particular linker sequence (GSGGS (SEQ ID NO.: 9)) n or (ASAAS (SEQ ID NO.: 8)) n, where n=1-5, 5-10, 10-20, etc. Derivatives of amino acids and peptides can be positioned between the two (or more) domains. A specific non-limiting example of an amino acid derivative is a lysine derivative.

Conjugates with or without a spacer or linker, or a third, fourth, fifth, sixth, seventh, etc. domain can be entirely composed of natural amino acids or synthetic, non-natural amino acids or amino acid analogues, or can include derivatized forms. In various embodiments, a conjugate includes in a antibody, Heavy (H) chain, Light (L) chainy, or fragment thereof, and/or a second (lytic) domain (and additional domains, when present), one or more D-amino acids, mixtures of D-amino acids and L-amino acids, or a sequence composed entirely of D-amino acid residues.

Conjugates can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., induce or stabilize a secondary structure, e.g., an alpha helix conformation. Conjugates include cyclic structures such as an end-to-end amide bond between the amino and carboxy-terminus of the molecule or intra- or inter-molecular disulfide bond(s). Conjugates may be modified in vitro or in vivo, e.g., post-translationally modified to include, for example, sugar or carbohydrate residues, phosphate groups, fatty acids, lipids, etc.

Specific examples of an addition include a third, fourth, fifth, sixth or seventh domain. Conjugates with two domains can therefore include one or more additional domains (third, fourth, fifth, sixth, seventh, etc.) covalently linked thereto to impart a distinct or complementary function or activity. Exemplary additional domains include domains facilitating isolation, which include, for example, metal chelating peptides such as polyhistidine tracts and histidine-tryptophan modules that allow purification on immobilized metals; protein A binding domains that allow purification on immobilized immunoglobulin; and domain utilized in the FLAGS extension/affinity purification system (Immunex Corp, Seattle WA). Optional inclusion of a cleavable sequence such as Factor Xa or enterokinase between a purification domain and the conjugate can be used to facilitate purification. For example, an expression vector can include a conjugate-encoding nucleic acid sequence linked to six histidine residues followed by a thioredoxin and an enterokinase cleavage site. The histidine residues facilitate detection and purification of the conjugate while the enterokinase cleavage site provides a means for purifying the construct from the remainder of the protein (see e.g., Kroll, DNA Cell. Biol. 12:441 (1993)).

Conjugate activity can be affected by various factors and therefore conjugates can be designed or optimized by taking into consideration one or more of these factors. Such factors include, for example, length, which can affect toxicity to cells. Cell killing activity of alpha helix forming lytic peptide domains can also depend on the stability of the helix. Linkers and spacers can affect membrane interaction of a lytic domain and the helical structure of a lytic domain. The charge of lytic peptide domains, which is determined in part by the particular amino acid residues present in the domain, also affects cell killing potency. The positioning of antibody, Heavy (H) chain, Light (L) chain, and second (lytic) domains (and additional domains, when present), relative to lytic domain (particular amino acid residue, or N- or C-terminus) also can affect cell killing activity of conjugates.

Conjugate in vivo half-life can be increased by constructing lytic peptide domains with one or more non-naturally occurring amino acids or derivatives. For example, conjugates with D-amino acids (e.g., up to 30%, 40%, 50%, 60%, or more of all residues are D-enantiomers) are resistant to serum proteolysis and therefore can be active for longer times thereby increasing in vivo potency. Furthermore, constructing lytic peptide domains with one or more non-naturally occurring amino acids or derivatives can reduce hemolytic activity. Such conjugates with D-enantiomers also have a greater tendency to be monomeric in solution-they do not significantly aggregate.

Peptides and peptidomimetics can be produced and isolated using methods known in the art. Peptides can be synthesized, whole or in part, using chemical methods known in the art (see, e.g., Caruthers (1980). Nucleic Acids Res. Symp. Ser. 215; Horn (1980); and Banga, A. K., Therapeutic Peptides and Proteins, Formulation, Processing and Delivery Systems (1995) Technomic Publishing Co., Lancaster, PA). Peptide synthesis can be performed using various solid-phase techniques (see, e.g., Roberge Science 269:202 (1995); Merrifield, Methods Enzymol. 289: 3 (1997)) and automated synthesis may be achieved, e.g., using the ABI 431A Peptide Synthesizer (Perkin Elmer) in accordance with the manufacturer's instructions. Peptides and peptide mimetics can also be synthesized using combinatorial methodologies. Synthetic residues and polypeptides incorporating mimetics can be synthesized using a variety of procedures and methodologies known in the art (see, e.g., Organic Syntheses Collective Volumes, Gilman, et al. (Eds) John Wiley & Sons, Inc., NY). Modified peptides can be produced by chemical modification methods (see, for example, Belousov, Nucleic Acids Res. 25:3440 (1997); Frenkel, Free Radic. Biol. Med. 19:373 (1995); and Blommers, Biochemistry 33:7886 (1994).

The invention further provides nucleic acids encoding the conjugates of the invention (and portions of antibodies, Heavy (H) chains, Light (L) chains, and fragments thereof, and second (lytic) domains, and additional domains, when present), and vectors that include nucleic acid encoding conjugates. Nucleic acid, which can also be referred to herein as a gene, polynucleotide, nucleotide sequence, primer, oligonucleotide or probe refers to natural or modified purine- and pyrimidine-containing polymers of any length, either polyribonucleotides or polydeoxyribonucleotides or mixed polyribo-polydeoxyribo nucleotides and α-anomeric forms thereof. The two or more purine- and pyrimidine-containing polymers are typically linked by a phosphoester bond or analog thereof. The terms can be used interchangeably to refer to all forms of nucleic acid, including deoxyribonucleic acid (DNA) and ribonucleic acid (RNA). The nucleic acids can be single strand, double, or triplex, linear or circular. Nucleic acids include genomic DNA, cDNA, and antisense. RNA nucleic acid can be spliced or unspliced mRNA, rRNA, RNA or antisense. Nucleic acids include naturally occurring, synthetic, as well as nucleotide analogues and derivatives.

As a result of the degeneracy of the genetic code, nucleic acids include sequences degenerate with respect to sequences encoding conjugates of the invention. Thus, degenerate nucleic acid sequences encoding conjugates are provided.

Nucleic acid can be produced using any of a variety of known standard cloning and chemical synthesis methods, and can be altered intentionally by site-directed mutagenesis or other recombinant techniques known to one skilled in the art. Purity of polynucleotides can be determined through sequencing, gel electrophoresis, UV spectrometry.

Nucleic acids may be inserted into a nucleic acid construct in which expression of the nucleic acid is influenced or regulated by an “expression control element,” referred to herein as an “expression cassette.” The term “expression control element” refers to one or more nucleic acid sequence elements that regulate or influence expression of a nucleic acid sequence to which it is operatively linked. An expression control element can include, as appropriate, promoters, enhancers, transcription terminators, gene silencers, a start codon (e.g., ATG) in front of a protein-encoding gene, etc.

An expression control element operatively linked to a nucleic acid sequence controls transcription and, as appropriate, translation of the nucleic acid sequence. The term “operatively linked” refers to a juxtaposition wherein the referenced components are in a relationship permitting them to function in their intended manner. Typically expression control elements are juxtaposed at the 5′ or the 3′ ends of the genes but can also be intronic.

Expression control elements include elements that activate transcription constitutively, that are inducible (i.e., require an external signal for activation), or derepressible (i.e., require a signal to turn transcription off; when the signal is no longer present, transcription is activated or “derepressed”). Also included in the expression cassettes of the invention are control elements sufficient to render gene expression controllable for specific cell-types or tissues (i.e., tissue-specific control elements). Typically, such elements are located upstream or downstream (i.e., 5′ and 3′) of the coding sequence. Promoters are generally positioned 5′ of the coding sequence. Promoters, produced by recombinant DNA or synthetic techniques, can be used to provide for transcription of the polynucleotides of the invention. A “promoter” is meant a minimal sequence element sufficient to direct transcription.

Nucleic acids may be inserted into a plasmid for propagation into a host cell and for subsequent genetic manipulation if desired. A plasmid is a nucleic acid that can be stably propagated in a host cell; plasmids may optionally contain expression control elements in order to drive expression of the nucleic acid. A vector is used herein synonymously with a plasmid and may also include an expression control element for expression in a host cell. Plasmids and vectors generally contain at least an origin of replication for propagation in a cell and a promoter. Plasmids and vectors are therefore useful for genetic manipulation of conjugate encoding nucleic acids, producing conjugates or antisense nucleic acid, and expressing conjugates in host cells and organisms, for example.

Bacterial system promoters include T7 and inducible promoters such as pL of bacteriophage λ, plac, ptrp, ptac (ptrp-lac hybrid promoter) and tetracycline responsive promoters. Insect cell system promoters include constitutive or inducible promoters (e.g., ecdysone). Mammalian cell constitutive promoters include SV40, RSV, bovine papilloma virus (BPV) and other virus promoters, or inducible promoters derived from the genome of mammalian cells (e.g., metallothionein IIA promoter; heat shock promoter) or from mammalian viruses (e.g., the adenovirus late promoter; the inducible mouse mammary tumor virus long terminal repeat). Alternatively, a retroviral genome can be genetically modified for introducing and directing expression of a conjugate in appropriate host cells.

Expression systems further include vectors designed for in vivo use. Particular non-limiting examples include adenoviral vectors (U.S. Pat. Nos. 5,700,470 and 5,731,172), adeno-associated vectors (U.S. Pat. No. 5,604,090), herpes simplex virus vectors (U.S. Pat. No. 5,501,979), retroviral vectors (U.S. Pat. Nos. 5,624,820, 5,693,508 and 5,674,703), BPV vectors (U.S. Pat. No. 5,719,054) and CMV vectors (U.S. Pat. No. 5,561,063).

Yeast vectors include constitutive and inducible promoters (see, e.g., Ausubel et al., In: Current Protocols in Molecular Biology, Vol. 2, Ch. 13, ed., Greene Publish. Assoc. & Wiley Interscience, 1988; Grant et al. Methods in Enzymology, 153:516 (1987), eds. Wu & Grossman; Bitter Methods in Enzymology, 152:673 (1987), eds. Berger & Kimmel, Acad. Press, N.Y.; and, Strathern et al., The Molecular Biology of the Yeast Saccharomyces (1982) eds. Cold Spring Harbor Press, Vols. I and II). A constitutive yeast promoter such as ADH or LEU2 or an inducible promoter such as GAL may be used (R. Rothstein In: DNA Cloning, A Practical Approach, Vol. 11, Ch. 3, ed. D. M. Glover, IRL Press, Wash., D.C., 1986). Vectors that facilitate integration of foreign nucleic acid sequences into a yeast chromosome, via homologous recombination for example, are known in the art. Yeast artificial chromosomes (YAC) are typically used when the inserted polynucleotides are too large for more conventional vectors (e.g., greater than about 12 Kb).

Expression vectors also can contain a selectable marker conferring resistance to a selective pressure or identifiable marker (e.g., beta-galactosidase), thereby allowing cells having the vector to be selected for, grown and expanded. Alternatively, a selectable marker can be on a second vector that is cotransfected into a host cell with a first vector containing a nucleic acid encoding a conjugate.

Selection systems include but are not limited to herpes simplex virus thymidine kinase gene (Wigler et al., Cell 11:223 (1977)), hypoxanthine-guanine phosphoribosyltransferase gene (Szybalska et al., Proc. Natl. Acad. Sci. USA 48:2026 (1962)), and adenine phosphoribosyltransferase (Lowy et al., Cell 22:817 (1980)) genes which can be employed in tk-, hgprt- or aprt-cells, respectively. Additionally, antimetabolite resistance can be used as the basis of selection for dhfr, which confers resistance to methotrexate (O'Hare et al., Proc. Natl. Acad. Sci. USA 78:1527 (1981)); the gpt gene, which confers resistance to mycophenolic acid (Mulligan et al., Proc. Natl. Acad. Sci. USA 78:2072 (1981)); neomycin gene, which confers resistance to aminoglycoside G-418 (Colberre-Garapin et al., J. Mol. Biol. 150: 1 (1981)); puromycin; and hygromycin gene, which confers resistance to hygromycin (Santerre et al., Gene 30:147 (1984)). Additional selectable genes include trpB, which allows cells to utilize indole in place of tryptophan; hisD, which allows cells to utilize histinol in place of histidine (Hartman et al., Proc. Natl. Acad. Sci. USA 85:8047 (1988)); and ODC (ornithine decarboxylase), which confers resistance to the ornithine decarboxylase inhibitor, 2-(difluoromethyl)-DL-ornithine, DFMO (McConlogue (1987) In: Current Communications in Molecular Biology, Cold Spring Harbor Laboratory).

Host cells that express conjugates, and host cells transformed with nucleic acids encoding conjugates (e.g., antibodies, Heavy (H) chains, Light (L) chains, and fragments thereof, and second (lytic) domains, and additional domains, when present), and vectors including a nucleic acid that encodes the conjugates are also provided. In one embodiment, a host cell is a prokaryotic cell. In another embodiment, a host cell is a eukaryotic cell. In various aspects, the eukaryotic cell is a yeast or mammalian (e.g., human, primate, etc.) cell.

As used herein, a “host cell” is a cell into which a nucleic acid is introduced that can be propagated, transcribed, or encoded conjugate expressed. The term also includes any progeny or subclones of the host cell. Host cells include cells that express conjugates.

Host cells include but are not limited to microorganisms such as bacteria and yeast; and plant, insect and mammalian cells. For example, bacteria transformed with recombinant bacteriophage nucleic acid, plasmid nucleic acid or cosmid nucleic acid expression vectors; yeast transformed with recombinant yeast expression vectors; plant cell systems infected with recombinant virus expression vectors (e.g., cauliflower mosaic virus, CaMV; tobacco mosaic virus, TMV) or transformed with recombinant plasmid expression vectors (e.g., Ti plasmid); insect cell systems infected with recombinant virus expression vectors (e.g., baculovirus); and animal cell systems infected with recombinant virus expression vectors (e.g., retroviruses, adenovirus, vaccinia virus), or transformed animal cell systems engineered for transient or stable propagation or expression.

Antibody and polypeptide conjugates, nucleic acids encoding conjugates, and vectors and host cells expressing conjugates or transformed with nucleic acids encoding conjugates include isolated and purified forms. The term “isolated,” when used as a modifier of an invention conjugate or composition, means that the composition is made by the hand of man or is separated, substantially completely or at least in part, from the naturally occurring in vivo environment. Generally, an isolated composition is substantially free of one or more materials with which it normally associates with in nature, for example, one or more protein, nucleic acid, lipid, carbohydrate, cell membrane. The term “isolated” does not exclude alternative physical forms of the composition, such as multimers/oligomers, variants, modifications or derivatized forms, or forms expressed in host cells produced by the hand of man. The term “isolated” also does not exclude forms (e.g., pharmaceutical formulations and combination compositions) in which there are combinations therein, any one of which is produced by the hand of man.

An “isolated” composition can also be “purified” when free of some, a substantial number of, most or all of the materials with which it typically associates with in nature. Thus, an isolated conjugate that also is substantially pure does not include polypeptides or polynucleotides present among millions of other sequences, such as proteins of a protein library or nucleic acids in a genomic or cDNA library, for example. A “purified” composition can be combined with one or more other molecules.

In accordance with the invention, there are provided mixtures of conjugates and combination compositions. In one embodiment, a mixture includes one or more conjugates and a pharmaceutically acceptable carrier or excipient. In another embodiment, a mixture includes one or more conjugates and an anti-cell proliferative, anti-tumor, anti-cancer, or anti-neoplastic treatment or agent. In a further embodiment, a mixture includes one or more conjugates and an immune enhancing agent. Combinations, such as one or more conjugates in a pharmaceutically acceptable carrier or excipient, with one or more of an anti-cell proliferative, anti-tumor, anti-cancer, or anti-neoplastic treatment or agent, and an immune enhancing treatment or agent, are also provided.

Conjugates of the invention, such as antibodies, Heavy (H) chains, Light (L) chains, and fragments thereof that bind to a target (e.g., a receptor), and second (lytic) domains, can be used to target cells for lysis, cell death or apoptosis. Such cells can be selectively targeted. For example a cell that expresses receptor such as Her2/neu or CD20 can be targeted by a conjugate and thereby be preferentially killed compared to cells that express little if any receptor.

In accordance with the invention, there are provided methods and uses of reducing or inhibiting proliferation of a cell that expresses a target (e.g., a receptor) and methods of reducing or inhibiting cell proliferation. In one embodiment, a method or use includes contacting a target expressing cell with a conjugate in an amount sufficient to reduce or inhibit proliferation of the cell. In another embodiment, a method includes contacting a target expressing cell with a conjugate in an amount sufficient to reduce or inhibit cell proliferation.

Also provided are methods and uses of reducing or inhibiting proliferation of a hyperproliferative cell that expresses a target (e.g., a receptor), and methods and uses of reducing or inhibiting proliferation of hyperproliferating cells that express a target (e.g., a receptor). In one embodiment, a method or use includes contacting a hyperproliferative target expressing cell or hyperproliferating target expressing cells with a conjugate in an amount sufficient to reduce or inhibit proliferation.

Further provided are methods and uses of reducing or inhibiting proliferation of a non-metastatic or metastatic neoplastic, cancer, tumor and malignant cells that express a target (e.g., a receptor). In one embodiment, a method or use includes contacting a neoplastic, cancer, tumor or malignant target expressing cell with a conjugate in an amount sufficient to reduce or inhibit proliferation of the target expressing cell.

Still further provided are methods and uses of reducing or inhibiting proliferation of a dormant or non-dividing non-metastatic or metastatic neoplastic, cancer, tumor and malignant cells that express a target (e.g., a receptor). In one embodiment, a method or use includes contacting a dormant or non-dividing neoplastic, cancer, tumor or malignant target expressing cell with a conjugate in an amount sufficient to reduce or inhibit proliferation of the dormant or non-dividing cell.

Additionally provided are methods and uses of selectively reducing or inhibiting proliferation of a cell (e.g., a hyperproliferating cell) that expresses a target (e.g., a receptor). In one embodiment, a method or use includes contacting the target expressing cell with a conjugate in an amount sufficient to reduce or inhibit proliferation of the cell (e.g., hyperproliferating cell), wherein the conjugate binds to a target (e.g., a receptor) expressed by the cell.

Yet additionally provided are methods and uses of selectively reducing or inhibiting proliferation of a neoplastic, tumor, cancer or malignant cell that expresses that expresses a target (e.g., a receptor). In one embodiment, a method or a use includes contacting the cell with a conjugate in an amount sufficient to reduce or inhibit proliferation of the neoplastic, tumor, cancer or malignant cell, wherein the conjugate binds to the target (e.g., a receptor) expressed by the cell.

The term “contacting” means direct or indirect binding or interaction between two or more entities (e.g., between a conjugate and a target and/or cell). Contacting as used herein includes in solution, in solid phase, in vitro, ex vivo, in a cell and in vivo. Contacting in vivo can be referred to as administering, or administration or delivery.

Cells to target for reducing or inhibiting proliferation, non-selectively or selectively, include cells that express a target (e.g., a receptor). Non-limiting exemplary cells include breast, ovarian, uterine, cervical, stomach, lung, gastric, colon, bladder, glial, dermal (e.g., melanocytes), hematologic and endometrial cells.

Conjugates and methods and uses of the invention are also applicable to treating undesirable or aberrant cell proliferation and hyperproliferative disorders, which include cells expressing a target (e.g., a receptor). Thus, in accordance with the invention, methods and uses of treating undesirable or aberrant cell proliferation and hyperproliferative disorders are provided. In one embodiment, a method or a use includes administering to a subject (in need of treatment) an amount of a conjugate sufficient to treat the undesirable or aberrant cell proliferation or the hyperproliferative disorder.

The term “hyperproliferative disorder” refers to any undesirable or aberrant cell survival (e.g., failure to undergo programmed cell death or apoptosis), growth or proliferation. Such disorders include benign hyperplasias, non-metastatic and metastatic neoplasias, cancers, tumors and malignancies. Undesirable or aberrant cell proliferation and hyperproliferative disorders can affect any cell, tissue, organ in a subject. Undesirable or aberrant cell proliferation and hyperproliferative disorders can be present in a subject, locally, regionally or systemically. A hyperproliferative disorder can arise from a multitude of tissues and organs, including but not limited to breast, lung (e.g., small cell or non-small cell), thyroid, head and neck, brain, nasopharynx, throat, nose or sinuses, lymphoid, adrenal gland, pituitary gland, thyroid, lymph, gastrointestinal (mouth, esophagus, stomach, duodenum, ileum, jejunum (small intestine), colon, rectum), genito-urinary tract (uterus, ovary, vagina, cervix, endometrium, fallopian tube, bladder, testicle, penis, prostate), kidney, pancreas, liver, bone, bone marrow, lymph, blood (hematologic), brain (glial), muscle, skin, dermal (e.g., melanocytes), and stem cells, which may or may not metastasize to other secondary sites, regions or locations.

Conjugates and methods and uses of the invention are also applicable to metastatic or non-metastatic tumor, cancer, malignancy or neoplasia of any cell, organ or tissue origin. Such disorders can affect virtually any cell or tissue type, e.g., carcinoma, sarcoma, melanoma, neural, and reticuloendothelial or hematopoietic neoplastic disorders (e.g., myeloma, lymphoma or leukemia).

As used herein, the terms “neoplasia” and “tumor” refer to a cell or population of cells whose growth, proliferation or survival is greater than growth, proliferation or survival of a normal counterpart cell, e.g. a cell proliferative or differentiative disorder. A tumor is a neoplasia that has formed a distinct mass or growth. A “cancer” or “malignancy” refers to a neoplasia or tumor that can invade adjacent spaces, tissues or organs. A “metastasis” refers to a neoplasia, tumor, cancer or malignancy that has disseminated or spread from its primary site to one or more secondary sites, locations or regions within the subject, in which the sites, locations or regions are distinct from the primary tumor or cancer. All or a portion of such cells can express a target (e.g., a receptor) can therefore be targeted with conjugates in accordance with the invention.

Neoplastic, tumor, cancer and malignant cells (metastatic or non-metastatic) include dormant or residual neoplastic, tumor, cancer and malignant cells, all or a portion of which express a target (e.g., a receptor). Such cells typically consist of remnant tumor cells that are not dividing (G0-G1 arrest). These cells can persist in a primary site or as disseminated neoplastic, tumor, cancer or malignant cells as a minimal residual disease. These dormant neoplastic, tumor, cancer or malignant cells remain asymptomatic, but can develop severe symptoms and death once these dormant cells proliferate. Invention methods can be used to reduce or inhibit proliferation of dormant neoplastic, tumor, cancer or malignant cells, which can in turn inhibit or reduce tumor or cancer relapse, or tumor or cancer metastasis or progression.

In accordance with the invention, methods of treating a subject having a metastatic or non-metastatic tumor, cancer, malignancy or neoplasia are provided. In one embodiment, a method includes administering to a subject (in need of treatment) an amount of a conjugate of sufficient to treat (e.g., reduce or inhibit proliferation) the metastatic or non-metastatic tumor, cancer, malignancy or neoplasia.

The metastatic or non-metastatic tumor, cancer, malignancy or neoplasia may be in any stage, e.g., early or advanced, such as a stage I, II, III, IV or V tumor. The metastatic or non-metastatic tumor, cancer, malignancy or neoplasia may have been subject to a prior treatment or be stabilized (non-progressing) or in remission.

In terms of metastasis, invention methods can be used to reduce or inhibit metastasis of a primary tumor or cancer to other sites, or the formation or establishment of metastatic tumors or cancers at other sites distal from the primary tumor or cancer thereby inhibiting or reducing tumor or cancer relapse or tumor or cancer progression. Thus, methods of the invention include, among other things, 1) reducing or inhibiting growth, proliferation, mobility or invasiveness of tumor or cancer cells that potentially or do develop metastases (e.g., disseminated tumor cells, DTC); 2) reducing or inhibiting formation or establishment of metastases arising from a primary tumor or cancer to one or more other sites, locations or regions distinct from the primary tumor or cancer; 3) reducing or inhibiting growth or proliferation of a metastasis at one or more other sites, locations or regions distinct from the primary tumor or cancer after a metastasis has formed or has been established; and 4) reducing or inhibiting formation or establishment of additional metastasis after the metastasis has been formed or established.

Cells of a metastatic or non-metastatic tumor, cancer, malignancy or neoplasia (all or a portion of which express a target (e.g., a receptor)) may be aggregated in a “solid” cell mass or be dispersed or diffused. A “solid” tumor refers to cancer, neoplasia or metastasis that typically aggregates together and forms a mass. Specific non-limiting examples include breast, ovarian, uterine, cervical, stomach, lung, gastric, colon, bladder, glial, dermal (e.g., melanocytes) and endometrial tumors/cancers.

Carcinomas, which refer to malignancies of epithelial or endocrine tissue, include respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from the uterus, cervix, lung, prostate, breast, head and neck, colon, pancreas, testes, adrenal, kidney, esophagus, stomach, liver and ovary. The term also includes carcinosarcomas, e.g., which include malignant tumors composed of carcinomatous and sarcomatous tissues. Adenocarcinoma includes a carcinoma of a glandular tissue, or in which the tumor forms a gland like structure.

Sarcomas refer to malignant tumors of mesenchymal cell origin. Exemplary sarcomas include for example, lymphosarcoma, liposarcoma, osteosarcoma, chondrosarcoma, leiomyosarcoma, rhabdomyosarcoma and fibrosarcoma.

Neural neoplasias include glioma, glioblastoma, meningioma, neuroblastoma, retinoblastoma, astrocytoma and oligodendrocytoma.

A “liquid tumor,” which refers to neoplasia that is dispersed or is diffuse in nature, as they do not typically form a solid mass. Particular examples include neoplasia of the reticuloendothelial or hematopoietic system, such as lymphomas, myelomas and leukemias. Non-limiting examples of leukemias include acute and chronic lymphoblastic, myeloblastic and multiple myeloma. Typically, such diseases arise from poorly differentiated acute leukemias, e.g., erythroblastic leukemia and acute megakaryoblastic leukemia. Specific myeloid disorders include, but are not limited to, acute promyeloid leukemia (APML), acute myelogenous leukemia (AML) and chronic myelogenous leukemia (CML). Lymphoid malignancies include, but are not limited to, acute lymphoblastic leukemia (ALL), which includes B-lineage ALL (B-ALL) and T-lineage ALL (T-ALL), chronic lymphocytic leukemia (CLL), prolymphocytic leukemia (PLL), hairy cell leukemia (HLL) and Waldenstroem's macroglobulinemia (WM). Specific malignant lymphomas include, non-Hodgkin lymphoma and variants, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

As disclosed herein, undesirable or aberrant cell proliferation or hyperproliferative disorders can occur in uterus, breast, vagina, cervix, endometrium and fallopian tube. Thus, in accordance with the invention, there are provided methods and uses of treating uterus, breast, vagina, cervix, endometrium and fallopian tube hyperproliferative disorders. In one embodiment, a method or use includes administering to a subject an amount of a conjugate sufficient to treat a uterus, breast, vagina, cervix, endometrium or fallopian tube hyperproliferative disorder.

Any composition, treatment, protocol, therapy or regimen having an anti-cell proliferative activity or effect can be combined with a conjugate or used in combination in a method or use of the invention. Conjugates and methods and uses of the invention therefore include anti-proliferative, anti-tumor, anti-cancer, anti-neoplastic and anti-metastatic treatments, protocols and therapies, which include any other composition, treatment, protocol or therapeutic regimen that inhibits, decreases, retards, slows, reduces or prevents a hyperproliferative disorder, such as tumor, cancer, malignant or neoplastic growth, progression, metastasis, proliferation or survival, or worsening in vitro or in vivo. Particular non-limiting examples of an anti-proliferative (e.g., tumor) therapy include chemotherapy, immunotherapy, radiotherapy (ionizing or chemical), local thermal (hyperthermia) therapy, surgical resection and vaccination. A conjugate can be administered prior to, substantially contemporaneously with or following administration or use of the anti-cell proliferative, anti-neoplastic, anti-tumor, anti-cancer, anti-metastatic or immune-enhancing treatment or therapy. A conjugate can be administered or used as a combination composition with the anti-cell proliferative, anti-neoplastic, anti-tumor, anti-cancer, anti-metastatic or immune-enhancing treatment or therapy, metastatic or non-metastatic tumor, cancer, malignancy or neoplasia.

Anti-proliferative, anti-neoplastic, anti-tumor, anti-cancer and anti-metastatic compositions, therapies, protocols or treatments include those that prevent, disrupt, interrupt, inhibit or delay cell cycle progression or cell proliferation; stimulate or enhance apoptosis or cell death, inhibit nucleic acid or protein synthesis or metabolism, inhibit cell division, or decrease, reduce or inhibit cell survival, or production or utilization of a necessary cell survival factor, growth factor or signaling pathway (extracellular or intracellular). Non-limiting examples of chemical agent classes having anti-cell proliferative, anti-neoplastic, anti-tumor, anti-cancer and anti-metastatic activities include alkylating agents, anti-metabolites, plant extracts, plant alkaloids, nitrosoureas, hormones, nucleoside and nucleotide analogues. Specific examples of drugs having anti-cell proliferative, anti-neoplastic, anti-tumor, anti-cancer and anti-metastatic activities include cyclophosphamide, azathioprine, cyclosporin A, prednisolone, melphalan, chlorambucil, mechlorethamine, busulphan, methotrexate, 6-mercaptopurine, thioguanine, 5-fluorouracil, cytosine arabinoside, AZT, 5-azacytidine (5-AZC) and 5-azacytidine related compounds such as decitabine (5-aza-2′deoxycytidine), cytarabine, 1-beta-D-arabinofuranosyl-5-azacytosine and dihydro-5-azacytidine, bleomycin, actinomycin D, mithramycin, mitomycin C, carmustine, lomustine, semustine, streptozotocin, hydroxyurea, cisplatin, mitotane, procarbazine, dacarbazine, a taxane (e.g., taxol or paclitaxel), vinblastine, vincristine, doxorubicin and dibromomannitol etc.

Additional agents that are applicable with conjugates and methods and uses are known to the skilled artisan and can be employed. For example, biologicals such as antibodies that are different from the antibodies used for conjugation, cell growth factors, cell survival factors, cell differentiative factors, cytokines and chemokines can be administered or used. Non-limiting examples of monoclonal antibodies include rituximab (Rituxan®), trastuzumab (Herceptin®), pertuzumab (Perjeta®)), bevacizumab (Avastin®), ranibizumab (Lucentis®), cetuximab (Erbitux®), alemtuzumab (Campath®), panitumumab (Vectibix®), ibritumomab tiuxetan (Zevalin®), tositumomab (Bexxar®), ipilimumab, zalutumumab, dalotuzumab, figitumumab, ramucirumab, galiximab, farletuzumab, ocrelizumab, ofatumumab (Arzerra®), tositumumab, ibritumomab, 2F2 (HuMax-CD20), 7D8, IgM2C6, IgG1 2C6, 11B8, B1, 2H7, LT20, 1F5 or AT80 daclizumab (Zenapax®), anti-LHRH receptor antibodies such as clone A9E4, F1G4, AT2G7, GNRH03, GNRHR2, etc. which can be used in combination with, inter alia, a conjugate in accordance with the invention.

Other targeted drugs that are applicable for use with the conjugates are kinase inhibitors e.g., imatinib (Gleevec®), gefitinib (Iressa®), bortzomib (Velcade®), lapatinib (Tykerb®), sunitinib (Sutent®), sorafenib (Nevaxar®), nilotinib (Tasigna®) etc. Non-limiting examples of cell growth factors, cell survival factors, cell differentiative factors, cytokines and chemokines include IL-2, IL-1α, IL-1β, IL-3, IL-6, IL-7, granulocyte-macrophage-colony stimulating factor (GMCSF), IFN-γ, IL-12, TNF-α, TNFβ, MIP-1α, MIP-1β, RANTES, SDF-1, MCP-1, MCP-2, MCP-3, MCP-4, eotaxin, eotaxin-2, I-309/TCA3, ATAC, HCC-1, HCC-2, HCC-3, LARC/MIP-3α, PARC, TARC, CKβ, CKβ6, CKβ7, CKβ8, CKβ9, CKβ11, CKβ12, C10, IL-8, GROα, GROβ, ENA-78, GCP-2, PBP/CTAPIII≡-TG/NAP-2, Mig, PBSF/SDF-1 and lymphotactin.

Additional non-limiting examples include immune-enhancing treatments and therapies, which include cell based therapies. In particular, immune-enhancing treatments and therapies include administering lymphocytes, plasma cells, macrophages, dendritic cells, NK cells and B-cells.

Methods and uses of treating a metastatic or non-metastatic tumor, cancer, malignancy or neoplasia, methods and uses of treating a subject in need of treatment due to having or at risk of having a metastatic or non-metastatic tumor, cancer, malignancy or neoplasia, and methods and uses of increasing effectiveness or improving an anti-proliferative, anti-tumor, anti-cancer, anti-neoplasia or anti-malignancy, therapy are provided. In respective embodiments, a method or use includes administering to a subject with or at risk of a metastatic or non-metastatic tumor, cancer, malignancy or neoplasia, an amount of a conjugate sufficient to treat the metastatic or non-metastatic tumor, cancer, malignancy or neoplasia; administering to the subject an amount of a conjugate sufficient to treat the subject; and administering to a subject that is undergoing or has undergone metastatic or non-metastatic tumor, cancer, malignancy or neoplasia therapy, an amount of a conjugate sufficient to increase effectiveness of the anti-proliferative, anti-tumor, anti-cancer, anti-neoplasia or anti-malignancy therapy.

Methods and uses of the invention may be practiced prior to (i.e. prophylaxis), concurrently with or after evidence of the presence of undesirable or aberrant cell proliferation or a hyperproliferative disorder, disease or condition begins (e.g., one or more symptoms). Administering or using a conjugate prior to, concurrently with or immediately following development of a symptom of undesirable or aberrant cell proliferation or a hyperproliferative disorder may decrease the occurrence, frequency, severity, progression, or duration of one or more symptoms of the undesirable or aberrant cell proliferation or a hyperproliferative disorder, disease or condition in the subject. In addition, administering or using a conjugate prior to, concurrently with or immediately following development of one or more symptoms of the undesirable or aberrant cell proliferation or a hyperproliferative disorder, disease or condition may inhibit, decrease or prevent the spread or dissemination of hyperproliferating cells (e.g., metastasis) to other sites, regions, tissues or organs in a subject, or establishment of hyperproliferating cells (e.g., metastasis) at other sites, regions, tissues or organs in a subject.

Conjugates and the methods and uses of the invention, such as treatment methods and uses, can provide a detectable or measurable therapeutic benefit or improvement to a subject. A therapeutic benefit or improvement is any measurable or detectable, objective or subjective, transient, temporary, or longer-term benefit to the subject or improvement in the condition, disorder or disease, an adverse symptom, consequence or underlying cause, of any degree, in a tissue, organ, cell or cell population of the subject.

Therapeutic benefits and improvements include, but are not limited to, reducing or decreasing occurrence, frequency, severity, progression, or duration of one or more symptoms or complications associated with a disorder, disease or condition, or an underlying cause or consequential effect of the disorder, disease or condition. Conjugates and methods and uses of the invention therefore include providing a therapeutic benefit or improvement to a subject.

In a method or use of the invention in which a therapeutic benefit or improvement is a desired outcome, a conjugate can be administered in a sufficient or effective amount to a subject in need thereof. An “amount sufficient” or “amount effective” refers to an amount that is anticipated to provide, in single or multiple doses, alone or in combination, with one or more other compositions (therapeutic agents such as a chemotheraputic or immune stimulating drug), treatments, protocols, or therapeutic regimens agents, a detectable response of any duration of time (long or short term), a desired outcome in or a benefit to a subject of any measurable or detectable degree or for any duration of time (e.g., for hours, days, months, years, or cured). The doses or “sufficient amount” or “effective amount” for treatment (e.g., to provide a therapeutic benefit or improvement) typically are effective to ameliorate a disorder, disease or condition, or one, multiple or all adverse symptoms, consequences or complications of the disorder, disease or condition, to a measurable extent, although reducing or inhibiting a progression or worsening of the disorder, disease or condition or a symptom, is considered a satisfactory outcome.

The term “ameliorate” means a detectable objective or subjective improvement in a subject's condition. A detectable improvement includes a subjective or objective reduction in the occurrence, frequency, severity, progression, or duration of a symptom caused by or associated with a disorder, disease or condition, an improvement in an underlying cause or a consequence of the disorder, disease or condition, or a reversal of the disorder, disease or condition.

Treatments or uses can therefore result in inhibiting, reducing or preventing a disorder, disease or condition, or an associated symptom or consequence, or underlying cause; inhibiting, reducing or preventing a progression or worsening of a disorder, disease, condition, symptom or consequence, or underlying cause; or further deterioration or occurrence of one or more additional symptoms of the disorder, disease condition, or symptom. Thus, a successful treatment outcome leads to a “therapeutic effect,” or “benefit” or inhibiting, reducing or preventing the occurrence, frequency, severity, progression, or duration of one or more symptoms or underlying causes or consequences of a condition, disorder, disease or symptom in the subject. Treatment methods and uses affecting one or more underlying causes of the condition, disorder, disease or symptom are therefore considered to be beneficial. Stabilizing or inhibiting progression or worsening of a disorder or condition is also a successful treatment outcome.

A therapeutic benefit or improvement therefore need not be complete ablation of any one, most or all symptoms, complications, consequences or underlying causes associated with the condition, disorder or disease. Thus, a satisfactory endpoint is achieved when there is a stabilization or an incremental improvement in a subject's condition, or a partial reduction in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of one or more of the physiological, biochemical or cellular manifestations or characteristics of the disorder or disease, over a short or long duration of time (hours, days, weeks, months, etc.).

In particular embodiments, a method or use of treatment results in partial or complete destruction of a metastatic or non-metastatic tumor, cancer, malignant or neoplastic cell mass, volume, size or numbers of cells; results in stimulating, inducing or increasing metastatic or non-metastatic tumor, cancer, malignant or neoplastic cell necrosis, lysis or apoptosis; results in reducing metastatic or non-metastatic tumor, cancer, malignant or neoplastic volume, size, cell mass; results in inhibiting or preventing progression or an increase in metastatic or non-metastatic tumor, cancer, malignant or neoplastic volume, mass, size or cell numbers; results in inhibiting or decreasing the spread or dissemination of hyperproliferating cells (e.g., metastasis) to other (secondary) sites, regions, tissues or organs in a subject, or establishment of hyperproliferating cells (e.g., metastasis) at other (secondary) sites, regions, tissues or organs in a subject; or results in prolonging lifespan of the subject. In additional particular embodiments, a method or use of treatment results in reducing or decreasing severity, duration or frequency of an adverse symptom or complication associated with or caused by the metastatic or non-metastatic tumor, cancer, malignancy or neoplasia.

An amount sufficient or an amount effective can but need not be provided in a single administration or dose and, can but need not be, administered alone or in combination with another composition (e.g., chemotherapeutic or immune enhancing or stimulating agent), treatment, protocol or therapeutic regimen. For example, the amount may be proportionally increased as indicated by the need of the subject, status of the disorder, disease or condition treated or the side effects of treatment. In addition, an amount sufficient or an amount effective need not be sufficient or effective if given in single or multiple doses without a second composition (e.g., chemotherapeutic or immune stimulating agent), treatment, protocol or therapeutic regimen, since additional doses, amounts or duration above and beyond such doses, or additional compositions (e.g., chemotherapeutic or immune stimulating agents), treatments, protocols or therapeutic regimens may be included in order to be considered effective or sufficient in a given subject. Amounts considered sufficient also include amounts that result in a reduction of the use of another treatment, therapeutic regimen or protocol.

An amount sufficient or an amount effective need not be effective in each and every subject treated, prophylactically or therapeutically, nor a majority of treated subjects in a given group or population. As is typical for treatment or therapeutic methods, some subjects will exhibit greater or less response to a given treatment, therapeutic regimen or protocol. An amount sufficient or an amount effective refers to sufficiency or effectiveness in a particular subject, not a group or the general population. Such amounts will depend in part upon the condition treated, such as the type or stage of undesirable or aberrant cell proliferation or hyperproliferative disorder (e.g., a metastatic or non-metastatic tumor, cancer, malignancy or neoplasia), the therapeutic effect desired, as well as the individual subject (e.g., the bioavailability within the subject, gender, age, etc.).

Particular non-limiting examples of therapeutic benefit or improvement for undesirable or aberrant cell proliferation, such as a hyperproliferative disorder (e.g., a metastatic or non-metastatic tumor, cancer, malignancy or neoplasia) include a reduction in cell size, mass or volume, inhibiting an increase in cell size, mass or volume, a slowing or inhibition of worsening or progression, stimulating cell necrosis, lysis or apoptosis, reducing or inhibiting neoplastic or tumor malignancy or metastasis, reducing mortality, and prolonging lifespan of a subject. Thus, inhibiting or delaying an increase in cell size, mass, volume or metastasis (stabilization) can increase lifespan (reduce mortality) even if only for a few days, weeks or months, even though complete ablation of the metastatic or non-metastatic tumor, cancer, malignancy or neoplasia has not occurred. Adverse symptoms and complications associated with a hyperproliferative disorder (e.g., a metastatic or non-metastatic tumor, cancer, malignancy or neoplasia) that can be reduced or decreased include, for example, pain, nausea, discomfort, lack of appetite, lethargy and weakness. A reduction in the occurrence, frequency, severity, progression, or duration of a symptom of undesirable or aberrant cell proliferation, such as a hyperproliferative disorder (e.g., a metastatic or non-metastatic tumor, cancer, malignancy or neoplasia), such as an improvement in subjective feeling (e.g., increased energy, appetite, reduced nausea, improved mobility or psychological well being, etc.), are therefore all examples of therapeutic benefit or improvement.

For example, a sufficient or effective amount of a conjugate is considered as having a therapeutic effect if administration results in less chemotherapeutic drug, radiation or immunotherapy being required for treatment of undesirable or aberrant cell proliferation, such as a hyperproliferative disorder (e.g., a metastatic or non-metastatic tumor, cancer, malignancy or neoplasia).

The term “subject” refers to animals, typically mammalian animals, such as humans, non human primates (apes, gibbons, chimpanzees, orangutans, macaques), domestic animals (dogs and cats), farm animals (horses, cows, goats, sheep, pigs) and experimental animal (mouse, rat, rabbit, guinea pig). Subjects include animal disease models, for example, animal models of undesirable or aberrant cell proliferation, such as a hyperproliferative disorder (e.g., a metastatic or non-metastatic tumor, cancer, malignancy or neoplasia) for analysis of conjugates in vivo.

Subjects appropriate for treatment include those having or at risk of having a metastatic or non-metastatic tumor, cancer, malignant or neoplastic cell, those undergoing as well as those who are undergoing or have undergone anti-proliferative (e.g., metastatic or non-metastatic tumor, cancer, malignancy or neoplasia) therapy, including subjects where the tumor is in remission. “At risk” subjects typically have risk factors associated with undesirable or aberrant cell proliferation, development of hyperplasia (e.g., a tumor).

Particular examples of at risk or candidate subjects include those with cells that express a target (e.g., a receptor) to which a conjugate can bind, particularly where cells targeted for necrosis, lysis, killing or destruction express greater numbers or amounts of a target (e.g., a receptor) than non-target cells. Such cells can be selectively or preferentially targeted for necrosis, lysis or killing.

At risk subjects also include those that are candidates for and those that have undergone surgical resection, chemotherapy, immunotherapy, ionizing or chemical radiotherapy, local or regional thermal (hyperthermia) therapy, or vaccination. The invention is therefore applicable to treating a subject who is at risk of a metastatic or non-metastatic tumor, cancer, malignancy or neoplasia or a complication associated with a metastatic or non-metastatic tumor, cancer, malignancy or neoplasia, for example, due to metastatic or non-metastatic tumor, cancer, malignancy or neoplasia reappearance or regrowth following a period of stability or remission.

Risk factors include gender, lifestyle (diet, smoking), occupation (medical and clinical personnel, agricultural and livestock workers), environmental factors (carcinogen exposure), family history (autoimmune disorders, diabetes, etc.), genetic predisposition, etc. For example, subjects at risk for developing melanoma include excess sun exposure (ultraviolet radiation), fair skin, high numbers of naevi (dysplastic nevus), patient phenotype, family history, or a history of a previous melanoma. Subjects at risk for developing cancer can therefore be identified by lifestyle, occupation, environmental factors, family history, and genetic screens for tumor associated genes, gene deletions or gene mutations. Subjects at risk for developing breast cancer lack Brcal, for example. Subjects at risk for developing colon cancer have early age or high frequency polyp formation, or deleted or mutated tumor suppressor genes, such as adenomatous polyposis coli (APC), for example.

Subjects also include those precluded from other treatments. For example, certain subjects may not be good candidates for surgical resection, chemotherapy, immunotherapy, ionizing or chemical radiotherapy, local or regional thermal (hyperthermia) therapy, or vaccination. Thus, candidate subjects for treatment in accordance with the invention include those that are not a candidate for surgical resection, chemotherapy, immunotherapy, ionizing or chemical radiotherapy, local or regional thermal (hyperthermia) therapy, or vaccination.

Conjugates may be formulated in a unit dose or unit dosage form. In a particular embodiment, a conjugate is in an amount anticipated to be effective to treat a subject having undesirable or aberrant cell proliferation or a hyperproliferative disorder. In an additional particular embodiment, a conjugate is in an amount anticipated to be effective to treat a subject having a metastatic or non-metastatic tumor, cancer, malignancy or neoplasia. Exemplary unit doses range from about 1-25, 25-250, 250-500, 500-1000, 1000-2500, 2500-5000, 5000-25,000, 5000-50,000 ug; and from about 1-25, 25-250, 250-500, 500-1000, 1000-2500 or 2500-5000, 5000-25,000, 5000-50,000 or 50,000-100,000 mg.

Conjugates and methods and uses of the invention may be contacted or provided in vitro, ex vivo or in vivo. Conjugates can be administered to provide the intended effect as a single or multiple dosages, for example, in an effective or sufficient amount. Exemplary doses range from about 1-25, 25-250, 250-500, 500-1000, 1000-2500 or 2500-5000, 5000-25,000, 5000-50,000, or 50,000-100,000 μg/kg, on consecutive days, or alternating days or intermittently. Single or multiple doses can be administered on consecutive days, alternating days or intermittently.

Conjugates can be administered and methods may be practiced via systemic, regional or local administration, by any route. For example, a conjugate can be administered systemically, regionally or locally, intravenously, orally (e.g., ingestion or inhalation), intramuscularly, intraperitoneally, intradermally, subcutaneously, intracavity, intracranially, transdermally (topical), parenterally, e.g. transmucosally or rectally. Compositions and methods and uses of the invention including pharmaceutical formulations can be administered via a (micro) encapsulated delivery system or packaged into an implant for administration.

The invention further provides conjugates and methods and uses in which the conjugates are included in pharmaceutical compositions. A pharmaceutical composition refers to “pharmaceutically acceptable” and “physiologically acceptable” carriers, diluents or excipients. As used herein, the term “pharmaceutically acceptable” and “physiologically acceptable,” when referring to carriers, diluents or excipients includes solvents (aqueous or non-aqueous), detergents, solutions, emulsions, dispersion media, coatings, isotonic and absorption promoting or delaying agents, compatible with pharmaceutical administration and with the other components of the formulation. Such formulations can be contained in a tablet (coated or uncoated), capsule (hard or soft), microbead, emulsion, powder, granule, crystal, suspension, syrup or elixir.

Pharmaceutical compositions can be formulated to be compatible with a particular route of administration or use. Compositions for parenteral, intradermal, or subcutaneous administration can include a sterile diluent, such as water, saline solution, fixed oils, polyethylene glycols, glycerine, propylene glycol or other synthetic solvents. The preparation may contain one or more preservatives to prevent microorganism growth (e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose).

Pharmaceutical compositions for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). The carrier can be a solvent or dispersion medium containing, for example, water, ethanol, polyol (e.g., glycerol, propylene glycol, and polyetheylene glycol), and suitable mixtures thereof. Fluidity can be maintained, for example, by the use of a coating such as lecithin, or by the use of surfactants. Antibacterial and antifungal agents include, for example, parabens, chlorobutanol, phenol, ascorbic acid and thimerosal. Including an agent that delays absorption, for example, aluminum monostearate and gelatin can prolonged absorption of injectable compositions. Polysorbate 20 and polysorbate 80 can be added into the formulation mixture, for example, up to 1%. Other non-limiting additives include histidine HCl, α,α-treahlose dehydrate.

Additional pharmaceutical formulations and delivery systems are known to the skilled artisan and are applicable in the methods of the invention (see, e.g., Remington's Pharmaceutical Sciences (1990) 18th ed., Mack Publishing Co., Easton, PA; The Merck Index (1996) 12th ed., Merck Publishing Group, Whitehouse, NJ; Pharmaceutical Principles of Solid Dosage Forms, Technonic Publishing Co., Inc., Lancaster, Pa., (1993); and Poznansky, et al., Drug Delivery Systems, R. L. Juliano, ed., Oxford, N.Y. (1980), pp. 253-315).

The invention provides kits including conjugates of the invention, combination compositions and pharmaceutical formulations thereof, packaged into suitable packaging material. A kit optionally includes a label or packaging insert including a description of the components or instructions for use in vitro, in vivo, or ex vivo, of the components therein. Exemplary instructions include instructions for reducing or inhibiting proliferation of a cell, reducing or inhibiting proliferation of undesirable or aberrant cells, such as a hyperproliferating cell, reducing or inhibiting proliferation of a metastatic or non-metastatic tumor, cancer, malignant or neoplastic cell, treating a subject having a hyperproliferative disorder, treating a subject having a metastatic or non-metastatic tumor, cancer, malignancy or neoplasia, or reducing fertility of an animal.

A kit can contain a collection of such components, e.g., two or more conjugates alone, or in combination with another therapeutically useful composition (e.g., an anti-proliferative or immune-enhancing drug).

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacturer location and date.

Labels or inserts can include information on a condition, disorder, disease or symptom for which a kit component may be used. Labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Exemplary instructions include, instructions for treating an undesirable or aberrant cell proliferation, hyperproliferating cells and disorders (e.g., metastatic or non-metastatic tumor, cancer, malignancy or neoplasia). Kits of the invention therefore can additionally include labels or instructions for practicing any of the methods and uses of the invention described herein.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Invention kits can additionally include other components. Each component of the kit can be enclosed within an individual container and all of the various containers can be within a single package. Invention kits can be designed for cold storage. Invention kits can further be designed to contain host cells expressing conjugates of the invention, or that contain nucleic acids encoding conjugates. The cells in the kit can be maintained under appropriate storage conditions until the cells are ready to be used. For example, a kit including one or more cells can contain appropriate cell storage medium so that the cells can be thawed and grown.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a”, “and,” and “the” include plural referents unless the context clearly indicates otherwise. Thus, for example, reference to “a conjugate” or “a target (e.g., a receptor),” or a “lytic domain” includes a plurality of such conjugates, targets, or lytic domains, and so forth.

As used herein, numerical values are often presented in a range format throughout this document. The use of a range format is merely for convenience and brevity and should not be construed as an inflexible limitation on the scope of the invention unless the context clearly indicates otherwise. Accordingly, a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and any numerical range within such a ranges, such as 1-2, 5-10, 10-50, 50-100, 100-500, 100-1000, 500-1000, 1000-2000, 1000-5000, etc. In a further example, reference to a range of KD 10⁻⁵ M to about KD 10⁻¹³ M includes any numerical value or range within or encompassing such values.

As also used herein a series of ranges are disclosed throughout this document. The use of a series of ranges include combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, and 150-171, includes ranges such as 5-20, 5-30, 5-40, 5-50, 5-75, 5-100, 5-150, 5-171, and 10-30, 10-40, 10-50, 10-75, 10-100, 10-150, 10-171, and 20-40, 20-50, 20-75, 20-100, 20-150, 20-171, and so forth.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

To determine, in in vitro studies, cytotoxicity of recombinantly produced antibody (as a antibody) scFv-C_{H}3 to Her-2 receptor conjugated to the lytic peptide, Phor18 (KFAKFAK KFAKFAK KFAK (SEQ ID NO.: 4)) or (KLAKLAK)₂KLAK (SEQ ID NO.: 74). Various linkers (GS and NRVRRS (SEQ ID NO.: 75)) and 1 or 2 molecules of lytic peptides per antibody molecule were studied.

Peptides studied were: Phor18-scFv-C_{H}3-Phor18 (2 molecules of Phor-18 joined at N- and C-terminal ends of the antibody, scFv-C_{H}3-GS-Phor18 (one molecule of Phor18 joined to the antibody at the C-terminus by GS linker, scFv-C_{H}3-GS-(KLAKLAK)₂KLAK (SEQ ID NO.: 74) (one molecule of (KLAKLAK)₂KLAK (SEQ ID NO.: 74) linked to the antibody at the C-terminus by GS linker, scFv-C_{H}3-NRVRRS (SEQ ID NO.: 75)-Phor18 (one molecule of Phor18 to the antibody at the C-terminus by NRVRRS linker, and scFv-C_{H}3-NRVRRS-(KLAKLAK)₂KLAK (SEQ ID NO.: 74) (one molecule of (KLAKLAK)₂KLAK (SEQ ID NO.: 74) to the antibody at the C-terminus by NRVRRS linker). Cytotoxicity was compared to a naked antibody (antibody without a lytic peptide) in Her-2 receptor positive cells (SKBR-3 and SKOV-3, human breast and ovarian cancer cells, respectively) and Her-2 receptor negative breast cancer cells (MDA-MB-231).

### Example 2

This example describes various materials and methods used in the studies described herein.

Materials: Recombinant DNA technique was used to synthesize anti-Her2/neu antibody as a recombinant antibody in Escherichia coli. The scFv-C_{H}3 antibody (Olafsen T. et al Protein Engineering, Design & Selection 17, 315-323, 2004) was conjugated via a peptide linker or without a linker as described in Table 1 to either Phor18 or an amphipathic, alpha-helical lytic peptide, (KLAKLAK)₂KLAK (SEQ ID NO.: 74) and analyzed for cytotoxicity in vitro. The plasmid was acquired through gene codon optimization. The gene was synthesized with a N-His tag sequence and the plasmid was subcloned into an E. coli bacteria expression vector pUC57. After expression optimization and evaluation the His-tag product was selected and 1 L of the bacteria expression product was purified in a one-step affinity purification. The sequences of the plasmid gene insertion for each construct is described in Table 1.

TABLE 1
Nucleotide sequence of the plasmid insertion for the production of each
recombinant Her2/neu antibody and antibody conjugate.

1. Her2/neu scFv-C_{H}3: (SEQ ID NO.: 76)

| | |
|---|---|
| 1 | CATATGCATC ACCACCACCA CCACGACGAC GACGACAAAG ATATTCAAAT GACCCAGTCC |
| 61 | CCGAGCAGCC TGAGTGCCTC CGTTGGCGAC CGCGTGACCA TTACGTGCCG TGCGAGCCAG |
| 121 | GATGTCAACA CCGCGGTGGC CTGGTATCAG CAAAAACCGG GCAAAGCGCC GAAACTGCTG |
| 181 | ATCTATTCAG CCTCGTTTCT GTACAGCGGT GTTCCGTCTC GTTTCAGCGG CTCTCGCAGT |
| 241 | GGTACCGATT TTACCCTGAC GATTAGCTCT CTGCAGCCGG AAGACTTTGC GACGTATTAC |
| 301 | TGCCAGCAAC ATTACACCAC GCCGCCGACC TTCGGCCAGG GTACGAAAGT GGAAATCAAA |
| 361 | GGTTCCACCT CAGGCGGTGG CAGTGGTGGC GGTTCCGGCG GTGGCGGTAG TTCCGAAGTT |
| 421 | CAGCTGGTCG AAAGTGGCGG TGGCCTGGTT CAACCGGGTG GCTCACTGCG TCTGTCGTGT |
| 481 | GCAGCAAGCG GTTTCAACAT CAAAGATACC TACATCCACT GGGTTCGTCA GGCGCCGGGC |
| 541 | AAAGGTCTGG AATGGGTCGC CCGCATTTAC CCGACCAATG GCTATACGCG TTACGCAGAT |
| 601 | AGCGTGAAAG GTCGCTTTAC CATCTCTGCG GACACCAGTA AAAACACGGC CTATCTGCAG |
| 661 | ATGAATAGCC TGCGTGCGGA AGATACGGCC GTTTATTACT GCTCTCGCTG GGGTGGCGAT |
| 721 | GGCTTCTATG CTATGGACTA CTGGGGCCAG GGTACCCTGG TGACGGTTTC ATCGGGTCAG |
| 781 | CCGCGTGAAC CGCAAGTGTA TACCCTGCCG CCGTCACGCG ATGAACTGAC GAAAAACCAG |
| 841 | GTGTCGCTGA CGTGTCTGGT TAAAGGCTTT TACCCGAGCG ACATCGCGGT TGAATGGGAA |
| 901 | TCTAATGGTC AACCGGAAAA CAATTATAAA ACCACGCCGC CGGTCCTGGA TAGTGACGGC |
| 961 | TCCTTTTTCC TGTACAGTAA ACTGACCGTG GATAAATCCC GTTGGCAGCA GGGTAACGTC |
| 1021 | TTCTCGTGTA GCGTGATGCA TGAAGCCCTG CATAATCACT ATACCCAGAA ATCTCTGAGT |
| 1081 | CTGTCCCCGG GCAAAGGTTC AACGTCGGGT GGCGGTTCCG GCGGTGGCTC AGGTGGCGGT |
| 1141 | GGCAGCTCTG GCCAACCGCG CGAACCGCAG GTTTACACCC TGCCGCCGAG CCGTGACGAA |
| 1201 | CTGACCAAAA ACCAAGTCAG CCTGACGTGC CTGGTGAAAG GCTTTTACCC GAGTGACATT |
| 1261 | GCAGTTGAAT GGGAATCCAA TGGTCAGCCG GAAAATAACT ACAAAACGAC GCCGCCGGTT |
| 1321 | CTGGATTCAG ACGGCTCGTT TTTCCTGTAC TCAAAACTGA CCGTCGATAA ATCGCGCTGG |
| 1381 | CAACAGGGTA ACGTTTTCAG CTGCTCTGTC ATGCACGAAG CCCTGCACAA CCATTATACC |
| 1441 | CAGAAAAGTC TGTCCCTGTC ACCGGGCAAA GAAGTGCAGC TGGTTGAATC TGGTGGCGGT |
| 1501 | CTGGTGCAAC CGGGCGGTTC GCTGCGTCTG AGCTGTGCAG CTTCTGGCTT TAATATTAAA |
| 1561 | GACACGTACA TCCACTGGGT GCGTCAGGCA CCGGGTAAAG GCCTGGAATG GGTTGCTCGT |
| 1621 | ATCTATCCGA CGAACGGTTA TACGCGTTAC GCCGATAGCG TCAAAGGCCG TTTTACCATC |
| 1681 | AGTGCAGACA CCTCCAAAAA CACGGCTTAT CTGCAGATGA ATAGTCTGCG TGCAGAAGAT |
| 1741 | ACCGCTGTTT ATTACTGCAG CCGCTGGGGC GGTGATGGCT TCTATGCAAT GGATTATTGG |
| 1801 | GGTCAAGGTA CCCTGGTCAC CGTGAGTTCC GGTTCGACCA GCGGCGGTGG CTCAGGTGGC |
| 1861 | GGTTCGGGCG GTGGCGGTTC ATCGGACATT CAGATGACGC AAAGCCCGAG CTCTCTGTCT |
| 1921 | GCGAGTGTTG GCGATCGTGT CACCATCACG TGTCGCGCCT CTCAGGACGT GAATACCGCA |
| 1981 | GTTGCTTGGT ACCAACAAAA ACCGGGCAAA GCACCGAAAC TGCTGATTTA CTCCGCTTCA |
| 2041 | TTCCTGTACA GCGGTGTGCC GTCTCGTTTT TCGGGCAGCC GCTCTGGTAC CGATTTCACC |
| 2101 | CTGACGATTA GTTCCCTGCA ACCGGAAGAT TTCGCCACCT ACTACTGCCA GCAACACTAT |
| 2161 | ACGACCCCGC CGACGTTTGG TCAGGGCACG AAAGTGGAAA TTAAATAATG AAAGCTT |

2. scFv-C_{H}3-GS-Phor18: (SEQ ID NO.: 77)

| | |
|---|---|
| 1 | CATATGCATC ACCACCACCA CCACGACGAC GACGACAAAG ATATTCAAAT GACCCAGTCC |
| 61 | CCGAGCAGCC TGAGTGCCTC CGTTGGCGAC CGCGTGACCA TTACGTGCCG TGCGAGCCAG |
| 121 | GATGTCAACA CCGCGGTGGC CTGGTATCAG CAAAAACCGG GCAAAGCGCC GAAACTGCTG |
| 181 | ATCTATTCAG CCTCGTTTCT GTACAGCGGT GTTCCGTCTC GTTTCAGCGG CTCTCGCAGT |
| 241 | GGTACCGATT TTACCCTGAC GATTAGCTCT CTGCAGCCGG AAGACTTTGC GACGTATTAC |
| 301 | TGCCAGCAAC ATTACACCAC GCCGCCGACC TTCGGCCAGG GTACGAAAGT GGAAATCAAA |
| 361 | GGTTCCACCT CAGGCGGTGG CAGTGGTGGC GGTTCCGGCG GTGGCGGTAG TTCCGAAGTT |
| 421 | CAGCTGGTCG AAAGTGGCGG TGGCCTGGTT CAACCGGGTG GCTCACTGCG TCTGTCGTGT |
| 481 | GCAGCAAGCG GTTTCAACAT CAAAGATACC TACATCCACT GGGTTCGTCA GGCGCCGGGC |
| 541 | AAAGGTCTGG AATGGGTCGC CCGCATTTAC CCGACCAATG GCTATACGCG TTACGCAGAT |
| 601 | AGCGTGAAAG GTCGCTTTAC CATCTCTGCG GACACCAGTA AAAACACGGC CTATCTGCAG |
| 661 | ATGAATAGCC TGCGTGCGGA AGATACGGCC GTTTATTACT GCTCTCGCTG GGGTGGCGAT |
| 721 | GGCTTCTATG CTATGGACTA CTGGGGCCAG GGTACCCTGG TGACGGTTTC ATCGGGTCAG |
| 781 | CCGCGTGAAC CGCAAGTGTA TACCCTGCCG CCGTCACGCG ATGAACTGAC GAAAAACCAG |
| 841 | GTGTCGCTGA CGTGTCTGGT TAAAGGCTTT TACCCGAGCG ACATCGCGGT TGAATGGGAA |
| 901 | TCTAATGGTC AACCGGAAAA CAATTATAAA ACCACGCCGC CGGTCCTGGA TAGTGACGGC |
| 961 | TCCTTTTTCC TGTACAGTAA ACTGACCGTG GATAAATCCC GTTGGCAGCA GGGTAACGTC |
| 1021 | TTCTCGTGTA GCGTGATGCA TGAAGCCCTG CATAATCACT ATACCCAGAA ATCTCTGAGT |
| 1081 | CTGTCCCCGG GCAAAGGTTC AACGTCGGGT GGCGGTTCCG GCGGTGGCTC AGGTGGCGGT |
| 1141 | GGCAGCTCTG GCCAACCGCG CGAACCGCAG GTTTACACCC TGCCGCCGAG CCGTGACGAA |
| 1201 | CTGACCAAAA ACCAAGTCAG CCTGACGTGC CTGGTGAAAG GCTTTTACCC GAGTGACATT |
| 1261 | GCAGTTGAAT GGGAATCCAA TGGTCAGCCG GAAAATAACT ACAAAACGAC GCCGCCGGTT |
| 1321 | CTGGATTCAG ACGGCTCGTT TTTCCTGTAC TCAAAACTGA CCGTCGATAA ATCGCGCTGG |
| 1381 | CAACAGGGTA ACGTTTTCAG CTGCTCTGTC ATGCACGAAG CCCTGCACAA CCATTATACC |
| 1441 | CAGAAAAGTC TGTCCCTGTC ACCGGGCAAA GAAGTGCAGC TGGTTGAATC TGGTGGCGGT |
| 1501 | CTGGTGCAAC CGGGCGGTTC GCTGCGTCTG AGCTGTGCAG CTTCTGGCTT TAATATTAAA |
| 1561 | GACACGTACA TCCACTGGGT GCGTCAGGCA CCGGGTAAAG GCCTGGAATG GGTTGCTCGT |
| 1621 | ATCTATCCGA CGAACGGTTA TACGCGTTAC GCCGATAGCG TCAAAGGCCG TTTTACCATC |
| 1681 | AGTGCAGACA CCTCCAAAAA CACGGCTTAT CTGCAGATGA ATAGTCTGCG TGCAGAAGAT |
| 1741 | ACCGCTGTTT ATTACTGCAG CCGCTGGGGC GGTGATGGCT TCTATGCAAT GGATTATTGG |
| 1801 | GGTCAAGGTA CCCTGGTCAC CGTGAGTTCC GGTTCGACCA GCGGCGGTGG CTCAGGTGGC |
| 1861 | GGTTCGGGCG GTGGCGGTTC ATCGGACATT CAGATGACGC AAAGCCCGAG CTCTCTGTCT |
| 1921 | GCGAGTGTTG GCGATCGTGT CACCATCACG TGTCGCGCCT CTCAGGACGT GAATACCGCA |
| 1981 | GTTGCTTGGT ACCAACAAAA ACCGGGCAAA GCACCGAAAC TGCTGATTTA CTCCGCTTCA |
| 2041 | TTCCTGTACA GCGGTGTGCC GTCTCGTTTT TCGGGCAGCC GCTCTGGTAC CGATTTCACC |
| 2101 | CTGACGATTA GTTCCCTGCA ACCGGAAGAT TTCGCCACCT ACTACTGCCA GCAACACTAT |
| 2161 | ACGACCCCGC CGACGTTTGG TCAGGGCACG AAAGTGGAAA TTAAAGGCAG CAAATTTGCG |
| 2221 | AAATTCGCCA AAAAATTCGC AAAATTCGCG AAAAAATTCG CGAAATAATG AAAGCTT |

3. scFv-C_{H}3-GS-(KLAKLAK)₂KLAK: (SEQ ID NO.: 78)

| | |
|---|---|
| 1 | CATATGGAAA ATCTGTATTT CCAAGGTGAT ATTCAAATGA CCCAGTCCCC GAGCAGCCTG |
| 61 | AGTGCCTCCG TTGGCGACCG CGTGACCATT ACGTGCCGTG CGAGCCAGGA TGTCAACACC |
| 121 | GCGGTGGCCT GGTATCAGCA AAAACCGGGC AAAGCGCCGA AACTGCTGAT CTATTCAGCC |
| 181 | TCGTTTCTGT ACAGCGGTGT TCCGTCTCGT TTCAGCGGCT CTCGCAGTGG TACCGATTTT |
| 241 | ACCCTGACGA TTAGCTCTCT GCAGCCGGAA GACTTTGCGA CGTATTACTG CCAGCAACAT |
| 301 | TACACCACGC CGCCGACCTT CGGCCAGGGT ACGAAAGTGG AAATCAAAGG TTCCACCTCA |
| 361 | GGCGGTGGCA GTGGTGGCGG TTCCGGCGGT GGCGGTAGTT CCGAAGTTCA GCTGGTCGAA |
| 421 | AGTGGCGGTG GCCTGGTTCA ACCGGGTGGC TCACTGCGTC TGTCGTGTGC AGCAAGCGGT |
| 481 | TTCAACATCA AAGATACCTA CATCCACTGG GTTCGTCAGG CGCCGGGCAA AGGTCTGGAA |
| 541 | TGGGTCGCCC GCATTTACCC GACCAATGGC TATACGCGTT ACGCAGATAG CGTGAAAGGT |
| 601 | CGCTTTACCA TCTCTGCGGA CACCAGTAAA AACACGGCCT ATCTGCAGAT GAATAGCCTG |
| 661 | CGTGCGGAAG ATACGGCCGT TTATTACTGC TCTCGCTGGG GTGGCGATGG CTTCTATGCT |
| 721 | ATGGACTACT GGGGCCAGGG TACCCTGGTG ACGGTTTCAT CGGGTCAGCC GCGTGAACCG |
| 781 | CAAGTGTATA CCCTGCCGCC GTCACGCGAT GAACTGACGA AAAACCAGGT GTCGCTGACG |
| 841 | TGTCTGGTTA AAGGCTTTTA CCCGAGCGAC ATCGCGGTTG AATGGGAATC TAATGGTCAA |
| 901 | CCGGAAAACA ATTATAAAAC CACGCCGCCG GTCCTGGATA GTGACGGCTC CTTTTTCCTG |
| 961 | TACAGTAAAC TGACCGTGGA TAAATCCCGT TGGCAGCAGG GTAACGTCTT CTCGTGTAGC |
| 1021 | GTGATGCATG AAGCCCTGCA TAATCACTAT ACCCAGAAAT CTCTGAGTCT GTCCCCGGGC |
| 1081 | AAAGGTTCAA CGTCGGGTGG CGGTTCCGGC GGTGGCTCAG GTGGCGGTGG CAGCTCTGGC |
| 1141 | CAACCGCGCG AACCGCAGGT TTACACCCTG CCGCCGAGCC GTGACGAACT GACCAAAAAC |
| 1201 | CAAGTCAGCC TGACGTGCCT GGTGAAAGGC TTTTACCCGA GTGACATTGC AGTTGAATGG |
| 1261 | GAATCCAATG GTCAGCCGGA AAATAACTAC AAAACGACGC CGCCGGTTCT GGATTCAGAC |
| 1321 | GGCTCGTTTT TCCTGTACTC AAAACTGACC GTCGATAAAT CGCGCTGGCA ACAGGGTAAC |
| 1381 | GTTTTCAGCT GCTCTGTCAT GCACGAAGCC CTGCACAACC ATTATACCCA GAAAAGTCTG |
| 1441 | TCCCTGTCAC CGGGCAAAGA AGTGCAGCTG GTTGAATCTG GTGGCGGTCT GGTGCAACCG |
| 1501 | GGCGGTTCGC TGCGTCTGAG CTGTGCAGCT TCTGGCTTTA ATATTAAAGA CACGTACATC |
| 1561 | CACTGGGTGC GTCAGGCACC GGGTAAAGGC CTGGAATGGG TTGCTCGTAT CTATCCGACG |
| 1621 | AACGGTTATA CGCGTTACGC CGATAGCGTC AAAGGCCGTT TTACCATCAG TGCAGACACC |
| 1681 | TCCAAAAACA CGGCTTATCT GCAGATGAAT AGTCTGCGTG CAGAAGATAC CGCTGTTTAT |
| 1741 | TACTGCAGCC GCTGGGGCGG TGATGGCTTC TATGCAATGG ATTATTGGGG TCAAGGTACC |
| 1801 | CTGGTCACCG TGAGTTCCGG TTCGACCAGC GGCGGTGGCT CAGGTGGCGG TTCGGGCGGT |
| 1861 | GGCGGTTCAT CGGACATTCA GATGACGCAA AGCCCGAGCT CTCTGTCTGC GAGTGTTGGC |
| 1921 | GATCGTGTCA CCATCACGTG TCGCGCCTCT CAGGACGTGA ATACCGCAGT TGCTTGGTAC |
| 1981 | CAACAAAAAC CGGGCAAAGC ACCGAAACTG CTGATTTACT CCGCTTCATT CCTGTACAGC |
| 2041 | GGTGTGCCGT CTCGTTTTTC GGGCAGCCGC TCTGGTACCG ATTTCACCCT GACGATTAGT |
| 2101 | TCCCTGCAAC CGGAAGATTT CGCCACCTAC TACTGCCAGC AACACTATAC GACCCCGCCG |
| 2161 | ACGTTTGGTC AGGGCACGAA AGTGGAAATT AAAGGCAGCA AACTGGCGAA ACTGGCCAAA |
| 2221 | AAACTGGCAA AACTGGCGAA AAAACTGGCG AAATAATGAA AGCTT |

4. scFv-CH₃-NRVRRS-Phor18: (SEQ ID NO.: 79)

| | |
|---|---|
| 1 | CATATGGAAA ATCTGTATTT CCAAGGTGAT ATTCAAATGA CCCAGTCCCC GAGCAGCCTG |
| 61 | AGTGCCTCCG TTGGCGACCG CGTGACCATT ACGTGCCGTG CGAGCCAGGA TGTCAACACC |
| 121 | GCGGTGGCCT GGTATCAGCA AAAACCGGGC AAAGCGCCGA AACTGCTGAT CTATTCAGCC |
| 181 | TCGTTTCTGT ACAGCGGTGT TCCGTCTCGT TTCAGCGGCT CTCGCAGTGG TACCGATTTT |
| 241 | ACCCTGACGA TTAGCTCTCT GCAGCCGGAA GACTTTGCGA CGTATTACTG CCAGCAACAT |
| 301 | TACACCACGC CGCCGACCTT CGGCCAGGGT ACGAAAGTGG AAATCAAAGG TTCCACCTCA |
| 361 | GGCGGTGGCA GTGGTGGCGG TTCCGGCGGT GGCGGTAGTT CCGAAGTTCA GCTGGTCGAA |
| 421 | AGTGGCGGTG GCCTGGTTCA ACCGGGTGGC TCACTGCGTC TGTCGTGTGC AGCAAGCGGT |
| 481 | TTCAACATCA AAGATACCTA CATCCACTGG GTTCGTCAGG CGCCGGGCAA AGGTCTGGAA |
| 541 | TGGGTCGCCC GCATTTACCC GACCAATGGC TATACGCGTT ACGCAGATAG CGTGAAAGGT |
| 601 | CGCTTTACCA TCTCTGCGGA CACCAGTAAA AACACGGCCT ATCTGCAGAT GAATAGCCTG |
| 661 | CGTGCGGAAG ATACGGCCGT TTATTACTGC TCTCGCTGGG GTGGCGATGG CTTCTATGCT |
| 721 | ATGGACTACT GGGGCCAGGG TACCCTGGTG ACGGTTTCAT CGGGTCAGCC GCGTGAACCG |
| 781 | CAAGTGTATA CCCTGCCGCC GTCACGCGAT GAACTGACGA AAAACCAGGT GTCGCTGACG |
| 841 | TGTCTGGTTA AAGGCTTTTA CCCGAGCGAC ATCGCGGTTG AATGGGAATC TAATGGTCAA |
| 901 | CCGGAAAACA ATTATAAAAC CACGCCGCCG GTCCTGGATA GTGACGGCTC CTTTTTCCTG |
| 961 | TACAGTAAAC TGACCGTGGA TAAATCCCGT TGGCAGCAGG GTAACGTCTT CTCGTGTAGC |
| 1021 | GTGATGCATG AAGCCCTGCA TAATCACTAT ACCCAGAAAT CTCTGAGTCT GTCCCCGGGC |
| 1081 | AAAGGTTCAA CGTCGGGTGG CGGTTCCGGC GGTGGCTCAG GTGGCGGTGG CAGCTCTGGC |
| 1141 | CAACCGCGCG AACCGCAGGT TTACACCCTG CCGCCGAGCC GTGACGAACT GACCAAAAAC |
| 1201 | CAAGTCAGCC TGACGTGCCT GGTGAAAGGC TTTTACCCGA GTGACATTGC AGTTGAATGG |
| 1261 | GAATCCAATG GTCAGCCGGA AAATAACTAC AAAACGACGC CGCCGGTTCT GGATTCAGAC |
| 1321 | GGCTCGTTTT TCCTGTACTC AAAACTGACC GTCGATAAAT CGCGCTGGCA ACAGGGTAAC |
| 1381 | GTTTTCAGCT GCTCTGTCAT GCACGAAGCC CTGCACAACC ATTATACCCA GAAAAGTCTG |
| 1441 | TCCCTGTCAC CGGGCAAAGA AGTGCAGCTG GTTGAATCTG GTGGCGGTCT GGTGCAACCG |
| 1501 | GGCGGTTCGC TGCGTCTGAG CTGTGCAGCT TCTGGCTTTA ATATTAAAGA CACGTACATC |
| 1561 | CACTGGGTGC GTCAGGCACC GGGTAAAGGC CTGGAATGGG TTGCTCGTAT CTATCCGACG |
| 1621 | AACGGTTATA CGCGTTACGC CGATAGCGTC AAAGGCCGTT TTACCATCAG TGCAGACACC |
| 1681 | TCCAAAAACA CGGCTTATCT GCAGATGAAT AGTCTGCGTG CAGAAGATAC CGCTGTTTAT |
| 1741 | TACTGCAGCC GCTGGGGCGG TGATGGCTTC TATGCAATGG ATTATTGGGG TCAAGGTACC |
| 1801 | CTGGTCACCG TGAGTTCCGG TTCGACCAGC GGCGGTGGCT CAGGTGGCGG TTCGGGCGGT |
| 1861 | GGCGGTTCAT CGGACATTCA GATGACGCAA AGCCCGAGCT CTCTGTCTGC GAGTGTTGGC |
| 1921 | GATCGTGTCA CCATCACGTG TCGCGCCTCT CAGGACGTGA ATACCGCAGT TGCTTGGTAC |
| 1981 | CAACAAAAAC CGGGCAAAGC ACCGAAACTG CTGATTTACT CCGCTTCATT CCTGTACAGC |
| 2041 | GGTGTGCCGT CTCGTTTTTC GGGCAGCCGC TCTGGTACCG ATTTCACCCT GACGATTAGT |
| 2101 | TCCCTGCAAC CGGAAGATTT CGCCACCTAC TACTGCCAGC AACACTATAC GACCCCGCCG |
| 2161 | ACGTTTGGTC AGGGCACGAA AGTGGAAATT AAAAACCGTG TGCGTCGCAG CAAATTTGCG |
| 2221 | AAATTCGCCA AAAAATTTGC AAAATTCGCT AAAAAATTTG CGAAATAATG AAAGCTT |

5. scFv-C_{H}3-NRVRRS-(KLAKLAK)₂KLAK: (SEQ ID NO.: 80)

| | |
|---|---|
| 1 | CATATGCATC ACCACCACCA CCACGACGAC GACGACAAAG ATATTCAAAT GACCCAGTCC |
| 61 | CCGAGCAGCC TGAGTGCCTC CGTTGGCGAC CGCGTGACCA TTACGTGCCG TGCGAGCCAG |
| 121 | GATGTCAACA CCGCGGTGGC CTGGTATCAG CAAAAACCGG GCAAAGCGCC GAAACTGCTG |
| 181 | ATCTATTCAG CCTCGTTTCT GTACAGCGGT GTTCCGTCTC GTTTCAGCGG CTCTCGCAGT |
| 241 | GGTACCGATT TTACCCTGAC GATTAGCTCT CTGCAGCCGG AAGACTTTGC GACGTATTAC |
| 301 | TGCCAGCAAC ATTACACCAC GCCGCCGACC TTCGGCCAGG GTACGAAAGT GGAAATCAAA |
| 361 | GGTTCCACCT CAGGCGGTGG CAGTGGTGGC GGTTCCGGCG GTGGCGGTAG TTCCGAAGTT |
| 421 | CAGCTGGTCG AAAGTGGCGG TGGCCTGGTT CAACCGGGTG GCTCACTGCG TCTGTCGTGT |
| 481 | GCAGCAAGCG GTTTCAACAT CAAAGATACC TACATCCACT GGGTTCGTCA GGCGCCGGGC |
| 541 | AAAGGTCTGG AATGGGTCGC CCGCATTTAC CCGACCAATG GCTATACGCG TTACGCAGAT |
| 601 | AGCGTGAAAG GTCGCTTTAC CATCTCTGCG GACACCAGTA AAAACACGGC CTATCTGCAG |
| 661 | ATGAATAGCC TGCGTGCGGA AGATACGGCC GTTTATTACT GCTCTCGCTG GGGTGGCGAT |
| 721 | GGCTTCTATG CTATGGACTA CTGGGGCCAG GGTACCCTGG TGACGGTTTC ATCGGGTCAG |
| 781 | CCGCGTGAAC CGCAAGTGTA TACCCTGCCG CCGTCACGCG ATGAACTGAC GAAAAACCAG |
| 841 | GTGTCGCTGA CGTGTCTGGT TAAAGGCTTT TACCCGAGCG ACATCGCGGT TGAATGGGAA |
| 901 | TCTAATGGTC AACCGGAAAA CAATTATAAA ACCACGCCGC CGGTCCTGGA TAGTGACGGC |
| 961 | TCCTTTTTCC TGTACAGTAA ACTGACCGTG GATAAATCCC GTTGGCAGCA GGGTAACGTC |
| 1021 | TTCTCGTGTA GCGTGATGCA TGAAGCCCTG CATAATCACT ATACCCAGAA ATCTCTGAGT |
| 1081 | CTGTCCCCGG GCAAAGGTTC AACGTCGGGT GGCGGTTCCG GCGGTGGCTC AGGTGGCGGT |
| 1141 | GGCAGCTCTG GCCAACCGCG CGAACCGCAG GTTTACACCC TGCCGCCGAG CCGTGACGAA |
| 1201 | CTGACCAAAA ACCAAGTCAG CCTGACGTGC CTGGTGAAAG GCTTTTACCC GAGTGACATT |
| 1261 | GCAGTTGAAT GGGAATCCAA TGGTCAGCCG GAAAATAACT ACAAAACGAC GCCGCCGGTT |
| 1321 | CTGGATTCAG ACGGCTCGTT TTTCCTGTAC TCAAAACTGA CCGTCGATAA ATCGCGCTGG |
| 1381 | CAACAGGGTA ACGTTTTCAG CTGCTCTGTC ATGCACGAAG CCCTGCACAA CCATTATACC |
| 1441 | CAGAAAAGTC TGTCCCTGTC ACCGGGCAAA GAAGTGCAGC TGGTTGAATC TGGTGGCGGT |
| 1501 | CTGGTGCAAC CGGGCGGTTC GCTGCGTCTG AGCTGTGCAG CTTCTGGCTT TAATATTAAA |
| 1561 | GACACGTACA TCCACTGGGT GCGTCAGGCA CCGGGTAAAG GCCTGGAATG GGTTGCTCGT |
| 1621 | ATCTATCCGA CGAACGGTTA TACGCGTTAC GCCGATAGCG TCAAAGGCCG TTTTACCATC |
| 1681 | AGTGCAGACA CCTCCAAAAA CACGGCTTAT CTGCAGATGA ATAGTCTGCG TGCAGAAGAT |
| 1741 | ACCGCTGTTT ATTACTGCAG CCGCTGGGGC GGTGATGGCT TCTATGCAAT GGATTATTGG |
| 1801 | GGTCAAGGTA CCCTGGTCAC CGTGAGTTCC GGTTCGACCA GCGGCGGTGG CTCAGGTGGC |
| 1861 | GGTTCGGGCG GTGGCGGTTC ATCGGACATT CAGATGACGC AAAGCCCGAG CTCTCTGTCT |
| 1921 | GCGAGTGTTG GCGATCGTGT CACCATCACG TGTCGCGCCT CTCAGGACGT GAATACCGCA |
| 1981 | GTTGCTTGGT ACCAACAAAA ACCGGGCAAA GCACCGAAAC TGCTGATTTA CTCCGCTTCA |
| 2041 | TTCCTGTACA GCGGTGTGCC GTCTCGTTTT TCGGGCAGCC GCTCTGGTAC CGATTTCACC |
| 2101 | CTGACGATTA GTTCCCTGCA ACCGGAAGAT TTCGCCACCT ACTACTGCCA GCAACACTAT |
| 2161 | ACGACCCCGC CGACGTTTGG TCAGGGCACG AAAGTGGAAA TTAAAAACCG TGTGCGTCGC |
| 2221 | AGCAAACTGG CGAAACTGGC CAAAAAACTG GCAAAACTGG CTAAAAAACT GGCGAAATAA |
| 2281 | TGAAAGCTT |

6. Phor18-scFv-C_{H}3-Phor18: (SEQ ID NO: 81)

| | |
|---|---|
| 1 | CATATGGAAA ATCTGTATTT CCAAGGTAAA TTTGCGAAAt TCGCCAAAAA ATTCGCAAAA |
| 61 | 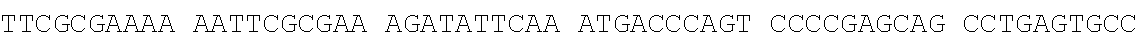 |
| 121 | TCCGTTGGCG ACCGCGTGAC CATTACGTGC CGTGCGAGCC AGGATGTCAA CACCGCGGTG |
| 181 | GCCTGGTATC AGCAAAAACC GGGCAAAGCG CCGAAACTGC TGATCTATTC AGCCTCGTTT |
| 241 | CTGTACAGCG GTGTTCCGTC TCGTTTCAGC GGCTCTCGCA GTGGTACCGA TTTTACCCTG |
| 301 | ACGATTAGCT CTCTGCAGCC GGAAGACTTT GCGACGTATT ACTGCCAGCA ACATTACACC |
| 361 | ACGCCGCCGA CCTTCGGCCA GGGTACGAAA GTGGAAATCA AAGGTTCCAC CTCAGGCGGT |
| 421 | GGCAGTGGTG GCGGTTCCGG CGGTGGCGGT AGTTCCGAAG TTCAGCTGGT CGAAAGTGGC |
| 481 | GGTGGCCTGG TTCAACCGGG TGGCTCACTG CGTCTGTCGT GTGCAGCAAG CGGTTTCAAC |
| 541 | ATCAAAGATA CCTACATCCA CTGGGTTCGT CAGGCGCCGG GCAAAGGTCT GGAATGGGTC |
| 601 | GCCCGCATTT ACCCGACCAA TGGCTATACG CGTTACGCAG ATAGCGTGAA AGGTCGCTTT |
| 661 | ACCATCTCTG CGGACACCAG TAAAAACACG GCCTATCTGC AGATGAATAG CCTGCGTGCG |
| 721 | GAAGATACGG CCGTTTATTA CTGCTCTCGC TGGGGTGGCG ATGGCTTCTA TGCTATGGAC |
| 781 | TACTGGGGCC AGGGTACCCT GGTGACGGTT TCATCGGGTC AGCCGCGTGA ACCGCAAGTG |
| 841 | TATACCCTGC CGCCGTCACG CGATGAACTG ACGAAAAACC AGGTGTCGCT GACGTGTCTG |
| 901 | GTTAAAGGCT TTTACCCGAG CGACATCGCG GTTGAATGGG AATCTAATGG TCAACCGGAA |
| 961 | AACAATTATA AAACCACGCC GCCGGTCCTG GATAGTGACG GCTCCTTTTT CCTGTACAGT |
| 1021 | AAACTGACCG TGGATAAATC CCGTTGGCAG CAGGGTAACG TCTTCTCGTG TAGCGTGATG |
| 1081 | CATGAAGCCC TGCATAATCA CTATACCCAG AAATCTCTGA GTCTGTCCCC GGGCAAAGGT |
| 1141 | TCAACGTCGG GTGGCGGTTC CGGCGGTGGC TCAGGTGGCG GTGGCAGCTC TGGCCAACCG |
| 1201 | CGCGAACCGC AGGTTTACAC CCTGCCGCCG AGCCGTGACG AACTGACCAA AAACCAAGTC |
| 1261 | AGCCTGACGT GCCTGGTGAA AGGCTTTTAC CCGAGTGACA TTGCAGTTGA ATGGGAATCC |
| 1321 | AATGGTCAGC CGGAAAATAA CTACAAAACG ACGCCGCCGG TTCTGGATTC AGACGGCTCG |
| 1381 | TTTTTCCTGT ACTCAAAACT GACCGTCGAT AAATCGCGCT GGCAACAGGG TAACGTTTTC |
| 1441 | AGCTGCTCTG TCATGCACGA AGCCCTGCAC AACCATTATA CCCAGAAAAG TCTGTCCCTG |
| 1501 | TCACCGGGCA AAGAAGTGCA GCTGGTTGAA TCTGGTGGCG GTCTGGTGCA ACCGGGCGGT |
| 1561 | TCGCTGCGTC TGAGCTGTGC AGCTTCTGGC TTTAATATTA AAGACACGTA CATCCACTGG |
| 1621 | GTGCGTCAGG CACCGGGTAA AGGCCTGGAA TGGGTTGCTC GTATCTATCC GACGAACGGT |
| 1681 | TATACGCGTT ACGCCGATAG CGTCAAAGGC CGTTTTACCA TCAGTGCAGA CACCTCCAAA |
| 1741 | AACACGGCTT ATCTGCAGAT GAATAGTCTG CGTGCAGAAG ATACCGCTGT TTATTACTGC |
| 1801 | AGCCGCTGGG GCGGTGATGG CTTCTATGCA ATGGATTATT GGGGTCAAGG TACCCTGGTC |
| 1861 | ACCGTGAGTT CCGGTTCGAC CAGCGGCGGT GGCTCAGGTG GCGGTTCGGG CGGTGGCGGT |
| 1921 | TCATCGGACA TTCAGATGAC GCAAAGCCCG AGCTCTCTGT CTGCGAGTGT TGGCGATCGT |
| 1981 | GTCACCATCA CGTGTCGCGC CTCTCAGGAC GTGAATACCG CAGTTGCTTG GTACCAACAA |
| 2041 | AAACCGGGCA AAGCACCGAA ACTGCTGATT TACTCCGCTT CATTCCTGTA CAGCGGTGTG |
| 2101 | CCGTCTCGTT TTTCGGGCAG CCGCTCTGGT ACCGATTTCA CCCTGACGAT TAGTTCCCTG |
| 2161 | CAACCGGAAG ATTTCGCCAC CTACTACTGC CAGCAACACT ATACGACCCC GCCGACGTTT |
| 2221 | GGTCAGGGCA CGAAAGTGGA AATTAAAAAA TTTGCGAAAT TCGCCAAAAA ATTCGCAAAA |
| 2281 | TTCGCGAAAA AATTCGCGAA ATAATGAAAG CTT |

Chemical conjugation of Phor18 to a monoclonal anti-Her2/neu antibody IgG1 (MAb): Purified antibody in phosphate buffered saline (PBS) is concentrated to a concentration of approximately 2 mg/mL. A 20 mM solution of N-succinidyl-3-(2-pyridylothio) propionate (SPDP) is freshly prepared in dimethylsulfoxide (DMSO), and added to the antibody solution in 20-fold excess. The mixture is incubated at room temperature for about 30 minutes to produce the antibody-linker intermediate. Excess unreacted SPDP is removed by size exclusion. The cytotoxic molecule containing cysteine is thoroughly reduced by reaction with a 10-fold excess of reductacryl reagent before mixing in 10-fold excess with the antibody-linker construct. The reaction is allowed to incubate at room temperature for 18 hours, then desalted to remove unreacted cytotoxin molecule. The solution is filter-sterilized before storage.

In vitro: In vitro cytotoxicity studies were performed to determine the cytotoxicity of the recombinant antibody preparations (conjugated and unconjugated) and lytic peptide, Phor18, was used in control incubations. Cells were prepared in 96 well plates using 2,000 cells/well and were allowed to attach for 48 hours. Phor18 in lyophilized form was freshly dissolved in saline and added into the multi-well plates at increasing concentrations of 0, 0.00001, 0.0001, 0.001, 0.01, 0.1, 1, 10, 25, and 100 μM. The Her2/neu-antibody-Phor18 conjugates (Phor18-scFv-C_{H}3-Phor18, scFv-C_{H}3 -GS-Phor18, scFv-C_{H}3-NRVRRS (SEQ ID NO.: 75)-Phor18, the Her2/neu-antibody-(KLAKLAK)₂KLAK (SEQ ID NO.: 74) (scFv-C_{H}3-GS-(KLAKLAK)₂KLAK (SEQ ID NO.: 74), and scFv-C_{H}3-NRVRRS (SEQ ID NO.: 75)-(KLAKLAK)₂KLAK) (SEQ ID NO.: 74), or scFv-C_{H}3 receptor antibody (naked) in Tris/HCL-buffer were diluted with saline and added to cells at increasing concentrations of 0, 0.0012, 0.012, 0.12, 1.2, 6.0, 12.0, 120, 360 and 720 nM. Incubations were conducted for 48 h at 37° C. Cell viability was determined using formazan conversion assays (MTT assays). Controls contained USP saline or 0.1% TritonX-100™ as reference for 0 and 100% cell death, respectively. All data were processed and analyzed using Graph Pad Prizm 4™ software (Graph Pad Prizm, Inc).

### Example 3

This example describes studies indicating that anti-Her2-Phor18 antibody conjugate killed Her2 expressing breast cancer cells.

As shown in Table 2, the anti-Her2-Phor18 antibody conjugates (Phor18-scFv-C_{H}3-Phor18, scFv-C_{H}3-GS-Phor18, scFv-C_{H}3-NRVRRS (SEQ ID NO.: 75)-Phor18) killed Her2/neu positive human breast cancer SKBR-3 and ovarian cancer SKOV-3 cell lines by 48 hours, whereas the Her2/neu negative human breast cancer MDA-MB-231 cell line was not killed. Evidence of cytotoxicity was observed microscopically at as early as 24 hours of incubation. As expected, unconjugated Phor18 showed only modest cytotoxicity.

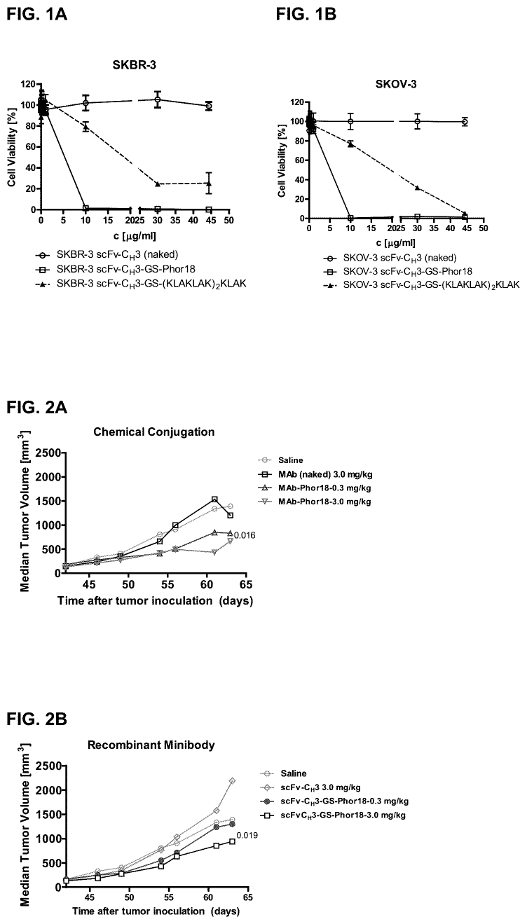

The HER2/neu antibody conjugated to the Phor18 was significantly more cytotoxic than antibody conjugated to the lytic peptide (KLAKLAK)₂KLAK (SEQ ID NO.: 74). (FIG. 1, Table 2). The Her2/neu negative MDA-MB-231 cells were not killed by any of the recombinant antibody-lytic peptide conjugates indicating that the cytotoxicity of the antibodies was mediated via Her2/neu receptors. The “naked” (unconjugated) antibody (scFv-C_{H}3) was not cytotoxic in all 3 cell lines indicating that the cell-killing properties of the antibody-lytic peptide conjugates were due to the presence of lytic peptide payload and sequence of the lytic peptide. Again, as expected, unconjugated Phor18 showed very minimal non-specific cytotoxicity in all cell lines (Table 2).

TABLE 2
In vitro cytotoxicity of anti-Her2-Phor18 antibody
conjugates (scFv-C_{H}3-Phor18 and-
scFv-C_{H}3-(KLAKLAK)₂KLAK (SEQ ID NO.: 74) conjugates,
Her2/neu scFv-C_{H}3 and unconjugated Phor18 in Her2/neu receptor
positive SKOV-3, SKBR-3 and Her2/neu receptor negative
MDA-MB-231 cancer cells. Values are IC₅₀ expressed in nM.

| | | | IC₅₀ [nM] |
|---|---|---|---|
| | IC₅₀ [nM] | IC₅₀ [nM] | MDA- |
| Recombinant Antibody Conjugate | SKOV-3 | SKBR-3 | MB-231 |
| Phor18-scFv-C_{H}3-Phor18 | 44.33 ± 9.2 | 51.56 ± 6.1 | >1000 |
| scFv-C_{H}3-GS-Phor18 | 27 ± 2.5 | 30 ± 1.9 | >1000 |
| scFv-C_{H}3-GS-(KLAKLAK)₂KLAK | 235 ± 6.5 | 246 ± 41 | >1000 |
| scFv-C_{H}3-NRVRRS-Phor18 | 29.3 ± 3.5 | 76.3 ± 16 | >1000 |
| scFv-C_{H}3-NRVRRS- | 247 ± 40.5 | 338 ± 8.7 | >1000 |
| (KLAKLAK)₂KLAK | | | |
| Her2/neu scFv-C_{H}3 | >1000 | >1000 | >1000 |
| Phor18 | 18,180 | 11,455 | 9,258 |

The results indicate that recombinantly produced Her2 antibody scFv-C_{H}3-Phor18 and Her2 antibody scFv-C_{H}3-(KLAKLAK)₂KLAK (SEQ ID NO.: 74) conjugates are active in the nanomolar range against Her2/neu receptor expressing cell lines. The unconjugated antibody or free lytic peptide (Phor18) were without effect indicating that conjugation of lytic peptides to ligands (e.g., antibodies) that bind to Her2/neu receptor to enhances cell cytotoxic potency.

### Example 4

This example includes a description of in vitro cytotoxicity studies of recombinantly produced antibody to Her-2 receptor conjugated to lytic peptide, Phor18 (KFAKFAK KFAKFAK KFAK (SEQ ID NO.: 4)) (scFv-C_{H}3-GS-Phor18), and a chemically conjugated MAb-Phor18 conjugate against Her2 positive ovarian cancer cell line SKOV-3.

Cells were prepared in 96 well plates using 5,000 cells/well and were allowed to attach for 48 hours. MAb-Phor18, scFv-C_{H}3-GS-Phor18, scFv-C_{H}3 were diluted in saline and added at increasing concentrations of 0, 0.0012, 0.012, 0.12, 1.2, 6.0, 12, 120, 360 and 720 nM, N=8 data points per concentration. Incubations were conducted for 24 h at 37° C. Cell viability was determined using formazan conversion assays (MTT assays). Controls contained USP saline or 0.1% TritonX-100™ as reference for 0 and 100% cell death, respectively.

Data were processed and analyzed using Graph Pad Prizm 4™ software (Graph Pad Prizm, Inc). Statistical analysis for significance was determined by a two-tailed Student's T-test. The whole MAb-Phor18 resulted in IC₅₀ values of 60.53±3.8 nM and scFv-C_{H}3-GS-Phor18 was 59.8±3.8 nM. The “naked” (unconjugated) antibodies (MAb and scFv-C_{H}3) were not cytotoxic. In vitro chemically linked HER2 antibody (MAb-Phor18) and recombinant Phor18 conjugate (scFv-C_{H}3-GS-Phor18) showed similar toxicity to SKOV-3 cells, whereas the naked recombinant antibody (scFv-C_{H}3) was not toxic.

### Example 5

This example describes an in vivo study in a mouse xenograft model of human ovarian cancer with various doses of anti-Her2-Phor18 antibody conjugates (scFv-C_{H}3-GS-Phor18, MAb-Phor18), naked whole antibody (MAb) and naked recombinant antibody (scFv-C_{H}3) treatments.

Female Nu/Nu mice were injected subcutaneously with a SKOV-3/Matrigel suspension (4×10⁶ cells). Tumor weights from mice that were killed on day 42 served as baseline. In brief, treatment started on day 43 after tumor cell injection on tumors of median tumor volume of 130.3±10.25 mm³ and continued on days 47, 50, 54, 57 and 60 as a single bolus injection into the lateral tail vein.

During the entire study tumor volumes were measured twice per week and body weights were determined. Final necropsy was conducted on day 64 after tumor cell injection where tumors were excised, weighed and fixed in formalin for histological evaluation.

Treatments were: saline control, whole naked monoclonal anti-Her2-antibody, MAb, (3 mg/kg), recombinant naked-Her2-antibody (scFv-C_{H}3) (3 mg/kg), scFv-C_{H}3-GS-Phor18 (0.3 and 3 mg/kg), MAb-Phor18 (0.3 and 3 mg/kg). Tumors from mice sacrificed at treatment start underwent immunohistochemistry evaluation of Her2/neu receptors. Each group consisted of 8-9 mice.

All groups of mice tolerated the injections well. No mice died as a consequence of injection.

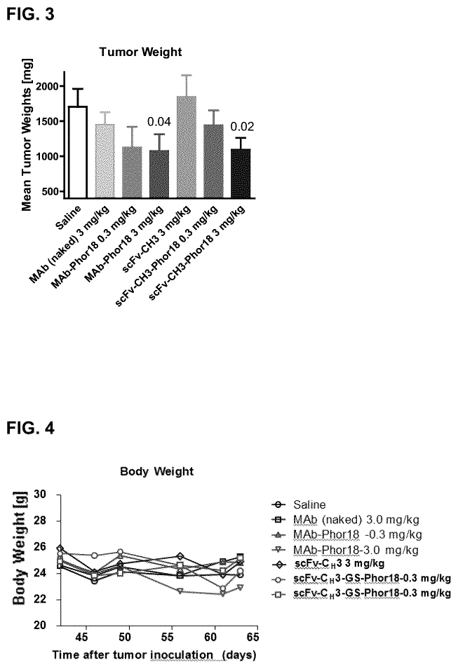

The effect of antibody conjugated Phor18 injections and naked antibodies on the primary tumors (volume and tumor weights, FIGS. 2A, 2B, and 3) and body weight is illustrated in FIG. 4. FIGS. 2A and 2B show median tumor volumes during the course of the study and mean tumor weights on day 64 for each individual treatment group for saline controls, and mice treated with MAb (naked) (3 mg/kg), scFv-C_{H}3 (3 mg/kg), scFv-C_{H}3-GS-Phor18 (0.3 and 3 mg/kg), MAb-Phor18 (0.3 and 3 mg/kg).

Tumor volumes and weights decreased significantly in all animals treated with 3 mg/kg MAb-Phor18 chemically linked (p<0.04) or recombinantly produced scFv-C_{H}3-Phor18 conjugates (p<0.02). Naked MAb or scFv-C_{H}3 were not decreasing tumor volumes or tumor weights compared to saline controls at doses of 3 mg/kg (FIGS. 2A, 2B, and 3). Statistical analysis was conducted in Graphpad prizm 4 using the Wilcoxon signed rank test. Body weights were stable in all treatment groups and control animals (FIG. 4).

### Example 6

This example describes in vitro cytotoxicity studies of recombinantly produced antibody to Her-2 receptor conjugated to lytic peptide, Phor18 (KFAKFAK KFAKFAK KFAK (SEQ ID NO.: 4)) against ovarian cancer cells.

scFv-C_{H}2-C_{H}3-GS-Phor18 (one molecule of Phor18 joined to the antibody at the C-terminus by GS linker, consisting of V_{L}-G linker-V_{H}-C_{H}2-C_{H}3-G linker-C_{H}3-C_{H}2-V_{H}-G linker-V_{L}-GS-(Phor18). Cytotoxicity was compared to a naked antibody (scFv-C_{H}2-C_{H}3; antibody without a lytic peptide) in Her-2 receptor positive cells (SKOV-3, human ovarian cancer cells).

Materials: Recombinant DNA technique was used to synthesize anti-Her2 antibody as an scFv-C_{H}2-C_{H}3 antibody in Escherichia coli. The antibody (Olafsen T. et al Protein Engineering, Design & Selection 17, 315-323, 2004) was conjugated via a peptide linker to either Phor18 and analyzed for cytotoxicity in vitro. The plasmid was acquired through gene codon optimization. The gene was synthesized with a N-His tag sequence and the plasmid was subcloned into an E. coli bacteria expression vector pUC57. After expression optimization and evaluation the His-tag product was selected and 1 L of the bacteria expression product was purified in a one-step affinity purification. The amino acid sequence of for each construct is described in Table 3.

```
TABLE 3
Amino Acid sequence for the production of each recombinant antibody,
(A) scFv-C_{H}2-C_{H}3; and antibody conjugate (B) scFv-C_{H}2-C_{H}3-GS-Phor18.
A) scFv-C_{H}2-C_{H}3; and antibody conjugate: (SEQ ID NO.: 82)
                10 20 30 40 50 60
DIQMTQSPSS LSASVGDRVT ITCRASQDVN TAVAWYQQKP GKAPKLLIYS ASFLYSGVPS
                70 80 90 100 110 120
RFSGSRSGTD FTLTISSLQP EDFATYYCQQ HYTTPPTFGQ GTKVEIKGST SGGGSGGGSG
              130 140 150 160 170 180
GGGSSEVQLV ESGGGLVQPG GSLRLSCAAS GFNIKDTYIH WVRQAPGKGL EWVARIYPTN
              190 200 210 220 230 240
GYTRYADSVK GRFTISADTS KNTAYLQMNS LRAEDTAVYY CSRWGGDGFY AMDYWGQGTL
              250 260 270 280 290 300
VTVSSPCPAP ELLGGPSVFL FPPKPKDTLM ISRTPEVTCV VVDVSHEDPE VKFNWYVDGV
              310 320 330 340 350 360
EVHNAKTKPR EEQYNSTYRV VSVLTVLHQD WLNGKEYKCK VSNKALPAPI EKTISKAKGQ
              370 380 390 400 410 420
PREPQVYTLP PSRDELTKNQ VSLTCLVKGF YPSDIAVEWE SNGQPENNYK TTPPVLDSDG
              430 440 450 460 470 480
SFFLYSKLTV DKSRWQQGNV FSCSVMHEAL HNHYTQKSLS LSPGKGSTSG GGSGGGSGGG
              490 500 510 520 530 540
GSSGQPREPQ VYTLPPSRDE LTKNQVSLTC LVKGFYPSDI AVEWESNGQP ENNYKTTPPV
              550 560 570 580 590 600
LDSDGSFFLY SKLTVDKSRW QQGNVFSCSV MHEALHNHYT QKSLSLSPGK PCPAPELLGG
              610 620 630 640 650 660
PSVFLFPPKP KDTLMISRTP EVTCVVVDVS HEDPEVKFNW YVDGVEVHNA KTKPREEQYN
              670 680 690 700 710 720
STYRVVSVLT VLHQDWLNGK EYKCKVSNKA LPAPIEKTIS KAKEVQLVES GGGLVQPGGS
              730 740 750 760 770 780
LRLSCAASGF NIKDTYIHWV RQAPGKGLEW VARIYPTNGY TRYADSVKGR FTISADTSKN
              790 800 810 820 830 840
TAYLQMNSLR AEDTAVYYCS RWGGDGFYAM DYWGQGTLVT VSSGSTSGGG SGGGSGGGGS
              850 860 870 880 890 900
SDIQMTQSPS SLSASVGDRV TITCRASQDV NTAVAWYQQK PGKAPKLLIY SASFLYSGVP
              910 920 930 940
SRFSGSRSGT DFTLTISSLQ PEDFATYYCQ QHYTTPPTFG QGTKVEIK
B) scFv-C_{H}2-C_{H}3-GS-Phor18: (SEQ ID NO.: 83)
                10 20 30 40 50 60
DIQMTQSPSS LSASVGDRVT ITCRASQDVN TAVAWYQQKP GKAPKLLIYS ASFLYSGVPS
                70 80 90 100 110 120
RFSGSRSGTD FTLTISSLQP EDFATYYCQQ HYTTPPTFGQ GTKVEIKGST SGGGSGGGSG
              130 140 150 160 170 180
GGGSSEVQLV ESGGGLVQPG GSLRLSCAAS GFNIKDTYIH WVRQAPGKGL EWVARIYPTN
              190 200 210 220 230 240
GYTRYADSVK GRFTISADTS KNTAYLQMNS LRAEDTAVYY CSRWGGDGFY AMDYWGQGTL
              250 260 270 280 290 300
VTVSSPCPAP ELLGGPSVFL FPPKPKDTLM ISRTPEVTCV VVDVSHEDPE VKFNWYVDGV
              310 320 330 340 350 360
EVHNAKTKPR EEQYNSTYRV VSVLTVLHQD WLNGKEYKCK VSNKALPAPI EKTISKAKGQ
              370 380 390 400 410 420
PREPQVYTLP PSRDELTKNQ VSLTCLVKGF YPSDIAVEWE SNGQPENNYK TTPPVLDSDG
              430 440 450 460 470 480
SFFLYSKLTV DKSRWQQGNV FSCSVMHEAL HNHYTQKSLS LSPGKGSTSG GGSGGGSGGG
              490 500 510 520 530 540
GSSGQPREPQ VYTLPPSRDE LTKNQVSLTC LVKGFYPSDI AVEWESNGQP ENNYKTTPPV
              550 560 570 580 590 600
LDSDGSFFLY SKLTVDKSRW QQGNVFSCSV MHEALHNHYT QKSLSLSPGK PCPAPELLGG
              610 620 630 640 650 660
PSVFLFPPKP KDTLMISRTP EVTCVVVDVS HEDPEVKFNW YVDGVEVHNA KTKPREEQYN
              670 680 690 700 710 720
STYRVVSVLT VLHQDWLNGK EYKCKVSNKA LPAPIEKTIS KAKEVQLVES GGGLVQPGGS
              730 740 750 760 770 780
LRLSCAASGF NIKDTYIHWV RQAPGKGLEW VARIYPTNGY TRYADSVKGR FTISADTSKN
              790 800 810 820 830 840
TAYLQMNSLR AEDTAVYYCS RWGGDGFYAM DYWGQGTLVT VSSGSTSGGG SGGGSGGGGS
              850 860 870 880 890 900
SDIQMTQSPS SLSASVGDRV TITCRASQDV NTAVAWYQQK PGKAPKLLIY SASFLYSGVP
              910 920 930 940 950 960
SRFSGSRSGT DFTLTISSLQ PEDFATYYCQ QHYTTPPTFG QGTKVEIKGS KFAKFAKKFA
KFAKKFAK
```

In vitro cytotoxicity studies were performed to determine cytotoxicity of the recombinant antibody preparations (scFv-C_{H}2-C_{H}3, scFv-C_{H}3, and scFv-C_{H}2-C_{H}3-GS-Phor18, scFv-C_{H}3-GS-Phor18). Her-2 receptor positive SKOV-3 cells were prepared in 96 well plates using 2,000 cells/well and were allowed to attach for 48 hours. The Her2-antibody-Phor18 conjugates (scFv-C_{H}2-C_{H}3-GS-Phor18, scFv-C_{H}3 GS-Phor18) or the naked antibodies (scFv-C_{H}2-C_{H}3, scFv-C_{H}3) in Tris/HCL-buffer were diluted with saline and added to cells at increasing concentrations of 0, 0.0012, 0.012, 0.12, 1.2, 6.0, 12.0, 120, 360 and 720 nM. Incubations were conducted for 48 h at 37° C. Cell viability was determined using Cell Titer Glo luminescent cell viability assay (Promega). Controls contained USP saline or 0.1% TritonX-100™ as reference for 0 and 100% cell death, respectively. All data were processed and analyzed using Graph Pad Prizm 4™ software (Graph Pad Prizm, Inc).

The Her2 antibody (scFv-C_{H}2-C_{H}3, scFv-C_{H}3) conjugated to the Phor18 resulted in IC₅₀ values of 53.7±0.63 nM for scFv-C_{H}3-Phor18 and 56.7±0.92 nM for scFv-C_{H}2-C_{H}3-Fv-Phor18. The “naked” (unconjugated) antibodies, scFv-C_{H}2-C_{H}3, scFv-C_{H}3, were not cytotoxic. In vitro recombinantly Phor18 conjugates show similar toxicity to SKOV-3 cells.

### Example 7

This example includes a description of the construction and expression of whole anti-Her2-IgG1-Phor18 antibody conjugates with defined location lytic domain (Phor18), and specified numbers of 2, 4 and 6 Phor18 lytic domains per antibody in a mammalian expression system. These conjugates are also referred to as antibody-drug conjugates (ADC).

Recombinant expression of whole IgG1 antibody-Phor18 (KFAKFAKKFAKFAKKFAK (SEQ ID NO.: 4)) conjugates in a mammalian system (CHO cells) was conducted using two different secretion signal sequences: a proprietary secretion signal sequence for antibody heavy and light chains, and human IgG kappa-light chain secretion signal. The expressed anti-Her2 IgG1 antibody (humanized variable light and heavy domains regions to Her-2 receptor) and the various antibody-Phor18 conjugates with stoichiometric ratios of Phor18: AB of 2, 4 and 6 were characterized using SDS PAGE, Western blot analyses, and surface Plasmon resonance (selected ADCs).

Yield, purity and cytotoxicity of recombinantly produced antibodies (as a full IgG1 antibody) with Heavy (H) or Light (L) chain C-terminal- or N-terminal-Phor18 conjugation was analyzed. Two, 4 and 6 molecules of lytic domains (Phor18) conjugated to whole antibody molecule were expressed. The amino acid sequences of the “unconjugated anti-Her2 antibody, anti-Her2 antibody heavy (H) and light (L) chains are shown in Table 4.

TABLE 4
Anti-Her2/neu antibody amino acid sequence from Drugbank.ca DB00072.
There was one additional amino acid in the C_{H}1 domain of the Anti-Her2
antibody in comparison to human IgG1 (underlined).

```
Anti-HER2/neu Light chain (naked L1): (SEQ ID NO.: 84)
                10 20 30 40 50 60
DIQMTQSPSS LSASVGDRVT ITCRASQDVN TAVAWYQQKP GKAPKLLIYS ASFLYSGVPS
                70 80 90 100 110 120
RFSGSRSGTD FTLTISSLQP EDFATYYCQQ HYTTPPTFGQ GTKVEIKRTV AAPSVFIFPP
              130 140 150 160 170 180
SDEQLKSGTA SVVCLLNNFY PREAKVQWKV DNALQSGNSQ ESVTEQDSKD STYSLSSTLT
              190 200 210
LSKADYEKHK VYACEVTHQG LSSPVTKSFN RGEC
Anti-HER2/neu Heavy chain 2 (naked H1): (SEQ ID NO.: 85)
                10 20 30 40 50 60
EVQLVESGGG LVQPGGSLRL SCAASGFNIK DTYIHWVRQA PGKGLEWVAR IYPTNGYTRY
                70 80 90 100 110 120
ADSVKGRFTI SADTSKNTAY LQMNSLRAED TAVYYCSRWG GDGFYAMDYW GQGTLVTVSS
              130 140 150 160 170 180
ASTKGPSVFP LAPSSKSTSG GTAALGCLVK DYFPEPVTVS WNSGALTSGV HTFPAVLQSS
              190 200 210 220 230 240
GLYSLSSVVT VPSSSLGTQT YICNVNHKPS NTKVDKKVEP PKSCDKTHTC PPCPAPELLG
              250 260 270 280 290 300
GPSVFLFPPK PKDTLMISRT PEVTCVVVDV SHEDPEVKFN WYVDGVEVHN AKTKPREEQY
              310 320 330 340 350 360
NSTYRVVSVL TVLHQDWLNG KEYKCKVSNK ALPAPIEKTI SKAKGQPREP QVYTLPPSRD
              370 380 390 400 410 420
ELTKNQVSLT CLVKGFYPSD IAVEWESNGQ PENNYKTTPP VLDSDGSFFL YSKLTVDKSR
              430 440 450
WQQGNVFSCS VMHEALHNHY TQKSLSLSPG K
```

The amino acid sequences of antibody-lytic peptide conjugate heavy (H) and light (L) chains are shown in Table 5. Gene synthesis was conducted at Genewiz, Inc (South Plainfield, NJ) using preferred codon usage for Chinese Hamster Ovary cells. The transcripts were ligated into the pUC57 bacterial plasmid.

TABLE 5
Amino acid sequences of lytic-peptide (Phor18, KFAKFAKKFAKFAKKFAK
(SEQ ID NO.: 4))-antibody heavy and light chain conjugates

```
Phor18-V_{L} Light Chain (L2): (SEQ ID NO.: 86)
                10 20 30 40 50 60
KFAKFAKKFA KFAKKFAKGS DIQMTQSPSS LSASVGDRVT ITCRASQDVN TAVAWYQQKP
                70 80 90 100 110 120
GKAPKLLIYS ASFLYSGVPS RFSGSRSGTD FTLTISSLQP EDFATYYCQQ HYTTPPTFGQ
              130 140 150 160 170 180
GTKVEIKRTV AAPSVFIFPP SDEQLKSGTA SVVCLLNNFY PREAKVQWKV DNALQSGNSQ
              190 200 210 220 230
ESVTEQDSKD STYSLSSTLT LSKADYEKHK VYACEVTHQG LSSPVTKSFN RGEC
C_{L}-Phor18 Light Chain (L3): (SEQ ID NO.: 87)
                10 20 30 40 50 60
DIQMTQSPSS LSASVGDRVT ITCRASQDVN TAVAWYQQKP GKAPKLLIYS ASFLYSGVPS
                70 80 90 100 110 120
RFSGSRSGTD FTLTISSLQP EDFATYYCQQ HYTTPPTFGQ GTKVEIKRTV AAPSVFIFPP
              130 140 150 160 170 180
SDEQLKSGTA SVVCLLNNFY PREAKVQWKV DNALQSGNSQ ESVTEQDSKD STYSLSSTLT
              190 200 210 220 230
LSKADYEKHK VYACEVTHQG LSSPVTKSFN RGECGSKFAK FAKKFAKFAK KFAKA
Phor18-V_{L}-C_{L}-Phor18 Light Chain (L4): (SEQ ID NO.: 88)
                10 20 30 40 50 60
KFAKFAKKFA KFAKKFAKGS DIQMTQSPSS LSASVGDRVT ITCRASQDVN TAVAWYQQKP
                70 80 90 100 110 120
GKAPKLLIYS ASFLYSGVPS RFSGSRSGTD FTLTISSLQP EDFATYYCQQ HYTTPPTFGQ
              130 140 150 160 170 180
GTKVEIKRTV AAPSVFIFPP SDEQLKSGTA SVVCLLNNFY PREAKVQWKV DNALQSGNSQ
              190 200 210 220 230 240
ESVTEQDSKD STYSLSSTLT LSKADYEKHK VYACEVTHQG LSSPVTKSFN RGECGSKFAK
              250
FAKKFAKFAK KFAKA
Phor18-VH Heavy Chain (H2) (SEQ ID NO.: 89)
                10 20 30 40 50 60
KFAKFAKKFA KFAKKFAKGS EVQLVESGGG LVQPGGSLRL SCAASGFNIK DTYIHWVRQA
                70 80 90 100 110 120
PGKGLEWVAR IYPTNGYTRY ADSVKGRFTI SADTSKNTAY LQMNSLRAED TAVYYCSRWG
              130 140 150 160 170 180
GDGFYAMDYW GQGTLVTVSS ASTKGPSVFP LAPSSKSTSG GTAALGCLVK DYFPEPVTVS
              190 200 210 220 230 240
WNSGALTSGV HTFPAVLQSS GLYSLSSVVT VPSSSLGTQT YICNVNHKPS NTKVDKKVEP
              250 260 270 280 290 300
PKSCDKTHTC PPCPAPELLG GPSVFLFPPK PKDTLMISRT PEVTCVVVDV SHEDPEVKFN
              310 320 330 340 350 360
WYVDGVEVHN AKTKPREEQY NSTYRVVSVL TVLHQDWLNG KEYKCKVSNK ALPAPIEKTI
              370 380 390 400 410 420
SKAKGQPREP QVYTLPPSRD ELTKNQVSLT CLVKGFYPSD IAVEWESNGQ PENNYKTTPP
              430 440 450 460 470
VLDSDGSFFL YSKLTVDKSR WQQGNVFSCS VMHEALHNHY TQKSLSLSPG K
C_{H}3-Phor18 Heavy Chain (H3): (SEQ ID NO.: 90)
                10 20 30 40 50 60
EVQLVESGGG LVQPGGSLRL SCAASGFNIK DTYIHWVRQA PGKGLEWVAR IYPTNGYTRY
                70 80 90 100 110 120
ADSVKGRFTI SADTSKNTAY LQMNSLRAED TAVYYCSRWG GDGFYAMDYW GQGTLVTVSS
              130 140 150 160 170 180
ASTKGPSVFP LAPSSKSTSG GTAALGCLVK DYFPEPVTVS WNSGALTSGV HTFPAVLQSS
              190 200 210 220 230 240
GLYSLSSVVT VPSSSLGTQT YICNVNHKPS NTKVDKKVEP PKSCDKTHTC PPCPAPELLG
              250 260 270 280 290 300
GPSVFLFPPK PKDTLMISRT PEVTCVVVDV SHEDPEVKFN WYVDGVEVHN AKTKPREEQY
              310 320 330 340 350 360
NSTYRVVSVL TVLHQDWLNG KEYKCKVSNK ALPAPIEKTI SKAKGQPREP QVYTLPPSRD
              370 380 390 400 410 420
ELTKNQVSLT CLVKGFYPSD IAVEWESNGQ PENNYKTTPP VLDSDGSFFL YSKLTVDKSR
              430 440 450 460 470
WQQGNVFSCS VMHEALHNHY TQKSLSLSPG KGSKFAKFAK KFAKFAKKFA KA
Phor18-V_{H}-C_{H}3-Phor18 Heavy Chain (H4): (SEQ ID NO.: 91)
                10 20 30 40 50 60
KFAKFAKKFA KFAKKFAKGS EVQLVESGGG LVQPGGSLRL SCAASGFNIK DTYIHWVRQA
                70 80 90 100 110 120
PGKGLEWVAR IYPTNGYTRY ADSVKGRFTI SADTSKNTAY LQMNSLRAED TAVYYCSRWG
              130 140 150 160 170 180
GDGFYAMDYW GQGTLVTVSS ASTKGPSVFP LAPSSKSTSG GTAALGCLVK DYFPEPVTVS
              190 200 210 220 230 240
WNSGALTSGV HTFPAVLQSS GLYSLSSVVT VPSSSLGTQT YICNVNHKPS NTKVDKKVEP
              250 260 270 280 290 300
PKSCDKTHTC PPCPAPELLG GPSVFLFPPK PKDTLMISRT PEVTCVVVDV SHEDPEVKFN
              310 320 330 340 350 360
WYVDGVEVHN AKTKPREEQY NSTYRVVSVL TVLHQDWLNG KEYKCKVSNK ALPAPIEKTI
              370 380 390 400 410 420
SKAKGQPREP QVYTLPPSRD ELTKNQVSLT CLVKGFYPSD IAVEWESNGQ PENNYKTTPP
              430 440 450 460 470 480
VLDSDGSFFL YSKLTVDKSR WQQGNVFSCS VMHEALHNHY TQKSLSLSPG KGSKFAKFAK
              490
KFAKFAKKFA KA
```

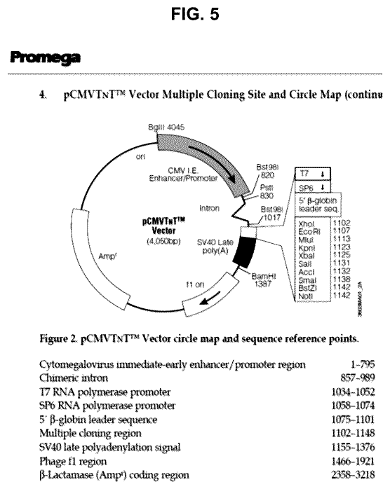

Heavy (H) and light (L) chain transcripts were synthesized out of Genewiz pUC57 plasmids using PCR with primers containing 5′ EcoRI and 3′ Xbal restriction sites for directional ligation into the multiple cloning site of the pCMVTnT mammalian expression plasmid (FIG. 5, Promega, Madison, WI, L5620, lot 14524919). Each transcript included a Kozak consensus sequence and a secretion signal at the 5′ end.

Anti-Her2/neu antibody was produced by Lonza Inc. (Cambridge, UK). A Chinese Hamster Ovary (CHO) cell mammalian expression system was used to produce naked (i.e., no lytic domain) anti-Her2/neu IgG1, and ADCs having a lytic domain (Phor18) at defined locations (N- or C-terminal) and in ratios of 2, 4 and 6 lytic domains per whole antibody.

In brief, Free-style CHO Suspension Cell Expression System (Invitrogen Life Sciences, Carlsbad, CA, cat #K9000-20) was used to grow FS-CHO cells (according to manufacturer's directions) in FreeStyle CHO expression medium supplemented with 8 mM glutamax and 5 ml/L penicillin/streptomycin. Cells were thawed into growth medium without penicillin/streptomycin that was prewarmed to 37° C. and equilibrated in an 8% CO₂ atmosphere and grown in 30 ml in shaker flasks at 125-135 rpm. Cell density was kept at or below 1×10⁶ cells/ml to avoid clumping.

FS-CHO cells were transfected according to the Invitrogen protocol. FS CHO cells were expanded for 7 or more days after thawing. Cells were doubling every 24 h. The day before transfection, clumps were removed and cells were pelleted and resuspended in P/S-free medium at 5×10⁵/ml. On the day of transfection, cells were adjusted to 9×10⁵/ml if necessary and viability was close to 99%. Each 500 ml spinner flask (VWR, cat #PBV125) of 180 ml cells was transfected with 180 μg of total plasmid DNA mixed with 180 μl of FSMax transfection reagent (Invitrogen, cat #16447-100). Cells were swirled rapidly while adding DNA mixture slowly. Ratios of H: L chains analyzed were 3:2, 1:1, and 2:3.

ADC secreted into the cell medium, harvested on day 4 to day 6 after transfection, was purified using protein A columns. Approximately 0.25 ml of protein A resin (Genscript L00210, capacity >20 mg IgG per ml resin) was used to isolate secreted ADCs from the FS CHO medium using the Genscript product protocol and buffer descriptions provided. ADC was eluted once with 0.5 ml low pH elution buffer of 0.1M glycine pH 2.5 and eluate was reapplied to the column and collected a second time. pH was adjusted to 6.5 with IM Tris pH 8.

To confirm the presence of protein, SDS-PAGE (4-15% TGX gels, Bio-Rad Labs, Hercules, CA, cat #456-1084) analyses was used. ADCs separated on gels were silver-stained (Sigma-Aldrich, St Louis, MO, Prot Sill kit). The anti-Phor18 rabbit polyclonal was from Covance (Denver, PA, cat #338983), and for detection an anti-rabbit-HRP was used (cat #111-035-046, Jackson ImmunoResearch, Philadelphia, PA). Detection of ADCs was conducted with an anti-human IgG from Jackson ImmunoResearch (cat #109-035-088). The presence of the (L) light chain was confirmed on reduced ADCs with HRP-mouse anti-human kappa (Invitrogen, cat #053920).

To formulate the ADCs, DPBS salts in a 20× solution and polysorbate 20 (PS20) were added to the protein A elution buffer (Tris-glycine, 50 mM-100 mM) at the following final concentrations to stabilize for storage at 4° C. and during freeze-thaw cycles: CaCl₂) 100 mg/L, MgCl2 (·6H2O) 100 mg/L, KCl 200 mg/L, NaCl 8 g/L, and 0.09 mg/ml PS20. To determine protein concentration, for some batches, the OD280 for each sample was determined on a spectrophotometer. ADC concentration [mg/ml] was calculated by using an extinction coefficient of 1.4 (based on amino acid sequence). For greater accuracy, ADC concentration was determined by anti-human IgG Elisa assay (Genway, 40-374-130037).

The first Anti-Her2/neu antibody-based ADCs were produced under the regulation of signal sequences and expression analysis was conducted. ADCs produced were: H1L1 (naked), H2L2 (Phor18-V_{L}-Phor18-V_{H}-IgG1), H2L1 (Phor18-V_{H}-IgG1), H1L2 (Phor18-V_{L}-IgG1), H1L3 (C_{L}-Phor18-IgG1) H3L1 (C_{H}3-Phor18-IgG1), H3L3 (C_{L}-Phor18-C_{H}3-Phor18-IgG1), H3L4 (Phor18-V_{L}-C_{L}-Phor18-C_{H}3-Phor18-IgG1), and H4L3 (Phor18-V_{H}-C_{L}-Phor18-C_{H}3-Phor18-IgG1). Based on spectrophotometric (OD ₂₈₀) analysis, average yields for ADCs were H2L2=0.12 mg/L and H1L3=0.8 mg/L

Table 6 shows the individual ADC descriptions, abbreviations, and the number and locations of lytic domains (Phor18).

TABLE 6
ADC descriptions and abbreviations

| | ADC | | Number of |
|---|---|---|---|
| | Abbre- | | Phor18 lytic |
| | viation | | sequences/ |
| Name of ADC | (H L) | Phor18 location | Antibody |
| IgG1 | H1L1 | None (‘Naked’) | 0 |
| Phor18-V_{L}-IgG1 | H1L2 | N-termini light chains (V_{L}) | 2 |
| C_{L}-Phor18-IgG1 | H1L3 | C-termini light chains (C_{L}) | 2 |
| Phor18-V_{L}-IgG1- | H1L4 | N-termini and | 4 |
| C_{L}-Phor18 | | C-termini light chains | |
| | | (V_{L}, C_{L}) | |
| Phor18-V_{L}- | H2L2 | N-termini heavy | 4 |
| Phor18-V_{H}-IgG1 | | and light chains | |
| | | (V_{H}, V_{L}) | |
| Phor18-V_{H}-IgG1 | H2L1 | N-termini heavy chains (V_{H}) | 2 |
| Phor18-V_{H}-C_{L}- | H2L3 | N-termini heavy | 4 |
| Phor18-IgG1 | | chains and C-termini | |
| | | light chains (V_{H},C_{L}) | |
| C_{H}3-Phor18-IgG1 | H3L1 | C-termini heavy chain (C_{H}3) | 2 |
| Phor18-V_{L}-C_{H}3- | H3L2 | N-termini light chains, | 4 |
| Phor18-IgG1 | | C-termini heavy | |
| | | chains (V_{L}, C_{H}3) | |
| C_{L}-Phor18-C_{H}3- | H3L3 | C-termini light and | 4 |
| Phor18-IgG1 | | heavy chains (C_{L}, | |
| | | C_{H}3) | |
| Phor18-V_{L}-C_{L}- | H3L4 | N-termini light | 6 |
| Phor18-C_{H}3- | | chains and C-termini | |
| Phor18-IgG1 | | both light and | |
| | | heavy chains (V_{L},C_{L}, | |
| | | C_{H}3) | |
| Phor18-V_{H}-C_{L}- | H4L3 | N-termini heavy | 6 |
| Phor18-C_{H}3- | | chains; C-termini both | |
| Phor18-IgG1 | | heavy and light chains | |
| | | (V_{H}, C_{L}, C_{H}3) | |
| Phor18-V_{H}-C_{H}3- | H4L1 | N-and C-termini heavy | 4 |
| Phor18-IgG1 | | chains (V_{H}, C_{H}3) | |
| Phor18-V_{H}- | H4L2 | N-termini heavy | 6 |
| Phor18V_{L}-C_{H}3- | | and light chains, C- | |
| Phor18-IgG1 | | termini heavy chains | |
| | | (V_{H}, V_{L}, C_{H}3) | |

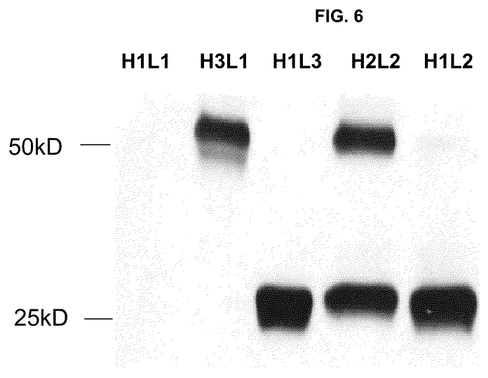

The quality of ADCs was analyzed on immunoblots of reduced antibodies, allowing heavy and light chains to be visualized. The presence of Phor18 was confirmed on the light (L) chain of H1L2 Phor18-V_{L}-IgG1 (H1L2), on the light (L) chain of H1L3 (C_{L}-Phor18-IgG1) and on the heavy (H) chain of H3L1 (C_{H}3-Phor18-IgG1), which has Phor18 only on the C-terminus of the light chain (FIG. 6).

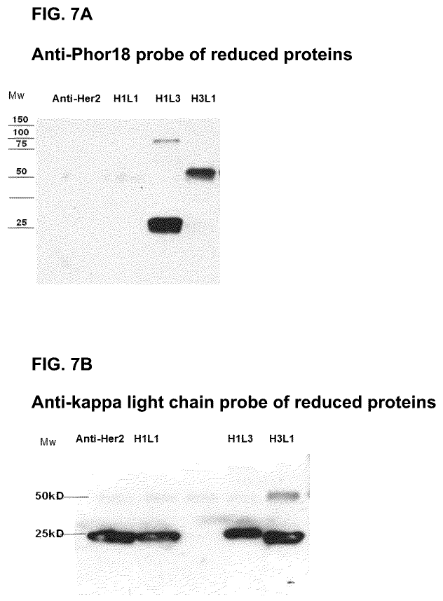

Presence of Phor18 on heavy and light chain was confirmed in westernblot analysis (FIG. 7A) for the heavy chains with Phor18 conjugation. H1L1 and anti-Her2/neu antibody, the naked antibody, served as negative control and did not show a band probing for Phor18. Presence of light chains in recombinantly produced antibodies and antibody conjugates were confirmed in western blot analysis (kappa light chains) for H1L1 (IgG1), H3L1 (C_{H}3-Phor18-IgG1), H1L3 (IgG1-C_{L}-Phor18) and H2L2 (Phor18-V_{L}-Phor18-V_{H}-IgG1) had both heavy and light chains based on IgG western blot analysis, kappa-light chain western blot analysis (FIG. 7B) and anti-Phor18 immuno blots (FIGS. 6 and 7A).

The data indicate that whole antibody-lytic domain (Phor18) conjugates can be produced recombinantly in a mammalian expression system having pre-determined stoichiometries of lytic domain (Phor18): AB of 2, 4 and 6, and lytic domain (Phor18) in pre-determined locations.

### Example 8

This example includes a description of the potency and specificity of the eight ADCs.

ADCs with anti-Her-2 receptor (IgG1) conjugated to 2, 4 or 6 Phor18 molecules at the N- or C-terminus were analyzed in vitro using a Her2/neu positive ovarian cancer cell line (SKOV-3 human ovarian cancer cells) and compared to “naked” antibody. Her-2 receptor negative, ER negative, PR negative human breast cancer cells (MDA-MB-231) served as control.

In brief, the SKOV-3 (Her2/neu positive, passage number pU51) and MDA-MB-231 (Her2/neu negative, ER negative, PR negative, pu 14) cells were seeded at a density of 2,000 cells per well in opaque plates in heat inactivated full medium using cell dissociation buffer. After 2 days, cells were replenished with fresh media (75 μl) and incubated with 25 μl of a 4× serial dilution of each ADC and naked antibody prepared in cell culture media were added.

Cells incubated for 4 hours were assayed for membrane integrity using a luminometric assay kit (Promega, Cytotox Glo G9292 lot #317872). Cell viability was determined was determined after 24, 48 and 72 hours using a luminescent assay kit (Promega, W, Cell Titer Glo, G 7572, lot 31511202). Controls for 100% cell viability (culture media) and 100% cell death (0.1% Triton X 100) were incubated under the same conditions.

Data were processed and analyzed to obtain IC₅₀ values using Graph Pad Prizm version 5.00 for Windows, GraphPad Software, San Diego California USA, www-graphpad-com (Graph Pad Prizm, Inc). Statistical analysis for significance was determined by a two-tailed Student's T-test. Each test was conducted using double plates of 2-3 wells each to achieve an N of 4-6 data points per time point.

The concentration of each ADC was determined spectrophotometrically (OD₂₈₀) and in some cases by IgG determination using ELISA assays. Each ADC and naked antibody were prepared from frozen stocks to produce as highest concentration 800 nM or lower depending on the initial concentration.

Serial dilutions were prepared in cell culture media to achieve a final concentration per well of 0, 0.001, 0.01, 0.1, 1, 10, 100 and 200 nM, corresponding to 0.00015, 0.0015, 0.015, 0.15, 1.5, 15, 30 and 60 μg/ml for concentrations determined spectrophotometrically. For studies that had IgG contents the highest possible concentration was tested followed by a 1:1 and 1:10 dilutions for each ADC.

Various time points were included to determine the activity of the recombinantly expressed ADCs. The earliest indication of activity was measured as effect on membrane integration after 4 hours of ADC exposure in Her2/neu positive SKOV-3 cells.

N-terminal conjugated ADC with Phor18 on the variable light chain (Phor18-V_{L}-IgG1) disintegrated the target cell membrane (IC₅₀=187.9±12.8 nM). C-terminal conjugated ADC (C_{H}3-Phor18-IgG1) with one Phor18 molecule had no detectable membrane activity. Naked anti-Her2-IgG1 also did not detetcably affect membrane integrity.

Table 7 summarizes the results of this activity study. ADCs showed high specificity for the target cell line SKOV-3 compared to the negative control (Her2/neu negative) MDA-MB-231.

Maximal toxicity levels were determined after 48 hours. The IC₅₀ values [nM] were in the low nanomolar range for N-terminus conjugated ADC Phor18-V_{H}-IgG1 (13.02±2.3 nM, 2 Phor18/AB, N-terminus) and H2L1 Phor18-V_{L}-IgG1 (6.9-3.4 nM, 2 Phor18/AB, N-terminus), compared to C-terminus conjugated ADC: C_{H}3-Phor18-IgG1 (27.4±5.1 nM, 2 Phor18/AB, H3L1, C-terminus) having 2 Phor18 molecules per antibody.

N-terminal conjugated ADCs with 4 molecules Phor18 on the N-terminus had IC₅₀ values of 0.54±0.2 nM (Phor18-V_{L}-Phor18-V_{H}-IgG1, 4 Phor18/AB, N-terminus, H2L2) compared to the C-terminus counterpart C_{L}-Phor18-C_{H}3-Phor18-IgG1 (36.3±10.6 nM, 4 Phor18/AB, H3L3, C-terminus, p<0.001). Lytic domain conjugation at the N-terminus was 70 fold more potent than conjugation at the C-terminus.

These data indicate that ADCs having N-terminal Phor18 conjugation were superior to C-terminal Phor18 conjugation for stoichiometric ratios of 2 and 4 molecules per antibody.

Increase of Phor18 conjugation from 2 to 4 molecules per antibody resulted in a 12 fold more potent ADC with conjugation at the N-terminus, but not at the C-terminus. ADCs conjugated with 6 Phor18 molecules per antibody showed about the same potency with IC₅₀ values of 1.1±0.2 nM for Phor18-V_{L}-C_{L}-Phor18-C_{H}3-Phor18-IgG1 (6 Phor18, N and C terminus, H3L4), and 1.1±0.4 nM for Phor18-V_{H}-C_{L}-Phor18-C_{H}3-Phor18-IgG1 (6 Phor18, H4L3).

Naked anti-Her2-IgG1 showed substantially less cell killing activity in SKOV-3 target cells after 48 hours (IC₅₀ values of 234.6±49 nM). None of the ADCs killed target negative MDA-MB-231 cells under the same conditions.

These data show that lytic domain (Phor18)-conjugated anti-Her-2 receptor antibodies (ADCs) are highly specific to Her2/neu expressing cells. Absence of the target receptor leaves cells unharmed.

The potency of lytic domain (Phor18) conjugated antibodies (ADCs) were dependent on the location of conjugation and the number of lytic domain (Phor18) molecules: the highest potency against target cells were observed for the N-terminal lytic domain (Phor18)-antibody conjugates with 2 and 4 lytic domain (Phor18) molecules per antibody. The N-terminal conjugation exhibited a 70 fold activity increase compared to the C-terminal lytic domain (Phor18)-antibody conjugation. N-terminal Phor18 conjugated ADCs were more potent compared to C-terminal Phor18 conjugates having both 2 and 4 Phor18 molecules per antibody. In addition, N-terminal conjugation showed measurable effects on reducing membrane integrity of the target cells SKOV-3.

Increasing the number of conjugated lytic domain (Phor18) molecules to 6 per antibody had maximal activities in the low nanomolar range. Phor18 conjugated ADCs were up to 433 fold more potent compared to the naked antibody.

TABLE 7
In vitro activity of ADCs and naked antibody produced in CHO cells

| | | | In vitro | |
|---|---|---|---|---|
| | | | activity after 48 hours | |
| | | Terminus of | IC₅₀ [nM] | IC₅₀ [nM] |
| | | Conjugation | SKOV-3 | MDA- |
| ADC and | | of | Her2/ | MB-231 |
| Naked antibody | ADC-ID | Phor18 to AB | neu (+) | Her2/neu(−) |
| IgG1 | H1L1 | None | 234.6 ± 49 | Not toxic |
| Phor18-V_{L}-IgG1 | H1L2 | N-termini, L | 6.9 ± 3.4 | Not toxic |
| chains | | chains | | |
| Phor18-V_{H}-IgG1 | H2L1 | N-termini, H | 13.02 ± 2.3 | Not toxic |
| | | chains | | |
| Phor18-V_{L}- | H2L2 | N-termini, H and | 0.54 ± 0.2 | Not toxic |
| Phor18-V_{H}-IgG1 | | L chains | | |
| C_{H}3-Phor18-IgG1 | H3L1 | C termini H | 27.4 ± 5.1 | Not toxic |
| | | chains | | |
| C_{L}-Phor18-C_{H}3- | H3L3 | C-termini H and | 36.3 ± 10.6 | Not toxic |
| Phor18-IgG1 | | L chains | | |
| Phor18-V_{L}- | H3L4 | N-termini L | 1.1 ± 0.2 | Not toxic |
| C_{L}-Phor18- | | chains and C | | |
| C_{H}3-Phor18-IgG1 | | chains H chains | | |
| Phor18-V_{H}- | H4L3 | N-termini H | 1.1 ± 0.4 | Not toxic |
| C_{L}-Phor18- | | chains and C- | | |
| C_{H}3-Phor18-IgG1 | | termini L chains | | |

### Example 9

This example includes a description of optimization of ADC expression using IgG kappa signal sequences. An alternate secretion signal peptide was evaluated for improvement of quality and expression levels of ADCs. To produce human IgG kappa signal sequence for expression of heavy and light chains with N-terminal Phor18, new gene synthesis for the L2 (Phor18-V_{L}), L4 (Phor18-V_{L}-C_{L}-Phor18), and H2 (Phor18-V_{H}) antibody heavy (H) and light (L) chain transcripts with the new signal peptide at the 5′ end was conducted by Genewiz, Inc. The amino acid sequence of the human IgG kappa signal peptide is MQTDTLLLWVLLLWVPGSTGA (SEQ ID NO.: 154) (Felgenhauer M, et al., Nucleic Acids Res., 18:4927 (1990)).

The Phor18-V_{L}-IgG1 and Phor18-V_{L}-Phor18-V_{H}-IgG1 ADCs produced with the igk secretion signal had a strong signal for light chains on immunoblots of reduced proteins probed with anti-C_{L} kappa and anti-Phor18. Because the IgG kappa signal peptide induced higher expression and quality, the igk transcripts were selected for ADC production. Production levels of ADCs expressed in CHO cells with the IgG kappa signal sequence, based on Elisa quantitation, were: H1L1=0.8 mg/L, C_{L}-Phor18-IgG1=0.8 mg/L, Phor18-V_{L}-IgG1=0.3 mg/L, and Phor18-V_{L}-Phor18-V_{H}-IgG1 =0.15 mg/L. Several additional batches of N-terminal Phor18 ADCs with IgG kappa signal sequence were produced and analyzed for expression level, quality and efficacy in vitro with comparable expression levels.

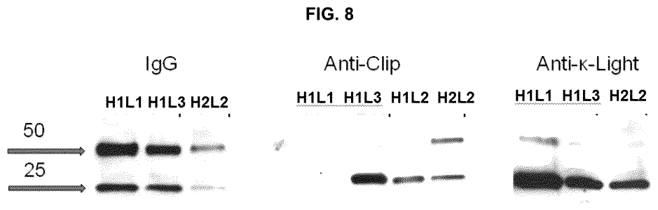

Phor18-V_{L}-IgG1 (H1L2), IgG1-C_{L}-Phor18-IgG1 (H1L3) and Phor18-V_{L}-Phor18-V_{H}-IgG1 (H2L2). These N-terminal and C-terminal conjugated Phor18 ADCs were analyzed for quality and purity by anti-Phor18 immunoblots, IgG and kappa light chain western blot analysis (FIG. 8). Phor18 presence was confirmed on the heavy and light chains through their molecular weights (FIG. 8).

Immunoblot analysis of ADCs showed that the Phor18-V_{L}-IgG1, C_{L}-Phor18-IgG1 and Phor18-V_{L}-Phor18-V_{H}-IgG1 ADCs produced with the igk secretion signal had heavy and light chains present with Phor18. Expression levels were lower than 0.5 mg/L for both Phor18-V_{L}-IgG1 and Phor18-V_{L}-Phor18-V_{H}-IgG1.

### Example 10

This example describes studies to characterize the binding kintetics of Her2/neu protein to Phor18-V_{L}-IgG1 and Phor18-V_{L}-Phor18-V_{H}-IgG1 as compared to the binding of Her2/neu protein to Anti-Her2/neu antibody.

Surface plasmon resonance studies were conducted on a BioRad ProteOn system using a GLM sensor chip coated with goat anti-human IgG. ADCs (Phor18-V_{L}-IgG1 and Phor18-V_{L}-Phor18-V_{H}-IgG1) and Anti-Her2/neu antibody were diluted to concentrations of 5 μg/ml and injected over the anti-human IgG surface for capture. Anti-Her2/neu antibody was captured from 150 RU to 1000 RU. Phor18-V_{L}-IgG1 and Phor18-V_{L}-Phor18-V_{H}-IgG1 capture levels were between 200 to 900 RU.

ErbB2/Her2/neu (Sino Biological #1004-H08H) was analyzed at 50 nM as the highest concentration in a three-fold dilution series (N=4). Data were collected at 25 degrees C. Responses from the target surfaces were subtracted by the reference surface and then globally fit to a 1:1 interaction model. Binding constants were determined as shown in Table 8.

TABLE 8
Binding constants determined at 25 degrees C.

| | Kₐ (M⁻¹s⁻¹) | Kₐ (s⁻¹) | K_{D} (pM) |
|---|---|---|---|
| Phor18-V_{L}-IgG1 | 2.20 × 10⁵ | 4.43 × 10⁻⁵ | 201 ± 24 |
| Phor18-V_{L}-Phor18-V_{H}-IgGI | 2.05 × 10⁵ | 3.55 × 10⁻⁵ | 175 ± 13 |
| Anti-Her2/neu antibody | 3.90 × 10⁵ | 3.28 × 10⁻⁵ | 84 ± 13 |

The results indicate that ErbB2 bound to Anti-Her2/neu antibody with an affinity of 84±13 pM and to Phor18-V_{L}-IgG1 and Phor18-V_{L}-Phor18-V_{H}-IgG1 with affinities that were approximately 2.5 and 2-fold weaker at 200±24 and 175±13 pM, respectively. The most significant difference seen was in the association rate for Anti-Her2/neu antibody being about two-fold faster than for Phor18-V_{L}-IgG1 and Phor18-V_{L}-Phor18-V_{H}-IgG1. Thus, the binding kinetics of Phor18-conjugated to a whole antibody are similar to Anti-Her2/neu antibody, indicating that binding properties are barely affected by the conjugation on the variable light and heavy chains.

### Example 11

This example describes studies to characterize and evaluate ADCs for cytotoxicity.

Different expression batches were prepared. ADC concentrations were determined from spectrophotometric measurements (OC₂₈₀) and in IgG quantification assays. Serial dilutions of selected ADCs with N-terminal Phor18 conjugation for 2 and 4 Phor18 molecules per antibody (Phor18-V_{L}-IgG1 and Phor18-V_{L}-Phor18-V_{H}-IgG1) were prepared as described in Example 10. The in vitro activity was based on IgG content of each preparation of Phor18-V_{L}-IgG1 and Phor18-V_{L}-Phor18-V_{H}-IgG1.

A 4h time point was analyzed to determine effects of the ADCs on cell membrane integrity (Table 8). The ADCs with stoichiometric ratios of 2 Phor18/AB and 4 Phor18/AB desintegrated cell membranes of SKOV-3 target positive cells after 4h exposure (IC₅₀ values of 21.5±1 for Phor18-V_{L}-IgG1 and 2.51±0.2 nM for Phor18-V_{L}-Phor18-V_{H}-IgG1), indicating membrane disruption. The membrane activity was 10 fold higher for N-terminal 4 Phor18 conjugated antibody.

Cell death was measured after 24 hours with IC₅₀ values in the low nanomolar range for Phor18-V_{L}-IgG1 (3.7±0.9 nM) and Phor18-V_{L}-Phor18-V_{H}-IgG1 (0.2±0.04 nM). The maximal potency of Phor18-V_{L}-I_{G}G1 and Phor18-V_{L}-Phor18-V_{H}-IgG1 was measured after 48 hours for Phor18-V_{L}-IgG1 (0.54±0.2 nM) and Phor18-V_{L}-Phor18-V_{H}-IgG1 (0.07-0.02 nM; p<0.0001). In both cases the in vitro potency for 4 Phor18 antibody conjugates (Phor18-V_{L}-Phor18-V_{H}-IgG1) was 10-20 fold higher than the 2 Phor18 antibody conjugate (Phor18-V_{L}-IgG1) (p<0.0001). Naked antibody killed Her2/neu positive target cells (SKOV-3) at 225.8±43 and 295.3±80.6 nM after 24 and 48 hours.

Consistent with previous results, Phor18-V_{L}-Phor18-V_{H}-IgG1 was the most active ADC showing a 10 fold higher activity than Phor18-V_{L}-IgG1. The Her2/neu negative control cell line MDA-MB-231 was not killed with either naked antibody or ADCs (Phor18-V_{L}-IgG1 or Phor18-V_{L}-Phor18-V_{H}-IgG1) (Table 9). The data demonstrate that higher numbers of Phor18 (four vs two) conjugated to the N-terminal domain of the antibody are most potent, compared to antibodies with a C-terminal Phor18 conjugation.

TABLE 9
In vitro activities of recombinantly produced
ADCs with N-terminal Phor18 conjugations of 2 and 4
peptides against Her2/neu positive ovarian cancer cell line.

| | In vitro activities Target Cells | | |
|---|---|---|---|
| | (SKOV-3) [IC₅₀-nM] | | |
| | 4 hr | | |
| | (Membrane | | |
| ADC and mAB | integrity) | 24 hr | 48 hr |
| Anti-Her2-IgG1 | intact | 225.8 ± 43 | 295.3 ± 80.6 |
| Phor18-V_{L}-IgG1 | 21.5 ± 1.0 | 3.7 ± 0.9 | 0.54 ± 0.2 |
| Phor18-V_{L}-Phor18-V_{H}- | 2.51 ± 0.2 | 0.2 ± 0.04 | 0.07 ± 0.02 |
| IgG1 | | | |

### Example 12

This example describes production of ADCs that bind to CD20.

CD 20 is expressed on the surface of B-cell malignancies and represents a surface target that is not internalized. Antibody fragment conjugates were produced in E. coli (single chain fragments and Phor18-conjugates) and Pichia pastoris (single chain dimers and Phor18-conjugates) and whole antibody conjugates with 2, 4, and 6 Phor18 molecules were expressed in CHO cells. Chemical conjugations with anti-CD20 IgG1 antibodies AT80 and MS4A1 were conducted.

Chemical Conjugation-AT80 (mouse IgG1, Tenovus) and MS4A1 (rituximab like, R&D) Purified antibodies IgG1 AT80 and IgG1 MS4A1 were obtained and used for the chemical conjugation with Phor18. The antibodies were in phosphate buffered saline (PBS) concentrated to a approximately 2 mg/mL. A 20 mM solution of SPDP was freshly prepared in DMSO, and added to the antibody solution in 20-fold excess. The mixture was incubated at room temperature for about 30 minutes to produce the antibody-linker intermediate. Excess unreacted SPDP is removed by size exclusion chromatography. The cytotoxin molecule containing cysteine was thoroughly reduced by reaction with a 10-fold excess of reductacryl reagent before mixing in 10-fold excess with the antibody-linker construct. The reaction is allowed to incubate at room temperature for 18 hours, then desalted to remove unreacted cytotoxin molecule. The solution is filter-sterilized before storage. Concentrations of final ADCs were for the AT80-Phor18 conjugate 0.76 mg/ml and for the MS4A1-Phor18 conjugate 0.34 mg/ml as determined by Bradford protein measurements. Typical number of Phor18 molecules per antibody using the SPDP method for conjugation is 3-5 molecules of Phor18 per antibody.

Two antibodies against CD20 chemically conjugated to Phor18 were analyzed because these bind to different domains of the extracellular CD20 loops. To compare, in in vitro studies, the cytotoxicity of two chemically conjugated CD20 targeting whole antibody IgG1-Phor18 conjugates with “naked” antibody (IgG1) in CD20 positive cells (Daudi and Raji, Burkitt's lymphoma). CD20 negative leukemia cells (U937) served as controls.

Naked antibody anti-CD20-IgG1-Phor18 were chemically conjugated (MS4A and AT80), Mw app. 158,000 g/mol at a concentrations of 0.34 mg/ml (MS4A-Phor18) and 0.76 mg/ml (AT-80-Phor18). Cell lines were obtained at the American Type Cell Collection (Mannassas, VA). Human Non-Hodgkin's lymphoma cells Daudi (CD20 positive, passage number p2), Raji (CD20 positive p 2) and human leukemia cell line U937 (CD20 negative, p 10) were seeded at a density of 3,000 cells per well in opaque plates in heat inactivated full medium. After 24 hours cells were replenished with fresh media (75 μl) and incubated with 25 μl of a 4× serial dilution of MS4A1-Phor18 and AT80-Phor18 of 0.001, 0.01, 0.1, 1, 10, 100 and 500 nM (N=6). Cells incubated for 2-5 hours were assayed for membrane integrity using a luminometric assay kit (Promega, Madison, WI, Cytotox Glo G9292 lot #301329). Cell viability was determined was determined after 24, and 48 hours using a luminescent assay kit (Promega, Madison, WI, Cell Titer Glo, G 7572, lot 30068102).

Chemically conjugated MS4A1-Phor18 and AT80-Phor18 were tested for their membrane activity. MS4A1-Phor18 was not active after 2 or 5 hours in CD20 positive cell lines Raji and Daudi, whereas AT-80-Phor18 destroyed membrane integrity in CD20 positive Daudi cells with a IC₅₀ value of 106.1±2.9 nM. Daudi cells were killed by the AT-80-Phor18 conjugate within 48 h with IC₅₀ values of 11.9±0.9 nM and Raji cells with IC₅₀ values of 6.3±1.02 nM. The CD20 negative control cell line U937 was not killed by either naked AT-80 antibody or AT-80-Phor18 conjugates.

The MS4A1-Phor18 conjugate showed low activity in Raji cells with IC₅₀ values of 267±13 and 227±11 nM after 24 and 46 hours. Daudi cells were much more sensitive to the MS4A1-Phor18 conjugate with IC₅₀ values of 10.6±2.1 and 3.0±1.2 nM. The naked MS4Al antibody was not toxic to either Raji or Daudi cell lines. The CD20 negative human leukemia cells (U937) were not killed by either of the ADCs (Table 10).

These data show that chemically conjugated Phor18 ADCs kill CD20 positive cells on contact, and do not appear to require internalization. Cytotoxicity is specific for CD20 target and depends on the binding domain of the ADC.

TABLE 10
In vitro activities of chemically conjugated anti-CD20 IgG1-Phor18 conjugates in CD20
positive Non-Hodgkin's lymphoma cell lines Raji and Daudi and the CD20 negative cell line U937

| | Anti-CD20- | Anti-CD20 | Anti-CD20- | Anti-CD20 |
|---|---|---|---|---|
| | Phor18 (AT80) | AT80 | Phor18 (MS4A1) | MS4A1 |
| Raji | [IC₅₀ nM] | [IC₅₀ nM] | [IC₅₀ nM] | [IC₅₀ nM] |
| 2 h | 120.8 ± 0.45 | Not toxic | ND | ND |
| 5 h (N = 6) | 138.2 ± 28.8 | Not toxic | ND | ND |
| 24 h | ND | ND | 267.1 ± 13 | Not toxic |
| 46 h | 6.3 ± 1.02 | ND | 227 ± 11 | Not toxic |
| | Anti-CD20- | | Anti-CD20- | |
| | Phor18 (AT80) | Anti-CD20 | Phor18 (MS4A1) | Anti-CD20 |
| Daudi | [IC₅₀ nM] | AT80 [IC₅₀ nM] | [IC₅₀ nM] | MS4A1 [IC₅₀ nM] |
| 2 h | 126.5 ± 12.1 | Not toxic | Not toxic | Not toxic |
| 5 h (N = 6) | 106.1 ± 2.9 | Not toxic | Not toxic | Not toxic |
| 24 h | ND | ND | 10.6 ± 2.1 | Not toxic |
| 46 h | 11.9 ± 0.9 | 115.5 ± 53 | 3.0 ± 1.2 | Not toxic |
| | Anti-CD20- | | Anti-CD20- | Anti-CD20 |
| U937 (CD20 | Phor18 (AT80) | Anti-CD20 | Phor18 (MS4A1) | MS4A1 |
| negative) | [IC₅₀ nM] | AT80 [IC₅₀ nM] | [IC₅₀ nM] | [IC₅₀ nM] |
| 2-46 h | Not toxic | Not toxic | Not toxic | Not toxic |

ND = not determined

### Example 13

This example describes a comparison of caspase activation of CD20 targeting ADCs.

CD20 targeting ADCs are not internalized. Initiation of cell death can be measured by determining apoptosis related pathways. Early apoptosis processes shows activation of caspases 3 and 7. Caspases are members of the cysteine aspartic acid-specific protease family and play key effector role in apoptosis in mammalian cells. The assay provides a luminogenic caspase-3/7 substrate, which contains the tetrapeptide sequence DEVD, in a reagent optimized for caspase activity, luciferase activity and cell lysis.

To compare, in in vitro studies, the caspase activation of chemically conjugated CD20 targeting whole antibody IgG1-Phor18 conjugate with “naked” antibody (IgG1-AT80) in CD20 positive cells (Daudi Burkitt's lymphoma).

Human Non-Hodgkin's lymphoma cells Daudi (CD20 positive, passage number p2), were seeded at a density of 3,000 cells per well in opaque plates in heat inactivated full medium. After 24 hours cells were fed with fresh media (75 μl) and incubated with AT80-Phor18 (0.76 mg/ml) or naked antibody AT-80 of 15 and 75 μg/ml (100 and 500 nM) (N=6). Staurosporine at 10 UM served as positive control for caspase 3/7 activation. After 5 hours of incubatin cultures were assayed for caspase 3/7 levels using a luminometric assay kit (Promega, Madison, WI, Caspase Glo 3/7 G811C lot #28731802).

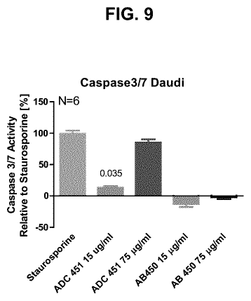

Caspase 3/7 activation was calculated from relative light units from luminometric signals. Staurosporine was set at 100% caspase 3/7 activation, cell suspension alone without additions of reagents served as 0% caspase levels. AT80-Phor18 elevated caspase 3/7 levels to 13=2.6% at 15 μg/ml and reached 85.7±5 at 75 μg/ml. Unconjugated AT-80 at 15 or 75 μg/ml concentrations lacked caspase activation (−12.3 and −2.3%). The highest dose of AT80-Phor18 resulted in a caspase activation that was comparable to Staurosporine (p=0.06) (FIG. 9).

Phor18-conjugated ADCs activate caspase 3/7 when bound to the target cells thus promoting apoptotic cell death within 5 hours.

### Example 14

This example describes expression constructs and characterization of single chain anti-CD20 Phor18 (scFv-Phor18) conjugates in E. coli.

A single chain Fv anti-CD20 fragment was designed by inserting Rituxan CDRs into the humanized variable regions of p185. A poly-histidine tag was added to the N-terminus of the protein for purification. The amino acid sequence is shown below with the inserted CDRs bolded. A poly-glycine flexible linker was inserted between the V_{L} and V_{H} domains and Phor18 was placed at the C-terminus after a GS linker.

```
Amino acid sequence of the scFv fragment (naked AB); CDRs in bold
(SEQ ID NO.: 92)
                10 20 30 40 50 60
HHHHHHDIQL TQSPAILSAS PGEKVTMTCR ASSSVSYIHW FQQKPGSSPK PWIYATSNLA
                70 80 90 100 110 120
SGVPVRFSGS GSGTSYSLTI SRVEAEDAAT YYCQQWTSNP PTFGGGTKLE IGSTSGGGSG
              130 140 150 160 170 180
GGSGGGGSSV QLQQPGAELV KPGASVKMSC KASGYTFTSY NMHWVKQTPG RGLEWIGAIY
              190 200 210 220 230 240
PGNGDTSYNQ KFKGKATLTA DKSSSTAYMQ LSSLTSEDSA VYYCARSTYY GGDWYFDVWG
QGTTVTVSS
Amino acid sequence of the scFv-Phor18 conjugate (C-terminus); CDRs in bold
(SEQ ID NO.: 93)
                10 20 30 40 50 60
HHHHHHDIQL TQSPAILSAS PGEKVTMTCR ASSSVSYIHW FQQKPGSSPK PWIYATSNLA
                70 80 90 100 110 120
SGVPVRFSGS GSGTSYSLTI SRVEAEDAAT YYCQQWTSNP PTFGGGTKLE IGSTSGGGSG
              130 140 150 160 170 180
GGSGGGGSSV QLQQPGAELV KPGASVKMSC KASGYTFTSY NMHWVKQTPG RGLEWIGAIY
              190 200 210 220 230 240
PGNGDTSYNQ KFKGKATLTA DKSSSTAYMQ LSSLTSEDSA VYYCARSTYY GGDWYFDVWG
              250 260 270
QGTTVTVSSG SKFAKFAKKF AKFAKKFAK
```

This antibody fragment was ordered from Genscript USA (Piscataway, NJ) for production in E. coli. Genscript used their pGS21 a expression plasmid for production in E. coli Arctic Express cells. Genscript isolated the protein from E. coli inclusion bodies.

The naked scFv fragment had was purified through affinity chromatography and resulted in a yield of 15 mg/L at a concentration of 0.31 mg/ml. the purity was determined as 85%. The molecular weight was determined using Coomassie stained SDS PAGE analysis with 27,088 g/mol. The Phor18 conjugate had a similar yield of 15 mg/L and a purity of 85% based on Coomassie stained SDS-Page. The molecular weight was measured at 29,349 g/mol. Both naked AB and ADC were provided in 50 mM Tris buffer, pH 8.0.

Anti-CD20 ScFv-Phor18 has a calculated molecular weight if 29.3 kD. The antibody fragment was expressed with a poly-histidine tag for purification. An immunoblot of the protein probed with anti-histidine and SDS-PAGE stained with coomassie blue indicated the molecular weight was in the correct range.

### Example 15

This example describes expression and characterization of recombinantly produced CD20 targeting Fv-C_{H}3-C_{H}3-Fv-Phor18 conjugates in E. coli:

A bivalent anti-CD20 minibody was designed by adding a flexible glycine-serine linker between the variable domains and between the C_{H}3 domains to result in a Fv-C_{H}3-C_{H}3-Fv minibody. A poly-histidine tag was added to the N-terminus of the protein for purification from inclusion bodies and Phor18 was placed at the C-terminus after a GS linker. The amino acid sequences are shown below with the inserted CDRs bolded in variable domains and the C_{H}3 domain are in lower case letters.

```
Amino acid sequence of the anti-CD20 minibody Fv-C_{H}3-C_{H}3-Fv; CDRs in bold
(SEQ ID NO.: 94)
                10 20 30 40 50 60
DIQLTQSPAI LSASPGEKVT MTCRASSSVS YIHWFQQKPG SSPKPWIYAT SNLASGVPVR
                70 80 90 100 110 120
FSGSGSGTSY SLTISRVEAE DAATYYCQQW TSNPPTFGGG TKLEIGSTSG GGSGGGSGGG
              130 140 150 160 170 180
GSSVQLQQPG AELVKPGASV KMSCKASGYT FTSYNMHWVK QTPGRGLEWI GAIYPGNGDT
              190 200 210 220 230 240
SYNQKFKGKA TLTADKSSST AYMQLSSLTS EDSAVYYCAR STYYGGDWYF DVWGQGTTVT
              250 260 270 280 290 300
VSSGQPREPQ VYTLPPSRDE LTKNQVSLTC LVKGFYPSDI AVEWESNGQP ENNYKTTPPV
              310 320 330 340 350 360
LDSDGSFFLY SKLTVDKSRW QQGNVFSCSV MHEALHNHYT QKSLSLSPGK GSTSGGGSGG
              370 380 390 400 410 420
GSGGGGSSGQ PREPQVYTLP PSRDELTKNQ VSLTCLVKGF YPSDIAVEWE SNGQPENNYK
              430 440 450 460 470 480
TTPPVLDSDG SFFLYSKLTV DKSRWQQGNV FSCSVMHEAL HNHYTqKSLS LSPGKVQLQQ
              490 500 510 520 530 540
PGAELVKPGA SVKMSCKASG YTFTSYNMHW VKQTPGRGLE WIGAIYPGNG DTSYNQKEKG
              550 560 570 580 590 600
KATLTADKSS STAYMQLSSL TSEDSAVYYC ARSTYYGGDW YFDVWGQGTT VTVSSGSTSG
              610 620 630 640 650 660
GGSGGGSGGG GSSDIQLTQS PAILSASPGE KVTMTCRASS SVSYIHWFQQ KPGSSPKPWI
              670 680 690 700 710
YATSNLASGV PVFSGSGSGT SYSLTISRVE AEDAATYYCQ QWTSNPPTFG GGTKLEI
Amino acid sequence of the anti-CD20-Phor18 minibody conjugate Fv-C_{H}3-C_{H}3-Fv-Phor18; CDRs
in bold
(SEQ ID NO.: 95)
                10 20 30 40 50 60
DIQLTQSPAI LSASPGEKVT MTCRASSSVS YIHWFQQKPG SSPKPWIYAT SNLASGVPVR
                70 80 90 100 110 120
FSGSGSGTSY SLTISRVEAE DAATYYCQQW TSNPPTFGGG TKLEIGSTSG GGSGGGSGGG
              130 140 150 160 170 180
GSSVQLQQPG AELVKPGASV KMSCKASGYT FTSYNMHWVK QTPGRGLEWI GAIYPGNGDT
              190 200 210 220 230 240
SYNQKFKGKA TLTADKSSST AYMQLSSLTS EDSAVYYCAR STYYGGDWYF DVWGQGTTVT
              250 260 270 280 290 300
VSSGQPREPQ VYTLPPSRDE LTKNQVSLTC LVKGFYPSDI AVEWESNGQP ENNYKTTPPV
              310 320 330 340 350 360
LDSDGSFFLY SKLTVDKSRW QQGNVFSCSV MHEALHNHYT QKSLSLSPGK GSTSGGGSGG
              370 380 390 400 410 420
GSGGGGSSGQ PREPQVYTLP PSRDELTKNQ VSLTCLVKGF YPSDIAVEWE SNGQPENNYK
              430 440 450 460 470 480
TTPPVLDSDG SFFLYSKLTV DKSRWQQGNV FSCSVMHEAL HNHYTQKSLS LSPGKVQLQQ
              490 500 510 520 530 540
PGAELVKPGA SVKMSCKASG YTFTSYNMHW VKQTPGRGLE WIGAIYPGNG DTSYNQKEKG
              550 560 570 580 590 600
KATLTADKSS STAYMQLSSL TSEDSAVYYC ARSTYYGGDW YFDVWGQGTT VTVSSGSTSG
              610 620 630 640 650 660
GGSGGGSGGG GSSDIQLTQS PAILSASPGE KVTMTCRASS SVSYIHWFQQ KPGSSPKPWI
              670 680 690 700 710 720
YATSNLASGV PVFSGSGSGT SYSLTISRVE AEDAATYYCQ QWTSNPPTFG GGTKLEIGSK
              730
FAKFAKKFAK FAKKFAK
```

The antibody fragment was obtained from Genscript USA (Piscataway, NJ) for production in E. coli. Genscript used their pGS21a expression plasmid for production in E. coli Arctic Express cells. Genscript isolated the protein from E. coli inclusion bodies.

The naked anti-CD20 minibody had was purified through affinity chromatography and resulted in a yield of 3 mg/L at a concentration of 0.23 mg/ml. the purity was determined as 80%. The molecular weight was determined using Coomassie stained SDS PAGE analysis with 78,988 g/mol. The Phor18 conjugate had a similar yield of 3 mg/L and a purity of 70% based on Coomassie stained SDS-Page. The molecular weight was measured at 93,515 g/mol. Both naked AB and ADC were provided in 50 mM Tris buffer, 150 mM NaCl, 15-20% glycerol, pH 8-9.5.

The naked single chain Fv and anti-CD20 minibody and scFv-Phor18 and anti CD20-Phor18 conjugates were expressed in E. coli. Distinct molecular weights that corresponded to each antibody fragment backbone were demonstrated on SDS-PAGE gels and Western blots.

### Example 16

This example describes the in vitro analysis of the scFv and anti-CD20 naked minibody and Phor18 conjugates in CD20 positive and CD20 negative cell lines.

To compare, in in vitro studies, the cytotoxicity of a recombinantly produced CD20 targeting scFv-Phor18 and anti-CD20-Phor18 minibody in a bacterial expression system with “naked” antibody (scFv and anti-CD20 minibody) in CD20 positive cells (Daudi, Burkitt's lymphoma). CD20 negative leukemia cells (U937) served as controls.

The concentration of each naked antibody fragment and ADC was determined according to Bradford. Naked antibody scFv Mw 27,088 g/mol at a concentration of 0.3 mg/ml, minibody Mw 78,988 g/mol, 0.231 mg/ml; Phor 18 conjugated antibodies were scFv-Phor18 (Mw 29,349 g/mol, 0.9 mg/ml) and minibody-Phor18 (Mw 93,515, 0.48 mg/ml). Cell lines were obtained at the American Type Cell Collection (Mannassas, VA). Human Non-Hodgkin's lymphoma cells Daudi (CD20 positive, passage number p8) and human leukemia cell line U937 (CD20 negative, p 16) were seeded at a density of 2,000 cells per well in opaque plates in heat inactivated full medium. After 24 hours cells were replenished with fresh media (75 μl) and incubated with 25 μl of a 4× serial dilution of scFv-Phor18 and minibody-Phor18 ADC and naked antibody fragment (scFv and minibody) prepared in cell culture media were added at concentrations of 0.0001, 0.001, 0.01, 0.1, 1, 10, 100, 200, and 500 nM (N=6). Cells incubated for 4 hours were assayed for membrane integrity using a luminometric assay kit (Promega, Madison, WI, Cytotox Glo G9292 lot #301329). Cell viability was determined was determined after 24, 48 and 72 hours using a luminescent assay kit (Promega, Madison, WI, Cell Titer Glo, G 7572, lot 30062102).

Controls for 100% cell viability (culture media) and 100% cell death (0.1% Triton X 100) incubated under the same conditions.

Data were processed and analyzed to obtain IC₅₀ values using Graph Pad Prizm version 5.00 for Windows, GraphPad Software, San Diego California USA, www-graphpad-com (Graph Pad Prizm, Inc). Statistical analysis for significance was determined by a two-tailed Student's T-test. Each test was conducted using 2 plates with 2-3 wells each to achieve an N of 4-6 data points per time point.

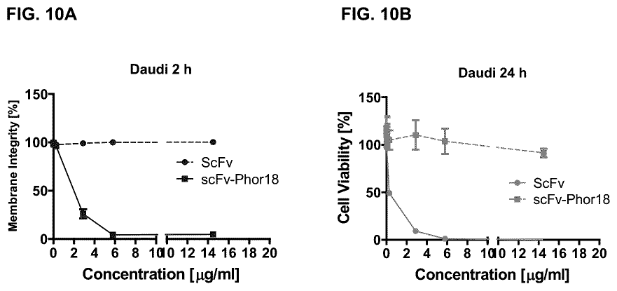

Recombinant scFv-Phor18 conjugates were expressed in E. coli. As shown in FIG. 10, Table 11, the anti-CD20-scFv-Phor18 conjugate destroyed membrane integrity in CD20 positive Daudi cells. Human Burkitts lymphoma cells (Daudi) were killed within 48 h, whereas the CD20 negative human leukemia cells (U937) were not killed. Naked scFv antibody did not kill any of the cell lines. Hill plot analysis of the cell viability data resulted in IC₅₀ values for the minibody conjugate of 10.02±0.5 nM after 24 h and 1.5±0.3 nM after 48 h in Daudi cells. Naked scFv was not toxic. The CD20 negative cell line U937 was not killed by either naked scFv or scFv-Phor18 or minibody-Pho18 conjugates. In vitro activities of the minibody-Phor18 conjugates were 10.5±0.5 and 3.9±1.6 nM in CD20 positive Daudi cells after 24 and 46 hours. Naked minibody was not toxic.

TABLE 11
In vitro toxicities of scFv-Phor18 and
Fv-C_{H}3-C_{H}3-Fv-Phor18, and scFv and Fv-C_{H}3-C_{H}3-
Fv targeting CD 20. Daudi cells (NHL) are positive for CD 20, U937
cells (leukemia) are negative for CD20.

| | | | | Fv-C_{H}3-C_{H}3- |
|---|---|---|---|---|
| | scFv | scFv-Phor18 | Fv-C_{H}3-C_{H}3-Fv | Fv-Phor18 |
| Daudi | [IC₅₀ nM] | [IC₅₀ nM] | [IC₅₀ nM] | [IC₅₀ nM] |
| 2 h | Not toxic | 109.8 ± 0.9 | ND | ND |
| 5 h (N = 8) | Not toxic | 111.6 ± 15.1 | ND | ND |
| 24 h (N = 6) | Not toxic | 10.02 ± 0.5 | Not toxic | 10.5 ± 0.5 |
| 46 h (N = 6) | Not toxic | 1.5 ± 0.3 | Not toxic | 3.6 ± 1.6 |
| U937 | | | | |
| 2-48 h | Not toxic | Not toxic | Not toxic | Not toxic |

ND = Not Determined

Potent scFv-Phor18 and Fv-C_{H}3-C_{H}3-Fv-Phor18 conjugates were expressed in E. coli and were more potent than naked scFv or Fv-C_{H}3-C_{H}3-Fv. CD20 targeted ADCs killed specifically target cells-cell death was independent on internalization. scFv-Phor18 or Fv-C_{H}3-C_{H}3-Fv-Phor18 targeted conjugates but not the naked scFv antibody fragment activated apoptotic pathways. C-terminus conjugated scFv and Fv-C_{H}3-C_{H}3-Fv conjugates showed similar activities in the nanomolar range after 24 hours.

### Example 17

This example describes a possible mechanism of action of the CD20 targeted ADC.

In in vitro studies, caspase activation of recombinantly expressed CD20 targeting scFv-Phor18 conjugate with “naked” antibody (scFv) in CD20 positive cells (Daudi Burkitt's lymphoma) was compared. Human Non-Hodgkin's lymphoma cells Daudi (CD20 positive, p 5) and Raji (CD20 positive, passage number p 3), were seeded at a density of 3,000 cells per well in opaque plates in heat inactivated full medium. After 24 hours cells were fed with fresh media (75 μl) and incubated with scFv-Phor18 (0.365 mg/ml) or naked antibody scFv of 5 and 15 μg/ml (100 and 500 nM) (N=6). Staurosporine at 10 μM served as positive control for caspase 3/7 activation. After 5 hours were assayed for caspase 3/7 activation using a luminometric assay kit (Promega, Madison, WI, Caspase Glo 3/7 G811C lot #28731802).

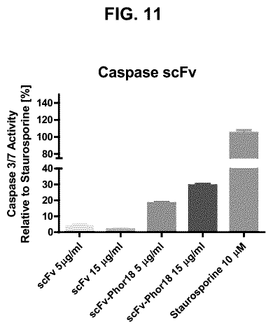

Relative caspase 3/7 activation was calculated from relative light units from luminometric signals. Staurosporine was set at 100% caspase 3/7 activation and no reagents for 0% activation controls. scFv-Phor18 at 5 μg/ml had elevated caspase 3/7 levels of 18.3=0.8 and 29.3=0.9% compared to Staurosporine (FIG. 11).

Phor18-conjugated ADCs activate caspase 3/7 in target cells thus promoting apoptotic cell death. Accordingly, antibody Phor18 conjugates as single chain or minibody fragments can be produced recombinantly and are active without internalization on target cells. Caspase activation through ADCs may be a possible mechanism of action.

### Example 18

This example describes the expression of CD20 targeted scFvFc Phor18 conjugates having Phor18 conjugated at the N or C terminus and having a stoichiometry of 1 and 2 Phor18 molecules per antibody fragment.

The recombinant series was produced by Genscript, USA (Piscataway, NJ) in E. coli using periplasmatic secretion. Transcripts were synthesized and codon usage was optimized for E. Coli The Genscript pGS-21a expression vector was used. The expressed proteins were directed to the periplasm of E. Coli cells by addition of a cleavable PelB bacterial signal sequence, composed of the amino acids MKYLLPTAAAGLLLLAAQPAMA (SEQ ID NO.: 101), to the N-terminus. The Rituxan CDRs were inserted into the humanized variable regions of p185 Herceptin and are shown in bold. Four glycines and a serine linked the variable regions for added flexibility. Phor18 was positioned at the N-terminus, the C-terminus, or both. The amino acid sequence of the antibody fragment are as follows:

Cleaved PelB N-terminal signal sequence MKYLLPTAAAGLLLLAAQPAMA (SEQ ID NO.: 96)

```
Amino acid sequences of scFv-Fc (naked antibody); CDRs in bold
(SEQ ID NO.: 97)
                10 20 30 40 50 60
DIQMTQSPSS LSASVGDRVT ITCRASSSVS YIHWYQQKPG KAPKLLIYAT SNLASGVPSR
                70 80 90 100 110 120
FSGSRSGTDF TLTISSLQPE DFATYYCQQW TSNPPTFGQG TKVEIKGGGG SEVQLVESGG
              130 140 150 160 170 180
GLVQPGGSLR LSCAASGYTF TSYNMHWVRQ APGKGLEWVA AIYPGNGDTS YNQKFKGRFT
              190 200 210 220 230 240
ISADTSKNTA YLQMNSLRAE DTAVYYCSRS TYYGGDWYFD VWGQGTLVTV SSVQPCPAPE
              250 260 270 280 290 300
LLGGPSVFLF PPKPKDTLMI SRTPEVTCVV VDVSHEDPEV KFNWYVDGVE VHNAKTKPRE
              310 320 330 340 350 360
EQYNSTYRVV SVLTVLHQDW LNGKEYKCKV SNKALPAPIE KTISKAKGQP REPQVYTLPP
              370 380 390 400 410 420
SRDELTKNQV SLTCLVKGFY PSDIAVEWES NGQPENNYKT TPPVLDSDGS FFLYSKLTVD
              430 440 450
KSRWQQGNVF SCSVMHEALH NHYTQKSLSL SPGK
Amino acid sequences of lytic-peptide Phor18-antibody conjugate; CDRs in bold
Phor18-scFv-Fc-Phor18: N- and C-terminal conjugation
(SEQ ID NO.: 98)
                10 20 30 40 50 60
KFAKFAKKFA KFAKKFAKGS DIQMTQSPSS LSASVGDRVT ITCRASSSVS YIHWYQQKPG
                70 80 90 100 110 120
KAPKLLIYAT SNLASGVPSR FSGSRSGTDF TLTISSLQPE DFATYYCQQW TSNPPTFGQG
              130 140 150 160 170 180
TKVEIKGGGG SEVQLVESGG GLVQPGGSLR LSCAASGYTF TSYNMHWVRQ APGKGLEWVA
              190 200 210 220 230 240
AIYPGNGDTS YNQKFKGRFT ISADTSKNTA YLQMNSLRAE DTAVYYCSRS TYYGGDWYFD
              250 260 270 280 290 300
VWGQGTLVTV SSVQPCPAPE LLGGPSVFLF PPKPKDTLMI SRTPEVTCVV VDVSHEDPEV
              310 320 330 340 350 360
KFNWYVDGVE VHNAKTKPRE EQYNSTYRVV SVLTVLHQDW LNGKEYKCKV SNKALPAPIE
              370 380 390 400 410 420
KTISKAKGQP REPQVYTLPP SRDELTKNQV SLTCLVKGFY PSDIAVEWES NGQPENNYKT
              430 440 450 460 470 480
TPPVLDSDGS FFLYSKLTVD KSRWQQGNVF SCSVMHEALH NHYTQKSLSL SPGKGSKFAK
              490
FAKKFAKFAK KFAK
Amino acid sequences of lytic-peptide Phor18-antibody conjugate; CDRs in bold
Phor18-scFv-Fc: N-terminal conjugation
(SEQ ID NO.: 99)
                10 20 30 40 50 60
KFAKFAKKFA KFAKKFAKGS DIQMTQSPSS LSASVGDRVT ITCRASSSVS YIHWYQQKPG
                70 80 90 100 110 120
KAPKLLIYAT SNLASGVPSR FSGSRSGTDF TLTISSLQPE DFATYYCQQW TSNPPTFGQG
              130 140 150 160 170 180
TKVEIKGGGG SEVQLVESGG GLVQPGGSLR LSCAASGYTF TSYNMHWVRQ APGKGLEWVA
              190 200 210 220 230 240
AIYPGNGDTS YNQKFKGRFT ISADTSKNTA YLQMNSLRAE DTAVYYCSRS TYYGGDWYFD
              250 260 270 280 290 300
VWGQGTLVTV SSVQPCPAPE LLGGPSVFLF PPKPKDTLMI SRTPEVTCVV VDVSHEDPEV
              310 320 330 340 350 360
KFNWYVDGVE VHNAKTKPRE EQYNSTYRVV SVLTVLHQDW LNGKEYKCKV SNKALPAPIE
              370 380 390 400 410 420
KTISKAKGQP REPQVYTLPP SRDELTKNQV SLTCLVKGFY PSDIAVEWES NGQPENNYKT
              430 440 450 460 470
TPPVLDSDGS FFLYSKLTVD KSRWQQGNVF SCSVMHEALH NHYTQKSLSL SPGK
Amino acid sequences of lytic-peptide Phor18-antibody conjugate; CDRs in bold
Phor18-scFv-Fc: C-terminal conjugation
(SEQ ID NO.: 100)
                10 20 30 40 50 60
DIQMTQSPSS LSASVGDRVT ITCRASSSVS YIHWYQQKPG KAPKLLIYAT SNLASGVPSR
                70 80 90 100 110 120
FSGSRSGTDF TLTISSLQPE DFATYYCQQW TSNPPTFGQG TKVEIKGGGG SEVQLVESGG
              130 140 150 160 170 180
GLVQPGGSLR LSCAASGYTF TSYNMHWVRQ APGKGLEWVA AIYPGNGDTS YNQKFKGRFT
              190 200 210 220 230 240
ISADTSKNTA YLQMNSLRAE DTAVYYCSRS TYYGGDWYFD VWGQGTLVTV SSVQPCPAPE
              250 260 270 280 290 300
LLGGPSVFLF PPKPKDTLMI SRTPEVTCVV VDVSHEDPEV KFNWYVDGVE VHNAKTKPRE
              310 320 330 340 350 360
EQYNSTYRVV SVLTVLHQDW LNGKEYKCKV SNKALPAPIE KTISKAKGQP REPQVYTLPP
              370 380 390 400 410 420
SRDELTKNQV SLTCLVKGFY PSDIAVEWES NGQPENNYKT TPPVLDSDGS FFLYSKLTVD
              430 440 450 460 470
KSRWQQGNVF SCSVMHEALH NHYTQKSLSL SPGKGSKFAK FAKKFAKFAK KFAK
```

The ADCs were purified over protein A affinity chromatography columns and stored frozen (−20 degrees C.). The concentration of each naked antibody fragment and ADC was determined spectrophotometrically (OD₂₈₀). Naked antibody scFv-Fc Mw 52,562 g/mol at a concentration of 1.1 mg/ml, Phor 18 conjugated antibodies were Phor18-V_{L}-scFvFc (Mw 54,685 g/mol, 1.0 mg/ml), Phor18-C_{H}3-scFvFc (Mw 54,685 g/mol, 0.8 mg/ml) formed single chains.

### Example 19

This example describes activity of scFv-Fc-Phor18 conjugates in in vitro studies.

The cytotoxicity of recombinantly produced CD20 targeting scFv-Fc-Phor18 conjugates in a E. coli expression system with Phor18 conjugations at N- or C-terminus and at N- and C-terminus was compared to “naked” antibody (scFv-Fc) in CD20 positive cells (Daudi, Burkitts lymphoma). CD20 negative leukemia cells (U937) served as controls. E. coli expressed ADCs represented single chains and were conjugated to 1 Phor18 molecule at the N-terminus of the V_{L} chain (Phor18-V_{L}-scFv-Fc), at the C-terminus of the C_{H}3 chain (scFv-Fc-C_{H}3-Phor18).

The concentration of each naked antibody fragment and ADC was determined spectrophotometrically (OD₂₈₀). Naked antibody scFv-Fc Mw 52,562 g/mol at a concentration of 1.1 mg/ml, Phor 18 conjugated antibodies were Phor18-V_{L}-scFvFc (Mw 54,685 g/mol, 1.0 mg/ml), scFv-Fc-C_{H}3-Phor18 (Mw 54,685 g/mol, 0.8 mg/ml). Human Non-Hodgkin's lymphoma cells Daudi (CD20 positive, passage number p7) and human leukemia cell line U937 (CD20 negative, p 6) were seeded at a density of 2,000 cells per well in opaque plates in heat inactivated full medium using cell dissociation buffer. After 24 hours cells were replenished with fresh media (75 μl) and incubated and incubated with 25 μl of a 4× serial dilution of each ADC and naked antibody prepared in cell culture media were added at concentrations of 0.01, 0.1, 1, 10, 100, 200 and 500 nM for scFvFc, Phor18-V_{L}-scFvFc and scFv-Fc-C_{H}3-Phor18.

Cells incubated for 4 hours were assayed for membrane integrity using a luminometric assay kit (Promega, Madison, WI, Cytotox Glo G9292 lot #26229601). Cell viability was determined was determined after 24 hours using a luminescent assay kit (Promega, Madison, WI, Cell Titer Glo, G 7572, lot 30731602). Controls for 100% cell viability (culture media) and 100% cell death (0.1% Triton X 100) incubated under the same conditions.

Data were processed and analyzed to obtain IC₅₀ values using Graph Pad Prizm version 5.00 for Windows, GraphPad Software, San Diego California USA, www-graphpad-com (Graph Pad Prizm, Inc). Statistical analysis for significance was determined by a two-tailed Student's T-test. Each test was conducted using 2 plates with 2-3 wells each to achieve an N of 4-6 data points per time point.

Recombinant ScFv-Fc-Phor18 conjugates with 1 Phor18 on the N-terminus or the C-terminus were expressed in E. coli. As shown in Table 12, the anti-CD20-Phor18 conjugate Phor18-V_{L}-scFvFc destroyed membrane integrity in CD20 positive Daudi cells after 4 h with IC₅₀ values of 277-37.5 nM. A tenfold higher value was obtained for the C-terminal conjugate for scFv-Fc-C_{H}3-Phor18 with 2558±259 nM. Naked scFvFc was not toxic.

Human Burkitts lymphoma cells (Daudi) were killed within 24 h with IC₅₀ values of 21.8±0.8 nM for Phor18-V_{L}-scFv-Fc, and 422.6±47.5 nM for scFv-Fc-C_{H}3-Phor18.

Naked scFv caused cell killing at 677.2±45.3 nM in the CD20 positive Daudi cell line. The CD20 negative human leukemia cells (U937) was not killed after 4 hours and showed compared to the target cell line lower sensitivity with IC₅₀ values of 495.4±35.2 nM for the naked scFvFc, 105.3±15.6 nM Phor18-V_{L}-scFv-Fc and 722.3±33.2 nM for the scFv-Fc-C_{H}3-Phor18 conjugates.

TABLE 12
In vitro toxicities of Phor18-V_{L}-scFv-Fc and scFv-Fc-C_{H}3-Phor18, and scFv-Fc targeting
CD 20. Daudi cells (NHL) are positive for CD 20, U937 cells (leukemia) are negative for CD20.

| | scFvFc | Phor18-V_{L}-ScFv-Fc | scFv-Fc-C_{H}3-Phor18 |
|---|---|---|---|
| Daudi | naked AB | IC₅₀ values [nM] | IC₅₀ values [nM] |
| 4 h (N = 8) | Not toxic | 277 ± 37.5 | 2558 ± 259 |
| 24 h (N = 8) | 677.2 ± 45.3 | 21.8 ± 0.8 | 422.6 ± 47.5 |
| | scFvFc | Phor18-V_{L}-ScFv-Fc | scFv-Fc-C_{H}3-Phor18 |
| U937 | naked AB | IC₅₀ values [nM] | IC₅₀ values [nM] |
| 4 h (N = 8) | Not toxic | Not toxic | Not toxic |
| 24 h (N = 8) | 495.4 ± 35.2 | 105.3 ± 15.6 | 722.3 ± 33.2 |

N-terminus conjugated Phor18 antibody fragments were more toxic compared to C-terminus conjugated Phor18 antibody fragments.

### Example 20

This example describes expression and activity of CD20 targeting scFv-Fc-Phor18 conjugates produced in Pichia Pastoris (yeast).

Four anti-CD20 single chain Fv-Fc antibody fragments were designed by inserting the Rituxan CDRs into the humanized variable regions of p185. The inserted CDRs are bolded in the amino acid sequences shown below. A poly-G linker was used between the variable regions and human constant domains hinge, C_{H}2 and C_{H}3. The order of the antibody fragment sequences shown below are as follows: naked antibody, C-terminal and N-terminal Phor18, N-terminal Phor18 only, C-terminal Phor18 only. The amino acid sequences for each construct are shown below:

```
Amino acid sequences of naked antibody; CDRs in bold
(SEQ ID NO.: 101)
                10 20 30 40 50 60
DIQMTQSPSS LSASVGDRVT ITCRASSSVS YIHWYQQKPG KAPKLLIYAT SNLASGVPSR
                70 80 90 100 110 120
FSGSRSGTDF TLTISSLQPE DFATYYCQQW TSNPPTFGQG TKVEIKGGGG SGGGGGGGG
              130 140 150 160 170 180
SEVQLVESGG GLVQPGGSLR LSCAASGYTF TSYNMHWVRQ APGKGLEWVA AIYPGNGDTS
              190 200 210 220 230 240
YNQKFKGRFT ISADTSKNTA YLQMNSLRAE DTAVYYCSRS TYYGGDWYFD VWGQGTLVTV
              250 260 270 280 290 300
SSTHTCPPCP APELLGGPSV FLFPPKPKDT LMISRTPEVT CVVVDVSHED PEVKFNWYVD
              310 320 330 340 350 360
GVEVHNAKTK PREEQYNSTY RVVSVLTVLH QDWLNGKEYK CKVSNKALPA PIEKTISKAK
              370 380 390 400 410 420
GQPREPQVYT LPPSRDELTK NQVSLTCLVK GFYPSDIAVE WESNGQPENN YKTTPPVLDS
              430 440 450 460
DGSFFLYSKL TVDKSRWQQG NVFSCSVMHE ALHNHYTQKS LSLSPGK
Amino acid sequences of conjugated antibody Phor18-V_{L}-scFv-Fc-C_{H}3-Phor18;
CDRs in bold
(SEQ ID NO.: 102)
                10 20 30 40 50 60
KFAKFAKKFA KFAKKFAKGS DIQMTQSPSS LSASVGDRVT ITCRASSSVS YIHWYQQKPG
                70 80 90 100 110 120
KAPKLLIYAT SNLASGVPSR FSGSRSGTDF TLTISSLQPE DFATYYCQQW TSNPPTFGQG
              130 140 150 160 170 180
TKVEIKGGGG SGGGGSGGGG SEVQLVESGG GLVQPGGSLR LSCAASGYTF TSYNMHWVRQ
              190 200 210 220 230 240
APGKGLEWVA AIYPGNGDTS YNQKFKGRFT ISADTSKNTA YLQMNSLRAE DTAVYYCSRS
              250 260 270 280 290 300
TYYGGDWYFD VWGQGTLVTV SSTHTCPPCP APELLGGPSV FLFPPKPKDT LMISRTPEVT
              310 320 330 340 350 360
CVVVDVSHED PEVKFNWYVD GVEVHNAKTK PREEQYNSTY RVVSVLTVLH QDWLNGKEYK
              370 380 390 400 410 420
CKVSNKALPA PIEKTISKAK GQPREPQVYT LPPSRDELTK NQVSLTCLVK GFYPSDIAVE
              430 440 450 460 470 480
WESNGQPENN YKTTPPVLDS DGSFFLYSKL TVDKSRWQQG NVFSCSVMHE ALHNHYTQKS
              490 500
LSLSPGKGSK AFKKAFKAFK KAFKAFK
Amino acid sequences of conjugated antibody Phor18-V_{L}-scFv-Fc; CDRs in bold
(SEQ ID NO.: 103)
                10 20 30 40 50 60
KFAKFAKKFA KFAKKFAKGS DIQMTQSPSS LSASVGDRVT ITCRASSSVS YIHWYQQKPG
                70 80 90 100 110 120
KAPKLLIYAT SNLASGVPSR FSGSRSGTDF TLTISSLQPE DFATYYCQQW TSNPPTFGQG
              130 140 150 160 170 180
TKVEIKGGGG SGGGGSGGGG SEVQLVESGG GLVQPGGSLR LSCAASGYTF TSYNMHWVRQ
              190 200 210 220 230 240
APGKGLEWVA AIYPGNGDTS YNQKFKGRFT ISADTSKNTA YLQMNSLRAE DTAVYYCSRS
              250 260 270 280 290 300
TYYGGDWYFD VWGQGTLVTV SSTHTCPPCP APELLGGPSV FLFPPKPKDT LMISRTPEVT
              310 320 330 340 350 360
CVVVDVSHED PEVKFNWYVD GVEVHNAKTK PREEQYNSTY RVVSVLTVLH QDWLNGKEYK
              370 380 390 400 410 420
CKVSNKALPA PIEKTISKAK GQPREPQVYT LPPSRDELTK NQVSLTCLVK GFYPSDIAVE
              430 440 450 460 470 480
WESNGQPENN YKTTPPVLDS DGSFFLYSKL TVDKSRWQQG NVESCSVMHE ALHNHYTQKS
LSLSPGK
Amino acid sequences of conjugated antibody scFv-Fc-C_{H}3-Phor18; CDRs in bold
(SEQ ID NO.: 104)
                10 20 30 40 50 60
DIQMTQSPSS LSASVGDRVT ITCRASSSVS YIHWYQQKPG KAPKLLIYAT SNLASGVPSR
                70 80 90 100 110 120
FSGSRSGTDF TLTISSLQPE DFATYYCQQW TSNPPTFGQG TKVEIKGGGG SGGGGSGGGG
              130 140 150 160 170 180
SEVQLVESGG GLVQPGGSLR LSCAASGYTF TSYNMHWVRQ APGKGLEWVA AIYPGNGDTS
              190 200 210 220 230 240
YNQKFKGRFT ISADTSKNTA YLQMNSLRAE DTAVYYCSRS TYYGGDWYFD VWGQGTLVTV
              250 260 270 280 290 300
SSTHTCPPCP APELLGGPSV FLFPPKPKDT LMISRTPEVT CVVVDVSHED PEVKFNWYVD
              310 320 330 340 350 360
GVEVHNAKTK PREEQYNSTY RVVSVLTVLH QDWLNGKEYK CKVSNKALPA PIEKTISKAK
              310 320 330 340 350 360
GQPREPQVYT LPPSRDELTK NQVSLTCLVK GFYPSDIAVE WESNGQPENN YKTTPPVLDS
              370 380 390 400 410 420
DGSFFLYSKL TVDKSRWQQG NVFSCSVMHE ALHNHYTQKS LSLSPGKGSK AFKKAFKAFK
KAFKAFK
```

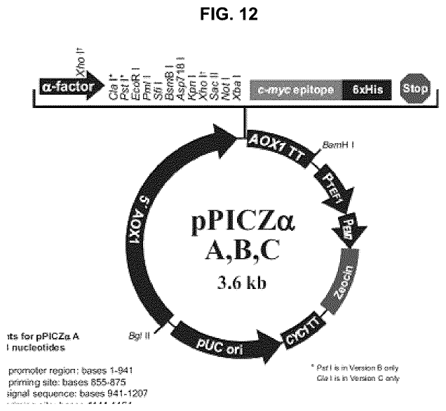

The gene synthesis for the four scFv-Fc fragments was ordered from Genescript USA and codons were optimized for production in Pichia Pastoris yeast strain GS115 at Genscript. Genescript subcloned each expression plasmid into the In Vitrogen yeast expression plasmid pPICZaA (cat #V195-20 lot #900479, FIG. 12). This expression plasmid has a yeast α-factor secretory signal so that the antibody protein could be isolated from medium.

Characterization of CD20 targeting scFv-Fc-Phor18 conjugates on silver stained SDS PAGE showed that Pichia pastoris expressed ADCs represented single chain dimers and were conjugated to 2 or 4 Phor18 molecules at the N-terminus of the V_{L} chain (Phor18-V_{L}-scFvFc), at the C-terminus of the C_{H}3 chain (scFvFc-C_{H}3-Phor18) and at the N-terminus of the V_{L} chains and the C-terminus of the C_{H}3 chain (Phor18-V_{L}-scFv-Fc-C_{H}3-Phor18).

The concentration of each naked antibody fragment and ADC was determined spectrophotometrically (OD₂₈₀). Naked antibody scFv-Fc (Mw 102,078 g/mol) at a concentration of 0.26 mg/ml, Phor 18 conjugated antibodies were Phor18-V_{L}-scFvFc (Mw 106,616 g/mol, 0.5 mg/ml), scFvFc-C_{H}3-Phor18 (Mw 106,616 g/mol, 0.17 mg/ml) and Phor18-V_{L}-scFv-Fc-C_{H}3-Phor18 (Mw 111,154 g/mol, 0.7 mg/ml).

To compare, in in vitro studies, the cytotoxicity of recombinantly produced CD20 targeting scFv-Fc-Phor18 conjugates in a yeast expression system with “naked” antibody (scFv-Fc) in CD20 positive cells (Daudi, Burkitts lymphoma). CD20 negative leukemia cells (U937) served as controls. Pichia expressed ADCs represented single chain dimers and were conjugated to 2 or 4 Phor18 molecules at the N-terminus of the V_{L} chain (Phor18-V_{L}-scFvFc), at the C-terminus of the C_{H}3 chain (scFvFc-C_{H}3-Phor18) and at the N-terminus of the V_{L} chains and the C-terminus of the C_{H}3 chain (Phor18-V_{L}-scFv-Fc-C_{H}3-Phor18).

Human Non-Hodgkin's lymphoma cells Daudi (CD20 positive, passage number p3) and human leukemia cell line U937 (CD20 negative, p 12) were seeded at a density of 2,000 cells per well in opaque plates in heat inactivated full medium using cell dissociation buffer. After 24 hours cells were replenished with fresh media (75 μl) and incubated with 25 μl of a 4× serial dilution of each ADC and naked antibody prepared in cell culture media were added at concentrations of 0.013, 0.133, 1.33, 13.3, 133, 266, and 633 nM for Phor18-V_{L}-scFv-Fc-C_{H}3-Phor18 and Phor18-V_{L}-scFvFc and 0.013-266 nM for the naked antibody scFv-Fc and scFvFc-C_{H}3-Phor18.

Cells incubated for 4 hours were assayed for membrane integrity using a luminometric assay kit (Promega, Madison, WI, Cytotox Glo G9292 lot #26229601). Cell viability was determined was determined after 24, and 48 hours using a luminescent assay kit (Promega, Madison, WI, Cell Titer Glo, G 7572, lot 31386501). Controls for 100% cell viability (culture media) and 100% cell death (0.1% Triton X 100) incubated under the same conditions.

Data were processed and analyzed to obtain IC₅₀ values using Graph Pad Prizm version 5.00 for Windows, GraphPad Software, San Diego California USA, www-graphpad-com (Graph Pad Prizm, Inc). Statistical analysis for significance was determined by a two-tailed Student's T-test. Each test was conducted using 2 plates with 2-3 wells each to achieve an N of 4-6 data points per time point.

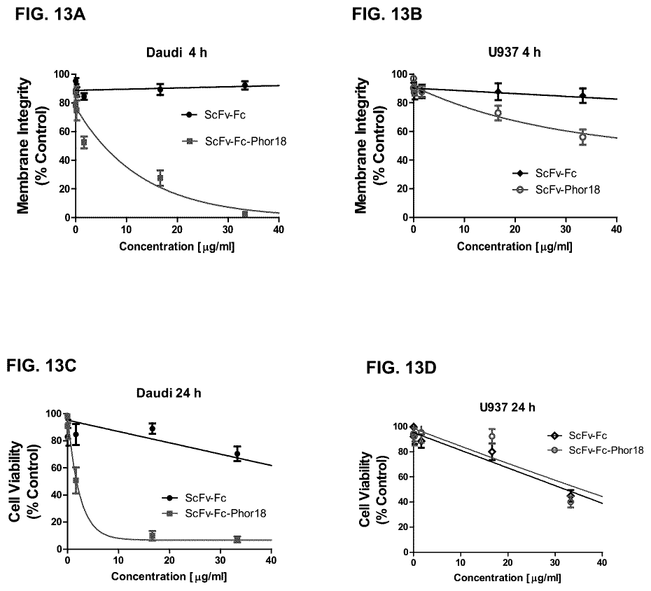

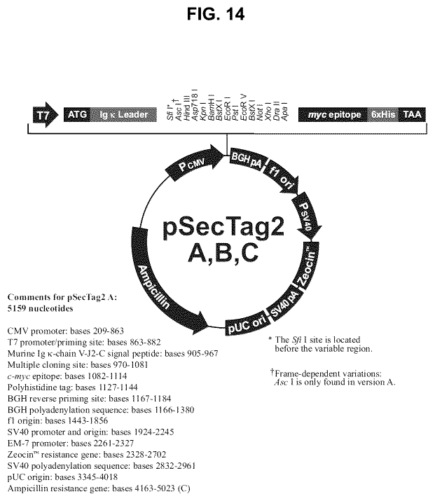

Recombinant ScFv-Fc-Phor18 conjugates were expressed in Pichia Pastoris. As shown in FIG. 13 and Table 13, the anti-CD20-Phor18 conjugates destroyed membrane integrity in CD20 positive Daudi cells after 4 h with IC₅₀ values of 6.2±2 nM for Phor18-V_{L}-scFv-Fc-C_{H}3-Phor18, and 23.5±2.5 nM for Phor18-V_{L}-scFvFc and 282.4±15 nM for scFv-Fc-C_{H}3-Phor18; unconjugated, naked scFvFc was not toxic. Human Burkitts lymphoma cells (Daudi) were killed within 24 h with IC₅₀ values of 9.9±2 nM for Phor18-V_{L}-scFv-Fc-C_{H}3-Phor18, and 18.8±4 nM for Phor18-V_{L}-scFvFc and 141.1±6 nM for scFv-Fc-C_{H}3-Phor18; naked scFvFc had a IC₅₀ value of 287±28 nM. The lowest IC₅₀ values were measured after 48 hours with 1.61±0.3 nM for Phor18-V_{L}-scFv-Fc-C_{H}3-Phor18, and 3.4±0.05 nM for Phor18-V_{L}-scFvFc and 106±10 nM for scFv-Fc-C_{H}3-Phor18; naked scFvFc had a IC₅₀ value of 339±26 nM. The CD20 negative human leukemia cells (U937) showed the same response to either conjugated and unconjugated ADC: after 4 hours no effects on membrane integrity, IC₅₀ values after 24 hours were comparable to naked scFv-Fc with 298±13.4, 246.1±14.8 for Phor18-V_{L}-SCFv-Fc-C_{H}3-Phor18, 875±81 for Phor18-V_{L}-scFvFc and 274±154 nM for scFv-Fc-C_{H}3-Phor18.

These data demonstrate that N-terminal conjugated ADCs are more potent than C-terminal conjugated ADCs. ADCs with 4 Phor18 molecules were more active than conjugates with 2 Phor18 molecules.

TABLE 13
In vitro toxicities of Phor18-V_{L}-scFv-Fc and scFv-Fc-C_{H}3-Phor18, Phor18-V_{L}-scFv-Fc-
C_{H}3-Phor18 and scFv-Fc targeting CD 20. Daudi cells (NHL) are positive for CD 20, U937 cells
(leukemia) are negative for CD20.

| | scFvFc | Phor18-V_{L}-scFv-Fc- | Phor18-V_{L}- | scFv-Fc-C_{H}3- |
|---|---|---|---|---|
| | (naked) | C_{H}3-Phor18 | scFv-Fc | Phor18 |
| Number of | 0 | 4 (N- and C-terminus) | 2 (N-terminus) | 2 (C-terminus) |
| Phor18 | | | | |
| | IC₅₀ values | | IC₅₀ values | IC₅₀ values |
| Daudi | [nM] | IC₅₀ values [nM] | [nM] | [nM] |
| 4 h (Membrane | Not toxic | 6.2 ± 2 | 23.5 ± 2.5 | 282.4 ± 15 |
| integrity) | | | | |
| 24 h | 287 ± 28 | 9.9 ± 2 | 18.8 ± 4 | 141.1 ± 6 |
| 48 h | 339 ± 26 | 1.6 ± 0.3 | 3.4 ± 0.05 | 106 ± 10 |
| | IC₅₀ values | | IC₅₀ values | IC₅₀ values |
| U937 | [nM] | IC₅₀ values [nM] | [nM] | [nM] |
| 4 h (Membrane | Not toxic | Not toxic | Not toxic | Not toxic |
| integrity) | | | | |
| 24 h | 298 ± 13.4 | 246.1 ± 14.8 | 875 ± 81 | 274 ± 154 |
| 48 h | 112.7 ± 4 | 117 ± 7 | Not toxic | 116 ± 4.5 |

Potent scFv-Fc-Phor18 conjugates with 2 and 4 Phor18 molecules at the N- or C-terminus and N- and C-terminus were expressed in Pichia Pastoris. The ADCs were more potent than naked scFv-Fc. ScFv-Fc-Phor18 conjugates destroyed membrane integrity of the target cells when N and C-terminus was conjugated or N-terminus was conjugated. C-terminus conjugation was 50-100 fold less active compared to N-terminus and C- and N-terminus conjugated ADCs. CD20 targeted ADCs killed specifically target cells-the cell death was independent on internalization. Increasing numbers of Phor18 on ADC resulted in increased potency.

### Example 21

This example describes IgG2-Phor18 conjugates produced in mammalian system:

CHO cell expressed CD20 targeting ADCs represented whole antibodies and were conjugated to 2, 4 and 6 Phor18 at different locations. The produced ADCs were characterized to confirm Phor18 presence on heavy and light chains, their molecular weights.

Cytotoxicity was determined in vitro using the recombinantly produced CD20 targeting IgG2-Phor18 conjugates from a CHO cell expression system and compared with “naked” antibody (Rituxan) in CD20 positive cells (Daudi, Burkitts lymphoma). CD20 negative leukemia cells (U937) served as controls.

The V_{L} and V_{H} domains of the anti-CD20 ADC are humanized sequences from anti-p185 Herceptin with the CDRs replaced by Rituxan anti-CD20 CDRs. Whole human IgG2 was used for the full antibody backbone with the λ isoform of the constant light (C_{L}) domain.

The IgG2 isoform was chosen to minimize FcR interactions and limit binding and killing of immune cells by Rituxan ADCs. Gene synthesis was conducted by Genscript USA Inc., Piscataway, NJ, with codon usage optimized for CHO cells. The amino acid sequences of the “unconjugated” anti-CD20 antibody Anti-CD20 antibody heavy (H) and light (L) chains are shown below. Rituxan CDRs in variable domains are shown in bold type.

Anti-CD20 Antibody Amino Acid Sequence of Light Chain and Heavy Chain with Rituxan CDRs

```
Light chain: V_{L}
(SEQ ID NO.: 105)
                10 20 30 40 50 60
MDIQMTQSPS SLSASVGDRV TITCRASSSV SYIHWYQQKP GKAPKLLIYA TSNLASGVPS
                70 80 90 100
RFSGSRSGTD FTLTISSLQP EDFATYYCQQ WTSNPPTFGQ GTKVEIKR
C_{L} (λ)
(SEQ ID NO.: 106)
                10 20 30 40 50 60
GQPKANPTVT LFPPSSEELQ ANKATLVCLI SDFYPGAVTV AWKADGSPVK AGVETTKPSK
                70 80 90 100
QSNNKYAASS YLSLTPEQWK SHRSYSCQVT HEGSTVEKTV APTECS
Heavy chain: V_{H}
(SEQ ID NO.: 107)
                10 20 30 40 50 60
MEVQLVESGG GLVQPGGSLR LSCAASGYTF TSYNMHWVRQ APGKGLEWVA AIYPGNGDTS
                70 80 90 100 110 120
YNQKFKGRFT ISADTSKNTA YLQMNSLRAE DTAVYYCSRS TYYGGDWYFD VWGQGTLVTV
SS
IGg2 C_{H}1 and hinge
(SEQ ID NO.: 108)
                10 20 30 40 50 60
ASTKGPSVFP LAPCSRSTSE STAALGCLVK DYFPEPVTVS WNSGALTSGV HTFPAVLQSS
                70 80 90 100 110
GLYSLSSVVT VPSSNFGTQT YTCNVDHKPS NTKVDKTVER KCCVECPPCP
IGg2 C_{H}2 and C_{H}3
(SEQ ID NO.: 109)
                10 20 30 40 50 60
APPVAGPSVF LFPPKPKDTL MISRTPEVTC VVVDVSHEDP EVQFNWYVDG VEVHNAKTKP
                70 80 90 100 110 120
REEQFNSTFR VVSVLTVVHQ DWLNGKEYKC KVSNKGLPAP IEKTISKTKG QPREPQVYTL
              130 140 150 160 170 180
PPSREEMTKN QVSLTCLVKG FYPSDIAVEW ESNGQPENNY KTTPPMLDSD GSFFLYSKLT
                190 200 210
VDKSRWQQGN VFSCSVMHEA LHNHYTQKSL SLSPGK
```

Recombinant expression of whole IgG2 antibody-Phor18 (KFAKFAKKFAKFAK KFAK (SEQ ID NO.: 4)) conjugates in a mammalian system (CHO cells). Heavy and light chain transcripts were synthesized out of the Genscript DNA transcripts using PCR primers with Ascl and EcoR1 restriction sites for directional cloning into the pSECTag2 mammalian expression plasmid (Invitrogen cat #V900-20, lot 842626). The pSecTag2 plasmid was chosen because it has a mammalian CMV promoter and an Igk signal sequence for secretion of the antibody. Because of the location of the multiple cloning site in the expression plasmid relative to the signal sequence, cleavage of the signal peptide in the expressed ADC protein leaves the following 6 amino acids on the N-terminus of each peptide because of the plasmid design: DAAQPA (SEQ ID NO.: 152).

Genscript ADC transcript DNA sequences are shown below for the full light chain and full heavy chain of the ADC with Phor18 (italics):

### Sequence of the Gene for Expression of CD20 Targeting Antibody-Phor18 Conjugates in CHO Cells

The full ADC light chain target sequence is:
(SEQ ID NO.: 110)
ATGAAGTTCGCAAAGTTCGCCAAAAAGTTCGCAAAGTTCGCAAAAAAGT
TCGCCAAAGGGTCAGATATTCAGATGACTCAGAGCCCCAGCTCCCTGTC
CGCATCTGTGGGCGACCGAGTCACTATCACCTGCCGAGCCTCTAGTTCA
GTGAGCTACATTCACTGGTATCAGCAGAAGCCTGGGAAAGCCCCAAAGC
TGCTCATCTACGCCACAAGCAACCTGGCTTCCGGTGTGCCTTCTAGGTT
CAGTGGGTCAAGAAGCGGTACAGACTTTACACTGACTATTAGCTCCCTC
CAGCCAGAGGATTTCGCCACTTACTATTGCCAGCAGTGGACTTCCAATC
CCCCTACCTTTGGCCAGGGAACAAAAGTGGAAATCAAGGGGCAGCCCAA
AGCTAACCCTACCGTCACACTGTTCCCACCCTCTAGTGAGGAACTCCAG
GCAAATAAGGCCACTCTGGTGTGTCTCATTTCCGACTTTTACCCCGGAG
CTGTGACCGTCGCTTGGAAGGCAGATGGCTCTCCAGTGAAAGCAGGAGT
CGAGACCACAAAACCCAGTAAGCAGTCAAACAATAAGTACGCCGCTTCA
AGCTATCTGAGTCTCACCCCTGAACAGTGGAAAAGCCATAGGTCCTATT
CTTGCCAGGTCACTCACGAAGGTAGCACTGTGGAAAAGACTGTCGCACC
AACCGAATGTAGCGGCTCCAAGGCTTTCAAGAAGGCCTTCAAGGCCTTC
AAGAAAGCATTCAAGGCCTTTAAATGATAA
The full ADC heavy chain target sequence is:
(SEQ ID NO.: 111)
ATGAAGTTCGCCAAATTTGCTAAGAAATTCGCAAAGTTTGCCAAGAAAT
TCGCTAAAGGCTCCGAAGTGCAGCTCGTCGAAAGCGGGGGGGGACTCGT
GCAGCCAGGGGGAAGCCTCAGACTCTCATGCGCCGCCTCAGGTTATACT
TTCACAAGCTACAACATGCACTGGGTCAGACAGGCACCTGGGAAGGGTC
TGGAGTGGGTGGCCGCTATCTACCCAGGCAACGGAGACACATCTTATAA
TCAGAAGTTCAAAGGCCGGTTTACTATTAGCGCAGATACATCCAAGAAC
ACTGCCTACCTGCAGATGAATAGCCTCCGGGCTGAAGACACTGCAGTGT
ACTATTGCAGTCGCTCAACCTACTATGGCGGAGACTGGTATTTCGATGT
GTGGGGGCAGGGTACTCTGGTCACCGTGAGCTCCGCCTCTACCAAGGGG
CCCAGTGTGTTTCCACTGGCTCCCTGCAGCCGGTCCACCTCTGAGAGTA
CAGCAGCCCTGGGTTGTCTCGTGAAAGATTACTTCCCTGAACCAGTCAC
CGTGTCCTGGAACTCTGGCGCTCTGACCAGCGGAGTCCACACATTTCCT
GCAGTGCTCCAGTCTAGTGGGCTGTACTCCCTCTCAAGCGTGGTCACAG
TCCCATCCTCTAATTTCGGTACTCAGACCTATACATGCAACGTGGACCA
TAAGCCCTCCAATACTAAGGTCGATAAAACCGTGGAGCGCAAATGCTGT
GTGGAATGCCCACCTTGTCCAGCACCACCAGTCGCTGGGCCTAGCGTGT
TCCTGTTTCCTCCAAAGCCAAAAGACACTCTCATGATCTCTCGAACTCC
CGAGGTCACCTGTGTGGTCGTGGACGTCAGTCACGAGGATCCTGAAGTC
CAGTTTAACTGGTACGTGGATGGAGTCGAAGTGCATAATGCAAAGACCA
AACCAAGGGAGGAACAGTTCAACTCAACCTTTAGAGTCGTGAGCGTGCT
GACAGTCGTGCATCAGGACTGGCTCAACGGGAAGGAGTATAAGTGCAAA
GTGTCTAATAAGGGTCTGCCCGCTCCTATCGAGAAAACAATTAGCAAGA
CTAAAGGACAGCCTCGAGAACCACAGGTGTACACACTGCCCCCTAGCAG
GGAGGAAATGACAAAGAACCAGGTCTCCCTGACTTGTCTCGTGAAAGGC
TTCTATCCCAGTGACATTGCCGTGGAGTGGGAATCAAATGGACAGCCTG
AGAACAATTACAAGACCACACCACCCATGCTGGACAGTGATGGCTCATT
CTTTCTGTATTCCAAGCTCACCGTGGATAAATCTAGGTGGCAGCAGGGA
AATGTCTTTTCATGTAGCGTGATGCACGAGGCTCTCCATAACCATTACA
CCCAGAAGTCCCTGTCACTCTCCCCCGGCAAAGGCTCCAAGGCTTTCAA
GAAGGCCTTCAAGGCCTTCAAGAAAGCATTCAAGGCCTTTAAATGATAA

PCR primers used to subclone the light chains and heavy chains are shown below.
ADC PCR primers:
Forward primers have the ASCI restriction site (GGCGCGCC) at the 5′ end.
Reverse primers have the EcoRI restriction site (GAATTC) at the 5′ end.

Light chain primers
(SEQ ID NOs.: 112-115)
480L for:
GGGGGCGCGCC GATATTCAGATGACTCAGAGCC (Tm = 55.6)
485Lfor:
GGGGGCGCGCC AAGTTCGCAAAGTTCGCCAA (Tm = 63)
480Lrev:
GGG GAATTC TTATCAGCTACATTCGGTTGGT(Tm = 58.65)
487Lrev:
GGG GAATTC TTATCATTTAAAGGCCTTGAATGCT (Tm = 61.37)
Mar18
Heavy chain primers
(SEQ ID NOs.: 116-118)
480Hfor:
GGG GGCGCGCC GAAGTGCAGCTCGTCGAAAG (Tm = 61)
485Hfor:
GGG GGCGCGCC AAGTTCGCCAAATTTGCTAAGA(Tm = 60.25)
480Hrev:
GGG GAATTC TTATCATTTGCCGGGGGA (Tm = 62)

The amino acid sequences for each of the naked IgG2 antibody and the Phor18-IgG2 conjugates with 2 Phor18 molecules (Phor18-V_{L} IgG2, Phor18-V_{H} IgG2), 4 Phor18 molecules (Phor18-V_{L}-Phor18V_{H}-IgG2), 6 Phor18 molecules (Phor18-V_{L}-C_{L}-Phor18-Phor18-V_{H}-IgG2) and 8 Phor18 molecules Phor18-V_{L}-C_{L}-Phor18-V_{H}-Phor18-C_{H}3-Phor18-IgG2 and Phor18-V_{L}-C_{L}-Phor18-V_{H}-Phor18-C_{H}3-IgG2) are shown below.

### Amino Acid Sequences of Naked Antibody, Lytic-Peptide Phor18-Antibody Heavy and Light Chain Conjugates

```
IgG2 (480) (naked)
(SEQ ID NO.: 119)
Light chain
                10 20 30 40 50 60
DIQMTQSPSS LSASVGDRVT ITCRASSSVS YIHWYQQKPG KAPKLLIYAT SNLASGVPSR
                70 80 90 100 110 120
FSGSRSGTDF TLTISSLQPE DFATYYCQQW TSNPPTFGQG TKVEIKGQPK ANPTVTLFPP
              130 140 150 160 170 180
SSEELQANKA TLVCLISDFY PGAVTVAWKA DGSPVKAGVE TTKPSKQSNN KYAASSYLSL
              190 200 210
TPEQWKSHRS YSCQVTHEGS TVEKTVAPTE CS
Heavy chain
(SEQ ID NO.: 120)
                10 20 30 40 50 60
EVQLVESGGG LVQPGGSLRL SCAASGYTFT SYNMHWVRQA PGKGLEWVAA IYPGNGDTSY
                70 80 90 100 110 120
NQKFKGRFTI SADTSKNTAY LQMNSLRAED TAVYYCSRST YYGGDWYFDV WGQGTLVTVS
              130 140 150 160 170 180
SASTKGPSVF PLAPCSRSTS ESTAALGCLV KDYFPEPVTV SWNSGALTSG VHTFPAVLQS
              190 200 210 220 230 240
SGLYSLSSVV TVPSSNFGTQ TYTCNVDHKP SNTKVDKTVE RKCCVECPPC PAPPVAGPSV
              250 260 270 280 290 300
FLFPPKPKDT LMISRTPEVT CVVVDVSHED PEVQFNWYVD GVEVHNAKTK PREEQFNSTF
              310 320 330 340 350 360
RVVSVLTVVH QDWLNGKEYK CKVSNKGLPA PIEKTISKTK GQPREPQVYT LPPSREEMTK
              370 380 390 400 410 420
NQVSLTCLVK GFYPSDIAVE WESNGQPENN YKTTPPMLDS DGSFFLYSKL TVDKSRWQQG
              430 440
NVFSCSVMHE ALHNHYTQKS LSLSPGK
Phor18-V_{L} IgG2 (481) (2 Phor18, N-terminus)
(SEQ ID NO.: 121)
Light chain
                10 20 30 40 50 60
MKFAKFAKKF AKFAKKFAKG SDIQMTQSPS SLSASVGDRV TITCRASSSV SYIHWYQQKP
                70 80 90 100 110 120
GKAPKLLIYA TSNLASGVPS RFSGSRSGTD FTLTISSLQP EDFATYYCQQ WTSNPPTFGQ
              130 140 150 160 170 180
GTKVEIKGQP KANPTVTLFP PSSEELQANK ATLVCLISDF YPGAVTVAWK ADGSPVKAGV
              190 200 210 220 230
ETTKPSKQSN NKYAASSYLS LTPEQWKSHR SYSCQVTHEG STVEKTVAPT ECS
Heavy chain
(SEQ ID NO.: 122)
                10 20 30 40 50 60
EVQLVESGGG LVQPGGSLRL SCAASGYTFT SYNMHWVRQA PGKGLEWVAA IYPGNGDTSY
                70 80 90 100 110 120
NQKFKGRFTI SADTSKNTAY LQMNSLRAED TAVYYCSRST YYGGDWYFDV WGQGTLVTVS
              130 140 150 160 170 180
SASTKGPSVF PLAPCSRSTS ESTAALGCLV KDYFPEPVTV SWNSGALTSG VHTFPAVLQS
              190 200 210 220 230 240
SGLYSLSSVV TVPSSNFGTQ TYTCNVDHKP SNTKVDKTVE RKCCVECPPC PAPPVAGPSV
              250 260 270 280 290 300
FLFPPKPKDT LMISRTPEVT CVVVDVSHED PEVQFNWYVD GVEVHNAKTK PREEQFNSTF
              310 320 330 340 350 360
RVVSVLTVVH QDWLNGKEYK CKVSNKGLPA PIEKTISKTK GQPREPQVYT LPPSREEMTK
              370 380 390 400 410 420
NQVSLTCLVK GFYPSDIAVE WESNGQPENN YKTTPPMLDS DGSFFLYSKL TVDKSRWQQG
              430 440
NVFSCSVMHE ALHNHYTQKS LSLSPGK
Phor18-V_{H} IgG2 (483) (2 Phor18, N-terminus)
(SEQ ID NO.: 123)
Light chain
                10 20 30 40 50 60
DIQMTQSPSS LSASVGDRVT ITCRASSSVS YIHWYQQKPG KAPKLLIYAT SNLASGVPSR
                70 80 90 100 110 120
FSGSRSGTDF TLTISSLQPE DFATYYCQQW TSNPPTFGQG TKVEIKGQPK ANPTVTLFPP
              130 140 150 160 170 180
SSEELQANKA TLVCLISDFY PGAVTVAWKA DGSPVKAGVE TTKPSKQSNN KYAASSYLSL
              190 200 210
TPEQWKSHRS YSCQVTHEGS TVEKTVAPTE CS
Heavy chain
(SEQ ID NO.: 124)
                10 20 30 40 50 60
MKFAKFAKKF AKFAKKFAKG SEVQLVESGG GLVQPGGSLR LSCAASGYTF TSYNMHWVRQ
                70 80 90 100 110 120
APGKGLEWVA AIYPGNGDTS YNQKFKGRFT ISADTSKNTA YLQMNSLRAE DTAVYYCSRS
              130 140 150 160 170 180
TYYGGDWYFD VWGQGTLVTV SSASTKGPSV FPLAPCSRST SESTAALGCL VKDYFPEPVT
              190 200 210 220 230 240
VSWNSGALTS GVHTFPAVLQ SSGLYSLSSV VTVPSSNFGT QTYTCNVDHK PSNTKVDKTV
              250 260 270 280 290 300
ERKCCVECPP CPAPPVAGPS VFLFPPKPKD TLMISRTPEV TCVVVDVSHE DPEVQFNWYV
              310 320 330 340 350 360
DGVEVHNAKT KPREEQFNST FRVVSVLTVV HQDWLNGKEY KCKVSNKGLP APIEKTISKT
              370 380 390 400 410 420
KGQPREPQVY TLPPSREEMT KNQVSLTCLV KGFYPSDIAV EWESNGQPEN NYKTTPPMLD
              430 440 450 460
SDGSFFLYSK LTVDKSRWQQ GNVFSCSVMH EALHNHYTQK SLSLSPGK
Phor18-V_{L}-Phor18V_{H}-IgG2 (485) (4 Phor18, 2 N-terminus)
(SEQ ID NO.: 125)
Light chain
                10 20 30 40 50 60
MKFAKFAKKF AKFAKKFAKG SDIQMTQSPS SLSASVGDRV TITCRASSSV SYIHWYQQKP
                70 80 90 100 110 120
GKAPKLLIYA TSNLASGVPS RFSGSRSGTD FTLTISSLQP EDFATYYCQQ WTSNPPTFGQ
              130 140 150 160 170 180
GTKVEIKGQP KANPTVTLFP PSSEELQANK ATLVCLISDF YPGAVTVAWK ADGSPVKAGV
              190 200 210 220 230
ETTKPSKQSN NKYAASSYLS LTPEQWKSHR SYSCQVTHEG STVEKTVAPT ECS
Heavy chain
(SEQ ID NO.: 126)
                10 20 30 40 50 60
MKFAKFAKKF AKFAKKFAKG SEVQLVESGG GLVQPGGSLR LSCAASGYTF TSYNMHWVRQ
                70 80 90 100 110 120
APGKGLEWVA AIYPGNGDTS YNQKFKGRFT ISADTSKNTA YLQMNSLRAE DTAVYYCSRS
              130 140 150 160 170 180
TYYGGDWYFD VWGQGTLVTV SSASTKGPSV FPLAPCSRST SESTAALGCL VKDYFPEPVT
              190 200 210 220 230 240
VSWNSGALTS GVHTFPAVLQ SSGLYSLSSV VTVPSSNFGT QTYTCNVDHK PSNTKVDKTV
              250 260 270 280 290 300
ERKCCVECPP CPAPPVAGPS VFLFPPKPKD TLMISRTPEV TCVVVDVSHE DPEVQFNWYV
              310 320 330 340 350 360
DGVEVHNAKT KPREEQFNST FRVVSVLTVV HQDWLNGKEY KCKVSNKGLP APIEKTISKT
              370 380 390 400 410 420
KGQPREPQVY TLPPSREEMT KNQVSLTCLV KGFYPSDIAV EWESNGQPEN NYKTTPPMLD
              430 440 450 460
SDGSFFLYSK LTVDKSRWQQ GNVFSCSVMH EALHNHYTQK SLSLSPGK
Phor18-V_{L}-C_{L}-Phor18-Phor18-V_{H}-IgG2 (487) (6 Phor18, 2 N and 1 C-terminus)
(SEQ ID NO.: 127)
Light chain
                10 20 30 40 50 60
MKFAKFAKKF AKFAKKFAKG SDIQMTQSPS SLSASVGDRV TITCRASSSV SYIHWYQQKP
                70 80 90 100 110 120
GKAPKLLIYA TSNLASGVPS RFSGSRSGTD FTLTISSLQP EDFATYYCQQ WTSNPPTFGQ
              130 140 150 160 170 180
GTKVEIKGQP KANPTVTLFP PSSEELQANK ATLVCLISDF YPGAVTVAWK ADGSPVKAGV
              190 200 210 220 230 240
ETTKPSKQSN NKYAASSYLS LTPEQWKSHR SYSCQVTHEG STVEKTVAPT ECSGSKAFKK
              250
AFKAFKKAFK AFK
Heavy chain
(SEQ ID NO.: 128)
                10 20 30 40 50 60
MKFAKFAKKF AKFAKKFAKG SEVQLVESGG GLVQPGGSLR LSCAASGYTF TSYNMHWVRQ
                70 80 90 100 110 120
APGKGLEWVA AIYPGNGDTS YNQKFKGRFT ISADTSKNTA YLQMNSLRAE DTAVYYCSRS
              130 140 150 160 170 180
TYYGGDWYFD VWGQGTLVTV SSASTKGPSV FPLAPCSRST SESTAALGCL VKDYFPEPVT
              190 200 210 220 230 240
VSWNSGALTS GVHTFPAVLQ SSGLYSLSSV VTVPSSNFGT QTYTCNVDHK PSNTKVDKTV
              250 260 270 280 290 300
ERKCCVECPP CPAPPVAGPS VFLFPPKPKD TLMISRTPEV TCVVVDVSHE DPEVQFNWYV
              310 320 330 340 350 360
DGVEVHNAKT KPREEQFNST FRVVSVLTVV HQDWLNGKEY KCKVSNKGLP APIEKTISKT
              370 380 390 400 410 420
KGQPREPQVY TLPPSREEMT KNQVSLTCLV KGFYPSDIAV EWESNGQPEN NYKTTPPMLD
              430 440 450 460
SDGSFFLYSK LTVDKSRWQQ GNVFSCSVMH EALHNHYTQK SLSLSPGK
Phor18-V_{L}-C_{L}-Phor18-V_{H}-Phor18-C_{H}3-Phor18-IgG2 (489)
(8 Phor18, 2 N and 2 C-terminus)
(SEQ ID NO.: 129)
Light chain
                10 20 30 40 50 60
MKFAKFAKKF AKFAKKFAKG SDIQMTQSPS SLSASVGDRV TITCRASSSV SYIHWYQQKP
                70 80 90 100 110 120
GKAPKLLIYA TSNLASGVPS RFSGSRSGTD FTLTISSLQP EDFATYYCQQ WTSNPPTFGQ
              130 140 150 160 170 180
GTKVEIKGQP KANPTVTLFP PSSEELQANK ATLVCLISDF YPGAVTVAWK ADGSPVKAGV
              190 200 210 220 230 240
ETTKPSKQSN NKYAASSYLS LTPEQWKSHR SYSCQVTHEG STVEKTVAPT ECSGSKAFKK
              250
AFKAFKKAFK AFK
Heavy chain
(SEQ ID NO.: 130)
                10 20 30 40 50 60
MKFAKFAKKF AKFAKKFAKG SEVQLVESGG GLVQPGGSLR LSCAASGYTF TSYNMHWVRQ
                70 80 90 100 110 120
APGKGLEWVA AIYPGNGDTS YNQKFKGRFT ISADTSKNTA YLQMNSLRAE DTAVYYCSRS
              130 140 150 160 170 180
TYYGGDWYFD VWGQGTLVTV SSASTKGPSV FPLAPCSRST SESTAALGCL VKDYFPEPVT
              190 200 210 220 230 240
VSWNSGALTS GVHTFPAVLQ SSGLYSLSSV VTVPSSNFGT QTYTCNVDHK PSNTKVDKTV
              250 260 270 280 290 300
ERKCCVECPP CPAPPVAGPS VFLFPPKPKD TLMISRTPEV TCVVVDVSHE DPEVQFNWYV
              310 320 330 340 350 360
DGVEVHNAKT KPREEQFNST FRVVSVLTVV HQDWLNGKEY KCKVSNKGLP APIEKTISKT
              370 380 390 400 410 420
KGQPREPQVY TLPPSREEMT KNQVSLTCLV KGFYPSDIAV EWESNGQPEN NYKTTPPMLD
              430 440 450 460 470 480
SDGSFFLYSK LTVDKSRWQQ GNVFSCSVMH EALHNHYTQK SLSLSPGKGS KAFKKAFKAF
KKAFKAFK
```

Invitrogen free-style suspension grown CHO cells (Free-style MAX CHO expression system cat #K9000-20) were transfected using Invitrogen protocols. Briefly, FS CHO cells were expanded for 6 to 7 days after thawing to rapid growth phase, doubling every 24 h. The day before transfection, clumps were removed, cells were pelleted and resuspended in P/S-free medium at 5×10⁵/ml. On the day of transfection, 30 ml cultures of cells were adjusted to 9×10⁵ /ml if necessary and viability should be close to 99%.

Each 125 ml spinner flask (VWR cat #PBV125) of 30 ml cells was transfected with 35 μg of total plasmid DNA mixed with 35 μl of FSMax transfection reagent (Invitrogen cat #16447-100). DNA should be at 1 mg/ml or higher in concentration. Cells were swirled rapidly while adding DNA mixture slowly. Ratios of H: L chains tested were 3:2, 1:1, and 2:3.

Protein was harvested on day 3 and day 6 after transfection. Approximately 0.25 ml of protein A resin (Genscript L00210, capacity >20 mg IgG per ml resin) was used to isolate secreted ADCs from the FS CHO medium using the Genscript protocol provided The expressed anti-CD20 IgG2 antibody (humanized variable light and heavy domains regions to CD20 receptor) and the various antibody-Phor18 conjugates with stoichiometric ratios of Phor18: AB of 2:1, 4:1 and 6:1 were characterized using SDS PAGE, and Western blot analyses (Table 14).

TABLE 14
ADC descriptions for CD20 targeting ADC

| | | | Number of |
|---|---|---|---|
| | | | Phor18/ |
| Name of ADC | ID | Phor18 location | Antibody |
| IgG2 | 480 | None (‘Naked’) | 0 |
| Phor18-V_{L}-IgG2 | 481 | N-termini light chains (V_{L}) | 2 |
| Phor18-V_{H}-IgG2 | 483 | N-termini heavy chains (V_{H}) | 2 |
| Phor18-V_{L}-Phor18- | 485 | N-termini heavy | 4 |
| V_{H}-IgG2 | | and light chains | |
| | | (V_{H}, V_{L}) | |
| Phor18-V_{L}- | 487 | N-termini and | 6 |
| Phor18-V_{H}-CL- | | C-termini light chains | |
| Phor18-IgG2 | | (V_{L}, V_{H}, C_{L}) | |
| Phor18-V_{L}-C_{L}- | 489 | N-termini and | 8 |
| Phor18-V_{H}- | | C-termini light chains | |
| Phor18-C_{H}3-Phor18-IgG2 | | (V_{L}, C_{L}, V_{H}, C_{H}3) | |

ADCs were probed on Western blots probed with anti-Phor18. All ADCs have a single Phor18 on the heavy and light chains. Yield, purity and cytotoxicity of recombinantly produced antibodies (as a full IgG2 antibody) with Heavy (H) or Light (L) chain C-terminal- or N-terminal-Phor18 conjugation was analyzed. Two, 4 and 6 molecules of lytic domains (Phor18) conjugated to whole antibody molecule were expressed.

The concentration of recombinantly produced antibody and antibody-Phor18 conjugates was determined using spectrophotometric measurements (OD₂₈₀) and were as follows: IgG2 (Mw 150,000; 1.096 mg/ml), Phor18-V_{L}-IgG2 (Mw 154,340 g/mol, 0.561 mg/ml), Phor18-V_{H}-IgG2 (Mw 154,340 g/mol, 0.1 mg/ml) and Phor18-V_{L}-Phor18V_{H}-IgG2 (Mw 158,400 g/mol, 0.561 mg/ml) and Phor18-V_{L}-Phor18V_{H}-C_{L}-Phor18-IgG2 (Mw 162,600, 0.07 mg/ml).

### Example 22

This example describes in vitro activity of recombinantly produced IgG2-Phor18 conjugates.

The cytotoxicity of recombinantly produced CD20 targeting IgG2-Phor18 conjugates in a mammalian expression system was compared with “naked” antibody (IgG2) in CD20 positive cells (Daudi, Burkitts lymphoma). CD20 negative leukemia cells (U937) served as controls. CHO cell expressed ADCs represented intact antibodies and were conjugated to 2, 4 and 6 Phor18 molecules at the N-terminus of the V_{L} chain at the N-terminus of the V_{H} chain, at the N-terminus of the V_{H} and V_{L} chain and at the N-terminus at the V_{L}, V_{H} and C-terminus of the C_{L} chain. The sequence descriptions are summarized in Table 14.

Human Non-Hodgkin's lymphoma cells Daudi (CD20 positive, passage number p6) and human leukemia cell line U937 (CD20 negative, p 10) were seeded at a density of 2,000 cells per well in opaque plates in heat inactivated full medium using cell dissociation buffer. After 24 hours cells were replenished with fresh media (75 μl) and incubated with 25 μl of a 4× serial dilution of each ADC and naked antibody prepared in cell culture media were added at concentrations between 0.001-200 nM for IgG2, Phor18-V_{L}-IgG2, Phor18-V_{H}-IgG2 and Phor18-V_{L}-Phor18V_{H}-IgG2, and 0.001-100 nM for Phor18-V_{L}-Phor18V_{H}-C_{L}-Phor18-IgG2.

Cells incubated for 4 hours were assayed for membrane integrity using a luminometric assay kit (Promega, Madison, WI, Cytotox Glo G9292 lot #317872). Cell viability was determined was determined after 24, and 48 hours using a luminescent assay kit (Promega, Madison, WI, Cell Titer Glo, G 7572, lot 336262).

Controls for 100% cell viability (culture media) and 100% cell death (0.1% Triton X 100) incubated under the same conditions.

Data were processed and analyzed to obtain IC₅₀ values using Graph Pad Prizm version 5.00 for Windows, GraphPad Software, San Diego California USA, www-graphpad-com (Graph Pad Prizm, Inc). Statistical analysis for significance was determined by a two-tailed Student's T-test. Each test was conducted using double plates of 2-3 wells each to achieve an N of 4-6 data points per time point.

TABLE 15
In vitro toxicities of anti-CD20 IgG2, Phor18-V_{L}-IgG2, Phor18-V_{H}-IgG2, Phor18-V_{L}-
Phor18V_{H}-IgG2 and Phor18-V_{L}-Phor18V_{H}-C_{L}-Phor18-IgG2, targeting CD 20. Daudi cells (NHL)
were positive for CD 20, U937 cells (leukemia) are negative for CD20.

| | | Phor18-V_{L}- | Phor18-V_{H}- | Phor18-V_{L}- | Phor18-V_{L}-Phor18V_{H}-C_{L}- |
|---|---|---|---|---|---|
| | | IgG2 | IgG2 | Phor18V_{H}-IgG2 | Phor18-IgG2 |
| | IgG2 | IC₅₀ [nM] | IC₅₀ [nM] | IC₅₀ [nM] | IC₅₀ [nM] |
| | naked | V_{L} N- | V_{H} N- | V_{H} and V_{H} N- | V_{L} and V_{H} N-terminus, |
| | | terminus | terminus | terminus | C_{L} C-terminus |
| | 0 | 2 Phor18 | 2 Phor18 | 4 Phor18 | 6 Phor18 |
| | IC₅₀ | | | | |
| Daudi | [nM] | IC₅₀ [nM] | IC₅₀ [nM] | IC₅₀ [nM] | IC₅₀ [nM] |
| 4 h | Not | ND | 1108 ± 198 | 109.6 ± 20.1 | 71.6 ± 29 |
| (Membrane | toxic | | | | |
| integrity) | | | | | |
| 24 h | Not | 824 ± 63 | 75.24 ± 37.5 | 40.1 ± 15.1 | 18.8 ± 5.9 |
| | toxic | | | | |
| 48 h | Not | >436 | 4.8 ± 2 | 20.4 ± 8.2 | 1.9 ± 0.2 |
| | toxic | | | | |
| | IC₅₀ | | | | |
| U937 | [nM] | IC₅₀ [nM] | IC₅₀ [nM] | IC₅₀ [nM] | IC₅₀ [nM] |
| 4 h (Membrane | Not | Not toxic | Not toxic | Not toxic | Not toxic |
| integrity) | toxic | | | | |
| 24 h | Not | Not toxic | Not toxic | Not toxic | Not toxic |
| | toxic | | | | |
| 48 h | Not | >1000 | Not toxic | 249.6 ± 29 | 258 ± 41 |
| | toxic | | | | |

ND = not determined

Destruction of membrane integrity was measured after 4 hours at nanomolar concentrations of 1,108±198 for Phor18-V_{H}-IgG2 (2 Phor18), 109.6±20.1 for Phor18-V_{L}-Phor18V_{H}-IgG2 (4 Phor18) and 71.6±29 for Phor18-V_{L}-Phor18V_{H}-C_{L}-Phor18-IgG2 (6 Phor18). Naked antibody was not toxic after 24 or 48 hours to the CD20 positive Daudi cells.

Killing of the target cells after 24 h expressed as IC₅₀ values [nM] were 824±63, 75.24±37.5, (2 Phor18), 40.1±15.1 (4 Phor18) and 18.8±5.9 (6 Phor18) nM. Maximal effects measured after 48 h (IC₅₀ [nM]>436 (Phor18-V_{L}-IgG2), 4.8±2 Phor18-V_{H}-IgG2 (2 Clips) and 20.4-8.2 nM Phor18-V_{L}-Phor18V_{H}-IgG2 (4 Clips), and 1.9±0.2 nM Phor18-V_{L}-Phor18V_{H}-C_{L}-Phor18-IgG2 (6 Phor18) respectively). The CD20 negative cell line (U937) was not killed by either naked antibodies or both 2 4, and 6 Phor18 conjugated ADCs after 4 and 24 hours. Toxicity levels after 48 hours were similar with IC₅₀ values of 249 and 258 nM. These data demonstrate that position of Phor18 determines the potency of a CD20 ADC with increased potency for Phor18-V_{H}-IgG2. Increase of Phor18 molecules per antibody increased the potency.

Whole antibody Phor18 conjugates with Phor18: antibody stoichiometries of 2, 4 and 6 were recombinantly expressed in CHO cells. These antibody drug conjugates had confirmed Phor18 molecules at heavy and light chains. They were active in CD20 positive cells in vitro with activities of membrane disintegration after 4 hours. After 48 hours single digit nanomolar activities were measured that were highest in ADC with 6 and 4 Phor18.

Non-internalizing CD20 antibody conjugates destroyed membrane integrity of target cells (Daudi) within 4 h. Cell death was observed after 24 h with IC₅₀ values in the low nanomolar range. CD20 negative cells (U937) were not killed. Full IgG2 ADCs having 4 and 6 clips had increased potency to target positive cells compared to 2 Phor18 conjugates. The positioning of Phor18 on the N-terminus generated ADCs with higher potencies. Increase of Phor18 numbers per antibody showed increased potencies.

Higher numbers of Phor18 (six vs four vs two) on the antibody conjugated to the N-terminal domain are more potent compared to naked antibodies.

### Example 23

This example includes target sequence information for representative target proteins to which antibody and polypeptide conjugates of the invention can be produced and naked antibodies and their Phor18 conjugates

```
ERBB2 (HER2/NEU) Isoform 1 [UniParc]:
(SEQ ID NO.: 131)
                10 20 30 40 50 60
MELAALCRWG LLLALLPPGA ASTQVCTGTD MKLRLPASPE THLDMLRHLY QGCQVVQGNL
                70 80 90 100 110 120
ELTYLPTNAS LSFLQDIQEV QGYVLIAHNQ VRQVPLQRLR IVRGTQLFED NYALAVLDNG
              130 140 150 160 170 180
DPLNNTTPVT GASPGGLREL QLRSLTEILK GGVLIQRNPQ LCYQDTILWK DIFHKNNQLA
              190 200 210 220 230 240
LTLIDTNRSR ACHPCSPMCK GSRCWGESSE DCQSLTRTVC AGGCARCKGP LPTDCCHEQC
              250 260 270 280 290 300
AAGCTGPKHS DCLACLHENH SGICELHCPA LVTYNTDTFE SMPNPEGRYT FGASCVTACP
              310 320 330 340 350 360
YNYLSTDVGS CTLVCPLHNQ EVTAEDGTQR CEKCSKPCAR VCYGLGMEHL REVRAVTSAN
              370 380 390 400 410 420
IQEFAGCKKI FGSLAFLPES FDGDPASNTA PLQPEQLQVF ETLEEITGYL YISAWPDSLP
              430 440 450 460 470 480
DLSVFQNLQV IRGRILHNGA YSLTLQGLGI SWLGLRSLRE LGSGLALIHH NTHLCFVHTV
              490 500 510 520 530 540
PWDQLFRNPH QALLHTANRP EDECVGEGLA CHQLCARGHC WGPGPTQCVN CSQFLRGQEC
              550 560 570 580 590 600
VEECRVLQGL PREYVNARHC LPCHPECQPQ NGSVTCFGPE ADQCVACAHY KDPPFCVARC
              610 620 630 640 650 660
PSGVKPDLSY MPIWKFPDEE GACQPCPINC THSCVDLDDK GCPAEQRASP LTSIISAVVG
              670 680 690 700 710 720
ILLVVVLGVV FGILIKRRQQ KIRKYTMRRL LQETELVEPL TPSGAMPNQA QMRILKETEL
              730 740 750 760 770 780
RKVKVLGSGA FGTVYKGIWI PDGENVKIPV AIKVLRENTS PKANKEILDE AYVMAGVGSP
              790 800 810 820 830 840
YVSRLLGICL TSTVQLVTQL MPYGCLLDHV RENRGRLGSQ DLLNWCMQIA KGMSYLEDVR
              850 860 870 880 890 900
LVHRDLAARN VLVKSPNHVK ITDFGLARLL DIDETEYHAD GGKVPIKWMA LESILRRRFT
              910 920 930 940 950 960
HQSDVWSYGV TVWELMTFGA KPYDGIPARE IPDLLEKGER LPQPPICTID VYMIMVKCWM
              970 980 990 1000 1010 1020
IDSECRPRER ELVSEFSRMA RDPQRFVVIQ NEDLGPASPL DSTFYRSLLE DDDMGDLVDA
            1030 1040 1050 1060 1070 1080
EEYLVPQQGF FCPDPAPGAG GMVHHRHRSS STRSGGGDLT LGLEPSEEEA PRSPLAPSEG
            1090 1100 1110 1120 1130 1140
AGSDVFDGDL GMGAAKGLQS LPTHDPSPLQ RYSEDPTVPL PSETDGYVAP LTCSPQPEYV
            1150 1160 1170 1180 1190 1200
NQPDVRPQPP SPREGPLPAA RPAGATLERP KTLSPGKNGV VKDVFAFGGA VENPEYLTPQ
            1210 1220 1230 1240 1250
GGAAPQPHPP PAFSPAFDNL YYWDQDPPER GAPPSTFKGT PTAENPEYLG LDVPV
```

CD19: B-lymphocyte surface antigen B4, component of the B-cell co-receptor, NCBI Reference Sequence: NP_001171569.1:

| ORIGIN | |
|---|---|
| (SEQ ID NO.: 132) | |
| 1 | mppprliffl lfltpmevrp eeplvvkvee gdnavlqclk gtsdgptqql twsresplkp |
| 61 | flklsiglpg lgihmrplai wlfifnvsqq mggfylcqpg ppsekawqpg wtvnvegsge |
| 121 | lfrwnvsdlg glgcglknrs segpsspsgk lmspklyvwa kdrpeiwege ppclpprdsl |
| 181 | nqslsqditm apgstlwlsc gvppdsvsrg plswthvhpk gpksllslel kddrpardmw |
| 241 | vmetglllpr ataqdagkyy chrgnltmsf hleitarpvl whwllrtggw kvsavtlayl |
| 301 | ifclcslvgi lhlqralvlr rkrkrmtdpt rrffkvtppp gsgpqnqygn vislptptsg |
| 361 | lgraqrwaag lggtapsygn pssdvqadga lgsrsppgvg peeeegegye epdseedsef |
| 421 | yendsnigqd qlsqdgsgye npedeplgpe dedsfsnaes yenedeeltq pvartmdfls |
| 481 | phgsawdpsr eatslagsqs yedmrgilya apqlrsirgq pgpnheedad syenmdnpdg |
| 541 | pdpawggggr mgtwstr |

CD20: a type III transmembrane protein found on B cells that forms a calcium channel in the cell membrane allowing for the influx of calcium required for cell activation; expressed in B-cell lymphomas, hairy cell leukemia, and B-cell chronic lymphocytic leukemia. Important for therapy of those diseases, as an antibody against CD20 exists: Rituximab. NCBI Reference Sequence

| NP_068769.2, MS4A1-P11836: | |
|---|---|
| (SEQ ID NO.: 133) | |
| 1 | mttprnsvng tfpaepmkgp iamqsgpkpl frrmsslvgp tqsffmresk tlgavqimng |
| 61 | lfhialggll mipagiyapi cvtvwyplwg gimyiisgsl laateknsrk clvkgkmimn |
| 121 | slslfaaisg milsimdiln ikishflkme slnfirahtp yiniyncepa npseknspst |
| 181 | qycysiqslf lgilsvmlif affqelviag ivenewkrtc srpksnivll saeekkeqti |
| 241 | eikeevvglt etssqpknee dieiipiqee eeeetetnfp eppqdqessp iendssp |

CD22: a sugar binding transmembrane protein that specifically binds sialic acid with an immunoglobulin (Ig) domain located at its N-terminus. It is a member of the immunoglobulin superfamily and the SIGLEC family. CD22 functions as an inhibitory receptor for B cell receptor (BCR) signaling. NCBI Reference Sequence NP_001172028.1:

| ORIGIN | |
|---|---|
| (SEQ ID NO.: 134) | |
| 1 | mhllgpwlll lvleylafsd sskwvfehpe tlyawegacv wipctyrald gdlesfilfh |
| 61 | npeynkntsk fdgtrlyest kdgkvpseqk rvqflgdknk nctlsihpvh lndsgqlglr |
| 121 | mesktekwme rihlnvserp fpphiqlppe iqesqevtlt clinfscygy piqlqwlleg |
| 181 | vpmrqaavts tsltiksvft rselkfspqw shhgkivtcq lqdadgkfls ndtvqlnvkh |
| 241 | tpkleikvtp sdaivregds vtmtcevsss npeyttvswl kdgtslkkqn tftlnlrevt |
| 301 | kdqsgkyccq vsndvgpgrs eevflqvqyp pkkvttviqn pmpiregdtv tlscnynssn |
| 361 | psvtryewkp hgaweepslg vlkiqnvgwd nttiacaacn swcswaspva lnvqyaprdv |
| 421 | rvrkikplse ihsgnsvslq cdfssshpke vqffwekngr llgkesqlnf dsispedags |
| 481 | yscwvnnsig qtaskawtle vlyaprilrv smspgdqvme gksatltces danppvshyt |
| 541 | wfdwnnqslp yhsqklrlep vkvqhsgayw cqgtnsvgkg rsplstltvy yspetigrrv |
| 601 | avglgsclai lilaicglkl qrrwkrtqsq qglgenssgq sffvrnkkvr raplsegphs |
| 661 | lgcynpmmed gisyttlrfp emniprtgda essemqrppp dcddtvtysa lhkrqvgdye |
| 721 | nvipdfpede gihyseliqf gvgerpqage nvdyvilkh |

CD23: a type II transmembrane protein found on mature B cells, monocytes, activated macrophages, eosinophils, platelets, and dendritic cells that enhances capture and processing of antigen complexed with IgE. NCBI Reference Sequence NP_001193948.2:

| ORIGIN | |
|---|---|
| (SEQ ID NO.: 135) | |
| 1 | mnppsqeiee lprrrccrrg tqivllglvt aalwaglltl lllwhwdttq slkqleeraa |
| 61 | rnvsqvsknl eshhgdqmaq ksqstqisqe leelraeqqr lksqdlelsw nlnglqadls |
| 121 | sfksqelner neasdllerl reevtklrme lqvssgfvon tcpekwinfq rkcyyfgkgt |
| 181 | kqwvharyac ddmegqlvsi hspeeqdflt khashtgswi glrnldlkge fiwvdgshvd |
| 241 | ysnwapgept srsqgedcvm mrgsgrwnda fcdrklgawv cdrlatctpp asegsaesmg |
| 301 | pdsrpdpdgr lptpsaplhs |

//

CD27: TNF-receptor. Present on the surface of resting memory B cells. NCBI Reference Sequence NP_001233.1:

| ORIGIN | |
|---|---|
| (SEQ ID NO.: 136) | |
| 1 | marphpwwlc vlgtlvglsa tpapkscper hywaqgklcc qmcepgtflv kdcdqhrkaa |
| 61 | qcdpcipgvs fspdhhtrph cescrhonsg llvrnctita naecacrngw qcrdkectec |
| 121 | dplpnpslta rssqalsphp qpthlpyvse mleartaghm qtladfrqlp artlsthwpp |
| 181 | qrslcssdfi rilvifsgmf lvftlagalf lhqrrkyrsn kgespvepae pcryscpree |
| 241 | egstipiqed yrkpepacsp |

CD28: present on all T-cells, and when matched with the appropriate ligand, labeled B7 which can be either CD80 or CD86, it has costimulatory effect on the T-cell. It is also expressed on Eosinophil granulocytes, especially after tissue infiltration. There its ligation leads to release of potent neurotoxins, IL-2 and IL-13 as well as IFN-γ. Checksum 1D9B6552A5878DOF: (SEQ ID NO.: 137)

```
                10 20 30 40 50 60
MLRLLLALNL FPSIQVTGNK ILVKQSPMLV AYDNAVNLSC KYSYNLFSRE FRASLHKGLD
                70 80 90 100 100 120
SAVEVCVVYG NYSQQLQVYS KTGFNCDGKL GNESVTFYLQ NLYVNQTDIY FCKIEVMYPP
              130 140 150 160 170 180
PYLDNEKSNG TIIHVKGKHL CPSPLFPGPS KPFWVLVVVG GVLACYSLLV TVAFIIFWVR
              190 200 210 220
SKRSRLLHSD YMNMTPRRPG PTRKHYQPYA PPRDFAAYRS
```

CD30: a type I transmembrane protein present on activated T and B cells that may play a role in cell activation and/or differentiation; expressed in Hodgkin disease, some T-cell lymphomas, and anaplastic large cell lymphomas. (SEQ ID NO.: 138)

```
                10 20 30 40 50 60
MRVLLAALGL LFLGALRAFP QDRPFEDTCH GNPSHYYDKA VRRCCYRCPM GLFPTQQCPQ
                70 80 90 100 100 120
RPTDCRKQCE PDYYLDEADR CTACVTCSRD DLVEKTPCAW NSSRVCECRP GMFCSTSAVN
              130 140 150 160 170 180
SCARCFFHSV CPAGMIVKFP GTAQKNTVCE PASPGVSPAC ASPENCKEPS SGTIPQAKPT
              190 200 210 220 230 240
PVSPATSSAS TMPVRGGTRL AQEAASKLTR APDSPSSVGR PSSDPGLSPT QPCPEGSGDC
              250 260 270 280 290 300
RKQCEPDYYL DEAGRCTACV SCSRDDLVEK TPCAWNSSRT CECRPGMICA TSATNSCARC
              310 320 330 340 350 360
VPYPICAAET VTKPQDMAEK DTTFEAPPLG TQPDCNPTPE NGEAPASTSP TQSLLVDSQA
              370 380 390 400 410 420
SKTLPIPTSA PVALSSTGKP VLDAGPVLFW VILVLVVVVG SSAFLLCHRR ACRKRIRQKL
              430 440 450 450 470 480
HLCYPVQTSQ PKLELVDSRP RRSSTQLRSG ASVTEPVAEE RGLMSQPLME TCHSVGAAYL
              490 500 510 520 530 540
ESLPLQDASP AGGPSSPRDL PEPRVSTEHT NNKIEKIYIM KADTVIVGTV KAELPEGRGL
              550 560 570 580 590
AGPAEPELEE ELEADHTPHY PEQETEPPLG SCSDVMLSVE EEGKEDPLPT AASGK
```

P28908-2, Tumor necrosis factor receptor superfamily member 8, Homo sapiens (SEQ ID NO.: 139):

```
                10 20 30 40 50 60
MSQPLMETCH SVGAAYLESL PLQDASPAGG PSSPRDLPEP RVSTEHTNNK IEKIYIMKAD
                70 80 90 100 100 120
TVIVGTVKAE LPEGRGLAGP AEPELEEELE ADHTPHYPEQ ETEPPLGSCS DVMLSVEEEG
              130
KEDPLPTAAS GK
```

CD31: PECAM-1, a cell adhesion molecule on platelets and endothelial cells (SEQ ID NO.: 140).

```
                10 20 30 40 50 60
MQPRWAQGAT MWLGVLLTLL LCSSLEGQEN SFTINSVDMK SLPDWTVQNG KNLTLQCFAD
                70 80 90 100 100 120
VSTTSHVKPQ HQMLFYKDDV LFYNISSMKS TESYFIPEVR IYDSGTYKCT VIVNNKEKTT
              130 140 150 160 170 180
AEYQLLVEGV PSPRVTLDKK EAIQGGIVRV NCSVPEEKAP IHFTIEKLEL NEKMVKLKRE
              190 200 210 220 230 240
KNSRDQNFVI LEFPVEEQDR VLSFRCQARI ISGIHMQTSE STKSELVTVT ESFSTPKFHI
              250 260 270 280 290 300
SPTGMIMEGA QLHIKCTIQV THLAQEFPEI IIQKDKAIVA HNRHGNKAVY SVMAMVEHSG
              310 320 330 340 350 360
NYTCKVESSR ISKVSSIVVN ITELFSKPEL ESSFTHLDQG ERLNLSCSIP GAPPANFTIQ
              370 380 390 400 410 420
KEDTIVSQTQ DFTKIASKSD SGTYICTAGI DKVVKKSNTV QIVVCEMLSQ PRISYDAQFE
              430 440 450 450 470 480
VIKGQTIEVR CESISGTLPI SYQLLKTSKV LENSTKNSND PAVEKDNPTE DVEYQCVADN
              490 500 510 520 530 540
CHSHAKMLSE VLRVKVIAPV DEVQISILSS KVVESGEDIV LQCAVNEGSG PITYKFYREK
              550 560 570 580 590 600
EGKPFYQMTS NATQAFWTKQ KASKEQEGEY YCTAFNRANH ASSVPRSKIL TVRVILAPWK
              610 620 630 640 650 660
KGLIAVVIIG VIIALLIIAA KCYFLRKAKA KQMPVEMSRP AVPLLNSNNE KMSDPNMEAN
              670 680 690 700 710 720
SHYGHNDDVR NHAMKPINDN KEPLNSDVQY TEVQVSSAES HKDLGKKDTE TVYSEVRKAV
              730
PDAVESRYSR TEGSLDGT
```

CD33: a marker of unknown function found on immature myeloid cells, including acute myeloid leukemia blasts and mature monocytes. P20138: (SEQ ID NO.: 141)

```
                10 20 30 40 50 60
MPLLLLLPLL WAGALAMDPN FWLQVQESVT VQEGLCVLVP CTFFHPIPYY DKNSPVHGYW
                70 80 90 100 100 120
FREGAIISRD SPVATNKLDQ EVQEETQGRF RLLGDPSRNN CSLSIVDARR RDNGSYFFRM
              130 140 150 160 170 180
ERGSTKYSYK SPQLSVHVTD LTHRPKILIP GTLEPGHSKN LTCSVSWACE QGTPPIFSWL
              190 200 210 220 230 240
SAAPTSLGPR TTHSSVLIIT PRPQDHGTNL TCQVKFAGAG VTTERTIQLN VTYVPQNPTT
              250 260 270 280 290 300
GIFPGDGSGK QETRAGVVHG AIGGAGVTAL LALCLCLIFF IVKTHRRKAA RTAVGRNDTH
              310 320 330 340 350 360
PTTGSASPKH QKKSKLHGPT ETSSCSGAAP TVEMDEELHY ASLNFHGMNP SKDTSTEYSE
VRTQ
```

CD34: stem cell marker, adhesion, found on hematopoietic precursors (found in high concentrations in umbilical cord blood), capillary endothelium, and embryonic fibroblasts. Isoform CD34-F: (SEQ ID NO.: 142)

```
                10 20 30 40 50 60
MLVRRGARAG PRMPRGWTAL CLLSLLPSGF MSLDNNGTAT PELPTQGTFS NVSTNVSYQE
                70 80 90 100 100 120
TTTPSTLGST SLHPVSQHGN EATTNITETT VKFTSTSVIT SVYGNTNSSV QSQTSVISTV
              130 140 150 160 170 180
FTTPANVSTP ETTLKPSLSP GNVSDLSTTS TSLATSPTKP YTSSSPILSD IKAEIKCSGI
              190 200 210 220 230 240
REVKLTQGIC LEQNKTSSCA EFKKDRGEGL ARVLCGEEQA DADAGAQVCS LLLAQSEVRP
              250 260 270 280 290 300
QCLLLVLANR TEISSKLQLM KKHQSDLKKL GILDFTEQDV ASHQSYSQKT LIALVTSGAL
              310 320 330 340 350 360
LAVLGITGYF LMNRRSWSPT GERLGEDPYY TENGGGQGYS SGPGTSPEAQ GKASVNRGAQ
              370 380
ENGTGQATSR NGHSARQHVV ADTEL
```

CD40: a costimulatory protein found on antigen presenting cells. CD40 combines with CD154 (CD40L) on T cells to induce antibody isotype switching in B cells. Isoform I: (SEQ ID NO.: 143)

```
                10 20 30 40 50 60
MVRLPLQCVL WGCLLTAVHP EPPTACREKQ YLINSQCCSL CQPGQKLVSD CTEFTETECL
                70 80 90 100 100 120
PCGESEFLDT WNRETHCHQH KYCDPNLGLR VQQKGTSETD TICTCEEGWH CTSEACESCV
              130 140 150 160 170 180
LHRSCSPGFG VKQIATGVSD TICEPCPVGF FSNVSSAFEK CHPWTSCETK DLVVQQAGTN
              190 200 210 220 230 240
KTDVVCGPQD RLRALVVIPI IFGILFAILL VLVFIKKVAK KPTNKAPHPK QEPQEINFPD
              250 260 270
DLPGSNTAAP VQETLHGCQP VTQEDGKESR ISVQERQ
CD52: P31358m
(SEQ ID NO.: 144)
                10 20 30 40 50 60
MKRFLFLLLT ISLLVMVQIQ TGLSGQNDTS QTSSPSASSN ISGGIFLFFV ANAIIHLFCF
S
Q9UJ81
(SEQ ID NO.: 145)
                10
MKRFLFLLLT ISLLVMVQ
```

CD 56: Neural cell adhesion molecule 1. Short name=N-CAM-1, Alternative name(s). CD_antigen=CD56. Isoform 1: (SEQ ID NO.: 146)

```
                10 20 30 40 50 60
MLQTKDLIWT LFFLGTAVSL QVDIVPSQGE ISVGESKFFL CQVAGDAKDK DISWFSPNGE
                70 80 90 100 100 120
KLTPNQQRIS VVWNDDSSST LTIYNANIDD AGIYKCVVTG EDGSESEATV NVKIFQKLMF
              130 140 150 160 170 180
KNAPTPQEFR EGEDAVIVCD VVSSLPPTII WKHKGRDVIL KKDVRFIVLS NNYLQIRGIK
              190 200 210 220 230 240
KTDEGTYRCE GRILARGEIN FKDIQVIVNV PPTIQARQNI VNATANLGQS VTLVCDAEGF
              250 260 270 280 290 300
PEPTMSWTKD GEQIEQEEDD EKYIFSDDSS QLTIKKVDKN DEAEYICIAE NKAGEQDATI
              310 320 330 340 350 360
HLKVFAKPKI TYVENQTAME LEEQVTLTCE ASGDPIPSIT WRTSTRNISS EEKASWTRPE
              370 380 390 400 410 420
KQETLDGHMV VRSHARVSSL TLKSIQYTDA GEYICTASNT IGQDSQSMYL EVQYAPKLQG
              430 440 450 460 470 480
PVAVYTWEGN QVNITCEVFA YPSATISWER DGQLLPSSNY SNIKIYNTPS ASYLEVTPDS
              490 500 510 520 530 540
ENDFGNYNCT AVNRIGQESL EFILVQADTP SSPSIDQVEP YSSTAQVQFD EPEATGGVPI
              550 560 570 580 590 600
LKYKAEWRAV GEEVWHSKWY DAKEASMEGI VTIVGLKPET TYAVRLAALN GKGLGEISAA
              610 620 630 640 650 660
SEFKTQPVQG EPSAPKLEGQ MGEDGNSIKV NLIKQDDGGS PIRHYLVRYR ALSSEWKPEI
              670 680 690 700 710 720
RLPSGSDHVM LKSLDWNAEY EVYVVAENQQ GKSKAAHFVF RTSAQPTAIP ANGSPTSGLS
              730 740 750 760 770 780
TGAIVGILIV IFVLLLVVVD ITCYFLNKCG LFMCIAVNLC GKAGPGAKGK DMEEGKAAFS
              790 800 810 820 830 840
KDESKEPIVE VRTEEERTPN HDGGKHTEPN ETTPLTEPEK GPVEAKPECQ ETETKPAPAE
              850
VKTVPNDATQ TKENESKA
```

CD70: Tumor necrosis factor ligand superfamily member 7. P32970 (CD70_HUMAN):

```
(SEQ ID NO.: 147)
                10 20 30 40 50 60
MPEEGSGCSV RRRPYGCVLR AALVPLVAGL VICLVVCIQR FAQAQQQLPL ESLGWDVAEL
                70 80 90 100 110 120
QLNHTGPQQD PRLYWQGGPA LGRSFLHGPE LDKGQLRIHR DGIYMVHIQV TLAICSSTTA
              130 140 150 160 170 180
SRHHPTTLAV GICSPASRSI SLLRLSFHQG CTIASQRLTP LARGDTLCTN LTGTLLPSRN
              190
TDETFFGVQW VRP
```

```
CD123: IL3RA. Isoform 1:
(SEQ ID NO.: 148)
                10 20 30 40 50 60
MVLLWLTLLL IALPCLLQTK EDPNPPITNL RMKAKAQQLT WDLNRNVTDI ECVKDADYSM
                70 80 90 100 110 120
PAVNNSYCQF GAISLCEVTN YTVRVANPPF STWILFPENS GKPWAGAENL TCWIHDVDFL
              130 140 150 160 170 180
SCSWAVGPGA PADVQYDLYL NVANRRQQYE CLHYKTDAQG TRIGCRFDDI SRLSSGSQSS
              190 200 210 200 230 240
HILVRGRSAA FGIPCTDKFV VFSQIEILTP PNMTAKCNKT HSFMHWKMRS HFNRKFRYEL
              250 260 270 280 290 300
QIQKRMQPVI TEQVRDRTSF QLLNPGTYTV QIRARERVYE FLSAWSTPQR FECDQEEGAN
              310 320 330 340 350 360
TRAWRTSLLI ALGTLLALVC VFVICRRYLV MQRLFPRIPH MKDPIGDSFQ NDKLVVWEAG
              370
KAGLEECLVT EVQVVQKT
```

CD154: The ligand for CD40. This is a costimulatory molecule that plays many roles, best known for activating B cells but also known to induce the activation of an APC in association with T cell receptor stimulation by MHC molecules on the APC. Q3L8U2: (SEQ ID NO.: 149)

```
(SEQ ID NO.: 149)
                10 20 30 40 50 60
MIETYNQTSP RSAATGLPIS MKIFMYLLTV FLITQMIGSA LFAVYLHRRL DKIEDERNLH
                70 80 90 100 110 120
EDFVFMKTIQ RCNTGERSLS LLNCEEIKSQ FEGFVKDIML NKEETKKENS FEMQKVLQWA
              130 140 150 160 170 180
EKGYYTMSNN LVTLENGKQL TVKRQGLYYI YAQVTFCSNR EASSQAPFIA SLCLKSPGRF
              190 200 210 200 230 240
ERILLRAANT HSSAKPCGQQ SIHLGGVFEL QPGASVFVNV TDPSQVSHGT GFTSFGLLKL
```

CD138: syndecan, a plasma cell-surface glycoprotein, known as syndecan-1. Syndecan functions as the alpha receptor for collagen, fibronectin and thrombospondin. P18827: (SEQ ID

```
(SEQ ID NO.: 150)
                10 20 30 40 50 60
MRRAALWLWL CALALSLQPA LPQIVATNLP PEDQDGSGDD SDNFSGSGAG ALQDITLSQQ
                70 80 90 100 110 120
TPSTWKDTQL LTAIPTSPEP TGLEATAAST STLPAGEGPK EGEAVVLPEV EPGLTAREQE
              130 140 150 160 170 180
ATPRPRETTQ LPTTHLASTT TATTAQEPAT SHPHRDMQPG HHETSTPAGP SQADLHTPHT
              190 200 210 200 230 240
EDGGPSATER AAEDGASSQL PAAEGSGEQD FTFETSGENT AVVAVEPDRR NQSPVDQGAT
              250 260 270 280 290 300
GASQGLLDRK EVLGGVIAGG LVGLIFAVCL VGFMLYRMKK KDEGSYSLEE PKQANGGAYQ
              310
KPTKQEEFYA KPTKQEEFYA
```

Oncofetoprotein-5T4 Trophoblast glycoprotein. NCBI Reference Sequence NP_001159864.1:

| ORIGIN | |
|---|---|
| (SEQ ID NO.: 151) | |
| 1 | mpggcsrgpa agdgrlrlar lalvllgwvs sssptssass fsssapflas avsaqpplpd |
| 61 | qcpalcecse aartvkcvnr nltevptdlp ayvrnlfltg nqlavlpaga farrpplael |
| 121 | aalnlsgsrl devragafeh lpslrqldls hnpladlspf afsgsnasvs apsplvelil |
| 181 | nhivppeder qnrsfegmvv aallagralq glrrlelasn hflylprdvl aqlpslrhld |
| 241 | lsnnslvslt yvsfrnlthl eslhlednal kvlhngtlae lqglphirvf ldnnpwvcdc |
| 301 | hmadmvtwlk etevvqgkdr ltcaypekmr nrvllelnsa dldcdpilpp slqtsyvflg |
| 361 | ivlaligaif llvlylnrkg ikkwmhnird acrdhmegyh yryeinadpr ltnlssnsdv |

1. An antibody conjugate, comprising an antibody that binds to CD33 or CD70 a target, wherein the antibody is linked to a lytic domain, wherein said lytic domain comprises or consists of a peptide selected from KFAKFAKKFAKFAKK, KFAKFAKKFAKFAKKF, KFAKFAKKFAKFAKKFA, KFAKFAKKFAKFAKKFAK, KFAKFAKKFAKFAKKFAKF and KFAKFAKKFAKFAKKFAKFA (SEQ ID NOs.: 1-6), or a peptide selected from KFAKFAKKFAKFAKK, KFAKFAKKFAKFAKKF, KFAKFAKKFAKFAKKFA, KFAKFAKKFAKFAKKFAK, KFAKFAKKFAKFAKKFAKF and KFAKFAKKFAKFAKKFAKFA (SEQ ID NOs.: 1-6) having one or more of the K residues substituted with any of an F or L residue, one or more of the F residues substituted with any of a K, A or L residue, or one or more of the A residues substituted with any of a K, F or L residue; and wherein the lytic domain is linked to a Heavy (H) chain of the antibody, or linked to a Light (L) chain of the antibody.

2. A polypeptide conjugate, comprising a Heavy (H) chain or a Light (L) chain of an antibody that binds to CD33 or CD70, wherein the Heavy (H) chain or the Light (L) chain is linked to a lytic domain, and wherein said lytic domain comprises or consists of a peptide selected from KFAKFAKKFAKFAKK, KFAKFAKKFAKFAKKF, KFAKFAKKFAKFAKKFA, KFAKFAKKFAKFAKKFAK, KFAKFAKKFAKFAKKFAKF and KFAKFAKKFAKFAKKFAKFA (SEQ ID NOs.: 1-6), or a peptide selected from KFAKFAKKFAKFAKK, KFAKFAKKFAKFAKKF, KFAKFAKKFAKFAKKFA, KFAKFAKKFAKFAKKFAK, KFAKFAKKFAKFAKKFAKF and KFAKFAKKFAKFAKKFAKFA (SEQ ID NOs.: 1-6) having one or more of the K residues substituted with any of an F or L residue, one or more of the F residues substituted with any of a K, A or L residue, or one or more of the A residues substituted with any of a K, F or L residue.

3. The conjugate of claim 1, wherein the lytic domain is linked to the amino (NH₂)-terminus of the Heavy (H) chain or linked to the carboxy (C)-terminus of the Heavy (H) chain.

4. The conjugate of claim 1, wherein the lytic domain is linked to the amino (NH₂)-terminus of the Light (L) chain or linked to the carboxy (C)-terminus of the Light (L) chain.

5. The conjugate of claim 1, wherein the conjugate comprises a plurality of lytic domains linked to the Heavy (H) chain or Light (L) chain, wherein at least one of the lytic domains is linked to the amino (NH₂)-terminus of the Heavy (H) chain, or is linked to the amino (NH₂)-terminus of the Light (L) chain.

6. The conjugate of claim 1, wherein the conjugate comprises a plurality of lytic domains linked to the Heavy (H) chain or Light (L) chain, wherein at least one of the lytic domains is linked to the carboxy (C)-terminus of the Heavy (H) chain, or is linked to the carboxy (C)-terminus of the Light (L) chain.

7. The conjugate of claim 1, wherein the conjugate comprises a plurality of lytic domains linked to the Heavy (H) chain or Light (L) chain, wherein at least one of the lytic domains is linked to the amino (NH₂)-terminus of the Heavy (H) chain and at least one of the lytic domains is linked to the amino (NH₂)-terminus of the Light (L) chain.

8. The conjugate of claim 1, wherein the conjugate comprises a plurality of lytic domains linked to the Heavy (H) chain or Light (L) chain, wherein at least one of the lytic domains is linked to the amino (NH₂)-terminus of the Heavy (H) chain, at least one of the lytic domains is linked to the amino (NH₂)-terminus of the Light (L) chain, and at least one of the lytic domains is linked to the carboxy (C)-terminus of the Heavy (H) chain or is linked to the carboxy (C)-terminus of the Light (L) chain.

9. The conjugate of claim 1, wherein the conjugate comprises a plurality of lytic domains linked to the Heavy (H) chain or Light (L) chain, wherein at least one of the lytic domains is linked to the amino (NH₂)-terminus of the Heavy (H) chain, at least one of the lytic domains is linked to the amino (NH₂)-terminus of the Light (L) chain, at least one of the lytic domains is linked to the carboxy (C)-terminus of the Heavy (H) chain and at least one of the lytic domains is linked to the carboxy (C)-terminus of the Light (L) chain.

10. The conjugate of claim 1, comprising one, two, three, four, five, six, seven or eight lytic domains linked to the Heavy (H) chain or Light (L) chain.

11. The conjugate of claim 1, comprising two, four, six, or eight lytic domains linked to the Heavy (H) chain and/or Light (L) chain.

12. The conjugate of claim 1, comprising a plurality of at least one lytic domain per Heavy or Light chain.

13. The conjugate of claim 12, wherein the lytic domains linked to the Heavy (H) chain or Light (L) chain, have an identical amino acid sequence or a different amino acid sequence.

14. The conjugate of claim 1, wherein said lytic domain is joined to said Heavy (H) chain or Light (L) chain immediately after the last amino acid at the amino (NH₂)-terminus or the carboxy (C)-terminus of the Heavy (H) chain or the Light (L) chain, thereby forming a continuous amino acid sequence between the lytic domain and the Heavy (H) chain or Light (L) chain.

15. The conjugate of claim 1, wherein said Heavy (H) chain or Light (L) chain and said lytic domain are joined by a covalent bond.

16. The conjugate of claim 1, wherein said Heavy (H) chain or Light (L) chain and said lytic domain are joined by a peptide sequence or a non-peptide linker or spacer.

17. A composition comprising the antibody conjugate of claim 1.

18. The composition of claim 17, further comprising an anti-cell proliferative or immune stimulating agent.

19. A pharmaceutical composition comprising the antibody conjugate of claim 1.

20. A polypeptide comprising a: 1) lytic domain linked to the amino (NH₂)-terminus of the antibody Heavy (H) chain of an antibody that binds to 33 or CD70; 2) a lytic domain linked to the amino (NH₂)-terminus of the antibody Light (L) chain; 3) a lytic domain linked to the carboxy (C)-terminus of the antibody Heavy (H) chain of an antibody that binds to CD33 or CD70; or 4) a lytic domain linked to the carboxy (C)-terminus of the antibody Light (L) chain.

21. A polypeptide comprising a: 1) lytic domain linked to the amino(NH₂)-terminus of the antibody Heavy (H) Chain; 2) a lytic domain linked to the amino(NH₂)-terminus of the antibody Light (L) Chain; 3) a lytic domain linked to the carboxy(C)-terminus of the antibody Heavy (H) Chain; or 4) a lytic domain linked to the carboxy(C)-terminus of the antibody Light (L) Chain, wherein said Heavy (H) Chain or Light (L) Chain or antibody is selected from any Heavy (H) Chain any Light (L) Chain any antibody or or any one or more CDR sequence set forth in any of Tables A, B, C, D, 4, 5, or Examples 14, 15, 18, 20 or 21.

22. A method of selectively reducing or inhibiting proliferation of a cell that expresses a target to which the antibody conjugate of claim 1 binds, comprising contacting the cell with the conjugate of claims 1 or 2 or polypeptide of claims 20 or 21 in an amount sufficient to reduce or inhibit proliferation of the cell that expresses the target.

23. A method of reducing or inhibiting proliferation of a hyperproliferating cell, comprising contacting a cell with the conjugate of claims 1 or 2 or polypeptide of claims 20 or 21 in an amount sufficient to reduce or inhibit proliferation of the hyperproliferating cell.

24. A method of reducing or inhibiting proliferation of a neoplastic, tumor, cancer or malignant cell, comprising contacting the cell with the conjugate of claims 1 or 2 or polypeptide of claims 20 or 21 in an amount sufficient to reduce or inhibit proliferation of the neoplastic, tumor, cancer or malignant cell.

25. The method of any of claims 1, 2, 20 or 21, wherein the cell expresses Her2/ neu.

26. A method of treating a subject having a hyperproliferative disorder, comprising administering to the subject an amount of the conjugate of claims 1 or 2 or polypeptide of claims 20 or 21 sufficient to treat the hyperproliferative disorder.

27. A method of treating a subject having a neoplasia, tumor, cancer or malignancy, comprising administering to the subject an amount of the conjugate of claims 1 or 2 or polypeptide of claims 1 or 2 sufficient to reduce or inhibit proliferation of the neoplasia, tumor, cancer or malignancy.


SEQUENCE LISTING (154 sequences)

SEQ ID NO: 1 - protein, 15 aa, synthetic construct
  REGION 1..15: note = Description of Artificial Sequence: Lytic peptide
KFAKFAKKFAKFAKK

SEQ ID NO: 2 - protein, 16 aa, synthetic construct
  REGION 1..16: note = Description of Artificial Sequence: Lytic peptide
KFAKFAKKFAKFAKKF

SEQ ID NO: 3 - protein, 17 aa, synthetic construct
  REGION 1..17: note = Description of Artificial Sequence: Lytic peptide
KFAKFAKKFAKFAKKFA

SEQ ID NO: 4 - protein, 18 aa, synthetic construct
  REGION 1..18: note = Description of Artificial Sequence: Lytic peptide
KFAKFAKKFAKFAKKFAK

SEQ ID NO: 5 - protein, 19 aa, synthetic construct
  REGION 1..19: note = Description of Artificial Sequence: Lytic peptide
KFAKFAKKFAKFAKKFAKF

SEQ ID NO: 6 - protein, 20 aa, synthetic construct
  REGION 1..20: note = Description of Artificial Sequence: Lytic peptide
KFAKFAKKFAKFAKKFAKFA

SEQ ID NO: 7 - protein, 28 aa, synthetic construct
  REGION 1..28: note = Description of Artificial Sequence: Lytic peptide
KFAKFAKKFAKFAKKFAKFAKKFAKFAK

SEQ ID NO: 8 - protein, 5 aa, synthetic construct
  REGION 1..5: note = Description of Artificial Sequence: Linker peptide
GSGGS

SEQ ID NO: 9 - protein, 5 aa, synthetic construct
  REGION 1..5: note = Description of Artificial Sequence: Linker peptide
ASAAS

SEQ ID NO: 10 - protein, 6 aa, synthetic construct
  REGION 1..6: note = Description of Artificial Sequence: Linker peptide
GSGRSA

SEQ ID NO: 11 - protein, 6 aa, synthetic construct
  REGION 1..6: note = Description of Artificial Sequence: Linker peptide
RVRRSV

SEQ ID NO: 12 - protein, 107 aa, synthetic construct
  REGION 1..107: note = Description of Artificial Sequence: Light chain mammalian humanized antibody
DIQMTQSPSSLSASVGDRVTITCRASQDVNTAVAWYQQKPGKAPKLLIYSASFLESGVPSRFSGSGSGTDFTLTISSLQPEDFATYYCQQHYTTPPTFGQGTKVEIK

SEQ ID NO: 13 - protein, 120 aa, synthetic construct
  REGION 1..120: note = Description of Artificial Sequence: Heavy chain mammalian humanized antibody
EVQLVESGGGLVQPGGSLRLSCAASGFNIKDTYIHWVRQAPGKGLEWVARIYPTNGYTRYADSVKGRFTISRDDSKNTLYLQMNSLRAEDTAVYYCARWGGDGFYAMDVWGQGTLVTVSS

SEQ ID NO: 14 - protein, 107 aa, synthetic construct; identical to SEQ ID NO: 12
  REGION 1..107: note = Description of Artificial Sequence: Light chain mammalian humanized antibody

SEQ ID NO: 15 - protein, 120 aa, synthetic construct
  REGION 1..120: note = Description of Artificial Sequence: Heavy chain mammalian humanized antibody
EVQLVESGGGLVQPGGSLRLSCAASGFNIKDTYIHWVRQAPGKGLEWVARIYPTNGYTRYADSVKGRFTISADDSKNTLYLQMNSLRAEDTAVYYCARWGGDGFYAMDVWGQGTLVTVSS

SEQ ID NO: 16 - protein, 107 aa, synthetic construct; identical to SEQ ID NO: 12
  REGION 1..107: note = Description of Artificial Sequence: Light chain mammalian humanized antibody

SEQ ID NO: 17 - protein, 120 aa, synthetic construct
  REGION 1..120: note = Description of Artificial Sequence: Heavy chain mammalian humanized antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 18 - protein, 107 aa, synthetic construct
  REGION 1..107: note = Description of Artificial Sequence: Light chain mammalian humanized antibody
DIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIK

SEQ ID NO: 19 - protein, 120 aa, synthetic construct
  REGION 1..120: note = Description of Artificial Sequence: Heavy chain mammalian humanized antibody
EVQLVESGGGLVQPGGSLRLSCAASGFNIKDTYIHWVRQAPGKGLEWVARIYPTNGYTRYADSVKGRFTISADTSKNTLYLQMNSLRAEDTAVYYCSRWGGDGFYAMDVWGQGTLVTVSS

SEQ ID NO: 20 - protein, 107 aa, synthetic construct; identical to SEQ ID NO: 18
  REGION 1..107: note = Description of Artificial Sequence: Light chain mammalian humanized antibody

SEQ ID NO: 21 - protein, 120 aa, synthetic construct; identical to SEQ ID NO: 17
  REGION 1..120: note = Description of Artificial Sequence: Heavy chain mammalian humanized antibody

SEQ ID NO: 22 - protein, 107 aa, synthetic construct [sequence omitted]

SEQ ID NO: 23 - protein, 120 aa, synthetic construct; identical to SEQ ID NO: 17
  REGION 1..120: note = Description of Artificial Sequence: Heavy chain mammalian humanized antibody

SEQ ID NO: 24 - protein, 107 aa, synthetic construct; identical to SEQ ID NO: 18
  REGION 1..107: note = Description of Artificial Sequence: Light chain mammalian humanized antibody

SEQ ID NO: 25 - protein, 120 aa, synthetic construct [sequence omitted]

SEQ ID NO: 26 - protein, 107 aa, synthetic construct [sequence omitted]

SEQ ID NO: 27 - protein, 120 aa, synthetic construct [sequence omitted]

SEQ ID NO: 28 - protein, 107 aa, synthetic construct [sequence omitted]

SEQ ID NO: 29 - protein, 120 aa, synthetic construct [sequence omitted]

SEQ ID NO: 30 - protein, 107 aa, synthetic construct [sequence omitted]

SEQ ID NO: 31 - protein, 120 aa, synthetic construct [sequence omitted]

SEQ ID NO: 32 - protein, 108 aa, synthetic construct
  REGION 1..108: note = Description of Artificial Sequence: Light chain mammalian humanized antibody
DIQMTQSPSSLSASVGDRVTITCRASQDVSSAVAWYQQKPGKAPKLLIDWSASFLYSGVPSRFSGSRSGTDFTLTISSLQPEDFATYYCQQHYTTPPTFGQGTKVEIK

SEQ ID NO: 33 - protein, 121 aa, synthetic construct
  REGION 1..121: note = Description of Artificial Sequence: Heavy chain mammalian humanized antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGWGPKLAMDYWGQGTLVTVSS

SEQ ID NO: 34 - protein, 216 aa, synthetic construct
  REGION 1..216: note = Description of Artificial Sequence: Light chain mammalian humanized antibody
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNNFYPREAKVQWKVDNALQSGENSQESVTEQDSKDSTYSLSSTLTLSKADYEKHKVYACEVTHQGLSSPVTKSFNRGEC

SEQ ID NO: 35 - protein, 448 aa, synthetic construct
  REGION 1..448: note = Description of Artificial Sequence: Heavy chain mammalian humanized antibody
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 217 aa, synthetic construct
  REGION 1..217: note = Description of Artificial Sequence: Light chain mammalian humanized antibody
VHSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 448 aa, synthetic construct
  REGION 1..448: note = Description of Artificial Sequence: Heavy chain mammalian humanized antibody
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLVPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALNHYTQKSLSLSPGK

SEQ ID NO: 38 - protein, 216 aa, synthetic construct; identical to SEQ ID NO: 34
  REGION 1..216: note = Description of Artificial Sequence: Light chain mammalian humanized antibody

SEQ ID NO: 39 - protein, 448 aa, synthetic construct; identical to SEQ ID NO: 35
  REGION 1..448: note = Description of Artificial Sequence: Heavy chain mammalian humanized antibody

SEQ ID NO: 40 - protein, 58 aa, synthetic construct [sequence omitted]

SEQ ID NO: 41 - protein, 58 aa, synthetic construct
  REGION 1..58: note = Description of Artificial Sequence: Mammalian humanized antibody
VDNKFNKELRQAYWEIQALPNLNWTQSRAFIRSLYDDPSQSANLLAEAKKLNDAQAPK

SEQ ID NO: 42 - protein, 58 aa, synthetic construct
  REGION 1..58: note = Description of Artificial Sequence: Mammalian humanized antibody
VDNKFNKEPKTAYWEIVKLPNLNPEQRRAFIRSLYDDPSQSANLLAEAKKLNDAQAPK

SEQ ID NO: 43 - protein, 58 aa, synthetic construct
  REGION 1..58: note = Description of Artificial Sequence: Mammalian humanized antibody
VDNKFNKEPREAYWEIQRLPNLNNKQKAAFIRSLYDDPSQSANLLAEAKKLNDAQAPK

SEQ ID NO: 44 - protein, 58 aa, synthetic construct
  REGION 1..58: note = Description of Artificial Sequence: Mammalian humanized antibody
VDNKFNKEWMTAGKEIYRLPNLNGTQVRAFIQSLSDDPSQSANLLAEAKKLNDAQAPK

SEQ ID NO: 45 - protein, 58 aa, synthetic construct
  REGION 1..58: note = Description of Artificial Sequence: Mammalian humanized antibody
VDNKFNKEWVQAGSEIYNLPNLNRAQMRAFIRSLSDDPSQSANLLAEAKKLNDAQAPK

SEQ ID NO: 46 - protein, 58 aa, synthetic construct
  REGION 1..58: note = Description of Artificial Sequence: Mammalian humanized antibody
VDNKFNKEIKQAFHEIVRLPNLNADQVRAFIYSLGDDPSQSANLLAEAKKLNDAQAPK

SEQ ID NO: 47 - protein, 57 aa, synthetic construct
  REGION 1..57: note = Description of Artificial Sequence: Mammalian humanized antibody
VDNKFNKEMVDAGAEIWRLPNLNAKQMAFIDSLGDDPSQSANLLAEAKKLNDAQAPK

SEQ ID NO: 48 - protein, 106 aa, synthetic construct
  REGION 1..106: note = Description of Artificial Sequence: Light chain antibody
DIQMTQSPSSLSASVGDRVTITCRASSSVSYIHWYQQKPGKAPKLLIYATSNLASGVPSRFSGSRSGTDFTLTISSLQPEDFATYYCQQWTSNPPTFGQGTKVEIK

SEQ ID NO: 49 - protein, 121 aa, synthetic construct
  REGION 1..121: note = Description of Artificial Sequence: Heavy chain antibody
EVQLVESGGGLVQPGGSLRLSCAASGYTFTSYNMHWVRQAPGKGLEWVAAIYPGNGDTSYNQKFKGRFTISADTSKNTAYLQMNSLRAEDTAVYYCSRSTYYGGDWYFDVWGQGTLVTVSS

SEQ ID NO: 50 - protein, 106 aa, synthetic construct [sequence omitted]

SEQ ID NO: 51 - protein, 120 aa, synthetic construct
  REGION 1..120: note = Description of Artificial Sequence: Heavy chain antibody
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVS

SEQ ID NO: 52 - protein, 106 aa, synthetic construct [sequence omitted]

SEQ ID NO: 53 - protein, 121 aa, synthetic construct
  REGION 1..121: note = Description of Artificial Sequence: Heavy chain antibody
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVS

SEQ ID NO: 54 - protein, 106 aa, synthetic construct [sequence omitted]

SEQ ID NO: 55 - protein, 121 aa, synthetic construct; identical to SEQ ID NO: 53
  REGION 1..121: note = Description of Artificial Sequence: Heavy chain antibody

SEQ ID NO: 56 - protein, 123 aa, synthetic construct
  REGION 1..123: note = Description of Artificial Sequence: Heavy chain antibody
AVQLVESGGGLVQPGRSLRLSCAASGFTFGDYTMHWVRQAPGLGLEWVSGISWNSGSIGYADSVLGRFTISRDNALNSLYLQMNSLRAEDTALYYCTLDNQYGSGSTYGLGVWGQGTLVTVSS

SEQ ID NO: 57 - protein, 107 aa, synthetic construct
  REGION 1..107: note = Description of Artificial Sequence: Light chain antibody
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIL

SEQ ID NO: 58 - protein, 141 aa, synthetic construct
  REGION 1..141: note = Description of Artificial Sequence: Heavy chain antibody
MFLGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 59 - protein, 126 aa, synthetic construct
  REGION 1..126: note = Description of Artificial Sequence: Light chain antibody
MEAPAQLLFLLLLWLPDTTGEIVLTQSPATLSLSPGERATLSCRASQSVSSYLAWYQQKPGQAPRLLIYDASNRATGIPARFSGSGSTDFTLTISSLEPEDFAVYYCQQRSNWPITFGQGTRLEIK

SEQ ID NO: 60 - protein, 144 aa, synthetic construct
  REGION 1..144: note = Description of Artificial Sequence: Heavy chain antibody
MELGLSWVFLVAILKGVQCEVQLVQSGGGLVHPGGSLRLSCTGSGFTFSYHAMHWVRQAPGKGLEWVSIIGTGGVTYYADSVKGRFTISRDNVKNSLYLQMNSLRAEDMAVYYCARDYYGAGSFYDGLYGMDVWGQGTTVTVSS

SEQ ID NO: 61 - protein, 127 aa, synthetic construct
  REGION 1..127: note = Description of Artificial Sequence: Light chain antibody
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSDWPLTFGGGTKVEIK

SEQ ID NO: 62 - protein, 141 aa, synthetic construct
  REGION 1..141: note = Description of Artificial Sequence: Heavy chain antibody
MELGLSWIFLLAILKGVQCEVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 63 - protein, 127 aa, synthetic construct
  REGION 1..127: note = Description of Artificial Sequence: Light chain antibody
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 64 - protein, 443 aa, synthetic construct
  REGION 1..443: note = Description of Artificial Sequence: Heavy chain antibody
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSR

SEQ ID NO: 65 - protein, 209 aa, synthetic construct
  REGION 1..209: note = Description of Artificial Sequence: Light chain antibody
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRADAAPTVFIFPPSDEQLKSGTASVVCLLNNFYPREAKVQWKVDNALQSGNSQESVTEQDSKDSTYSLSSTLTLSKADYEKHKVYACEVTHQGLSSPVTKSFN

SEQ ID NO: 66 - protein, 451 aa, synthetic construct
  REGION 1..451: note = Description of Artificial Sequence: Heavy chain antibody
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 213 aa, synthetic construct
  REGION 1..213: note = Description of Artificial Sequence: Light chain antibody
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 447 aa, synthetic construct
  REGION 1..447: note = Description of Artificial Sequence: Heavy chain antibody
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 210 aa, synthetic construct
  REGION 1..210: note = Description of Artificial Sequence: Light chain antibody
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 70 - DNA, 396 bp, synthetic construct
  misc_feature 1..396: note = Description of Artificial Sequence: Immunoglobulin light chain
  source 1..396: mol_type = other DNA
atggattcacaggcccaggttcttatattgctgctgctatgggtatctggtacctgtggggacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatctttacacgttcggaggggggaccaagctggaaataaaa

SEQ ID NO: 71 - protein, 130 aa, synthetic construct
  REGION 1..130: note = Description of Artificial Sequence: Immunoglobulin light chain
MDSQAQVLLLLLWVSGTCGDIVMSQSPSSLAVSAGEKVTMSCKSSQSLLNSRTRKNYLAWYQQKPGQSPKLLIYWASRESGVPDRFTGSGSGTDFTLTISSVQAEDLAVYYCKQSYNLYTFGGGTKLEIK

SEQ ID NO: 72 - DNA, 354 bp, synthetic construct
  misc_feature 1..354: note = Description of Artificial Sequence: Immunoglobulin heavy chain
  source 1..354: mol_type = other DNA
caggtgcagctgaaggagtcaggacctggcctggtggcaccctcacagagcctgtccatcacatgcactgtctctgggttctcattatccagatatagtgtacactgggttcgccagcctccaggaaagggcctggagtggctgggaatgatatggggtggtggaagcacagactataattcagctctcaaatccagactgagcatcagcaaggacaactccaagagccaagttttcttaaaaatgaacagtctgcaaactgatgacacagccatgtactactgtgccagaggcaatgatggttactactcgtttgcttactggggccaagggactctggtcactgtctcttca

SEQ ID NO: 73 - protein, 118 aa, synthetic construct
  REGION 1..118: note = Description of Artificial Sequence: Immunoglobulin heavy chain
QVQLKESGPGLVAPSQSLSITCTVSGFSLSRYSVHWVRQPPGKGLEWLGMIWGGGSTDYNSALKSRLSISKDNSKSQVFLKMNSLQTDDTAMYYCARGNDGYYSFAYWGQGTLVTVSS

SEQ ID NO: 74 - protein, 12 aa, synthetic construct
  REGION 1..12: note = Description of Artificial Sequence: Lytic peptide
KLAKLAKKKLAK

SEQ ID NO: 75 - protein, 6 aa, synthetic construct
  REGION 1..6: note = Description of Artificial Sequence: Linker
NRVRRS

SEQ ID NO: 76 - DNA, 2217 bp, synthetic construct
  misc_feature 1..2217: note = Description of Artificial Sequence: Her2/neu antibody
  source 1..2217: mol_type = other DNA
catatgcatcaccaccaccaccacgacgacgacgacaaagatattcaaatgacccagtccccgagcagcctgagtgcctccgttggcgaccgcgtgaccattacgtgccgtgcgagccaggatgtcaacaccgcggtggcctggtatcagcaaaaaccgggcaaagcgccgaaactgctgatctattcagcctcgtttctgtacagcggtgttccgtctcgtttcagcggctctcgcagtggtaccgattttaccctgacgattagctctctgcagccggaagactttgcgacgtattactgccagcaacattacaccacgccgccgaccttcggccagggtacgaaagtggaaatcaaaggttccacctcaggcggtggcagtggtggcggttccggcggtggcggtagttccgaagttcagctggtcgaaagtggcggtggcctggttcaaccgggtggctcactgcgtctgtcgtgtgcagcaagcggtttcaacatcaaagatacctacatccactgggttcgtcaggcgccgggcaaaggtctggaatgggtcgcccgcatttacccgaccaatggctatacgcgttacgcagatagcgtgaaaggtcgctttaccatctctgcggacaccagtaaaaacacggcctatctgcagatgaatagcctgcgtgcggaagatacggccgtttattactgctctcgctggggtggcgatggcttctatgctatggactactggggccagggtaccctggtgacggtttcatcgggtcagccgcgtgaaccgcaagtgtataccctgccgccgtcacgcgatgaactgacgaaaaaccaggtgtcgctgacgtgtctggttaaaggcttttacccgagcgacatcgcggttgaatgggaatctaatggtcaaccggaaaacaattataaaaccacgccgccggtcctggatagtgacggctcctttttcctgtacagtaaactgaccgtggataaatcccgttggcagcagggtaacgtcttctcgtgtagcgtgatgcatgaagccctgcataatcactatacccagaaatctctgagtctgtccccgggcaaaggttcaacgtcgggtggcggttccggcggtggctcaggtggcggtggcagctctggccaaccgcgcgaaccgcaggtttacaccctgccgccgagccgtgacgaactgaccaaaaaccaagtcagcctgacgtgcctggtgaaaggcttttacccgagtgacattgcagttgaatgggaatccaatggtcagccggaaaataactacaaaacgacgccgccggttctggattcagacggctcgtttttcctgtactcaaaactgaccgtcgataaatcgcgctggcaacagggtaacgttttcagctgctctgtcatgcacgaagccctgcacaaccattatacccagaaaagtctgtccctgtcaccgggcaaagaagtgcagctggttgaatctggtggcggtctggtgcaaccgggcggttcgctgcgtctgagctgtgcagcttctggctttaatattaaagacacgtacatccactgggtgcgtcaggcaccgggtaaaggcctggaatgggttgctcgtatctatccgacgaacggttatacgcgttacgccgatagcgtcaaaggccgttttaccatcagtgcagacacctccaaaaacacggcttatctgcagatgaatagtctgcgtgcagaagataccgctgtttattactgcagccgctggggcggtgatggcttctatgcaatggattattggggtcaaggtaccctggtcaccgtgagttccggttcgaccagcggcggtggctcaggtggcggttcgggcggtggcggttcatcggacattcagatgacgcaaagcccgagctctctgtctgcgagtgttggcgatcgtgtcaccatcacgtgtcgcgcctctcaggacgtgaataccgcagttgcttggtaccaacaaaaaccgggcaaagcaccgaaactgctgatttactccgcttcattcctgtacagcggtgtgccgtctcgtttttcgggcagccgctctggtaccgatttcaccctgacgattagttccctgcaaccggaagatttcgccacctactactgccagcaacactatacgaccccgccgacgtttggtcagggcacgaaagtggaaattaaataatgaaagctt

SEQ ID NO: 77 - DNA, 2277 bp, synthetic construct
  misc_feature 1..2277: note = Description of Artificial Sequence: Her2/neu antibody
  source 1..2277: mol_type = other DNA
catatgcatcaccaccaccaccacgacgacgacgacaaagatattcaaatgacccagtccccgagcagcctgagtgcctccgttggcgaccgcgtgaccattacgtgccgtgcgagccaggatgtcaacaccgcggtggcctggtatcagcaaaaaccgggcaaagcgccgaaactgctgatctattcagcctcgtttctgtacagcggtgttccgtctcgtttcagcggctctcgcagtggtaccgattttaccctgacgattagctctctgcagccggaagactttgcgacgtattactgccagcaacattacaccacgccgccgaccttcggccagggtacgaaagtggaaatcaaaggttccacctcaggcggtggcagtggtggcggttccggcggtggcggtagttccgaagttcagctggtcgaaagtggcggtggcctggttcaaccgggtggctcactgcgtctgtcgtgtgcagcaagcggtttcaacatcaaagatacctacatccactgggttcgtcaggcgccgggcaaaggtctggaatgggtcgcccgcatttacccgaccaatggctatacgcgttacgcagatagcgtgaaaggtcgctttaccatctctgcggacaccagtaaaaacacggcctatctgcagatgaatagcctgcgtgcggaagatacggccgtttattactgctctcgctggggtggcgatggcttctatgctatggactactggggccagggtaccctggtgacggtttcatcgggtcagccgcgtgaaccgcaagtgtataccctgccgccgtcacgcgatgaactgacgaaaaaccaggtgtcgctgacgtgtctggttaaaggcttttacccgagcgacatcgcggttgaatgggaatctaatggtcaaccggaaaacaattataaaaccacgccgccggtcctggatagtgacggctcctttttcctgtacagtaaactgaccgtggataaatcccgttggcagcagggtaacgtcttctcgtgtagcgtgatgcatgaagccctgcataatcactatacccagaaatctctgagtctgtccccgggcaaaggttcaacgtcgggtggcggttccggcggtggctcaggtggcggtggcagctctggccaaccgcgcgaaccgcaggtttacaccctgccgccgagccgtgacgaactgaccaaaaaccaagtcagcctgacgtgcctggtgaaaggcttttacccgagtgacattgcagttgaatgggaatccaatggtcagccggaaaataactacaaaacgacgccgccggttctggattcagacggctcgtttttcctgtactcaaaactgaccgtcgataaatcgcgctggcaacagggtaacgttttcagctgctctgtcatgcacgaagccctgcacaaccattatacccagaaaagtctgtccctgtcaccgggcaaagaagtgcagctggttgaatctggtggcggtctggtgcaaccgggcggttcgctgcgtctgagctgtgcagcttctggctttaatattaaagacacgtacatccactgggtgcgtcaggcaccgggtaaaggcctggaatgggttgctcgtatctatccgacgaacggttatacgcgttacgccgatagcgtcaaaggccgttttaccatcagtgcagacacctccaaaaacacggcttatctgcagatgaatagtctgcgtgcagaagataccgctgtttattactgcagccgctggggcggtgatggcttctatgcaatggattattggggtcaaggtaccctggtcaccgtgagttccggttcgaccagcggcggtggctcaggtggcggttcgggcggtggcggttcatcggacattcagatgacgcaaagcccgagctctctgtctgcgagtgttggcgatcgtgtcaccatcacgtgtcgcgcctctcaggacgtgaataccgcagttgcttggtaccaacaaaaaccgggcaaagcaccgaaactgctgatttactccgcttcattcctgtacagcggtgtgccgtctcgtttttcgggcagccgctctggtaccgatttcaccctgacgattagttccctgcaaccggaagatttcgccacctactactgccagcaacactatacgaccccgccgacgtttggtcagggcacgaaagtggaaattaaaggcagcaaatttgcgaaattcgccaaaaaattcgcaaaattcgcgaaaaaattcgcgaaataatgaaagctt

SEQ ID NO: 78 - DNA, 2265 bp, synthetic construct
  misc_feature 1..2265: note = Description of Artificial Sequence: Her2/neu antibody
  source 1..2265: mol_type = other DNA
catatggaaaatctgtatttccaaggtgatattcaaatgacccagtccccgagcagcctgagtgcctccgttggcgaccgcgtgaccattacgtgccgtgcgagccaggatgtcaacaccgcggtggcctggtatcagcaaaaaccgggcaaagcgccgaaactgctgatctattcagcctcgtttctgtacagcggtgttccgtctcgtttcagcggctctcgcagtggtaccgattttaccctgacgattagctctctgcagccggaagactttgcgacgtattactgccagcaacattacaccacgccgccgaccttcggccagggtacgaaagtggaaatcaaaggttccacctcaggcggtggcagtggtggcggttccggcggtggcggtagttccgaagttcagctggtcgaaagtggcggtggcctggttcaaccgggtggctcactgcgtctgtcgtgtgcagcaagcggtttcaacatcaaagatacctacatccactgggttcgtcaggcgccgggcaaaggtctggaatgggtcgcccgcatttacccgaccaatggctatacgcgttacgcagatagcgtgaaaggtcgctttaccatctctgcggacaccagtaaaaacacggcctatctgcagatgaatagcctgcgtgcggaagatacggccgtttattactgctctcgctggggtggcgatggcttctatgctatggactactggggccagggtaccctggtgacggtttcatcgggtcagccgcgtgaaccgcaagtgtataccctgccgccgtcacgcgatgaactgacgaaaaaccaggtgtcgctgacgtgtctggttaaaggcttttacccgagcgacatcgcggttgaatgggaatctaatggtcaaccggaaaacaattataaaaccacgccgccggtcctggatagtgacggctcctttttcctgtacagtaaactgaccgtggataaatcccgttggcagcagggtaacgtcttctcgtgtagcgtgatgcatgaagccctgcataatcactatacccagaaatctctgagtctgtccccgggcaaaggttcaacgtcgggtggcggttccggcggtggctcaggtggcggtggcagctctggccaaccgcgcgaaccgcaggtttacaccctgccgccgagccgtgacgaactgaccaaaaaccaagtcagcctgacgtgcctggtgaaaggcttttacccgagtgacattgcagttgaatgggaatccaatggtcagccggaaaataactacaaaacgacgccgccggttctggattcagacggctcgtttttcctgtactcaaaactgaccgtcgataaatcgcgctggcaacagggtaacgttttcagctgctctgtcatgcacgaagccctgcacaaccattatacccagaaaagtctgtccctgtcaccgggcaaagaagtgcagctggttgaatctggtggcggtctggtgcaaccgggcggttcgctgcgtctgagctgtgcagcttctggctttaatattaaagacacgtacatccactgggtgcgtcaggcaccgggtaaaggcctggaatgggttgctcgtatctatccgacgaacggttatacgcgttacgccgatagcgtcaaaggccgttttaccatcagtgcagacacctccaaaaacacggcttatctgcagatgaatagtctgcgtgcagaagataccgctgtttattactgcagccgctggggcggtgatggcttctatgcaatggattattggggtcaaggtaccctggtcaccgtgagttccggttcgaccagcggcggtggctcaggtggcggttcgggcggtggcggttcatcggacattcagatgacgcaaagcccgagctctctgtctgcgagtgttggcgatcgtgtcaccatcacgtgtcgcgcctctcaggacgtgaataccgcagttgcttggtaccaacaaaaaccgggcaaagcaccgaaactgctgatttactccgcttcattcctgtacagcggtgtgccgtctcgtttttcgggcagccgctctggtaccgatttcaccctgacgattagttccctgcaaccggaagatttcgccacctactactgccagcaacactatacgaccccgccgacgtttggtcagggcacgaaagtggaaattaaaggcagcaaactggcgaaactggccaaaaaactggcaaaactggcgaaaaaactggcgaaataatgaaagctt

SEQ ID NO: 79 - DNA, 2277 bp, synthetic construct
  misc_feature 1..2277: note = Description of Artificial Sequence: Her2/neu antibody
  source 1..2277: mol_type = other DNA
catatggaaaatctgtatttccaaggtgatattcaaatgacccagtccccgagcagcctgagtgcctccgttggcgaccgcgtgaccattacgtgccgtgcgagccaggatgtcaacaccgcggtggcctggtatcagcaaaaaccgggcaaagcgccgaaactgctgatctattcagcctcgtttctgtacagcggtgttccgtctcgtttcagcggctctcgcagtggtaccgattttaccctgacgattagctctctgcagccggaagactttgcgacgtattactgccagcaacattacaccacgccgccgaccttcggccagggtacgaaagtggaaatcaaaggttccacctcaggcggtggcagtggtggcggttccggcggtggcggtagttccgaagttcagctggtcgaaagtggcggtggcctggttcaaccgggtggctcactgcgtctgtcgtgtgcagcaagcggtttcaacatcaaagatacctacatccactgggttcgtcaggcgccgggcaaaggtctggaatgggtcgcccgcatttacccgaccaatggctatacgcgttacgcagatagcgtgaaaggtcgctttaccatctctgcggacaccagtaaaaacacggcctatctgcagatgaatagcctgcgtgcggaagatacggccgtttattactgctctcgctggggtggcgatggcttctatgctatggactactggggccagggtaccctggtgacggtttcatcgggtcagccgcgtgaaccgcaagtgtataccctgccgccgtcacgcgatgaactgacgaaaaaccaggtgtcgctgacgtgtctggttaaaggcttttacccgagcgacatcgcggttgaatgggaatctaatggtcaaccggaaaacaattataaaaccacgccgccggtcctggatagtgacggctcctttttcctgtacagtaaactgaccgtggataaatcccgttggcagcagggtaacgtcttctcgtgtagcgtgatgcatgaagccctgcataatcactatacccagaaatctctgagtctgtccccgggcaaaggttcaacgtcgggtggcggttccggcggtggctcaggtggcggtggcagctctggccaaccgcgcgaaccgcaggtttacaccctgccgccgagccgtgacgaactgaccaaaaaccaagtcagcctgacgtgcctggtgaaaggcttttacccgagtgacattgcagttgaatgggaatccaatggtcagccggaaaataactacaaaacgacgccgccggttctggattcagacggctcgtttttcctgtactcaaaactgaccgtcgataaatcgcgctggcaacagggtaacgttttcagctgctctgtcatgcacgaagccctgcacaaccattatacccagaaaagtctgtccctgtcaccgggcaaagaagtgcagctggttgaatctggtggcggtctggtgcaaccgggcggttcgctgcgtctgagctgtgcagcttctggctttaatattaaagacacgtacatccactgggtgcgtcaggcaccgggtaaaggcctggaatgggttgctcgtatctatccgacgaacggttatacgcgttacgccgatagcgtcaaaggccgttttaccatcagtgcagacacctccaaaaacacggcttatctgcagatgaatagtctgcgtgcagaagataccgctgtttattactgcagccgctggggcggtgatggcttctatgcaatggattattggggtcaaggtaccctggtcaccgtgagttccggttcgaccagcggcggtggctcaggtggcggttcgggcggtggcggttcatcggacattcagatgacgcaaagcccgagctctctgtctgcgagtgttggcgatcgtgtcaccatcacgtgtcgcgcctctcaggacgtgaataccgcagttgcttggtaccaacaaaaaccgggcaaagcaccgaaactgctgatttactccgcttcattcctgtacagcggtgtgccgtctcgtttttcgggcagccgctctggtaccgatttcaccctgacgattagttccctgcaaccggaagatttcgccacctactactgccagcaacactatacgaccccgccgacgtttggtcagggcacgaaagtggaaattaaaaaccgtgtgcgtcgcagcaaatttgcgaaattcgccaaaaaatttgcaaaattcgctaaaaaatttgcgaaataatgaaagctt

SEQ ID NO: 80 - DNA, 2289 bp, synthetic construct
  misc_feature 1..2289: note = Description of Artificial Sequence: Her2/neu antibody
  source 1..2289: mol_type = other DNA
catatgcatcaccaccaccaccacgacgacgacgacaaagatattcaaatgacccagtccccgagcagcctgagtgcctccgttggcgaccgcgtgaccattacgtgccgtgcgagccaggatgtcaacaccgcggtggcctggtatcagcaaaaaccgggcaaagcgccgaaactgctgatctattcagcctcgtttctgtacagcggtgttccgtctcgtttcagcggctctcgcagtggtaccgattttaccctgacgattagctctctgcagccggaagactttgcgacgtattactgccagcaacattacaccacgccgccgaccttcggccagggtacgaaagtggaaatcaaaggttccacctcaggcggtggcagtggtggcggttccggcggtggcggtagttccgaagttcagctggtcgaaagtggcggtggcctggttcaaccgggtggctcactgcgtctgtcgtgtgcagcaagcggtttcaacatcaaagatacctacatccactgggttcgtcaggcgccgggcaaaggtctggaatgggtcgcccgcatttacccgaccaatggctatacgcgttacgcagatagcgtgaaaggtcgctttaccatctctgcggacaccagtaaaaacacggcctatctgcagatgaatagcctgcgtgcggaagatacggccgtttattactgctctcgctggggtggcgatggcttctatgctatggactactggggccagggtaccctggtgacggtttcatcgggtcagccgcgtgaaccgcaagtgtataccctgccgccgtcacgcgatgaactgacgaaaaaccaggtgtcgctgacgtgtctggttaaaggcttttacccgagcgacatcgcggttgaatgggaatctaatggtcaaccggaaaacaattataaaaccacgccgccggtcctggatagtgacggctcctttttcctgtacagtaaactgaccgtggataaatcccgttggcagcagggtaacgtcttctcgtgtagcgtgatgcatgaagccctgcataatcactatacccagaaatctctgagtctgtccccgggcaaaggttcaacgtcgggtggcggttccggcggtggctcaggtggcggtggcagctctggccaaccgcgcgaaccgcaggtttacaccctgccgccgagccgtgacgaactgaccaaaaaccaagtcagcctgacgtgcctggtgaaaggcttttacccgagtgacattgcagttgaatgggaatccaatggtcagccggaaaataactacaaaacgacgccgccggttctggattcagacggctcgtttttcctgtactcaaaactgaccgtcgataaatcgcgctggcaacagggtaacgttttcagctgctctgtcatgcacgaagccctgcacaaccattatacccagaaaagtctgtccctgtcaccgggcaaagaagtgcagctggttgaatctggtggcggtctggtgcaaccgggcggttcgctgcgtctgagctgtgcagcttctggctttaatattaaagacacgtacatccactgggtgcgtcaggcaccgggtaaaggcctggaatgggttgctcgtatctatccgacgaacggttatacgcgttacgccgatagcgtcaaaggccgttttaccatcagtgcagacacctccaaaaacacggcttatctgcagatgaatagtctgcgtgcagaagataccgctgtttattactgcagccgctggggcggtgatggcttctatgcaatggattattggggtcaaggtaccctggtcaccgtgagttccggttcgaccagcggcggtggctcaggtggcggttcgggcggtggcggttcatcggacattcagatgacgcaaagcccgagctctctgtctgcgagtgttggcgatcgtgtcaccatcacgtgtcgcgcctctcaggacgtgaataccgcagttgcttggtaccaacaaaaaccgggcaaagcaccgaaactgctgatttactccgcttcattcctgtacagcggtgtgccgtctcgtttttcgggcagccgctctggtaccgatttcaccctgacgattagttccctgcaaccggaagatttcgccacctactactgccagcaacactatacgaccccgccgacgtttggtcagggcacgaaagtggaaattaaaaaccgtgtgcgtcgcagcaaactggcgaaactggccaaaaaactggcaaaactggctaaaaaactggcgaaataatgaaagctt

SEQ ID NO: 81 - DNA, 2313 bp, synthetic construct
  misc_feature 1..2313: note = Description of Artificial Sequence: Her2/neu antibody
  source 1..2313: mol_type = other DNA
catatggaaaatctgtatttccaaggtaaatttgcgaaattcgccaaaaaattcgcaaaattcgcgaaaaaattcgcgaaagatattcaaatgacccagtccccgagcagcctgagtgcctccgttggcgaccgcgtgaccattacgtgccgtgcgagccaggatgtcaacaccgcggtggcctggtatcagcaaaaaccgggcaaagcgccgaaactgctgatctattcagcctcgtttctgtacagcggtgttccgtctcgtttcagcggctctcgcagtggtaccgattttaccctgacgattagctctctgcagccggaagactttgcgacgtattactgccagcaacattacaccacgccgccgaccttcggccagggtacgaaagtggaaatcaaaggttccacctcaggcggtggcagtggtggcggttccggcggtggcggtagttccgaagttcagctggtcgaaagtggcggtggcctggttcaaccgggtggctcactgcgtctgtcgtgtgcagcaagcggtttcaacatcaaagatacctacatccactgggttcgtcaggcgccgggcaaaggtctggaatgggtcgcccgcatttacccgaccaatggctatacgcgttacgcagatagcgtgaaaggtcgctttaccatctctgcggacaccagtaaaaacacggcctatctgcagatgaatagcctgcgtgcggaagatacggccgtttattactgctctcgctggggtggcgatggcttctatgctatggactactggggccagggtaccctggtgacggtttcatcgggtcagccgcgtgaaccgcaagtgtataccctgccgccgtcacgcgatgaactgacgaaaaaccaggtgtcgctgacgtgtctggttaaaggcttttacccgagcgacatcgcggttgaatgggaatctaatggtcaaccggaaaacaattataaaaccacgccgccggtcctggatagtgacggctcctttttcctgtacagtaaactgaccgtggataaatcccgttggcagcagggtaacgtcttctcgtgtagcgtgatgcatgaagccctgcataatcactatacccagaaatctctgagtctgtccccgggcaaaggttcaacgtcgggtggcggttccggcggtggctcaggtggcggtggcagctctggccaaccgcgcgaaccgcaggtttacaccctgccgccgagccgtgacgaactgaccaaaaaccaagtcagcctgacgtgcctggtgaaaggcttttacccgagtgacattgcagttgaatgggaatccaatggtcagccggaaaataactacaaaacgacgccgccggttctggattcagacggctcgtttttcctgtactcaaaactgaccgtcgataaatcgcgctggcaacagggtaacgttttcagctgctctgtcatgcacgaagccctgcacaaccattatacccagaaaagtctgtccctgtcaccgggcaaagaagtgcagctggttgaatctggtggcggtctggtgcaaccgggcggttcgctgcgtctgagctgtgcagcttctggctttaatattaaagacacgtacatccactgggtgcgtcaggcaccgggtaaaggcctggaatgggttgctcgtatctatccgacgaacggttatacgcgttacgccgatagcgtcaaaggccgttttaccatcagtgcagacacctccaaaaacacggcttatctgcagatgaatagtctgcgtgcagaagataccgctgtttattactgcagccgctggggcggtgatggcttctatgcaatggattattggggtcaaggtaccctggtcaccgtgagttccggttcgaccagcggcggtggctcaggtggcggttcgggcggtggcggttcatcggacattcagatgacgcaaagcccgagctctctgtctgcgagtgttggcgatcgtgtcaccatcacgtgtcgcgcctctcaggacgtgaataccgcagttgcttggtaccaacaaaaaccgggcaaagcaccgaaactgctgatttactccgcttcattcctgtacagcggtgtgccgtctcgtttttcgggcagccgctctggtaccgatttcaccctgacgattagttccctgcaaccggaagatttcgccacctactactgccagcaacactatacgaccccgccgacgtttggtcagggcacgaaagtggaaattaaaaaatttgcgaaattcgccaaaaaattcgcaaaattcgcgaaaaaattcgcgaaataatgaaagctt

SEQ ID NO: 82 - protein, 948 aa, synthetic construct
  REGION 1..948: note = Description of Artificial Sequence: Recombinant anti-Her2 antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSTSGGGSGGGSGGGGSSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGGGSGGGSGGGGSSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 83 - protein, 968 aa, synthetic construct
  REGION 1..968: note = Description of Artificial Sequence: Lytic peptide conjugate
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGGGSGGGSGGGGSSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSTSGGGSGGGSGGGGSSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGGGSGGGSGGGGSSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSKFAKFAKKFAKFAKKFAK

SEQ ID NO: 84 - protein, 214 aa, synthetic construct
  REGION 1..214: note = Description of Artificial Sequence: Anti-Her2/neu light chain antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 451 aa, synthetic construct
  REGION 1..451: note = Description of Artificial Sequence: Her2/neu heavy chain antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 234 aa, synthetic construct
  REGION 1..234: note = Description of Artificial Sequence: Light chain antibody
KFAKFAKKFAKFAKKFAKGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 235 aa, synthetic construct
  REGION 1..235: note = Description of Artificial Sequence: Light chain antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSKFAKFAKKFAKFAKKFAKA

SEQ ID NO: 88 - protein, 255 aa, synthetic construct
  REGION 1..255: note = Description of Artificial Sequence: Light chain antibody
KFAKFAKKFAKFAKKFAKGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSKFAKFAKKFAKFAKKFAKA

SEQ ID NO: 89 - protein, 471 aa, synthetic construct
  REGION 1..471: note = Description of Artificial Sequence: Heavy chain antibody
KFAKFAKKFAKFAKKFAKGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 472 aa, synthetic construct
  REGION 1..472: note = Description of Artificial Sequence: Heavy chain antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSKFAKFAKKFAKFAKKFAKA

SEQ ID NO: 91 - protein, 492 aa, synthetic construct
  REGION 1..492: note = Description of Artificial Sequence: Heavy chain antibody
KFAKFAKKFAKFAKKFAKGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSKFAKFAKKFAKFAKKFAKA

SEQ ID NO: 92 - protein, 249 aa, synthetic construct
  REGION 1..249: note = Description of Artificial Sequence: scFv fragment
HHHHHHDIQLTQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIGSTSGGGSGGGSGGGGSSVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFDVWGQGTTVTVSS

SEQ ID NO: 93 - protein, 269 aa, synthetic construct
  REGION 1..269: note = Description of Artificial Sequence: scFv-Phor18 conjugate
HHHHHHDIQLTQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIGSTSGGGSGGGSGGGGSSVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFDVWGQGTTVTVSSGSKFAKFAKKFAKFAKKFAK

SEQ ID NO: 94 - protein, 717 aa, synthetic construct
  REGION 1..717: note = Description of Artificial Sequence: Anti-CD20 mimibody
DIQLTQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIGSTSGGGSGGGSGGGGSSVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFDVWGQGTTVTVSSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSTSGGGSGGGSGGGGSSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFDVWGQGTTVTVSSGSTSGGGSGGGSGGGGSSDIQLTQSPAILSASPGEKVTMTCRASSSVSYIHWFQQKPGSSPKPWIYATSNLASGVPVFSGSGSGTSYSLTISRVEAEDAATYYCQQWTSNPPTFGGGTKLEI

SEQ ID NO: 95 - protein, 737 aa, synthetic construct
  REGION 1..737: note = Description of Artificial Sequence: Anti-CD20-Phor18 mimibody conjugate
DIQLTQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIGSTSGGGSGGGSGGGGSSVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFDVWGQGTTVTVSSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSTSGGGSGGGSGGGGSSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFDVWGQGTTVTVSSGSTSGGGSGGGSGGGGSSDIQLTQSPAILSASPGEKVTMTCRASSSVSYIHWFQQKPGSSPKPWIYATSNLASGVPVFSGSGSGTSYSLTISRVEAEDAATYYCQQWTSNPPTFGGGTKLEIGSKFAKFAKKFAKFAKKFAK

SEQ ID NO: 96 - protein, 22 aa, synthetic construct
  REGION 1..22: note = Description of Artificial Sequence: Signal sequence
MKYLLPTAAAGLLLLAAQPAMA

SEQ ID NO: 97 - protein, 454 aa, synthetic construct
  REGION 1..454: note = Description of Artificial Sequence: Naked antibody
DIQMTQSPSSLSASVGDRVTITCRASSSVSYIHWYQQKPGKAPKLLIYATSNLASGVPSRFSGSRSGTDFTLTISSLQPEDFATYYCQQWTSNPPTFGQGTKVEIKGGGGSEVQLVESGGGLVQPGGSLRLSCAASGYTFTSYNMHWVRQAPGKGLEWVAAIYPGNGDTSYNQKFKGRFTISADTSKNTAYLQMNSLRAEDTAVYYCSRSTYYGGDWYFDVWGQGTLVTVSSVQPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 494 aa, synthetic construct
  REGION 1..494: note = Description of Artificial Sequence: Lytic peptide
KFAKFAKKFAKFAKKFAKGSDIQMTQSPSSLSASVGDRVTITCRASSSVSYIHWYQQKPGKAPKLLIYATSNLASGVPSRFSGSRSGTDFTLTISSLQPEDFATYYCQQWTSNPPTFGQGTKVEIKGGGGSEVQLVESGGGLVQPGGSLRLSCAASGYTFTSYNMHWVRQAPGKGLEWVAAIYPGNGDTSYNQKFKGRFTISADTSKNTAYLQMNSLRAEDTAVYYCSRSTYYGGDWYFDVWGQGTLVTVSSVQPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSKFAKFAKKFAKFAKKFAK

SEQ ID NO: 99 - protein, 474 aa, synthetic construct
  REGION 1..474: note = Description of Artificial Sequence: Lytic peptide
KFAKFAKKFAKFAKKFAKGSDIQMTQSPSSLSASVGDRVTITCRASSSVSYIHWYQQKPGKAPKLLIYATSNLASGVPSRFSGSRSGTDFTLTISSLQPEDFATYYCQQWTSNPPTFGQGTKVEIKGGGGSEVQLVESGGGLVQPGGSLRLSCAASGYTFTSYNMHWVRQAPGKGLEWVAAIYPGNGDTSYNQKFKGRFTISADTSKNTAYLQMNSLRAEDTAVYYCSRSTYYGGDWYFDVWGQGTLVTVSSVQPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 474 aa, synthetic construct
  REGION 1..474: note = Description of Artificial Sequence: Lytic peptide
DIQMTQSPSSLSASVGDRVTITCRASSSVSYIHWYQQKPGKAPKLLIYATSNLASGVPSRFSGSRSGTDFTLTISSLQPEDFATYYCQQWTSNPPTFGQGTKVEIKGGGGSEVQLVESGGGLVQPGGSLRLSCAASGYTFTSYNMHWVRQAPGKGLEWVAAIYPGNGDTSYNQKFKGRFTISADTSKNTAYLQMNSLRAEDTAVYYCSRSTYYGGDWYFDVWGQGTLVTVSSVQPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSKFAKFAKKFAKFAKKFAK

SEQ ID NO: 101 - protein, 467 aa, synthetic construct
  REGION 1..467: note = Description of Artificial Sequence: Naked antibody
DIQMTQSPSSLSASVGDRVTITCRASSSVSYIHWYQQKPGKAPKLLIYATSNLASGVPSRFSGSRSGTDFTLTISSLQPEDFATYYCQQWTSNPPTFGQGTKVEIKGGGGSGGGGSGGGGSEVQLVESGGGLVQPGGSLRLSCAASGYTFTSYNMHWVRQAPGKGLEWVAAIYPGNGDTSYNQKFKGRFTISADTSKNTAYLQMNSLRAEDTAVYYCSRSTYYGGDWYFDVWGQGTLVTVSSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 507 aa, synthetic construct
  REGION 1..507: note = Description of Artificial Sequence: Conjugated antibody
KFAKFAKKFAKFAKKFAKGSDIQMTQSPSSLSASVGDRVTITCRASSSVSYIHWYQQKPGKAPKLLIYATSNLASGVPSRFSGSRSGTDFTLTISSLQPEDFATYYCQQWTSNPPTFGQGTKVEIKGGGGSGGGGSGGGGSEVQLVESGGGLVQPGGSLRLSCAASGYTFTSYNMHWVRQAPGKGLEWVAAIYPGNGDTSYNQKFKGRFTISADTSKNTAYLQMNSLRAEDTAVYYCSRSTYYGGDWYFDVWGQGTLVTVSSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSKAFKKAFKAFKKAFKAFK

SEQ ID NO: 103 - protein, 487 aa, synthetic construct
  REGION 1..487: note = Description of Artificial Sequence: Conjugated antibody
KFAKFAKKFAKFAKKFAKGSDIQMTQSPSSLSASVGDRVTITCRASSSVSYIHWYQQKPGKAPKLLIYATSNLASGVPSRFSGSRSGTDFTLTISSLQPEDFATYYCQQWTSNPPTFGQGTKVEIKGGGGSGGGGSGGGGSEVQLVESGGGLVQPGGSLRLSCAASGYTFTSYNMHWVRQAPGKGLEWVAAIYPGNGDTSYNQKFKGRFTISADTSKNTAYLQMNSLRAEDTAVYYCSRSTYYGGDWYFDVWGQGTLVTVSSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 487 aa, synthetic construct
  REGION 1..487: note = Description of Artificial Sequence: Conjugated antibody
DIQMTQSPSSLSASVGDRVTITCRASSSVSYIHWYQQKPGKAPKLLIYATSNLASGVPSRFSGSRSGTDFTLTISSLQPEDFATYYCQQWTSNPPTFGQGTKVEIKGGGGSGGGGSGGGGSEVQLVESGGGLVQPGGSLRLSCAASGYTFTSYNMHWVRQAPGKGLEWVAAIYPGNGDTSYNQKFKGRFTISADTSKNTAYLQMNSLRAEDTAVYYCSRSTYYGGDWYFDVWGQGTLVTVSSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSKAFKKAFKAFKKAFKAFK

SEQ ID NO: 105 - protein, 108 aa, synthetic construct
  REGION 1..108: note = Description of Artificial Sequence: Anti-CD20 light chain antibody
MDIQMTQSPSSLSASVGDRVTITCRASSSVSYIHWYQQKPGKAPKLLIYATSNLASGVPSRFSGSRSGTDFTLTISSLQPEDFATYYCQQWTSNPPTFGQGTKVEIKR

SEQ ID NO: 106 - protein, 106 aa, synthetic construct
  REGION 1..106: note = Description of Artificial Sequence: Anti-CD20 light chain antibody
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 107 - protein, 122 aa, synthetic construct
  REGION 1..122: note = Description of Artificial Sequence: Anti-CD20 heavy chain antibody
MEVQLVESGGGLVQPGGSLRLSCAASGYTFTSYNMHWVRQAPGKGLEWVAAIYPGNGDTSYNQKFKGRFTISADTSKNTAYLQMNSLRAEDTAVYYCSRSTYYGGDWYFDVWGQGTLVTVSS

SEQ ID NO: 108 - protein, 110 aa, synthetic construct
  REGION 1..110: note = Description of Artificial Sequence: Anti-CD20 hinge antibody
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCP

SEQ ID NO: 109 - protein, 216 aa, synthetic construct
  REGION 1..216: note = Description of Artificial Sequence: Anti-CD20 heavy chain antibody
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 110 - DNA, 765 bp, synthetic construct
  misc_feature 1..765: note = Description of Artificial Sequence: ADC light chain target
  source 1..765: mol_type = other DNA
atgaagttcgcaaagttcgccaaaaagttcgcaaagttcgcaaaaaagttcgccaaagggtcagatattcagatgactcagagccccagctccctgtccgcatctgtgggcgaccgagtcactatcacctgccgagcctctagttcagtgagctacattcactggtatcagcagaagcctgggaaagccccaaagctgctcatctacgccacaagcaacctggcttccggtgtgccttctaggttcagtgggtcaagaagcggtacagactttacactgactattagctccctccagccagaggatttcgccacttactattgccagcagtggacttccaatccccctacctttggccagggaacaaaagtggaaatcaaggggcagcccaaagctaaccctaccgtcacactgttcccaccctctagtgaggaactccaggcaaataaggccactctggtgtgtctcatttccgacttttaccccggagctgtgaccgtcgcttggaaggcagatggctctccagtgaaagcaggagtcgagaccacaaaacccagtaagcagtcaaacaataagtacgccgcttcaagctatctgagtctcacccctgaacagtggaaaagccataggtcctattcttgccaggtcactcacgaaggtagcactgtggaaaagactgtcgcaccaaccgaatgtagcggctccaaggctttcaagaaggccttcaaggccttcaagaaagcattcaaggcctttaaatgataa

SEQ ID NO: 111 - DNA, 1470 bp, synthetic construct
  misc_feature 1..1470: note = Description of Artificial Sequence: ADC heavy chain target
  source 1..1470: mol_type = other DNA
atgaagttcgccaaatttgctaagaaattcgcaaagtttgccaagaaattcgctaaaggctccgaagtgcagctcgtcgaaagcggggggggactcgtgcagccagggggaagcctcagactctcatgcgccgcctcaggttatactttcacaagctacaacatgcactgggtcagacaggcacctgggaagggtctggagtgggtggccgctatctacccaggcaacggagacacatcttataatcagaagttcaaaggccggtttactattagcgcagatacatccaagaacactgcctacctgcagatgaatagcctccgggctgaagacactgcagtgtactattgcagtcgctcaacctactatggcggagactggtatttcgatgtgtgggggcagggtactctggtcaccgtgagctccgcctctaccaaggggcccagtgtgtttccactggctccctgcagccggtccacctctgagagtacagcagccctgggttgtctcgtgaaagattacttccctgaaccagtcaccgtgtcctggaactctggcgctctgaccagcggagtccacacatttcctgcagtgctccagtctagtgggctgtactccctctcaagcgtggtcacagtcccatcctctaatttcggtactcagacctatacatgcaacgtggaccataagccctccaatactaaggtcgataaaaccgtggagcgcaaatgctgtgtggaatgcccaccttgtccagcaccaccagtcgctgggcctagcgtgttcctgtttcctccaaagccaaaagacactctcatgatctctcgaactcccgaggtcacctgtgtggtcgtggacgtcagtcacgaggatcctgaagtccagtttaactggtacgtggatggagtcgaagtgcataatgcaaagaccaaaccaagggaggaacagttcaactcaacctttagagtcgtgagcgtgctgacagtcgtgcatcaggactggctcaacgggaaggagtataagtgcaaagtgtctaataagggtctgcccgctcctatcgagaaaacaattagcaagactaaaggacagcctcgagaaccacaggtgtacacactgccccctagcagggaggaaatgacaaagaaccaggtctccctgacttgtctcgtgaaaggcttctatcccagtgacattgccgtggagtgggaatcaaatggacagcctgagaacaattacaagaccacaccacccatgctggacagtgatggctcattctttctgtattccaagctcaccgtggataaatctaggtggcagcagggaaatgtcttttcatgtagcgtgatgcacgaggctctccataaccattacacccagaagtccctgtcactctcccccggcaaaggctccaaggctttcaagaaggccttcaaggccttcaagaaagcattcaaggcctttaaatgataa

SEQ ID NO: 112 - DNA, 33 bp, synthetic construct
  misc_feature 1..33: note = Description of Artificial Sequence: ADC light chain primer
  source 1..33: mol_type = other DNA
gggggcgcgccgatattcagatgactcagagcc

SEQ ID NO: 113 - DNA, 31 bp, synthetic construct
  misc_feature 1..31: note = Description of Artificial Sequence: ADC light chain primer
  source 1..31: mol_type = other DNA
gggggcgcgccaagttcgcaaagttcgccaa

SEQ ID NO: 114 - DNA, 31 bp, synthetic construct
  misc_feature 1..31: note = Description of Artificial Sequence: ADC light chain primer
  source 1..31: mol_type = other DNA
ggggaattcttatcagctacattcggttggt

SEQ ID NO: 115 - DNA, 34 bp, synthetic construct
  misc_feature 1..34: note = Description of Artificial Sequence: ADC light chain primer
  source 1..34: mol_type = other DNA
ggggaattcttatcatttaaaggccttgaatgct

SEQ ID NO: 116 - DNA, 31 bp, synthetic construct
  misc_feature 1..31: note = Description of Artificial Sequence: ADC heavy chain primer
  source 1..31: mol_type = other DNA
gggggcgcgccgaagtgcagctcgtcgaaag

SEQ ID NO: 117 - DNA, 33 bp, synthetic construct
  misc_feature 1..33: note = Description of Artificial Sequence: ADC heavy chain primer
  source 1..33: mol_type = other DNA
gggggcgcgccaagttcgccaaatttgctaaga

SEQ ID NO: 118 - DNA, 27 bp, synthetic construct
  misc_feature 1..27: note = Description of Artificial Sequence: ADC heavy chain primer
  source 1..27: mol_type = other DNA
ggggaattcttatcatttgccggggga

SEQ ID NO: 119 - protein, 212 aa, synthetic construct
  REGION 1..212: note = Description of Artificial Sequence: Light chain naked antibody
DIQMTQSPSSLSASVGDRVTITCRASSSVSYIHWYQQKPGKAPKLLIYATSNLASGVPSRFSGSRSGTDFTLTISSLQPEDFATYYCQQWTSNPPTFGQGTKVEIKGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 120 - protein, 447 aa, synthetic construct
  REGION 1..447: note = Description of Artificial Sequence: Heavy chain naked antibody
EVQLVESGGGLVQPGGSLRLSCAASGYTFTSYNMHWVRQAPGKGLEWVAAIYPGNGDTSYNQKFKGRFTISADTSKNTAYLQMNSLRAEDTAVYYCSRSTYYGGDWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 121 - protein, 233 aa, synthetic construct
  REGION 1..233: note = Description of Artificial Sequence: Light chain naked antibody
MKFAKFAKKFAKFAKKFAKGSDIQMTQSPSSLSASVGDRVTITCRASSSVSYIHWYQQKPGKAPKLLIYATSNLASGVPSRFSGSRSGTDFTLTISSLQPEDFATYYCQQWTSNPPTFGQGTKVEIKGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 122 - protein, 447 aa, synthetic construct; identical to SEQ ID NO: 120
  REGION 1..447: note = Description of Artificial Sequence: Heavy chain naked antibody

SEQ ID NO: 123 - protein, 212 aa, synthetic construct; identical to SEQ ID NO: 119
  REGION 1..212: note = Description of Artificial Sequence: Light chain naked antibody

SEQ ID NO: 124 - protein, 468 aa, synthetic construct
  REGION 1..468: note = Description of Artificial Sequence: Heavy chain naked antibody
MKFAKFAKKFAKFAKKFAKGSEVQLVESGGGLVQPGGSLRLSCAASGYTFTSYNMHWVRQAPGKGLEWVAAIYPGNGDTSYNQKFKGRFTISADTSKNTAYLQMNSLRAEDTAVYYCSRSTYYGGDWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 125 - protein, 233 aa, synthetic construct; identical to SEQ ID NO: 121
  REGION 1..233: note = Description of Artificial Sequence: Light chain naked antibody

SEQ ID NO: 126 - protein, 468 aa, synthetic construct; identical to SEQ ID NO: 124
  REGION 1..468: note = Description of Artificial Sequence: Heavy chain naked antibody

SEQ ID NO: 127 - protein, 253 aa, synthetic construct
  REGION 1..253: note = Description of Artificial Sequence: Light chain naked antibody
MKFAKFAKKFAKFAKKFAKGSDIQMTQSPSSLSASVGDRVTITCRASSSVSYIHWYQQKPGKAPKLLIYATSNLASGVPSRFSGSRSGTDFTLTISSLQPEDFATYYCQQWTSNPPTFGQGTKVEIKGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSGSKAFKKAFKAFKKAFKAFK

SEQ ID NO: 128 - protein, 468 aa, synthetic construct; identical to SEQ ID NO: 124
  REGION 1..468: note = Description of Artificial Sequence: Heavy chain naked antibody

SEQ ID NO: 129 - protein, 253 aa, synthetic construct; identical to SEQ ID NO: 127
  REGION 1..253: note = Description of Artificial Sequence: Light chain naked antibody

SEQ ID NO: 130 - protein, 488 aa, synthetic construct
  REGION 1..488: note = Description of Artificial Sequence: Heavy chain naked antibody
MKFAKFAKKFAKFAKKFAKGSEVQLVESGGGLVQPGGSLRLSCAASGYTFTSYNMHWVRQAPGKGLEWVAAIYPGNGDTSYNQKFKGRFTISADTSKNTAYLQMNSLRAEDTAVYYCSRSTYYGGDWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGSKAFKKAFKAFKKAFKAFK

SEQ ID NO: 131 - protein, 1255 aa, synthetic construct [sequence omitted]

SEQ ID NO: 132 - protein, 557 aa, synthetic construct
  REGION 1..557: note = Description of Artificial Sequence: Target sequence
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLAGSQSYEDMRGILYAAPQLRSIRGQPGPNHEEDADSYENMDNPDGPDPAWGGGGRMGTWSTR

SEQ ID NO: 133 - protein, 297 aa, synthetic construct
  REGION 1..297: note = Description of Artificial Sequence: Target sequence
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 134 - protein, 759 aa, synthetic construct
  REGION 1..759: note = Description of Artificial Sequence: Target sequence
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 135 - protein, 320 aa, synthetic construct
  REGION 1..320: note = Description of Artificial Sequence: Target sequence
MNPPSQEIEELPRRRCCRRGTQIVLLGLVTAALWAGLLTLLLLWHWDTTQSLKQLEERAARNVSQVSKNLESHHGDQMAQKSQSTQISQELEELRAEQQRLKSQDLELSWNLNGLQADLSSFKSQELNERNEASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHS

SEQ ID NO: 136 - protein, 260 aa, synthetic construct
  REGION 1..260: note = Description of Artificial Sequence: Target sequence
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP

SEQ ID NO: 137 - protein, 220 aa, synthetic construct
  REGION 1..220: note = Description of Artificial Sequence: Target sequence
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 138 - protein, 595 aa, synthetic construct
  REGION 1..595: note = Description of Artificial Sequence: Target sequence
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 139 - protein, 132 aa, synthetic construct
  REGION 1..132: note = Description of Artificial Sequence: Target sequence
MSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 140 - protein, 738 aa, synthetic construct
  REGION 1..738: note = Description of Artificial Sequence: Target sequence
MQPRWAQGATMWLGVLLTLLLCSSLEGQENSFTINSVDMKSLPDWTVQNGKNLTLQCFADVSTTSHVKPQHQMLFYKDDVLFYNISSMKSTESYFIPEVRIYDSGTYKCTVIVNNKEKTTAEYQLLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLRVKVIAPVDEVQISILSSKVVESGEDIVLQCAVNEGSGPITYKFYREKEGKPFYQMTSNATQAFWTKQKASKEQEGEYYCTAFNRANHASSVPRSKILTVRVILAPWKKGLIAVVIIGVIIALLIIAAKCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 141 - protein, 364 aa, synthetic construct
  REGION 1..364: note = Description of Artificial Sequence: Target sequence
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 142 - protein, 385 aa, synthetic construct
  REGION 1..385: note = Description of Artificial Sequence: Target sequence
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLGEDPYYTENGGGQGYSSGPGTSPEAQGKASVNRGAQENGTGQATSRNGHSARQHVVADTEL

SEQ ID NO: 143 - protein, 277 aa, synthetic construct
  REGION 1..277: note = Description of Artificial Sequence: Target sequence
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 144 - protein, 61 aa, synthetic construct
  REGION 1..61: note = Description of Artificial Sequence: Target sequence
MKRFLFLLLTISLLVMVQIQTGLSGQNDTSQTSSPSASSNISGGIFLFFVANAIIHLFCFS

SEQ ID NO: 145 - protein, 18 aa, synthetic construct
  REGION 1..18: note = Description of Artificial Sequence: Target sequence
MKRFLFLLLTISLLVMVQ

SEQ ID NO: 146 - protein, 858 aa, synthetic construct
  REGION 1..858: note = Description of Artificial Sequence: Target sequence
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIVLSNNYLQIRGIKKTDEGTYRCEGRILARGEINFKDIQVIVNVPPTIQARQNIVNATANLGQSVTLVCDAEGFPEPTMSWTKDGEQIEQEEDDEKYIFSDDSSQLTIKKVDKNDEAEYICIAENKAGEQDATIHLKVFAKPKITYVENQTAMELEEQVTLTCEASGDPIPSITWRTSTRNISSEEKASWTRPEKQETLDGHMVVRSHARVSSLTLKSIQYTDAGEYICTASNTIGQDSQSMYLEVQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVQGEPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPANGSPTSGLSTGAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKA

SEQ ID NO: 147 - protein, 193 aa, synthetic construct
  REGION 1..193: note = Description of Artificial Sequence: Target sequence
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 148 - protein, 378 aa, synthetic construct
  REGION 1..378: note = Description of Artificial Sequence: Target sequence
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 149 - protein, 240 aa, synthetic construct
  REGION 1..240: note = Description of Artificial Sequence: Target sequence
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 150 - protein, 320 aa, synthetic construct
  REGION 1..320: note = Description of Artificial Sequence: Target sequence
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYAKPTKQEEFYA

SEQ ID NO: 151 - protein, 420 aa, synthetic construct
  REGION 1..420: note = Description of Artificial Sequence: Target sequence
MPGGCSRGPAAGDGRLRLARLALVLLGWVSSSSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFAFSGSNASVSAPSPLVELILNHIVPPEDERQNRSFEGMVVAALLAGRALQGLRRLELASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 152 - protein, 6 aa, synthetic construct
  REGION 1..6: note = Description of Artificial Sequence: Signal peptide
DAAQPA

SEQ ID NO: 153 - protein, 7 aa, synthetic construct
  REGION 1..7: note = Description of Artificial Sequence: Motif
PNNPNNP

SEQ ID NO: 154 - protein, 21 aa, Homo sapiens
MQTDTLLLWVLLLWVPGSTGA